(19) United States
(12) Patent Application Publication
(10) Pub. No.: US 2012/0157401 A1
(43) Pub. Date: Jun. 21, 2012

(54) METHODS FOR TREATING NEUROFIBROMATOSIS

(75) Inventors: Liangxian Cao, Parlin, NJ (US); Thomas W. Davis, South Orange, NJ (US); Harry H. Miao, Wellsley, MA (US); Langdon Miller, Seattle, WA (US); Marla Weetall, Morristown, NJ (US)

(73) Assignee: PTC Therapeutics, Inc.

(21) Appl. No.: 13/321,242

(22) PCT Filed: May 27, 2010
(86) PCT No.: PCT/US10/36364

§ 371 Date: Mar. 9, 2012

Related U.S. Application Data
(60) Provisional application No. 61/181,650, filed on May 27, 2009.

(51) Int. Cl. A61K 31/437; A61K 31/496; A61K 31/541; A61P 25/00; A61K 31/506; A61K 31/695; A61K 31/706; A61K 31/501; A61K 31/5377; A61K 31/53

(52) U.S. Cl. 514/43; 514/292; 514/232.8; 514/253.03; 514/228.2; 514/245; 514/274; 514/63; 514/252.04

(57) ABSTRACT

Methods for treating neurofibromatosis involving the administration of a compound that selectively inhibits pathological production of human VEGF are described. The compound can be administered as a single-agent therapy or in combination with one or more additional therapies to a human in need of such treatment.

### CROSS REFERENCE TO RELATED APPLICATIONS

This application claims the benefit of priority to U.S. Provisional Patent Application 61/181,650, filed May 27, 2009, incorporated herein by reference in its entirety and for all purposes.

### 1. INTRODUCTION

Methods for treating neurofibromatosis involving the administration of a compound that selectively inhibits pathological production of human vascular endothelial growth factor (VEGF) are described. The compound can be administered as a single-agent therapy or in combination with one or more additional therapies to a human in need of such treatment.

### 2. BACKGROUND

Neurofibromatosis (NF) is a genetic disorder of the nervous system that primarily affects the development and growth of neural (nerve) tissues, and that causes tumors called neurofibromas to grow along nerves in the body. These tumors, as they grow, can press on vital areas of the body, causing problems in the way the body functions. Although many affected persons inherit the disorder, between 30 to 50 percent of new cases arise spontaneously through gene mutation.

NF is often diagnosed in childhood, occasionally in infancy (in children with severe cases), but usually around 3-16 years of age. Effects of the disease vary widely—some children live almost unaffected by the condition; rarely, others might be severely disabled. Neurofibromas often first appear in childhood, especially during puberty. Many neurofibromas can be removed. Although usually benign (noncancerous), an estimated 3%-5% become cancerous.

Types of NF include NF type 1 (NF1), NF type 2 (NF2), and Schwannomatosis. NF1 is more common, occurring in 1 of every 4,000 births and affecting an estimated 100,000 Americans (see, “Neurofibromatosis Fact Sheet,” National Institute of Neurological Disorders and Stroke (NINDS), NIH Publication No. 06-2126). It is also known as von Recklinghausen disease. Mutation of a gene on chromosome 17q11.2 called neurofibromin is the underlying genetic defect associated with NF1. Neurofibromin encodes a large protein called Neurofibromin, which can suppress the action of Ras, an oncoprotein promoting tumor growth. Phenotypic manifestations of NF1 include presence of light brown skin spots at birth or during childhood, neurofibromas (tumors that grow along nerves under the skin), plexiform neurofibromas (tumors involving multiple nerves), spinal cord and optic nerve tumors, and learning disabilities. Current treatment and/or management of NF1 include surgery, and in some cases radiation or chemotherapy when tumors become malignant (3 to 5 percent of all cases). Treatments for other conditions associated with NF1 are aimed at controlling or relieving symptoms, e.g., headache and epileptic seizures are treated with medications.

NF2 is an autosomal dominant genetic disorder associated with neurologic, ophthalmologic, and cutaneous abnormalities. NF2 is caused by inactivation of the tumor suppressor gene, NF2, which encodes the protein Merlin (a.k.a. schwannomin). Typically, patients present with symptoms such as hearing loss, tinnitus, visual impairment, imbalance, or painful skin lesions. A formal diagnosis is established by the presence of bilateral vestibular schwannoma (VS) or unilateral VS in the patient in optional conjunction with the presence of NF2-associated tumors (e.g., meningiomas, schwannomas, ependymomas, glioma, or neurofibroma), posterior cataracts, or a family history of other NF2-related tumors. In addition to the morbidity associated with auditory and vestibular deficits, patients may experience other neurologic dysfunction related to VS growth (e.g., due to compression of other cranial nerves). Skull-base tumors (including VS and meningiomas) are of particular concern in NF2 because they can lead to lower cranial nerve dysfunction and death.

Surgery is the primary treatment option, yet surgical removal of all tumors is not possible or advisable in certain cases; furthermore, surgery often introduces significant post operative risks, including hearing loss, facial weakness, and dysphagia. Patients who have had prior surgeries may be extremely reticent to undergo further operative intervention. Irradiation of tumors has been advocated by some groups but can be associated with chronic neurologic dysfunction or malignant transformation. Currently, no effective medical therapy is available.

Schwannomatosis has been linked to mutations in the SMARCB1 (hSnf5/INI1) tumor suppressor gene (see, e.g., Boyd et al., Clin. Genet., October 2008, 74(4):358-66). Schwannomatosis shares many features with the better-known forms of NF, however, current evidence suggests that it is a distinct genetic disease, separate from NF1 and NF2. Multiple schwannomas, or tumors of nerve sheaths, are seen in schwannomatosis, but not the characteristic vestibular (ear nerve) tumors seen in NF2. Patients with schwannomatosis develop tumors on the sheaths, or coverings, of their nerves (see, e.g., MacCollin et al., Neurology, 2005, 64:1838-1845).

### 3. SUMMARY

Methods for treating neurofibromatosis (hereinafter “NF”), e.g., NF type I (“NF1”), NF type 2 (“NF2”), or schwannomatosis, are described involving the administration of compounds having the formulas set forth herein (“Compound”) to a human subject in need of such treatment. Preferably, the Compound used in the therapeutic method demonstrates one or more of the following activities as determined in cell culture and/or animal model systems, such as those described herein: (a) selective inhibition of the pathological production of human VEGF; (b) inhibition of tumor angiogenesis, tumor-related inflammation, tumor-related edema, and/or tumor growth; and/or (c) prolongation of the G1/S phase of cell cycle.

The Compound can be administered as a single-agent therapy to a human in need of such treatment. Alternatively, the Compound can be administered in combination with one or more additional therapies to a human in need of such treatment. Such therapies may include the use of anti-cancer agents (e.g., cytotoxic agents, anti-angiogenesis agents, tyrosine kinase inhibitors, or other enzyme inhibitors).

Despite differences in the genetic basis for NF1, NF2, and schwannomatosis, the therapies described herein should be effective because they are aimed at interfering with basic mechanisms required for manifestation of each disease (i.e., uncontrolled growth of tumors or inflammation or edema associated with tumors). While not bound by any theory, the therapies described are based, in part, on the pharmacodynamic activities of the Compounds as measured in cell culture and in animal models; in particular, these include: (a) selective inhibition of the pathological production of human VEGF; (b) inhibition of tumor angiogenesis, tumor-related inflammation, tumor-related edema, and/or tumor growth; and/or (c) prolongation of the G1/S phase of the cell cycle of tumor cells.

These pharmacologic activities contribute to limiting solid tumor growth, tumor-related inflammation, and/or tumor-related edema in several ways. For example, inhibition of pathological production of human VEGF by the tumor will inhibit tumor angiogenesis, thereby limiting vascularization and further growth of solid tumors. An additional benefit is achieved for tumors that respond to VEGF as a growth factor—in such cases, the Compound can limit proliferation of such tumor cells independent of their angiogenic status, that is angiogenesis and vascularization need not be present for the Compound to limit proliferation of the tumor cells. Because the process of tumorigenesis can result in inflammation and edema, a Compound may limit such inflammation or edema. Finally, the prolongation of cell cycle may contribute to the induction of apoptotic death of the tumor cells, and/or allow for increased efficacy when the Compound is used in combination with a therapy or therapies (e.g., chemotherapeutic agents or radiation) that interfere with nucleic acid synthesis during the cell cycle (e.g., the G1/S phase).

Thus, in specific embodiments, the methods for treating NF can result in inhibition or reduction of the pathological production of human VEGF (including intratumoral VEGF production), thus reducing human VEGF concentrations in biological specimens of an afflicted subject; inhibition of tumor angiogenesis, tumor-related inflammation, tumor-related edema, and/or tumor growth in the subject; stabilization or reduction of tumor volume or tumor burden in the subject; stabilization or reduction of peritumoral inflammation or edema in the subject; reduction of the concentrations of angiogenic or inflammatory mediators in biological specimens (e.g., plasma, serum, cerebral spinal fluid, urine, or any other biofluids); and/or a delayed or prolonged G1/S phase of the cell cycle (i.e., the period between the late resting or pre-DNA synthesis phase, and the early DNA synthesis phase) in tumor cells of the subject.

Existing antiangiogenic therapies that have been developed for other diseases (e.g., certain cancers, retinopathies including macular degeneration and the like) are directed at neutralizing VEGF activity (e.g., using anti-VEGF antibodies), or inhibiting downstream effects of VEGF signaling (e.g., using tyrosine kinase inhibitors to block the signaling activity of the VEGF receptor). As a result, these existing antiangiogenic therapies neutralize or inhibit physiological or homeostatic VEGF, as well as pathologically produced human VEGF, activity resulting in side effects that, while tolerated for the treatment of life-threatening cancers or to prevent or slow the development of hearing loss or blindness, may not be acceptable for the treatment of NF. Since the Compounds used in the therapeutic methods described herein selectively inhibit pathologic production of human VEGF (e.g., by the tumor), and do not disturb the production of human VEGF under physiological conditions, side effects that are unacceptable for the treatment of NF should be reduced.

The efficacy of the therapeutic intervention is supported by the data presented herein, demonstrating that: the Compounds inhibit the pathological production of human VEGF (see Section 9.1 et. seq., infra); the Compounds inhibit tumor angiogenesis and tumor growth (see Section 9.2 et. seq., infra); the Compounds delay cell cycle by prolonging the G1/S phase (see Section 9.3 et. seq., infra); the Compounds can be administered safely to human subjects (see Section 10.2 et. seq., infra); and the Compounds may inhibit the growth of xenograft human NF1 tumors in animal model systems (see Section 9.2.5 et. seq. and Section 12, infra).

3.1 Definitions

As used herein, the terms “therapies” and “therapy” can refer to any protocol(s), method(s), compositions, formulations, and/or agent(s) that can be used in the prevention, treatment, management, or amelioration of a condition or disorder or symptom thereof (e.g., cancer or a symptom or condition associated therewith, or NF or a symptom or condition associated therewith). In certain embodiments, the terms “therapies” and “therapy” refer to drug therapy, adjuvant therapy, radiation, surgery, biological therapy, supportive therapy, and/or other therapies useful in treatment, management, prevention, or amelioration of a condition or disorder or a symptom thereof (e.g., cancer or a symptom or condition associated therewith, or NF or a symptom or condition associated therewith). In certain embodiments, the term “therapy” refers to a therapy other than a Compound or pharmaceutical composition thereof. In specific embodiments, an “additional therapy” and “additional therapies” refer to a therapy other than a treatment using a Compound or pharmaceutical composition. In a specific embodiment, a therapy includes the use of a Compound as an adjuvant therapy. For example, using a Compound in conjunction with a drug therapy, biological therapy, surgery, and/or supportive therapy.

As used herein, the term “elderly human” refers to a human 65 years or older.

As used herein, the term “human adult” refers to a human that is 18 years or older.

As used herein, the term “human child” refers to a human that is 1 year to 18 years old.

As used herein, the term “human infant” refers to a newborn to 1 year old year human.

As used herein, the term “human toddler” refers to a human that is 1 year to 3 years old.

As used herein, the term “subject” and “patient” are used interchangeably to refer to an individual. In a specific embodiment, the individual is a human. See Section 5.3 infra for more information concerning patients treated for NF in accordance with the methods provided herein.

As used herein, the term “effective amount” in the context of administering a Compound to a subject refers to the amount of a Compound that results in a beneficial or therapeutic effect. In specific embodiments, an “effective amount” of a Compound refers to an amount of a Compound which is sufficient to achieve at least one, two, three, four or more of the following effects: (i) the reduction or amelioration of the severity of one or more NF symptoms; (ii) the reduction in the duration of one or more symptoms associated with NF; (iii) the prevention in the recurrence of a tumor or a symptom associated with NF; (iv) the regression of tumors and/or one or more symptoms associated therewith; (v) the reduction in hospitalization of a subject; (vi) the reduction in hospitalization length; (vii) the increase in the survival of a subject; (viii) the inhibition of the progression of tumors and/or one or more symptoms associated therewith; (ix) the enhancement or improvement of the therapeutic effect of another therapy; (x) a reduction or elimination of hearing loss, tinnitus, visual impairment, imbalance, or painful skin lesions associated with NF; (xi) a reduction in the growth of a tumor or neoplasm associated with NF; (xii) a decrease in tumor size (e.g., volume or diameter) of neurofibromas, plexiform neurofibromas, and/or NF2-associated tumors (e.g., meningiomas, schwannomas, ependymomas, etc.); (xiii) a reduction in the formation of a newly formed tumor, for example an NF-associated tumor such as neurofibromas, plexiform neurofibromas, or NF2-associated tumors (e.g., meningiomas, schwannomas, ependymomas, etc.); (xiv) eradication, removal, or control of primary, regional and/or metastatic tumors associated with NF; (xv) ease in removal of tumors by reducing vascularization prior to surgery; (xvi) a decrease in the number or size of metastases; (xvii) a reduction in mortality; (xviii) an increase in the tumor-free survival rate of patients; (xix) an increase in relapse free survival; (xx) an increase in the number of patients in remission; (xxi) a decrease in hospitalization rate; (xxii) the size of the tumor is maintained and does not increase or increases by less than the increase of a tumor after administration of a standard therapy as measured by conventional methods available to one of skill in the art, such as magnetic resonance imaging (MRI), dynamic contrast-enhanced MRI (DCE-MRI), X-ray, and computed tomography (CT) scan, or a positron emission tomography (PET) scan; (xxiii) the prevention of the development or onset of one or more symptoms associated with NF; (xxiv) an increase in the length of remission in patients; (xxv) the reduction in the number of symptoms associated with NF; (xxvi) an increase in symptom-free survival of NF patients; (xxvii) improvement in neural function, e.g., hearing, balance, tinnitus, or vision; (xxviii) inhibition or reduction in pathological production of VEGF; (xxix) stabilization or reduction of peritumoral inflammation or edema in a subject; (xxx) reduction of the concentration of VEGF or other angiogenic or inflammatory mediators (e.g., cytokines or interleukins) in biological specimens (e.g., plasma, serum, cerebral spinal fluid, urine, or any other biofluids); (xxxi) reduction of the concentration of P1GF, VEGF-C, VEGF-D, VEGFR, IL-6, and/or IL-8 in biological specimens (e.g., plasma, serum, cerebral spinal fluid, urine, or any other biofluids); (xxxii) inhibition or decrease in tumor metabolism or perfusion; (xxxiii) inhibition or decrease in angiogenesis or vascularization; (xxxiv) improvement in quality of life as assessed by methods well known in the art, e.g., tinnitus questionnaires; and/or (xxxv) an improvement in hearing, hearing function, or word recognition. In specific embodiments, an “effective amount” of a Compound refers to an amount of a Compound specified herein, e.g., in section 5.4 below.

Concentrations, amounts, cell counts, percentages and other numerical values may be presented herein in a range format. It is to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the Compounds provided herein include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art, see for example, Remington's Pharmaceutical Sciences, 18^{th} eds., Mack Publishing, Easton Pa. (1990) or Remington: The Science and Practice of Pharmacy, 19^{th} eds., Mack Publishing, Easton Pa. (1995).

As used herein, the term “alkyl” generally refers to saturated hydrocarbyl radicals of straight or branched configuration including, but not limited to methyl, ethyl, n-propyl, isopropyl, n-butyl, isobutyl, sec-butyl, tert-butyl, n-pentyl, n-hexyl, n-heptyl, octyl, n-octyl, and the like. In some embodiments, alkyl substituents can be C₁ to C₈, C₁ to C₆, or C₁ to C₄ alkyl. Alkyl may be optionally substituted where allowed by available valences, for example, with one or more halogen or alkoxy substituents. For instance, halogen substituted alkyl may be selected from haloalkyl, dihaloalkyl, trihaloalkyl and the like.

As used herein, the term “cycloalkyl” generally refers to a saturated or partially unsaturated non-aromatic carbocyclic ring. Representative examples of cycloalkyl include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, cyclooctadienyl, indanyl and the like. Cycloalkyl may be optionally substituted where allowed by available valences. In certain embodiments, cycloalkyl is selected from C₃-C₂₀cycloalkyl, C₃-C₁₄cycloalkyl, C₅-C₈cycloalkyl, C₃-C₈cycloalkyl and the like.

As used herein, the term “alkenyl” generally refers to linear or branched alkyl radicals having one or more carbon-carbon double bonds, such as C₂ to C₈ and C₂ to C₆ alkenyl, including 3-propenyl and the like, and may be optionally substituted where allowed by available valences.

As used herein, the term “alkynyl” generally refers to linear or branched alkyl radicals having one or more carbon-carbon triple bonds, such as C₂ to C₈ and C₂ to C₆ alkynyl, including hex-3-yne and the like and may be optionally substituted where allowed by available valences.

As used herein, the term “aryl” refers to a monocarbocyclic, bicarbocyclic or polycarbocyclic aromatic ring structure. Included in the scope of aryl are aromatic rings having from six to twenty carbon atoms. Aryl ring structures include compounds having one or more ring structures, such as mono-, bi-, or tricyclic compounds. Examples of aryl include phenyl, tolyl, anthracenyl, fluorenyl, indenyl, azulenyl, phenanthrenyl (i.e., phenanthrene), napthyl (i.e., napthalene) and the like. In certain embodiments, aryl may be optionally substituted where allowed by available valences. In one embodiment, aryl is an optionally substituted phenyl or naphthyl.

As used herein, the term “heteroaryl” refers to monocyclic, bicyclic or polycyclic aromatic ring structures in which one or more atoms in the ring, is an element other than carbon (heteroatom). Heteroatoms are typically O, S or N atoms. Included within the scope of heteroaryl, and independently selectable, are O, N, and S heteroaryl ring structures. The ring structure may include compounds having one or more ring structures, such as mono-, bi-, or tricyclic compounds. In some embodiments, heteroaryl may be selected from ring structures that contain one or more heteroatoms, two or more heteroatoms, three or more heteroatoms, or four or more heteroatoms. In one embodiment, the heteroaryl is a 5 to 10 membered or 5 to 12 membered heteroaryl. Heteroaryl ring structures may be selected from those that contain five or more atoms, six or more atoms, or eight or more atoms. Examples of heteroaryl ring structures include, but are not limited to: acridinyl, benzimidazolyl, benzoxazolyl, benzofuranyl, benzothiazolyl, benzothienyl, 1,3-diazinyl, 1,2-diazinyl, 1,2-diazolyl, 1,4-diazanaphthalenyl, furanyl, furazanyl, imidazolyl, indazolyl, indolyl, isoxazolyl, isoquinolinyl, isothiazolyl, isoindolyl, oxadiazolyl, oxazolyl, purinyl, pyridazinyl, pyrazolyl, pyridinyl, pyrazinyl, pyrimidinyl, pyrrolyl, quinazolinyl, quinolinyl, quinoxalinyl, thiazole-2(3H)imine, 1,3,4,-thiadiazole-2(3H)-imine-yl, thiazolyl, thiophenyl, 1,3,5-triazinyl, 1,2,4-triazinyl, 1,2,3-triazinyl, tetrazolyl and the like. In certain embodiments, heteroaryl may be optionally substituted where allowed by available valences.

As used herein, the term “heteroaryl” refers to monocyclic, bicyclic or polycyclic aromatic ring structures in which one or more atoms in the ring, is an element other than carbon (heteroatom). Heteroatoms are typically O, S or N atoms. Included within the scope of heteroaryl, and independently selectable, are O, N, and S heteroaryl ring structures. The ring structure may include compounds having one or more ring structures, such as mono-, bi-, or tricyclic compounds. In some embodiments, heteroaryl may be selected from ring structures that contain one or more heteroatoms, two or more heteroatoms, three or more heteroatoms, or four or more heteroatoms. In one embodiment, the heteroaryl is a 5 to 10 membered or 5 to 12 membered heteroaryl. Heteroaryl ring structures may be selected from those that contain five or more atoms, six or more atoms, or eight or more atoms. Examples of heteroaryl ring structures include, but are not limited to: acridinyl, benzimidazolyl, benzoxazolyl, benzofuranyl, benzothiazolyl, benzothienyl, 1,3-diazinyl, 1,2-diazinyl, 1,2-diazolyl, 1,4-diazanaphthalenyl, furanyl, furazanyl, imidazolyl, indazolyl, indolyl, isoxazolyl, isoquinolinyl, isothiazolyl, isoindolyl, oxadiazolyl, oxazolyl, purinyl, pyridazinyl, pyrazolyl, pyridinyl, pyrazinyl, pyrimidinyl, pyrrolyl, quinazolinyl, quinolinyl, quinoxalinyl, thiazole-2(3H)imine, 1,3,4,-thiadiazole-2(3H)-imine-yl, thiazolyl, thiophenyl, 1,3,5-triazinyl, 1,2,4-triazinyl, 1,2,3-triazinyl, tetrazolyl and the like. In certain embodiments, heteroaryl may be optionally substituted where allowed by available valences.

As used herein, the term “alkoxy” generally refers to a structure of the formula: —O—R. In certain embodiments, R may be an optionally substituted straight or branched alkyl, such as a C₁ to C₅ alkyl.

As used herein, the term “alkylthio” generally refers to a structure of the formula: —S—R. In certain embodiments, R may be an optionally substituted straight or branched alkyl, such as a C₁ to C₅ alkyl.

As used herein, the term “amino” generally refers to a structure of the formula: —NRR′. In certain embodiments, R and R′ independently may be H or an optionally substituted straight or branched alkyl, such as a C₁ to C₅ alkyl. In one embodiment, “thiazoleamino” refers to an amino, wherein at least one of R or R′ is a 2-thiazolyl, 3-thiazolyl or 4-thiazolyl. In one embodiment, “alkylamino” refers to an amino, wherein at least one of R or R′ is an optionally substituted straight or branched C₁ to C₅ alkyl.

As used herein, the term “acetamino” generally refers to a structure of the formula: —NR(C(═O)CH₃), wherein R may be H or an optionally substituted straight or branched alkyl, such as a C₁ to C₅ alkyl.

As used herein, the term “acetamide” generally refers to a structure of the formula: C(═O)NH₂.

As used herein, the term “sulfonyl” generally refers to a structure of the formula: —SO₂R, wherein R can be H or an optional substituent including, but not limited to straight or branched C₁ to C₆ alkyl, aryl, heteroaryl, cycloalkyl, or heterocycle. In one embodiment, “alkylsulfonyl” refers to a structure of the formula: —SO₂R, wherein R is an optionally substituted straight or branched C₁ to C₆ alkyl.

As used herein, the term “oxo” generally refers to a structure of the formula: (═O).

As used herein, the term “phenyloxy” generally refers to a structure of the formula: —O-phenyl, wherein phenyl can be optionally substituted.

For the purposes of this disclosure, the terms “halogen” or “halo” refer to substituents independently selected from fluorine, chlorine, bromine, and iodine.

As used herein, the terms “Compound” or “Compound provided herein” generally refer to a compound described in Section 5.1 or Example 6. In one embodiment, the terms refer to a compound of Formula I, II, III or IV. In another embodiment, the terms refer to a compound of Formula Ia, IIa, IIIa or IVa. In a specific embodiment, the terms refer to a compound depicted in Table 1. In one embodiment, the terms refer to a Compound disclosed in WO2005/089764, e.g., Compounds in the table on pages 26-98; WO2006/113703, e.g., Compounds in the table on pages 29-102; WO2008/127715, e.g., Compounds in the table on pages 52-126; WO2008/127714, e.g., Compounds in the table on pages 48-123; and U.S. Provisional Patent Application 61/181,653, entitled: METHODS FOR TREATING CANCER AND NON-NEOPLASTIC CONDITIONS, filed May 27, 2009, all of which are herewith incorporated by reference in their entirety. In one embodiment, the terms refer to a particular enantiomer, such as an R or S enantiomer of a “Compound” or “Compound provided herein”. In one embodiment, the terms refer to an R or S enantiomer of a compound of Formula I, II, III or IV. In another embodiment, the terms refer to an R or S enantiomer of a compound of Formula Ia, IIa, IIIa or IVa. In a specific embodiment, the terms refer to an R or S enantiomer of a compound depicted in Table 1. The “Compound” or “Compound provided herein” may comprise one or more asymmetric carbon atoms, i.e. n asymmetric carbon atoms, having either R or S configuration as determined by a person skilled in the art. It is understood that the terms “Compound” or “Compound provided herein” encompass all possible stereoisomers that may be generated based on all asymmetric carbon atoms. For example, if a Compound has two (n=2) assymetric carbon atoms, the terms “Compound” or “Compound provided herein” encompass all four, i.e. 2ⁿ=2²=4, stereoisomers (R,S; R,R; S,S; S;R). The “Compound” or “Compound provided herein” may be a substantially pure (e.g., about 90%, about 95%, about 98%, about 99%, or about 99.9% pure) single stereoisomer or a mixture of two or more stereoisomers.

As used herein, the terms “self-microemulsifying drug delivery system” (SMEDDS) or “self-emulsifying drug delivery system” (SEDDS) mean a composition that contains an active agent herein defined in intimate admixture with pharmaceutically acceptable excipients such that the system is capable of dissolving the active agent to the desired concentration and producing colloidal structures by spontaneously forming a microemulsion when diluted with an aqueous medium, for example water, or in gastric juices. The colloidal structures can be solid or liquid particles including droplets and nanoparticles. In a SEDDS or SMEDDS system the type of microemulsion produced will be either clear or turbid depending on drug loading and the type of surfactant used.

As used herein, “microemulsion” means a slightly opaque, opalescent, non-opaque or substantially non-opaque colloidal dispersion (i.e. “clear”) that is formed spontaneously or substantially spontaneously when its components are brought into contact with an aqueous medium. A microemulsion is thermodynamically stable and typically contains dispersed droplets of a mean diameter less than about 200 nm (2000 Å). Generally microemulsions comprise droplets or liquid nanoparticles that have a mean diameter of less than about 150 nm (1500 Å); typically less than 100 nm, generally greater than 10 nm, wherein the dispersion may be thermodynamically stable over a time period of up to about 24 hours.

As used herein, the terms “pathologic,” “pathological” or “pathologically-induced,” in the context of the production of VEGF described herein, refer to the stress-induced expression of VEGF protein. In one embodiment, oncongenic transformation-induced expression of VEGF protein by tumor cells or other cells in the tumor environment is encompassed by the terms. In another embodiment, hypoxia-induced expression of VEGF protein in a chronic or traumatic inflammatory condition is encompassed by the terms. In another embodiment, in response to environmental stimuli, cells that disregulate or overproduce VEGF protein is also encompassed by the terms. As applicable, expression of VEGF protein supports inflammation, angiogenesis and tumor growth. The inhibition or reduction in pathological production of VEGF protein by a Compound can be assessed in cell culture and/or animal models as described herein.

As used herein, the term “about” means a range around a given value wherein the resulting value is substantially the same as the expressly recited value. In one embodiment, “about” means within 25% of a given value or range. For example, the phrase “about 70% by weight” comprises at least all values from 52% to 88% by weight. In another embodiment, the term “about” means within 10% of a given value or range. For example, the phrase “about 70% by weight” comprises at least all values from 63% to 77% by weight. In another embodiment, the term “about” means within 7% of a given value or range. For example, the phrase “about 70% by weight” comprises at least all values from 65% to 75% by weight.

### 4. DESCRIPTION OF FIGURES

FIG. 1. ELISA Evaluation of Inhibition of Soluble VEGF_{121/165} Production by Compound #10 during Hypoxia or Normoxia in HeLa Cells. The results shown are from assays performed in triplicate. The acronyms have the following definitions: ELISA=enzyme-linked immunosorbent assay; SE=standard error; and, VEGF=vascular endothelial growth factor.

FIG. 2. ELISA Evaluation of Inhibition of Soluble VEGF_{121/165} Production by Compound #10 during Hypoxia or Normoxia in Keratinocytes. The results shown are from assays performed in duplicate. The acronyms have the following definitions: ELISA=enzyme-linked immunosorbent assay; SE=standard error; and, VEGF=vascular endothelial growth factor.

FIG. 3. In Cell Western Evaluation of Inhibition of Matrix Associated VEGF_{189/206} Production in HT1080 Cells. The results shown are from assays performed in duplicate. The acronyms have the following definitions: SE=standard error; and, VEGF=vascular endothelial growth factor.

FIG. 4. Western Blot Evaluation of Inhibition of Matrix Associated VEGF_{189/206} Production in HT1080 Cells.

FIG. 5. Reduction of Intratumoral VEGF by Compound #10 in Nude Mice Bearing HT1080 Xenografts. The symbol “*” represents a p value of p<0.05, signifying that the differences in treated mice were significantly different from tumor size in vehicle-treated mice (ANOVA, followed by individual comparisons to vehicle). The acronyms have the following definitions: ANOVA=analysis of variance; BID=2 times per day; QD=1 time per day; SE=standard error; and, VEGF=vascular endothelial growth factor.

FIG. 6. Reduction of Tumor Induced Plasma VEGF by Compound #10 in Nude Mice Bearing HT1080 Xenografts. The symbol “*” represents a p value of p<0.05, signifying that the differences in treated mice were significantly different from tumor size in vehicle-treated mice (ANOVA, followed by individual comparisons to vehicle). The acronyms have the following definitions: ANOVA=analysis of variance; BID=2 times per day; QD=1 time per day; SE=standard error; and, VEGF=vascular endothelial growth factor.

FIG. 7A-B. Inhibition of Tumor Angiogenesis by Compound #10 in Nude Mice Bearing HT1080 Xenografts. FIG. 7A. The effect of vehicle on an immunostain using an anti-murine CD31 antibody specific for endothelial cells. FIG. 7B. The effect of Compound #10 on an immunostain using an anti-murine CD31 antibody specific for endothelial cells.

FIG. 8. Inhibition of Tumor Growth by Compound #10 in Nude Mice Bearing HT1080 Xenografts. The symbol “*” represents a p value of p<0.05, signifying that the differences in treated mice were significantly different from tumor size in vehicle-treated mice (ANOVA, followed by individual comparisons to vehicle). The acronyms have the following definitions: ANOVA=analysis of variance; BID=2 times per day; QD=1 time per day; and, SE=standard error.

FIG. 9. Time Course of Inhibition of Tumor Growth by Compound #10, Bevacizumab, and Doxorubicin in Nude Mice Bearing HT1080 Xenografts. The symbol “*” represents a p value of p<0.05, signifying that the differences in treated mice were significantly different from tumor size in vehicle-treated mice (ANOVA, followed by individual comparisons to vehicle). The acronyms have the following definitions: ANOVA=analysis of variance; IP=intraperitoneal; QD=1 time per day; and, SE=standard error.

FIG. 10A-B. Time Course of Inhibition of Tumor Induced Plasma VEGF Concentrations by Compound #10, Bevacizumab, and Doxorubicin in Nude Mice Bearing HT1080 Xenografts. FIG. 10A. The effect on absolute values of plasma human VEGF concentrations. FIG. 10A. The effect on values of plasma human VEGF concentrations expressed as a ratio relative to tumor volume. The symbol “*” represents a p value of p<0.05, signifying that the differences in treated mice were significantly different from tumor size in vehicle-treated mice (ANOVA, followed by individual comparisons to vehicle). The acronyms have the following definitions: IP=intraperitoneal; QD=once per day; SE=standard error; and, VEGF=vascular endothelial growth factor.

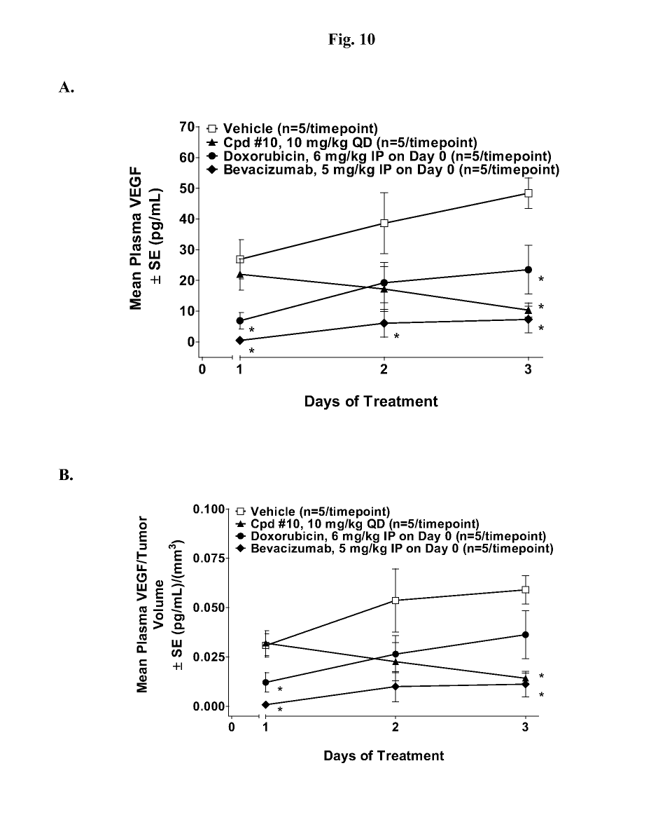

FIG. 11A-B. Inhibition of Tumor Growth by Compound #10 at 5 Weeks in Nude Mice Bearing Orthotopically Implanted SKNEP or SY5Y Xenograft. FIG. 11A. The effect on weight of an SY5Y tumor for mice treated with vehicle and Compound #10. FIG. 11B. The effect on weight of an SKNEP tumor for mice treated with vehicle and Compound #10. The symbol “*” represents a p value of p<0.05, signifying that the differences in treated mice were significantly different from tumor size in vehicle-treated mice (Student's t-test). The acronyms have the following definitions: SE=standard error.

FIG. 12A-G. Cell Cycle Effects in HT1080 Cells by Compound #10 Concentration. Histograms depicting relative DNA content in HT1080 cells under normoxic conditions after treatment with varying concentrations of Compound #10 compared to vehicle. FIG. 12A. Histogram showing the effect of treatment with vehicle. FIGS. 12B-G. Histograms showing the effect of treatment with Compound #10 at 0.3 nm, 1 nm, 3 nm, 10 nm, 30 nm and 100 nm, respectively. The acronyms have the following definitions: G₁=gap 1 phase (resting or pre-DNA synthesis phase—2 chromosomes present); G₂=gap 2 phase (gap between DNA synthesis and mitosis—4 chromosomes present); S=synthesis phase (DNA synthesis ongoing); and, PI=propidium iodide.

FIG. 13A-F. Cell Cycle Effects in HT1080 Cells by Time from Discontinuation of Compound #10. Histograms depicting relative DNA content in HT1080 cells under normoxic conditions after discontinuation of treatment with Compound #10 compared to vehicle. FIG. 13A. Histogram showing the effect of treatment with vehicle. FIGS. 13B-F. Histograms showing the effect of discontinuation of treatment with Compound #10 at 0 hours, 2 hours, 5 hours, 8 hours and 26 hours, respectively. The acronyms have the following definitions: G₁=gap 1 phase (resting or pre-DNA synthesis phase—2 chromosomes present); G₂=gap 2 phase (gap between DNA synthesis and mitosis—4 chromosomes present); S=synthesis phase (DNA synthesis ongoing); and, PI=propidium iodide.

FIG. 14. BrdU Labeling of Cells from HT1080 Xenografts Grown in Nude Mice. The effect of treatment with Compound #10 compared to vehicle and a positive and negative control, doxorubicin and bevacizumab, respectively. The tumors with adequate BrdU staining (>3%) were included in analyses. The symbol “*” represents a p value of p<0.05, signifying that the differences in treated mice were significantly different from tumor size in vehicle-treated mice (ANOVA, followed by Dunnett's test relative to vehicle). The acronyms have the following definitions: ANOVA=analysis of variance; BrdU=bromodeoxyuridine; and, SE=standard error.

FIG. 15. Plasma Concentrations of Compound #10 by Dose Level after Stage 1 of a Study in Healthy Volunteers. The acronyms have the following definitions: BID=2 times per day; and, SD=standard deviation.

FIG. 16. Plasma Concentrations of Compound #10 by Dose Level after Stage 2 of a Study in Healthy Volunteers. The acronyms have the following definitions: TID=3 times per day; and, SD=standard deviation.

FIG. 17A-B. FIG. 17A: Absolute Physiologic VEGF A Plasma and Serum Concentrations: Stage 1 of Multiple dose Study; FIG. 17B: Change from Baseline in Physiologically-Induced VEGF-A Plasma and Serum VEGF Concentrations: Stage 1 of Multiple-dose Study. The acronyms have the following definitions: VEGF=vascular endothelial growth factor; and, SEM=standard error of the mean.

FIG. 18A-B. FIG. 18A: Absolute VEGF-A Plasma and Serum Concentrations: Stage 2 of Multiple-dose Study; FIG. 18B: Change from Baseline in VEGF-A Plasma and Serum VEGF Concentrations: Stage 2 of Multiple-dose Study. The acronyms have the following definitions: VEGF=vascular endothelial growth factor; and, SEM=standard error of the mean.

FIG. 19. Change in Total Tumor Volume Induced by Compound #10 in Nude Mice Bearing MDA-MB-468 Xenografts. The symbol “*” represents a p value of p<0.01, signifying that the differences in treated mice were significantly different from tumor size in vehicle-treated mice (Student's t-test). The acronyms have the following definitions: SE=standard error.

FIG. 20. Change in Necrotic Tumor Volume Induced by Compound #10 in Nude Mice Bearing MDA-MB-468 Xenografts. The symbol “*” represents a p value of p<0.01, signifying that the differences in treated mice were significantly different from tumor size in vehicle-treated mice (Student's t-test). The acronyms have the following definitions: SE=standard error.

FIG. 21. Change in Non-Necrotic Tumor Volume Induced by Compound #10 in Nude Mice Bearing MDA-MB-468 Xenografts. The symbol “*” represents a p value of p<0.01, signifying that the differences in treated mice were significantly different from tumor size in vehicle-treated mice (Student's t-test). The acronyms have the following definitions: SE=standard error.

FIG. 22. Change in fBV Induced by Compound #10 in Non Necrotic Tissue in Nude Mice Bearing MDA-MB-468 Xenografts. The symbol “**” represents a p value of p<0.01, signifying that the differences in treated mice were significantly different from tumor size in vehicle-treated mice (Student's t-test). The acronyms have the following definitions: fBV=fractional blood volume; and, SE=standard error.

FIG. 23. Change in Kₜᵣₐₙₛ Induced by Compound #10 in Non Necrotic Tissue in Nude Mice Bearing MDA-MB-468 Xenografts. The symbol “*” represents a p value of p<0.01, signifying that the differences in treated mice were significantly different from tumor size in vehicle-treated mice (Student's t-test). The acronyms have the following definitions: Kₜᵣₐₙₛ=volume transfer coefficient; and, SE=standard error.

FIG. 24A-B. Cell Cycle Delay After Overnight Exposure to Compound 1205. Histograms depicting relative DNA content in HT1080 cells under normoxic conditions after treatment with Compound 1205 compared to vehicle. FIG. 24A. Histogram showing the effect of treatment with Compound 1205 at 10 nm. FIG. 24B. Histogram showing the effect of treatment with vehicle.

FIG. 25. Dose Response of Compound 1205 and Compound #10: Inhibition of the Production of Hypoxia-Induced VEGF in HeLa Cells.

FIG. 26. Inhibition of HT1080 Tumor Growth by Compound #10, 1205 and 1330. The symbol “++” represents a p value of p=0.051, signifying the difference in tumor size in Compound #10 treated mice from tumor size in vehicle-treated mice (Student's t-test) on Day 11. The symbol “**” represents a p value of p<0.05, signifying that the differences in tumor size in Compound 1205 (S,S diastereoisomer) treated mice were significantly different from tumor size in vehicle-treated mice and that the differences in tumor size in Compound 1205 (S,S diastereoisomer) treated mice were significantly different from tumor size in Compound 1330 (R,S diastereoisomer)-treated mice (ANOVA, multiple comparisons).

FIG. 27A-B. Effect of Compound 1205 on Intra-Tumor Human VEGF Levels. FIG. 27A. Effect of treatment with vehicle and Compound 1205 on intra-tumor VEGF levels for Study #21 (target tumor size: 1200 mm³) and Study #23 (target tumor size: 1500 mm³). FIG. 27B. Intra-tumor VEGF levels normalized to tumor size.

FIG. 28. Effect of Compound 1205 on Levels of Homeostatic Plasma Human VEGF for Study #21 and Study #23.

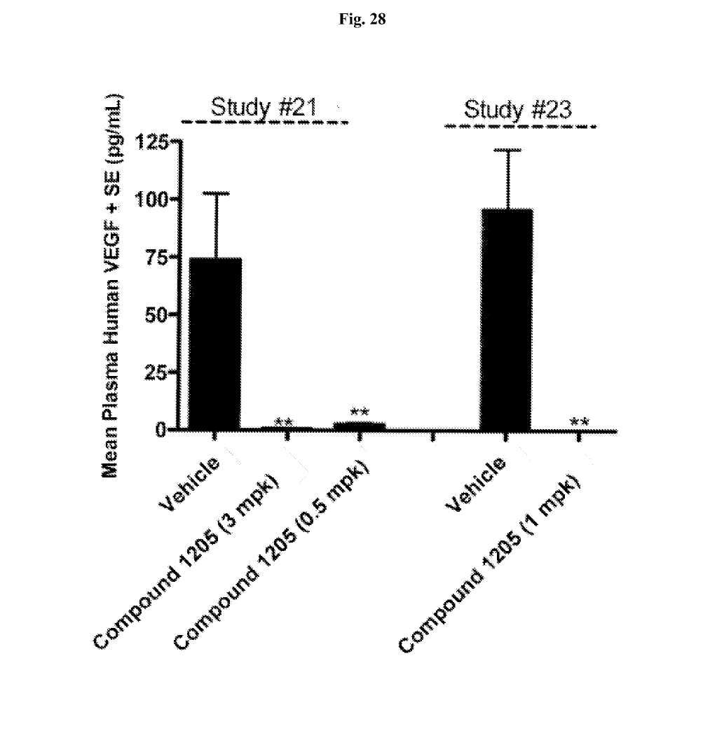

FIG. 29A-F. Treatment of BrdU labeled HT1080 cells with increasing doses of Compound #10. FIG. 29A. The effect of DMSO control on percentage of cells residing in S-phase. FIGS. 29B-F. The effect of increasing concentration of Compound #10 at 1 nm, 3 nm, 10 nm, 30 nm and 100 nm, respectively, on percentage of cells residing in S-phase.

FIG. 30A-B. FIG. 30A. The percentage of cells incorporating BrdU. FIG. 30B. The relative level of BrdU at each Compound #10 concentration.

FIG. 31A-B-C. BrdU Histogram and Quantification: FIG. 31(A). Histograms of DNA content demonstrating that the cell cycle distribution for HT1080 spheroids treated for 24 hours is not affected by exposure to Compound #10; FIG. 31(A)(i). Data.001 shows the control results; FIG. 31(A)(ii). Data.002 shows the results of exposure at 5 nm Compound #10; and, FIG. 31(A)(iii). Data.003 shows the results of exposure at 50 nm Compound #10. FIG. 31(B). BrdU quantification indicating the fraction of cells actively synthesizing DNA;

FIG. 31(B)(i). The effect of the DMSO control; FIG. 31(B)(ii). Represents the Data.001 results; and, FIG. 31(B)(iii). Represents the Data.003 results. FIG. 31(C) A graphical representation of the percentage of cells that incorporated BrdU (i.e., the cells in S-phase) after treatment with Compound #10 at various concentrations.

FIG. 32A-B-C. BrdU Histogram and Quantification: FIG. 32(A). Histograms of DNA content demonstrating that the cell cycle distribution for HT1080 spheroids treated for 48 hours is not affected by exposure to Compound #10; FIG. 32(A)(i). Data.004 shows the control results; FIG. 32(A)(ii). Data.005 shows the results of exposure at 10 nm Compound #10; and, FIG. 32(A)(iii). Data.006 shows the results of exposure at 50 nm Compound #10. FIG. 32(B). BrdU quantification indicating the fraction of cells actively synthesizing DNA;

FIG. 32(B)(i). Represents the Data.004 results; FIG. 32(B)(ii). Represents the Data.005 results; and, FIG. 32(B)(iii). Represents the Data.006 results. FIG. 32(C) A graphical representation of the percentage of cells that incorporated BrdU (i.e., the cells in S-phase) after treatment with Compound #10 at various concentrations.

FIG. 33. The effect of Compound #10 on Anchorage Independent Colony Formation.

FIG. 34. The effect of Compound #10 administration on serum VEGF-A levels in patients. The effect of Compound #10 reduced serum VEGF-A levels in patients.

FIG. 35. The effect of Compound #10 administration on plasma VEGF-A levels in patients, where the plasma VEGF-A concentration is provided on a log₁₀scale. The effect of Compound #10 reduced plasma VEGF-A levels in patients.

FIG. 36. The effect of Compound #10 administration on tumor perfusion in patients as determined by DCE-MRI, where the baseline K-trans was in a range of 0.0065 to 0.1005. The effect of Compound #10 reduced tumor perfusion in patients.

FIG. 37. The effect of Compound #10 administration on serum IL-6 levels in patients, where the symbol “*” represents the Lower Limit Of Quantification at 4.0 pg/mL imputed for Below the Quantification Limit of the assay (BQL) results. The effect of Compound #10 reduced serum IL-6 levels in patients.

FIG. 38. The effect of Compound #10 administration on plasma IL-6 levels in patients, where the symbol “*” represents the Lower Limit Of Quantification at 4.0 pg/mL imputed for Below the Quantification Limit of the assay (BQL) results. The effect of Compound #10 reduced plasma IL-6 levels in patients.

FIG. 39. The effect of Compound #10 administration on hearing function in patients, where increases in the pure tone threshold average reflect loss of hearing acuity and increases in BAER Wave V latency reflect increased electrical response to auditory stimuli. The effect of Compound #10 stabilized hearing function in patients while maintaining average word recognition score and average pure tone threshold.

FIG. 40. The effect of Compound #10 administration for one patient. The combined data for one patient showing the effect of Compound #10 administration in reducing and stabilizing tumor volume and maintaining average word recognition score and average pure tone threshold.

### 5. DETAILED DESCRIPTION

Presented herein are methods for treating NF (e.g., NF1, NF2, or schwannomatosis). Unless specified otherwise, as used hereinafter, NF includes, e.g., NF1, NF2, and schwannomatosis. In one aspect, the methods for treating NF involve the administration of a Compound, as a single-agent therapy, to a patient in need thereof. In a specific embodiment, presented herein is a method for treating NF, comprising administering to a patient in need thereof an effective amount of a Compound, as a single agent. In another embodiment, presented herein is a method for treating NF, comprising administering to a patient in need thereof a pharmaceutical composition comprising a Compound, as the single active ingredient, and a pharmaceutically acceptable carrier, excipient or vehicle.

In another aspect, the methods for treating NF involve the administration of a Compound in combination with another therapy (e.g., one or more additional therapies that do not comprise a Compound, or that comprise a different Compound) to a patient in need thereof. Such methods may involve administering a Compound prior to, concurrent with, or subsequent to administration of the additional therapy. In certain embodiments, such methods have an additive or synergistic effect. In a specific embodiment, presented herein is a method for treating NF, comprising administering to a patient in need thereof an effective amount of a Compound and an effective amount of another therapy.

In certain embodiments, the concentration of VEGF or other angiogenic or inflammatory mediators in biological specimens (e.g., plasma, serum, cerebral spinal fluid, urine, or any other biofluids) of a patient is monitored before, during and/or after a course of treatment involving the administration of a Compound or a pharmaceutical composition thereof to the patient. In certain embodiments, the tumoral blood flow or metabolism, or peritumoral inflammation or edema in a patient is monitored before, during and/or after a course of treatment involving the administration of a Compound or a pharmaceutical composition. The dosage, frequency and/or length of administration of a Compound or a pharmaceutical composition thereof to a patient may also be modified as a result of the concentration of VEGF or other angiogenic or inflammatory mediators, or tumoral blood flow or metabolism, or peritumoral inflammation or edema as assessed by imaging techniques. Alternatively, changes in one or more of these monitoring parameters (e.g., concentration of VEGF or other angiogenic or inflammatory mediators, or tumoral blood flow or metabolism, or peritumoral inflammation or edema) might indicate that the course of treatment involving the administration of the Compound or pharmaceutical composition thereof is effective in treating NF.

In a specific embodiment, presented herein is a method for treating NF, comprising: (a) administering to a patient in need thereof one or more doses of a Compound or a pharmaceutical composition thereof; and (b) monitoring the concentration of VEGF or other angiogenic, or inflammatory mediators (e.g., detected in biological specimens such as plasma, serum, cerebral spinal fluid, urine, or other biofluids), or monitoring tumoral blood flow or metabolism, or peritumoral inflammation or edema before and/or after step (a). In specific embodiments, step (b) comprises monitoring the concentration of one or more inflammatory mediators including, but not limited to, cytokines and interleukins such as IL-6 and IL-8. In particular embodiments, step (b) comprises monitoring the concentration of VEGFR, P1GF, VEGF-C, and/or VEGF-D. In certain embodiments, the monitoring step (b) is carried out before and/or after a certain number of doses (e.g., 1, 2, 4, 6, 8, 10, 12, 14, 15, or 20 doses, or more doses; or 2 to 4, 2 to 8, 2 to 20 or 2 to 30 doses) or a certain time period (e.g., 1, 2, 3, 4, 5, 6, or 7 days; or 1, 2, 3, 4, 5, 10, 15, 20, 30, 40, 45, 48, or 50 weeks), of administering the Compound. In certain embodiments, one or more of these monitoring parameters are detected prior to administration of the Compound or pharmaceutical composition thereof. In specific embodiments, a decrease in the concentration of VEGF or other angiogenic or inflammatory mediators or a change in tumoral blood flow or metabolism, or peritumoral inflammation or edema following administration of the Compound or pharmaceutical composition thereof indicates that the course of treatment is effective for treating NF. In some embodiments, a change in the concentration of VEGF or other angiogenic or inflammatory mediators or a change in tumoral blood flow or metabolism, or peritumoral inflammation or edema following administration of the Compound or pharmaceutical composition thereof may indicate that the dosage, frequency and/or length of administration of the Compound or a pharmaceutical composition thereof may be adjusted (e.g., increased, reduced or maintained).

The concentration of VEGF or other angiogenic or inflammatory mediators or a change in tumor blood flow or metabolism, or peritumoral inflammation or edema of a patient may be detected by any technique known to one of skill in the art. In certain embodiments, the method for detecting the concentration of VEGF or other angiogenic or inflammatory mediators in a patient involves obtaining a tissue or fluid sample from the patient and detecting the concentration of VEGF or the other angiogenic or the inflammatory mediators in the biological sample (e.g., from plasma serum sample, cerebral spinal fluid, urine, or other biofluids) that has been subjected to certain types of treatment (e.g., centrifugation) and detection by use of immunological techniques, such as ELISA. In a specific embodiment, the ELISA described herein, e.g., in the working examples in Section 9 et seq., may be used to detect the concentration of VEGF or other angiogenic or inflammatory mediators, in a biological sample (e.g., from plasma serum, cerebral spinal fluid, urine, or any other biofluids) that has been subjected to certain types of treatment (e.g., centrifugation). Other techniques known in the art that may be used to detect the concentration of VEGF or other angiogenic or inflammatory mediators, in a biological sample, include multiplex or proteomic assays. In a specific embodiment, a CT scan, a MRI scan, or a PET scan may be used to detect the tumor blood flow or metabolism, or peritumoral inflammation or edema or inflammation.

In specific embodiments, the methods for treating NF provided herein alleviate or manage one, two or more symptoms associated with NF. Alleviating or managing one, two or more symptoms of NF may be used as a clinical endpoint for efficacy of a Compound for treating NF. In some embodiments, the methods for treating NF provided herein reduce the duration and/or severity of one or more symptoms associated with NF. In some embodiments, the methods for treating NF provided herein inhibit the onset, progression and/or recurrence of one or more symptoms associated with NF. In some embodiments, the methods for treating NF provided herein reduce the number of symptoms associated with NF.

Symptoms associated with NF1 include, but are not limited to: light brown skin spots, neurofibromas (tumors that grow along nerves under the skin), plexiform neurofibromas (tumors involving multiple nerves), spinal cord and optic nerve tumors, learning disabilities (such as attention deficit hyperactivity disorder (ADHD)), freckling in the area of the armpit or the groin, growths on the iris of the eye (known as Lisch nodules or iris hamartomas), a tumor on the optic nerve (optic glioma), abnormal development of the spine (scoliosis), the temple (sphenoid) bone of the skull, or the tibia (one of the long bones of the shin), larger than normal head circumference, shorter than average height, hydrocephalus (the abnormal buildup of fluid in the brain), headache, epilepsy, cardiovascular complications associated with NF1, including congenital heart defects, high blood pressure (hypertension), and constricted, blocked, or damaged blood vessels (vasculopathy), poor linguistic and/or visual-spatial skills, and poor reading, spelling, and/or math skills.

Symptoms associated with NF2 include, but are not limited to: hearing loss, loss in hearing function, tinnitus, visual impairment (such as vision loss from cataracts), imbalance, painful skin lesions or tumors, weakness in an arm or leg, seizures, vertigo, facial weakness/paralysis, bilateral VS, unilateral VS, NF2 associated tumors (e.g., meningiomas, schwannomas, ependymomas), posterior cataracts, neurologic dysfunction related to VS growth (e.g., due to compression of other cranial nerves), skull-base tumors (including VS and meningiomas), and intracranial meningiomas (tumors growing on the covering of the brain).

Symptoms associated with schwannomatosis include, but are not limited to, multiple schwannomas, or tumors of nerve sheaths, but not vestibular tumors.

The methods for treating NF provided herein inhibit or reduce pathological production of human VEGF. In specific embodiments, the methods for treating NF provided herein selectively inhibit pathologic production of human VEGF (e.g., by the tumor), but do not disturb the physiological activity of human VEGF protein. Preferably, the methods for treating NF provided herein do not significantly inhibit or reduce physiological or homeostatic production of human VEGF. For example, blood pressure, protein levels in urine, and bleeding are maintained within normal ranges in treated subjects. In a specific embodiment, the treatment does not result in adverse events as defined in Cancer Therapy Evaluation Program, Common Terminology Criteria for Adverse Events, Version 3.0, DCTD, NCI, NIH, DHHS Mar. 31, 2003 (cstep.cancer.gov), publish date Aug. 9, 2006, which is incorporated by reference herein in its entirety. In other embodiments, the methods for treating NF provided herein do not result in adverse events of grade 2 or greater as defined in the Cancer Therapy Evaluation Program, Common Terminology Criteria for Adverse Events, Version 3.0, supra.

In specific embodiments, the methods for treating NF provided herein inhibit or reduce pathological angiogenesis and/or tumor growth. In certain embodiments, the methods for treating NF provided herein prolong or delay the G1/S or late G1/S phase of cell cycle (i.e., the period between the late resting or pre-DNA synthesis phase, and the early DNA synthesis phase).

In particular embodiments, the methods for treating NF provided herein inhibit, reduce, diminish, arrest, or stabilize a tumor associated with NF or a symptom thereof. In other embodiments, the methods for treating NF provided herein inhibit, reduce, diminish, arrest, or stabilize the blood flow, metabolism, peritumoral inflammation or peritumoral edema in a tumor associated with NF or a symptom thereof. In some embodiments, the methods for treating NF provided herein reduce, ameliorate, or alleviate the severity of NF and/or a symptom thereof. In particular embodiments, the methods for treating NF provided herein cause the regression of an NF tumor, tumor blood flow, tumor metabolism, or peritumoral inflammation or edema, and/or a symptom associated with NF. In other embodiments, the methods for treating NF provided herein reduce hospitalization (e.g., the frequency or duration of hospitalization) of a subject diagnosed with NF. In some embodiments, the methods for treating NF provided herein reduce hospitalization length of a subject diagnosed with NF. In certain embodiments, the methods provided herein increase the survival of a subject diagnosed with NF. In particular embodiments, the methods for treating NF provided herein inhibit or reduce the progression of one or more tumors or a symptom associated therewith.

In specific embodiments, the methods for treating NF provided herein enhance or improve the therapeutic effect of another therapy (e.g., an anti-cancer agent, radiation, drug therapy such as chemotherapy, or surgery). In certain embodiments, the methods for treating NF provided herein involve the use of a Compound as an adjuvant therapy. In certain embodiments, the methods for treating NF provided herein improve the ease in removal of tumors (e.g., enhance resectability of the tumors) by reducing vascularization prior to surgery. In particular embodiments, the methods for treating NF provided herein reduce vascularization after surgery, for example, reduce vascularization of the remaining tumor mass not removed by surgery. In some embodiments, the methods for treating NF provided herein prevent recurrence, e.g., recurrence of vascularization and/or tumor growth.

In specific embodiments, the methods for treating NF provided herein reduce or eliminate one, two, or more of the following: hearing loss, tinnitus, visual impairment, imbalance, or painful skin lesions, associated with NF. In some embodiments, the methods for treating NF provided herein improve hearing, hearing function and/or word recognition in a patient diagnosed with NF. In some embodiments, the methods for treating NF provided herein reduce the growth of a tumor or neoplasm associated with NF. In other embodiments, the methods for treating NF provided herein decrease tumor size of neurofibromas, plexiform neurofibromas, and/or NF2-associated tumors (e.g., meningiomas, schwannomas, or ependymomas). In certain embodiments, the methods for treating NF provided herein reduce the formation of a tumor such as a neurofibroma, a plexiform neurofibroma, or an NF2-associated tumor (e.g., meningioma, schwannoma, or ependymoma). In certain embodiments, the methods for treating NF provided herein eradicate, remove, or control primary, regional and/or metastatic tumors associated with NF. In other embodiments, the methods for treating NF provided herein decrease the number or size of metastases associated with NF. In particular embodiments, the methods for treating NF provided herein reduce the mortality of subjects diagnosed with NF. In other embodiments, the methods for treating NF provided herein increase the tumor-free survival rate of patients diagnosed with NF. In some embodiments, the methods for treating NF provided herein increase relapse-free survival. In certain embodiments, the methods for treating NF provided herein increase the number of patients in remission or decrease the hospitalization rate. In other embodiments, the methods for treating NF provided herein maintain the size of the tumor so that it does not increase, or so that it increases by less than the increase of a tumor after administration of a standard therapy as measured by methods available to one of skill in the art, such as X-ray, CT Scan, MRI or PET Scan. In other embodiments, the methods for treating NF provided herein prevent the development or onset of one or more symptoms associated with NF. In other embodiments, the methods for treating NF provided herein increase the length of remission in patients. In particular embodiments, the methods for treating NF provided herein increase symptom-free survival of NF patients. In some embodiments, the methods for treating NF provided herein do not cure NF in patients, but prevent the progression or worsening of the disease. In specific embodiments, the methods for treating NF achieve one or more of the clinical endpoints set forth in the working examples in Section 11 et seq. In particular embodiments, the methods for treating NF achieve one or more of the following: (i) improvement in neural function, e.g., hearing, balance, tinnitus, or vision; (ii) inhibition or reduction in pathological production of VEGF; (iii) stabilization or reduction of peritumoral inflammation or edema in a subject; (iv) reduction of the concentration of VEGF or other angiogenic or inflammatory mediators (e.g., cytokines or interleukins) in biological specimens (e.g., plasma, serum, cerebral spinal fluid, urine, or any other biofluids); (v) reduction of the concentration of P1GF, VEGF-C, VEGF-D, VEGFR, IL-6, and/or IL-8 in biological specimens (e.g., plasma, serum, cerebral spinal fluid, urine, or any other biofluids); (vi) inhibition or decrease in tumor metabolism or perfusion; (vii) inhibition or decrease in angiogenesis or vascularization; (viii) improvement in quality of life as assessed by methods well known in the art, e.g., tinnitus questionnaires.

In certain aspects, the methods for treating NF provided herein reduce the tumor size (e.g., volume or diameter) in a subject as determined by methods well known in the art, e.g., MRI. Three dimensional volumetric measurement performed by MRI has been shown to be sensitive and consistent in assessing tumor size (see, e.g., Harris et al., Neurosurgery, June 2008, 62(6): 1314-9), and thus may be employed to assess tumor shrinkage in the methods provided herein. In specific embodiments, the methods for treating NF provided herein reduce the tumor volume or tumor size (e.g., diameter) in a subject by at least about 20% as assessed by methods well known in the art, e.g., MRI. In certain embodiments, the methods for treating NF provided herein reduce the tumor size (e.g., volume or diameter) in a subject by at least about 10%, 15%, 20%, 25%, 30%, 35%, 40%, 45%, 50%, 55%, 60%, 65%, 80%, 85%, 90%, 95%, or 100%, relative to the tumor size prior to administration of a Compound, as assessed by methods well known in the art, e.g., MRI. In particular embodiments, the methods for treating NF provided herein reduce the tumor size (e.g., volume or diameter) in a subject by at least an amount in a range of from about 10% to about 100%, as assessed by methods well known in the art, e.g., MRI. In particular embodiments, the methods for treating NF provided herein reduce the tumor size (e.g., volume or diameter) in a subject by an amount in a range of from about 5% to 20%, 10% to 30%, 15% to 40%, 15% to 50%, 20% to 30%, 20% to 40%, 20% to 50%, 30% to 60%, 30% to 70%, 30% to 80%, 30% to 90%, 30% to 95%, 30% to 99%, 40% to 100%, or any range in between, relative to the tumor size prior to administration of a Compound, as assessed by methods well known in the art, e.g., MRI.

In some aspects, the methods for treating NF provided herein improve or enhance/increase hearing or hearing function in a subject. Such improvements in hearing or hearing function may be as assessed, e.g., by relative changes in word recognition score and hearing response, change in pure tone average, change in latency of wave V on click-evoked Brainstem Auditory Evoked Responses (BAERs), and/or change in distortion-product otoacoustic emission (OAE) levels at frequencies above 1 KHz (e.g., frequencies in the range of 1.1 KHz to 2 KHz, 1.1 KHz to 4 KHz, or 1.1 KHz to 5 KHz), if present. Assessments of word recognition and pure tone thresholds can be direct measures of patient function that define symptomatic consequences of the presence of VS (see, e.g., Halpin et al., Otol. Neurotol., Jan. 2006, 27(1):110-6). Standard clinical criteria for defining hearing response (H-R) based on a 50-word hearing test (see, e.g., Thornton et al., J. Speech Hear. Res., September 1978, 21(3): 507-18) can be employed for word recognition tests. Standard methods for measurement of BAERs and OAEs have been described, see, e.g., Lalwani et al., Am. J. Otol., 1998, 19(3):352-357; and Telischi et al., Laryngoscope., 1995, 105(7): 675-82. In measuring BAERs, electrodes are placed on a patient's scalp and on each earlobe, and clicking noises are then sent through earphones. The electrodes monitor the brain's response to the clicking noises and record the response on a graph. Pure-tone averages can be calculated as the average of the pure-tone thresholds at 500, 1000, 2000, and 3000 Hz. In a specific embodiment, an improvement in hearing or hearing function may be assessed using the methods provided in the working examples in Section 11 et seq.

In specific embodiments, the methods for treating NF provided herein improve hearing or hearing function in a subject as assessed, e.g., by relative changes in word recognition score and hearing response, change in pure tone average, change in latency of wave V on click-evoked BAERs, and/or change in distortion-product OAE levels at frequencies above 1 KHz (e.g., frequencies in the range of 1.1 KHz to 2 KHz, 1.1 KHz to 4 KHz, or 1.1 KHz to 5 KHz). In certain embodiments, the methods for treating NF provided herein improve hearing or hearing function in a subject as determined by an improvement (e.g., change from abnormal to normal classification) in the latency of wave V on click-evoked BAERs and/or improvement in the distortion-product OAE levels at frequencies above 1 KHz (e.g., frequencies in the range of 1.1 KHz to 2 KHz, 1.1 KHz to 4 KHz, or 1.1 KHz to 5 KHz). In certain embodiments, the methods for treating NF provided herein improve hearing or hearing function in a subject as determined by an increase in the word recognition score above the 95% critical difference threshold taking as a reference the baseline word discrimination score as assessed by methods well known in the art, e.g., 50-word recognition test (see, e.g., Thornton et al., J. Speech Hear. Res., September 1978, 21(3): 507-18; Halpin C, Rauch S D. Otol Neurotol. 2006 Jan. 27(1): 110-6; and working examples in Section 11 et seq.). Word recognition on a 50-word hearing test is scored from 0 to 100% by increments of 2%, as shown in Table 31.

In some aspects, the methods for treating NF provided herein improve or increase word recognition by a subject as assessed by methods well known in the art, e.g., a word recognition test known in the art. For example, determination of word recognition scores using a standardized list of test words, e.g., the 50-item Central Institute for the Deaf [CID] list W-22, recorded) (Thornton et al., J. Speech Hear Res., September 1978, 21(3): 507-18; Halpin C, Rauch S D. Otol Neurotol. 2006 Jan. 27(1): 110-6) may be employed in the methods provided herein. In specific embodiments, the methods for treating NF provided herein increase word recognition by a subject by an increase in the word recognition score above the 95% critical difference threshold taking as a reference the baseline word discrimination score, as assessed by methods well known in the art, e.g., word recognition test. Word recognition on a 50-word hearing test is scored from 0 to 100% by increments of 2%, as shown in Table 31.

In particular aspects, the methods for treating NF provided herein inhibit or decrease tumor perfusion in a subject as assessed by methods well known in the art, e.g., dynamic contrast-enhanced MRI (DCE-MRI). Standard protocols for DCE-MRI have been described (see., e.g., Morgan et al., J. Clin. Oncol., Nov. 1, 2003, 21(21):3955-64; Leach et al., Br. J. Cancer, May 9, 2005, 92(9):1599-610; Liu et al., J. Clin. Oncol., August 2005, 23(24): 5464-73; and Thomas et al., J. Clin. Oncol., Jun. 20, 2005, 23(18):4162-71) and can be applied in the methods provided herein. In specific embodiments, the methods for treating NF provided herein inhibit or decrease tumor perfusion in a subject by at least about 5%, 10%, 15%, 20%, 25%, 30%, 35%, 40%, 45%, 50%, 55%, 60%, 65%, 80%, 85%, 90%, 95%, or 100%, relative to tumor perfusion prior to administration of a Compound, as assessed by methods well known in the art, e.g., DCE-MRI.

In particular aspects, the methods for treating NF provided herein inhibit or decrease tumor metabolism in a subject as assessed by methods well known in the art, e.g., PET scanning Standard protocols for PET scanning or have been described and can be applied in the methods provided herein. In specific embodiments, the methods for treating NF provided herein inhibit or decrease tumor metabolism in a subject by at least about 5%, 10%, 15%, 20%, 25%, 30%, 35%, 40%, 45%, 50%, 55%, 60%, 65%, 80%, 85%, 90%, 95%, or 100%, relative to tumor metabolism prior to administration of a Compound, as assessed by methods well known in the art, e.g., PET scanning. In particular embodiments, the methods for treating NF provided herein inhibit or decrease tumor metabolism in a subject in the range of about 10% to 100%, relative to tumor metabolism prior to administration of a Compound, or any range in between, as assessed by methods well known in the art, e.g., PET scan.

In specific aspects, the methods for treating NF provided herein result in improvements in tinnitus in a subject with symptomatic VS. Improvements in tinnitus can be assessed by methods well known in the art, e.g., standardized scales for the evaluation of tinnitus loudness and/or the Tinnitus Reaction Questionnaire (“TRQ”) (see, e.g., Klockhoff et al., Acta Otolaryngol. April 1967, 63(4): 347-65; McCombe et al., Clin Otolaryngol Allied Sci., October 2001, 26(5): 388-93; and Wilson et al., J Speech Hear Res., February 1991, 34(1):197-201). For example, the loudness/severity of the tinnitus can be rated according to the criteria modified from Klockhoff et al., Acta Otolaryngol. April 1967, 63(4): 347-65, which is incorporated herein by reference in its entirety. In specific embodiments, the scale for grading of tinnitus severity may be as follows: 0=Tinnitus is not perceived at all; 1=Tinnitus is perceived slightly and periodically; 2=Tinnitus is perceived slightly and continuously; or moderately and periodically; 3=Tinnitus is perceived slightly to moderately and continuously; 4=Tinnitus is perceived moderately, or slightly to severely, and continuously; 5=Tinnitus is perceived from moderately to severely and continuously; and 6=Tinnitus is perceived severely and continuously (see, e.g., Table 30 in Section 11 et seq.). In particular embodiments, the methods for treating NF provided herein result in improvements in the tinnitus score of a subject with symptomatic VS by 1, 2, 3, 4, or 5 score(s), as assessed by methods well known in the art, e.g., tinnitus test using, e.g., the scale described above and in Table 30 in Section 11 et seq.).

In specific aspects, the methods for treating NF provided herein decrease the concentration of VEGF or other angiogenic or inflammatory mediators (e.g., cytokines or interleukins, such as IL-6) in a subject as assessed by methods well known in the art, e.g., ELISA. In specific embodiments, the methods for treating NF provided herein decrease the concentration of VEGF or other angiogenic or inflammatory mediators (e.g., cytokines or interleukins, such as IL-6) in a subject by at least about 5%, 10%, 15%, 20%, 25%, 30%, 35%, 40%, 45%, 50%, 55%, 60%, 65%, 80%, 85%, 90%, 95%, or 100%, relative to the respective concentration prior to administration of a Compound, as assessed by methods well known in the art, e.g., ELISA. In particular embodiments, the methods for treating NF provided herein decrease the concentration of VEGF or other angiogenic or inflammatory mediators (e.g., cytokines or interleukins, such as IL-6) in a subject in the range of about 5% to 20%, 10% to 20%, 10% to 30%, 15% to 40%, 15% to 50%, 20% to 30%, 20% to 40%, 20% to 50%, 30% to 60%, 30% to 70%, 30% to 80%, 30% to 90%, 30% to 99%, 30% to 100%, or any range in between, relative to the respective concentration prior to administration of a Compound, as assessed by methods well known in the art, e.g., ELISA.

In specific aspects, the methods for treating NF provided herein decrease the concentrations of P1GF, VEGF-C, VEGF-D, IL-6, and/or IL-8 in a subject as assessed by methods well known in the art, e.g., ELISA. In specific embodiments, the methods for treating NF provided herein decrease the concentrations of P1GF, VEGF-C, VEGF-D, IL-6, and/or IL-8 in a subject by at least about 5%, 10%, 15%, 20%, 25%, 30%, 35%, 40%, 45%, 50%, 55%, 60%, 65%, 80%, 85%, 90%, 95%, or 100%, relative to the respective concentration prior to administration of a Compound, as assessed by methods well known in the art, e.g., ELISA. In particular embodiments, the methods for treating NF provided herein decrease the concentrations of P1GF, VEGF-C, VEGF-D, IL-6, and/or IL-8 in a subject in the range of about 5% to 20%, 10% to 30%, 15% to 40%, 15% to 50%, 20% to 30%, 20% to 40%, 20% to 50%, 30% to 60%, 30% to 70%, 30% to 80%, 30% to 90%, 30% to 99%, 30% to 100%, or any range in between, relative to the respective concentration prior to administration of a Compound, as assessed by methods well known in the art, e.g., ELISA.

In specific embodiments, the methods for treating NF provided herein inhibit or decrease pathological production of VEGF by at least about 5%, 10%, 15%, 20%, 25%, 30%, 35%, 40%, 45%, 50%, 55%, 60%, 65%, 80%, 85%, 90%, 95%, or 100%, relative to the pathological production of VEGF observed prior to administration of a Compound, as assessed by methods well known in the art, e.g., ELISA. In particular embodiments, the methods for treating NF provided herein inhibit or decrease pathological production of VEGF in the range of about 5% to 20%, 10% to 30%, 15% to 40%, 15% to 50%, 20% to 30%, 20% to 40%, 20% to 50%, 30% to 60%, 30% to 70%, 30% to 80%, 30% to 90%, 30% to 99%, 30% to 100%, or any range in between, relative to the pathological production of VEGF observed prior to administration of a Compound, as assessed by methods well known in the art, e.g., ELISA.

In specific embodiments, the methods for treating NF provided herein inhibit or reduce angiogenesis or vascularization, by at least about 5%, 10%, 15%, 20%, 25%, 30%, 35%, 40%, 45%, 50%, 55%, 60%, 65%, 80%, 85%, 90%, 95%, or 100%, relative to angiogenesis or vascularization observed prior to administration of a Compound, as assessed by methods well known in the art, e.g., MRI scan, CT scan, PET scan. In particular embodiments, the methods for treating NF provided herein inhibit or reduce angiogenesis or vascularization, in the range of about 5% to 20%, 10% to 20%, 10% to 30%, 15% to 40%, 15% to 50%, 20% to 30%, 20% to 40%, 20% to 50%, 30% to 60%, 30% to 70%, 30% to 80%, 30% to 90%, 30% to 99%, 30% to 100%, or any range in between, relative to angiogenesis or vascularization observed prior to administration of a Compound, as assessed by methods well known in the art.

In specific embodiments, the methods for treating NF provided herein inhibit or reduce inflammation, by at least about 5%, 10%, 15%, 20%, 25%, 30%, 35%, 40%, 45%, 50%, 55%, 60%, 65%, 80%, 85%, 90%, 95%, or 100%, relative to inflammation observed prior to administration of a Compound, or any percentage in between, as assessed by methods well known in the art, e.g., CT scan, MRI scan, or PET scan. In particular embodiments, the methods for treating NF provided herein inhibit or reduce inflammation, in the range of about 5% to 15%, 10% to 20%, 10% to 30%, 15% to 40%, 15% to 50%, 20% to 30%, 20% to 40%, 20% to 50%, 30% to 60%, 30% to 70%, 30% to 80%, 30% to 90%, 30% to 99%, 30% to 100%, or any range in between, relative to inflammation observed prior to administration of a Compound, or any percentage in between, as assessed by methods well known in the art, e.g., CT scan, MRI scan, or PET scan.

In specific embodiments, the methods for treating NF provided herein inhibit or reduce edema, by at least about 5%, 10%, 15%, 20%, 25%, 30%, 35%, 40%, 45%, 50%, 55%, 60%, 65%, 80%, 85%, 90%, 95%, or 100%, relative to the edema observed prior to administration of a Compound, or any percentage in between, as assessed by methods well known in the art, e.g., CT scan, MRI scan, or PET scan. In particular embodiments, the methods for treating NF provided herein inhibit or reduce edema, in the range of about 5% to 15%, 10% to 20%, 10% to 30%, 15% to 40%, 15% to 50%, 20% to 30%, 20% to 40%, 20% to 50%, 30% to 60%, 30% to 70%, 30% to 80%, 30% to 90%, 30% to 99%, 30% to 100%, or any range in between, relative to the edema observed prior to administration of a Compound, or any percentage in between, as assessed by methods well known in the art, e.g., CT scan, MRI scan, or PET scan.

In specific embodiments, the methods for treating NF provided herein minimize the severity and/or frequency of one or more side effects observed with current anti-angiogenesis therapies. In certain embodiments, the methods for treating NF provided herein do not cause one or more side effects observed with current anti-angiogenesis therapies. Such side effects include, but are not limited to, bleeding, arterial and venous thrombosis, hypertension, delayed wound healing, asymptomatic proteinuria, nasal septal perforation, reversible posterior leukoencephalopathy syndrome in association with hypertension, light-headedness, ataxia, headache, hoarseness, nausea, vomiting, diarrhea, rash, subungual hemorrhage, myelosuppression, fatigue, hypothyroidism, QT interval prolongation, and heart failure.

5.1 Compounds

In one embodiment, provided herein are Compounds having Formula (I):

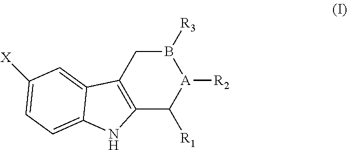

or a pharmaceutically acceptable salt, racemate or stereoisomer thereof, wherein,
X is hydrogen; C₁ to C₆ alkyl optionally substituted with one or more halogen substituents;
hydroxyl; halogen; or C₁ to C₅ alkoxy optionally substituted with aryl;
A is CH or N;
B is CH or N, with the proviso that at least one of A or B is N, and that when A is N, B is CH;
R₁ is hydroxyl; C₁ to C₈ alkyl optionally substituted with alkylthio, 5 to 10 membered heteroaryl, or aryl optionally substituted with one or more independently selected Rₒ substituents; C₂ to C₈ alkyenyl; C₂ to C₈ alkynyl; 3 to 12 membered heterocycle optionally substituted with one or more substituents independently selected from halogen, oxo, amino, alkylamino, acetamino, thio, or alkylthio; 5 to 12 membered heteroaryl optionally substituted with one or more substituents independently selected from halogen, oxo, amino, alkylamino, acetamino, thio, or alkylthio; or aryl, optionally substituted with one or more independently selected Rₒ substituents;
Rₒ is a halogen; cyano; nitro; sulfonyl optionally substituted with C₁ to C₆ alkyl or 3 to 10 membered heterocycle; amino optionally substituted with C₁ to C₆ alkyl, —C(O)—R_{b}, —C(O)O—R_{b}, sulfonyl, alkylsulfonyl, 3 to 10 membered heterocycle optionally substituted with —C(O)O—Rₙ; —C(O)—NH—R_{b}; 5 to 6 membered heterocycle; 5 to 6 membered heteroaryl; C₁ to C₆ alkyl optionally substituted with one or more substituents independently selected from hydroxyl, halogen, amino, or 3 to 12 membered heterocycle wherein amino and 3 to 12 membered heterocycle are optionally substituted with one or more C₁ to C₄ alkyl substituents optionally substituted with one or more substituents independently selected from C₁ to C₄ alkoxy, amino, alkylamino, or 5 to 10 membered heterocycle; —C(O)—Rₙ; or —ORₐ;
Rₐ is hydrogen; C₂ to C₈ alkylene; —C(O)—Rₙ; —C(O)O—R_{b}; —C(O)—NH—R_{b}; C₃-C₁₄cycloalkyl; aryl; heteroaryl; heterocyclyl; C₁ to C₈ alkyl optionally substituted with one or more substituents independently selected from hydroxyl, halogen, C₁ to C₄ alkoxy, amino, alkylamino, acetamide, —C(O)—R_{b}, —C(O)O—R_{b}, aryl, 3 to 12 membered heterocycle, or 5 to 12 membered heteroaryl, further wherein the alkylamino is optionally substituted with hydroxyl, C₁ to C₄ alkoxy, or 5 to 12 membered heteroaryl optionally substituted with C₁ to C₄ alkyl, further wherein the acetamide is optionally substituted with C₁ to C₄ alkoxy, sulfonyl, or alkylsulfonyl, further wherein the 3 to 12 membered heterocycle is optionally substituted with C₁ to C₄ alkyl optionally substituted with hydroxyl, —C(O)—Rₙ, —C(O)O—Rₙ, or oxo, further wherein the amino is optionally substituted with C₁ to C₄ alkoxycarbonyl, imidazole, isothiazole, pyrazole, pyridine, pyrazine, pyrimidine, pyrrole, thiazole or sulfonyl substituted with C₁ to C₆ alkyl, wherein pyridine and thiazole are each optionally substituted with C₁ to C₄ alkyl;
R_{b} is hydroxyl; amino; alkylamino optionally substituted with hydroxyl, amino, alkylamino, C₁ to C₄ alkoxy, 3 to 12 membered heterocycle optionally substituted with one or more independently selected C₁ to C₆ alkyl, oxo, —C(O)O—Rₙ, or 5 to 12 membered heteroaryl optionally substituted with C₁ to C₄ alkyl; C₁ to C₄ alkoxy; C₂ to C₈ alkenyl; C₂ to C₈ alkynyl; aryl, wherein the aryl is optionally substituted with one or more substituents independently selected from halogen or C₁ to C₄ alkoxy; 5 to 12 membered heteroaryl; 3 to 12 membered heterocycle optionally substituted with one or more substituents independently selected from acetamide, —C(O)O—Rₙ, 5 to 6 membered heterocycle, or C₁ to C₆ alkyl optionally substituted with hydroxyl, C₁ to C₄ alkoxy, amino, or alkylamino; or C₁ to C₈ alkyl optionally substituted with one or more substituents independently selected from C₁ to C₄ alkoxy, aryl, amino, or 3 to 12 membered heterocycle, wherein the amino and 3 to 12 membered heterocycle are optionally substituted with one or more substituents independently selected from C₁ to C₆ alkyl, oxo, or —C(O)O—Rₙ;
R₂ is hydrogen; hydroxyl; 5 to 10 membered heteroaryl; C₁ to C₈ alkyl optionally substituted with hydroxyl, C₁ to C₄ alkoxy, 3 to 10 membered heterocycle, 5 to 10 membered heteroaryl, or aryl; —C(O)—R_{c}; —C(O)O—R_{d}; —C(O)—N(R_{d}R_{d}); —C(S)—N(R_{d}R_{d}); —C(S)—O—Rₑ; —S(O₂)—Rₑ; —C(NRₑ)—S—Rₑ; or —C(S)—S—R_{f};
R_{c} is hydrogen; amino optionally substituted with one or more substituents independently selected from C₁ to C₆ alkyl or aryl; aryl optionally substituted with one or more substituents independently selected from halogen, haloalkyl, hydroxyl, C₁ to C₄ alkoxy, or C₁ to C₆ alkyl; —C(O)—Rₙ; 5 to 6 membered heterocycle optionally substituted with —C(O)—Rₙ; 5 to 6 membered heteroaryl; thiazoleamino; C₁ to C₈ alkyl optionally substituted with one or more substituents independently selected from halogen, C₁ to C₄ alkoxy, phenyloxy, aryl, —C(O)—Rₙ, —O—C(O)—Rₙ, hydroxyl, or amino optionally substituted with —C(O)O—Rₙ;
R_{d} is independently hydrogen; C₂ to C₈ alkenyl; C₂ to C₈ alkynyl; aryl optionally substituted with one or more substituents independently selected from halogen, nitro, C₁ to C₆ alkyl, —C(O)O—Rₑ, or —ORₑ; or C₁ to C₈ alkyl optionally substituted with one or more substituents independently selected from halogen, C₁ to C₄ alkyl, C₁ to C₄ alkoxy, phenyloxy, aryl, 5 to 6 membered heteroaryl, —C(O)—Rₙ, —C(O)O—Rₙ, or hydroxyl, wherein the aryl is optionally substituted with one or more substituents independently selected from halogen or haloalkyl;
Rₑ is hydrogen; C₁ to C₆ alkyl optionally substituted with one or more substituents independently selected from halogen or alkoxy; or aryl optionally substituted with one or more substituents independently selected from halogen or alkoxy;
R_{f} is C₁ to C₆ alkyl optionally substituted with one or more substituents independently selected from halogen, hydroxyl, C₁ to C₄ alkoxy, cyano, aryl, or —C(O)—Rₙ, wherein the alkoxy is optionally substituted with one or more C₁ to C₄ alkoxy substituents and the aryl is optionally substituted with one or more substituents independently selected from halogen, hydroxyl, C₁ to C₄ alkoxy, cyano, or C₁ to C₆ alkyl;
Rₙ is hydroxyl, C₁ to C₄ alkoxy, amino, or C₁ to C₆ alkyl;
R₃ is hydrogen or —C(O)—R_{g}; and
R_{g} is hydroxyl; amino optionally substituted with cycloalkyl or 5 to 10 membered heteroaryl; or 5 to 10 membered heterocycle, wherein the 5 to 10 membered heterocycle is optionally substituted with —C(O)—Rₙ.

In one embodiment, the compound of Formula (I) is other than:
(R)-1-(benzo[d][1,3]dioxol-5-yl)-2,3,4,9-tetrahydro-1H-pyrido[3,4-b]indole,
1-(benzo[d][1,3]dioxol-5-yl)-N-benzyl-3,4-dihydro-1H-pyrido[3,4-b]indole-2(9H)-carbothioamide,
(R)-1-(benzo[d][1,3]dioxol-5-yl)-N-benzyl-3,4-dihydro-1H-pyrido[3,4-b]indole-2(9H)-carbothioamide,
1-phenyl-2,3,4,9-tetrahydro-1H-pyrido[3,4-b]indole,
(R)-1-(benzo[d][1,3]dioxol-5-yl)-N-benzyl-3,4-dihydro-1H-pyrido[3,4-b]indole-2(9H)-carboxamide,
N-benzyl-1-phenyl-3,4-dihydro-1H-pyrido[3,4-b]indole-2(9H)-carboxamide,
N,1-diphenyl-3,4-dihydro-1H-pyrido[3,4-b]indole-2(9H)-carboxamide,
N-(naphthalen-1-yl)-1-phenyl-3,4-dihydro-1H-pyrido[3,4-b]indole-2(9H)-carboxamide,
1-(benzo[d][1,3]dioxol-5-yl)-N-cyclohexyl-3,4-dihydro-1H-pyrido[3,4-b]indole-2(9H)-carboxamide,
1-(benzo[d][1,3]dioxol-5-yl)-N-phenyl-3,4-dihydro-1H-pyrido[3,4-b]indole-2(9H)-carboxamide,
1-(3-chloro-4-methoxyphenyl)-N-phenyl-3,4-dihydro-1H-pyrido[3,4-b]indole-2(9H)-carboxamide,
(R)-1-(benzo[d][1,3]dioxol-5-yl)-N—((R)-1-phenylethyl)-3,4-dihydro-1H-pyrido[3,4-b]indole-2(9H)-carboxamide,
(R)-1-(benzo[d][1,3]dioxol-5-yl)-N—((S)-1-phenylethyl)-3,4-dihydro-1H-pyrido[3,4-b]indole-2(9H)-carboxamide,
(R)-1-(benzo[d][1,3]dioxol-5-yl)-N-benzoyl-3,4-dihydro-1H-pyrido[3,4-b]indole-2(9H)-carboxamide,
(R)—N-(1-(benzo[d][1,3]dioxol-5-yl)-2,3,4,9-tetrahydro-1H-pyrido[3,4-b]indole-2-carbonothioyl)benzamide,
benzyl 1-(benzo[d][1,3]dioxol-5-yl)-3,4-dihydro-1H-pyrido[3,4-b]indole-2(9H)-carboxylate,
(R)-benzyl 1-(benzo[d][1,3]dioxol-5-yl)-3,4-dihydro-1H-pyrido[3,4-b]indole-2(9H)-carboxylate,
methyl 1-phenyl-3,4-dihydro-1H-pyrido[3,4-b]indole-2(9H)-carboxylate,
methyl 5-oxo-5-(1-phenyl-3,4-dihydro-1H-pyrido[3,4-b]indol-2(9H)-yl)pentanoate,
5-(1-(3-chloro-4-methoxyphenyl)-3,4-dihydro-1H-pyrido[3,4-b]indol-2(9H)-yl)-5-oxopentanoic acid,
5-(1-(benzo[d][1,3]dioxol-5-yl)-3,4-dihydro-1H-pyrido[3,4-b]indol-2(9H)-yl)-5-oxopentanoic acid,
3-(2-aminophenyl)-1-(1-(benzo[d][1,3]dioxol-5-yl)-3,4-dihydro-1H-pyrido[3,4-b]indol-2(9H)-yl)propan-1-one,
(R)-1-(benzo[d][1,3]dioxol-5-yl)-N-(2-chlorobenzyl)-3,4-dihydro-1H-pyrido[3,4-b]indole-2(9H)-carbothioamide,
(R)-1-(benzo[d][1,3]dioxol-5-yl)-N-(2,4-dichlorobenzyl)-3,4-dihydro-1H-pyrido[3,4-b]indole-2(9H)-carbothioamide,
(R)-1-(benzo[d][1,3]dioxol-5-yl)-N-(2-fluorobenzyl)-3,4-dihydro-1H-pyrido[3,4-b]indole-2(9H)-carbothioamide,
(R)-1-(benzo[d][1,3]dioxol-5-yl)-N—((S)-1-phenylethyl)-3,4-dihydro-1H-pyrido[3,4-b]indole-2(9H)-carbothioamide,
(R)-4-((1-(benzo[d][1,3]dioxol-5-yl)-2,3,4,9-tetrahydro-1H-pyrido[3,4-b]indole-2-carbothioamido)methyl)benzoic acid,
(R)-methyl 4-((1-(benzo[d][1,3]dioxol-5-yl)-2,3,4,9-tetrahydro-1H-pyrido[3,4-b]indole-2-carbothioamido)methyl)benzoate,
(R)-3-((1-(benzo[d][1,3]dioxol-5-yl)-2,3,4,9-tetrahydro-1H-pyrido[3,4-b]indole-2-carbothioamido)methyl)benzoic acid,
(R)-methyl 3-((1-(benzo[d][1,3]dioxol-5-yl)-2,3,4,9-tetrahydro-1H-pyrido[3,4-b]indole-2-carbothioamido)methyl)benzoate,
(R)-1-(benzo[d][1,3]dioxol-5-yl)-N-(4-chloro-3-(trifluoromethyl)phenyl)-3,4-dihydro-1H-pyrido[3,4-b]indole-2(9H)-carbothioamide,
(R)-1-(benzo[d][1,3]dioxol-5-yl)-N-(2-(trifluoromethyl)phenyl)-3,4-dihydro-1H-pyrido[3,4-b]indole-2(9H)-carbothioamide,
(R)-1-(benzo[d][1,3]dioxol-5-yl)-N-(3-fluorobenzyl)-3,4-dihydro-1H-pyrido[3,4-b]indole-2(9H)-carbothioamide,
(R)-1-(benzo[d][1,3]dioxol-5-yl)-N-(4-chlorobenzyl)-3,4-dihydro-1H-pyrido[3,4-b]indole-2(9H)-carbothioamide,
(R)-1-(benzo[d][1,3]dioxol-5-yl)-N-(3,4-dichlorobenzyl)-3,4-dihydro-1H-pyrido[3,4-b]indole-2(9H)-carbothioamide,
(R)-1-(benzo[d][1,3]dioxol-5-yl)-N-(4-fluorobenzyl)-3,4-dihydro-1H-pyrido[3,4-b]indole-2(9H)-carbothioamide,
(R)-1-(benzo[d][1,3]dioxol-5-yl)-N-(3,4-dimethylbenzyl)-3,4-dihydro-1H-pyrido[3,4-b]indole-2(9H)-carbothioamide,
(R)-1-(benzo[d][1,3]dioxol-5-yl)-N-(3-chlorobenzyl)-3,4-dihydro-1H-pyrido[3,4-b]indole-2(9H)-carbothioamide,
(R)-1-(benzo[d][1,3]dioxol-5-yl)-N-(naphthalen-1-ylmethyl)-3,4-dihydro-1H-pyrido[3,4-b]indole-2(9H)-carbothioamide,
(3,4-difluorophenyl)-(1-phenyl-1,3,4,9-tetrahydro-β-carbolin-2-yl)-methanone,
6-methoxy-1,2,3,4-tetrahydronorharmane,
1,2,3,4-tetrahydronorharman-3-carboxylic acid,
6-methoxy-1,2,3,4-tetrahydronorharman-1-carboxylic acid,
1-(4-methoxyphenyl)-1,2,3,4-tetrahydronorharman-3-carboxylic acid,
1-methyl-1,2,3,4-tetrahydronorharman-3-carboxylic acid,
1-methyl-1,2,3,4-tetrahydronorharman-1,3-dicarboxylic acid,
1-(diethylmethyl)-1,2,3,4-tetrahydronorharman-3-carboxylic acid,
(6-bromo-1,2,3,4-tetrahydronorharman-1-yl)-3-propionic acid,
1-isobutyl-1,2,3,4-tetrahydronorharman-3-carboxylic acid,
1-phenyl-1,2,3,4-tetrahydronorharman-3-carboxylic acid,
1-propyl-1,2,3,4-tetrahydronorharman-3-carboxylic acid,
1-methyl-1-methoxycarbonyl-6-benzyloxy-1,2,3,4-tetrahydronorharmane,
1-methyl-1-methoxycarbonyl-6-methoxy-1,2,3,4-tetrahydronorharmane,
1-methyl-1-methoxycarbonyl-6-hydroxy-1,2,3,4-tetrahydronorharmane,
1-methyl-1-methoxycarbonyl-6-chloro-1,2,3,4-tetrahydronorharmane,
1-methyl-1-methoxycarbonyl-6-bromo-1,2,3,4-tetrahydronorharmane,
1-methyl-2-N-acetyl-6-methoxy-1,2,3,4-tetrahydro-β-carboline,
2-N-acetyl-1,2,3,4-tetrahydro-β-carboline,
1-methyl-2-N-acetyl-6-methoxy-1,2,3,4-tetrahydro-β-carboline,
4-chlorobenzyl(1S,3R)-1-(2,4-dichlorophenyl)-1,2,3,4-tetrahydro-β-carboline-3-carboxamide,
(3R)-1-(1-benzylindol-3-yl)-2-tert-butoxycarbonyl-1,2,3,4-tetrahydro-β-carboline-3-carboxylic acid,
(3R)-1-(1-butylindol-3-yl)-2-tert-butoxycarbonyl-1,2,3,4-tetrahydro-β-carboline-3-carboxylic acid,
(1S,3R)-1-(indol-3-yl)-2-tert-butoxycarbonyl-1,2,3,4-tetrahydro-β-carboline-3-carboxylic acid,
(1S,3R)-1-(1-methylindol-3-yl)-2-tert-butoxycarbonyl-1,2,3,4-tetrahydro-β-carboline-3-carboxylic acid,
benzothiazol-2-yl (1S,3R)-1-cyclohexyl-2-tert-butoxycarbonyl-1,2,3,4-tetrahydro-β-carboline-3-carboxylic acid,
benzothiazol-2-yl (1S,3R)-1-cyclohexyl-1,2,3,4-tetrahydro-β-carboline-3-carboxylic acid,
1-(4-chlorophenyl)-1,2,3,4-tetrahydro-β-carboline,
1-(4-bromophenyl)-1,2,3,4-tetrahydro-β-carboline,
1-(4-nitrophenyl)-1,2,3,4-tetrahydro-β-carboline,
1-(4-dimethylaminophenyl)-1,2,3,4-tetrahydro-β-carboline,
1-(4-diethylaminophenyl)-1,2,3,4-tetrahydro-β-carboline,
1-(2,4-dimethoxyphenyl)-1,2,3,4-tetrahydro-β-carboline,
1-(3,4-dimethoxyphenyl)-1,2,3,4-tetrahydro-β-carboline,
1-(2,5-dimethoxyphenyl)-1,2,3,4-tetrahydro-β-carboline,
1-(3,5-dimethoxyphenyl)-1,2,3,4-tetrahydro-β-carboline,
1-(3,4,5-trimethoxyphenyl)-1,2,3,4-tetrahydro-β-carboline,
1-(4-nitrobenzo[d][1,3]dioxol-5-yl)-1,2,3,4-tetrahydro-β-carboline,
1-(2-fluorenyl)-1,2,3,4-tetrahydro-β-carboline,
1-(9-ethyl-9H-carbazol-3-yl)-1,2,3,4-tetrahydro-β-carboline,
6-chloro-1-(4-methylphenyl)-2,3,4,9-tetrahydro-1H-β-carboline, methyl 6-chloro-1-(4-methylphenyl)-1,3,4,9-tetrahydro-2H-β-carboline-2-carboxylate,
6-chloro-1-(4-methylphenyl)-2-(3-phenylpropanoyl)-2,3,4,9-tetrahydro-1H-β-carboline, phenylmethyl 6-chloro-1-(4-methylphenyl)-1,3,4,9-tetrahydro-2H-β-carboline-2-carboxylate,
6-fluoro-1-(4-methylphenyl)-2,3,4,9-tetrahydro-1H-β-carboline,
methyl 6-fluoro-1-(4-methylphenyl)-1,3,4,9-tetrahydro-2H-β-carboline-2-carboxylate,
6-fluoro-1-(4-methylphenyl)-2-(3-phenylpropanoyl)-2,3,4,9-tetrahydro-1H-β-carboline,
phenylmethyl 6-fluoro-1-(4-methylphenyl)-1,3,4,9-tetrahydro-2H-β-carboline-2-carboxylate,
6-bromo-1-(4-methylphenyl)-2,3,4,9-tetrahydro-1H-β-carboline,
methyl 6-bromo-1-(4-methylphenyl)-1,3,4,9-tetrahydro-2H-β-carboline-2-carboxylate,
6-bromo-1-(4-methylphenyl)-2-(3-phenylpropanoyl)-2,3,4,9-tetrahydro-1H-β-carboline,
phenylmethyl 6-bromo-1-(4-methylphenyl)-1,3,4,9-tetrahydro-2H-β-carboline-2-carboxylate,
(1R)-6-chloro-1-(4-methylphenyl)-2-(3-phenylpropanoyl)-2,3,4,9-tetrahydro-1H-β-carboline,
(1S)-6-chloro-1-(4-methylphenyl)-2-(3-phenylpropanoyl)-2,3,4,9-tetrahydro-1H-β-carboline,
1-(4-methylphenyl)-2-(methylsulfonyl)-2,3,4,9-tetrahydro-1H-β-carboline,
2-acetyl-1-(4-methylphenyl)-2,3,4,9-tetrahydro-1H-β-carboline,
1-(4-methylphenyl)-2-(3-phenylpropanoyl)-2,3,4,9-tetrahydro-1H-β-carboline,
6-(methyloxy)-1-(4-methylphenyl)-2-(3-phenylpropanoyl)-2,3,4,9-tetrahydro-1H-β-carboline,
6-methyl-1-(4-methylphenyl)-2-(3-phenylpropanoyl)-2,3,4,9-tetrahydro-1H-β-carboline,
(1R/1S)-1-(2,3-dihydro-1-benzofuran-5-yl)-2,3,4,9-tetrahydro-1H-β-carboline, or
1-(1,3-benzodioxol-5-yl)-2-(2-pyrimidinyl)-2,3,4,9-tetrahydro-1H-β-carboline.

As will be evident to one of skill in the art, Compounds provided herein comprise at least one stereocenter, and may exist as a racemic mixture or as an enantiomerically pure composition. In one embodiment, a Compound provided herein is the (S) isomer, in an enantiomerically pure composition. In certain embodiments, the enantiomeric excess (e.e.) is about 90%, about 95%, about 99% or about 99.9% or greater.

In another embodiment, provided herein are Compounds having Formula (II):

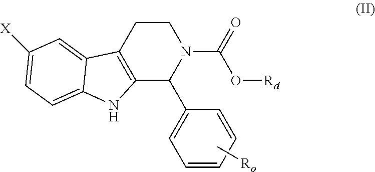

or a pharmaceutically acceptable salt, racemate or stereoisomer thereof, wherein,
X is hydrogen; C₁ to C₆ alkyl optionally substituted with one or more halogen substituents; hydroxyl; halogen; or C₁ to C₅ alkoxy optionally substituted with phenyl;
Rₒ is halogen; cyano; nitro; sulfonyl substituted with C₁ to C₆ alkyl or morpholinyl; amino optionally substituted with C₁ to C₆ alkyl, C(O)R_{b}, —C(O)O—R_{b}, alkylsulfonyl, morpholinyl or tetrahydropyranyl; C₁ to C₆ alkyl optionally substituted with one or more substituents independently selected from hydroxyl, halogen or amino; C(O)—Rₙ; or —ORₐ;
Rₐ is hydrogen; C₂ to C₈ alkenyl; —C(O)—Rₙ; —C(O)O—R_{b}; —C(O)—NH—R_{b}; C₁ to C₈ alkyl optionally substituted with one or more substituents independently selected from hydroxyl, halogen, C₁ to C₄ alkoxy, C₁ to C₄ alkoxy C₁ to C₄ alkoxy, amino, alkylamino, dialkylamino, acetamide, —C(O)—R_{b}, —C(O)O—R_{b}, aryl, morpholinyl, thiomorpholinyl, pyrrolidinyl, piperidinyl, piperazinyl, 1,3-dioxolan-2-one, oxiranyl, tetrahydrofuranyl, tetrahydropyranyl, 1,2,3-triazole, 1,2,4-triazole, furan, imidazole, isoxazole, isothiazole, oxazole, pyrazole, thiazole, thiophene or tetrazole;
   wherein amino is optionally substituted with C₁ to C₄ alkoxycarbonyl, imidazole, isothiazole, pyrazole, pyridine, pyrazine, pyrimidine, pyrrole, thiazole or sulfonyl substituted with C₁ to C₆ alkyl, wherein pyridine and thiazole are each optionally substituted with C₁ to C₄ alkyl;
   wherein alkylamino and dialkylamino are each optionally substituted on alkyl with hydroxyl, C₁ to C₄ alkoxy, imidazole, pyrazole, pyrrole or tetrazole; and,
   wherein morpholinyl, thiomorpholinyl, pyrrolidinyl, piperidinyl, piperazinyl and oxiranyl are each optionally substituted with —C(O)—Rₙ, —C(O)O—Rₙ, or C₁ to C₄ alkyl,
   wherein C₁ to C₄ alkyl is optionally substituted with hydroxyl;
R_{b} is hydroxyl; amino; alkylamino, optionally substituted on alkyl with hydroxyl, amino, alkylamino or C₁ to C₄ alkoxy; C₁ to C₄ alkoxy; C₂ to C₈ alkenyl; C₂ to C₈ alkynyl; aryl optionally substituted with one or more substituents independently selected from halogen and C₁ to C₄ alkoxy; furan; or C₁ to C₈ alkyl optionally substituted with one or more substituents independently selected from C₁ to C₄ alkoxy, aryl, amino, morpholinyl, piperidinyl or piperazinyl;
R_{d} is aryl optionally substituted with one or more substituents independently selected from halogen, nitro, C₁ to C₆ alkyl, —C(O)O—Rₑ, and —ORₑ;
Rₑ is hydrogen; C₁ to C₆ alkyl optionally substituted with one or more substituents independently selected from halogen and alkoxy; or phenyl, wherein phenyl is optionally substituted with one or more substituents independently selected from halogen and alkoxy; and
Rₙ is hydroxyl, C₁ to C₄ alkoxy, amino or C₁ to C₆ alkyl.

In another embodiment, provided herein are Compounds having Formula (II):

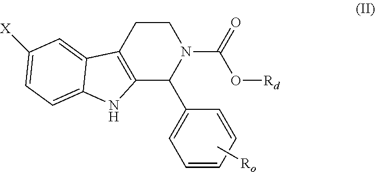

or a pharmaceutically acceptable salt, racemate or stereoisomer thereof, wherein,
X is halogen;
Rₒ is halogen, substituted or unsubstituted C₁ to C₈ alkyl or ORₐ;
Rₐ is H, C₁ to C₈ alkyl optionally substituted with one or more substituents independently selected from hydroxyl and halogen; and
R_{d} is phenyl optionally substituted with one or more alkoxy or halogen substituents.

In one embodiment, X is chloro or bromo.

In one embodiment, R_{d} is chloro or bromo.

In one embodiment, Rₒ is ORₐ.

In one embodiment, Rₐ is methyl, ethyl, propyl, isopropyl, butyl, or pentyl, each optionally substituted with one or more hydroxyl substituents.

In another embodiment, provided herein are Compounds having Formula (II):

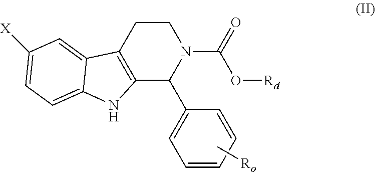

or a pharmaceutically acceptable salt, racemate or stereoisomer thereof, wherein,
X is halogen;
Rₒ is halogen, substituted or unsubstituted C₁ to C₈ alkyl or ORₐ;
Rₐ is H, or C₁ to C₈ alkyl optionally substituted with one or more substituents independently selected from hydroxyl and halogen; and
R_{d} is phenyl optionally substituted with one or more halogen substituents.

In another embodiment, provided herein are Compounds having Formula (III):

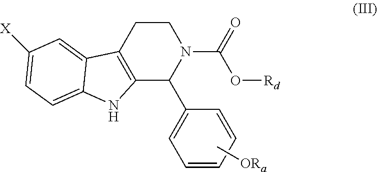

or a pharmaceutically acceptable salt, racemate or stereoisomer thereof, wherein,
X is halogen;
Rₐ is H, C₁ to C₈ alkyl optionally substituted with one or more substituents independently selected from hydroxyl and halogen; and
R_{d} is phenyl substituted with one or more halogen substituents.

In another embodiment, provided herein are Compounds having Formula (IV):

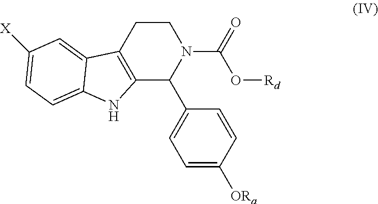

or a pharmaceutically acceptable salt, racemate or stereoisomer thereof, wherein,
X is halogen;
Rₐ is H, C₁ to C₈ alkyl optionally substituted with one or more substituents independently selected from hydroxyl and halogen; and
R_{d} is phenyl substituted with one or more halogen substituents.

In another embodiment, provided herein are Compounds having Formula (IV):

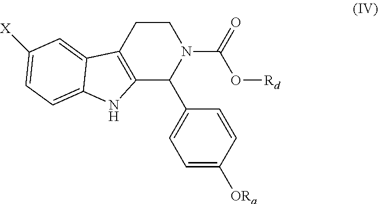

or a pharmaceutically acceptable salt, racemate or stereoisomer thereof, wherein,
X is halogen;
Rₐ is H, C₁ to C₈ alkyl optionally substituted with one or more substituents independently selected from hydroxyl and halogen; and
R_{d} is phenyl substituted on a para position with a halogen substituent.

In another embodiment, the Compounds set forth above having a formula selected from Formula (Ia), Formula (IIa), Formula (IIIa) and Formula (IVa):

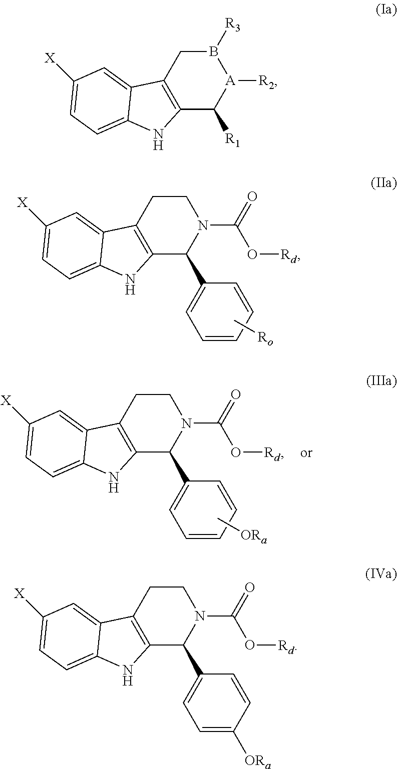

Illustrative examples of Compounds or a pharmaceutically acceptable salt, racemate or stereoisomer thereof provided herein include:

TABLE 1

| | |
|---|---|
| 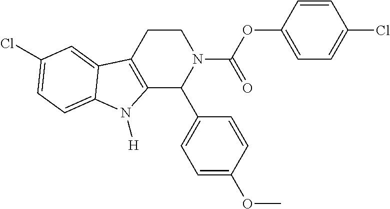 | 10 |
| 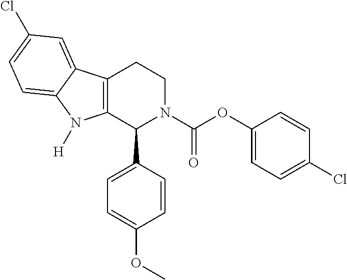 | #10 |
| 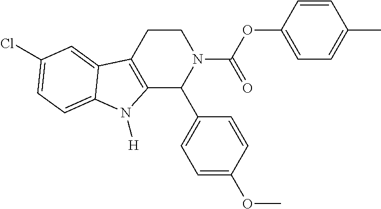 | 17 |
| 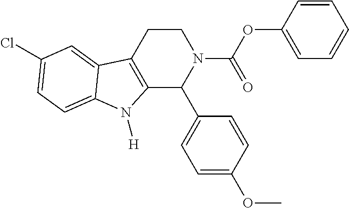 | 60 |
| 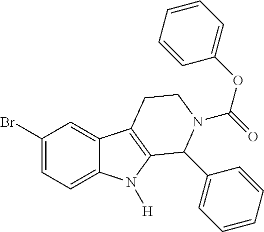 | 76 |
| 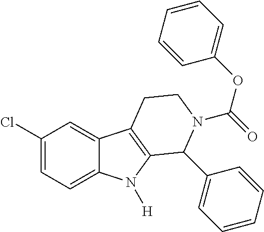 | 121 |
| 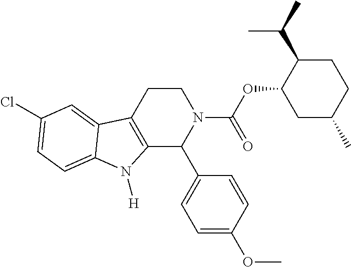 | 192 |
| 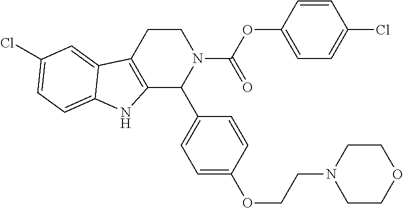 | 331 |
| 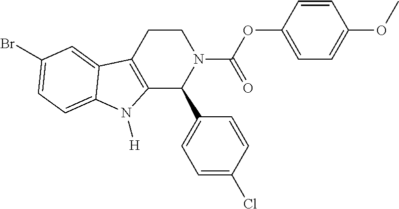 | 332 |
| 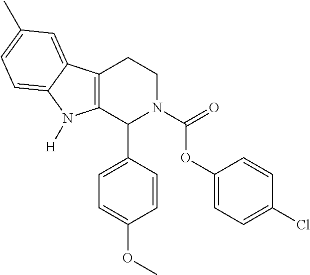 | 341 |
| 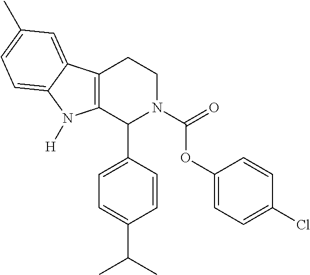 | 344 |
| 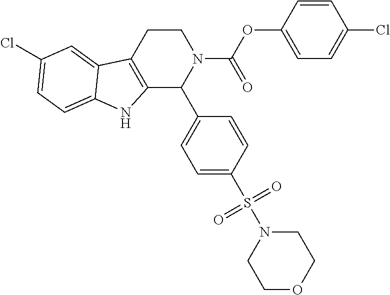 | 346 |
| 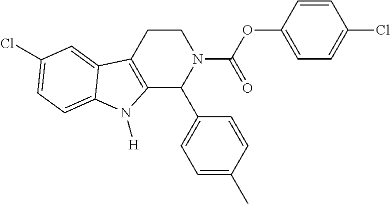 | 347 |
| 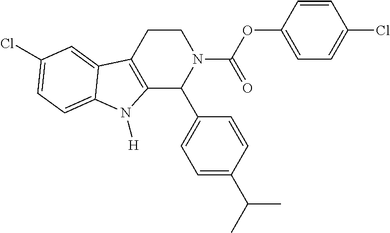 | 348 |
| 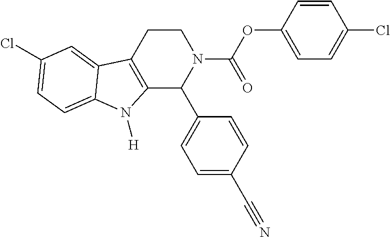 | 350 |
| 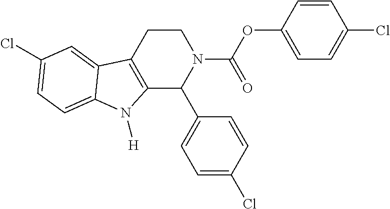 | 351 |
| 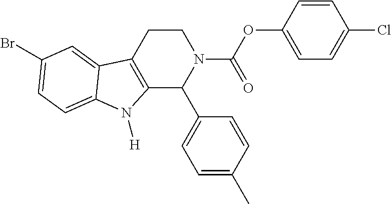 | 353 |
| 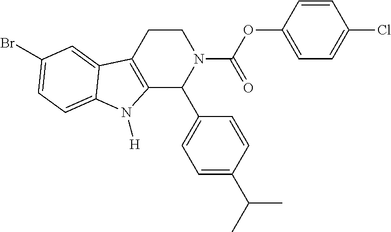 | 354 |
| 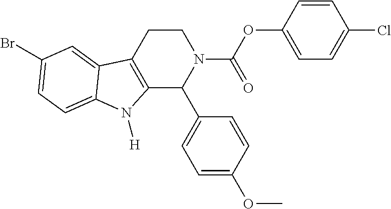 | 355 |
| 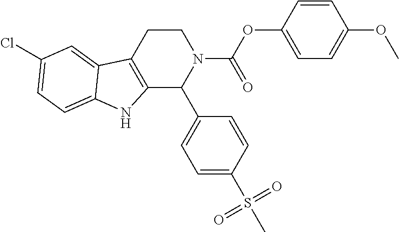 | 359 |
| 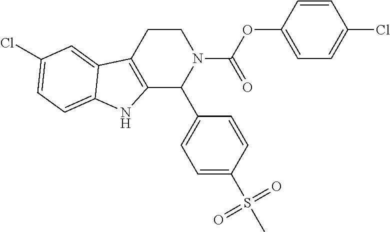 | 360 |
| 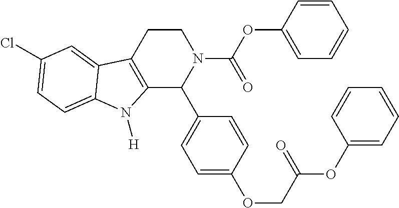 | 366 |
| 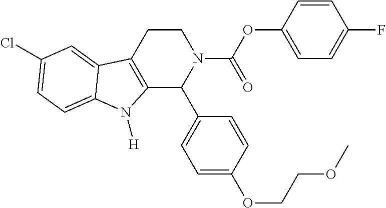 | 388 |
| 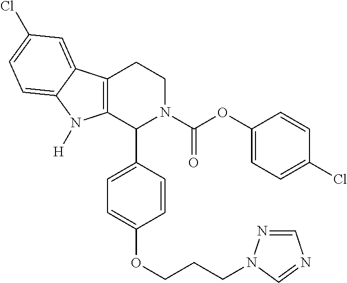 | 391 |
| 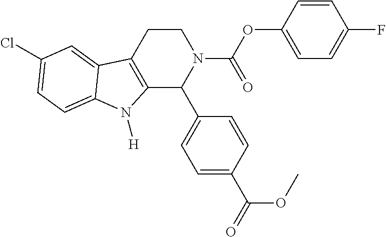 | 395 |
| 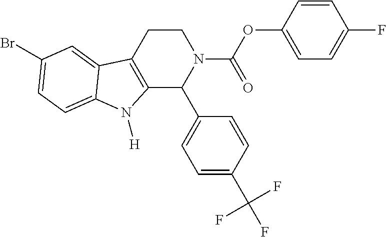 | 397 |
| 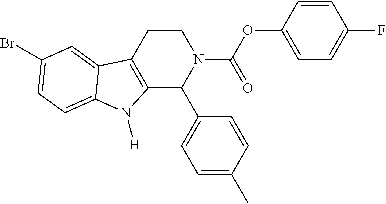 | 398 |
| 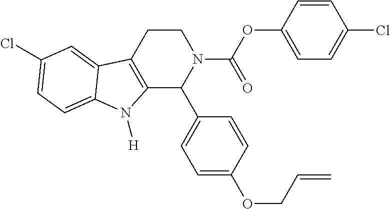 | 400 |
| 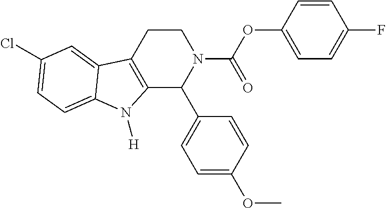 | 401 |
| 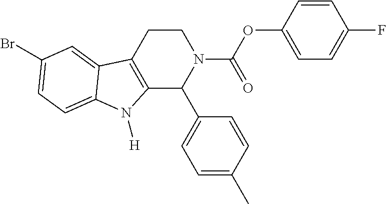 | 403 |
| 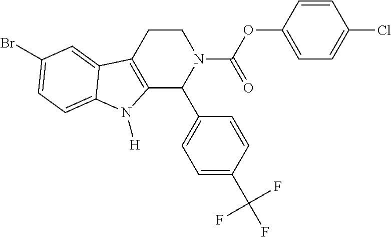 | 405 |
| 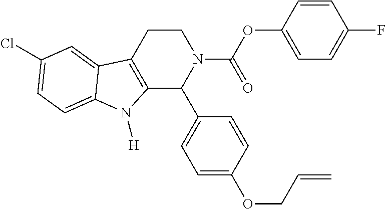 | 409 |
| 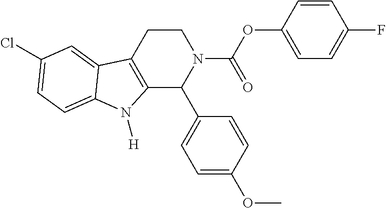 | 410 |
| 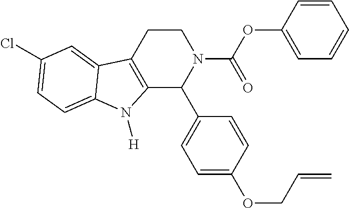 | 413 |
| 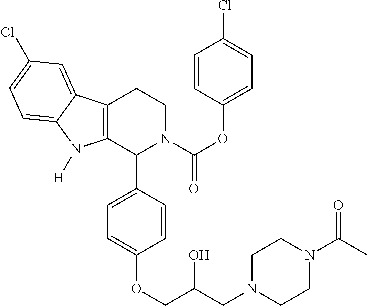 | 415 |
| 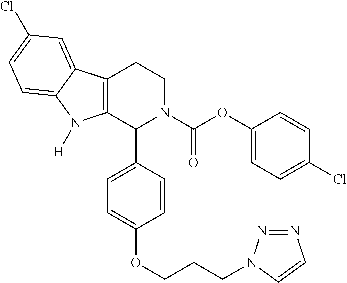 | 417 |
| 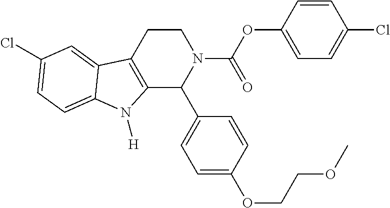 | 418 |
| 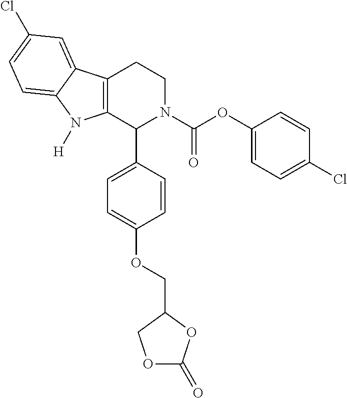 | 421 |
| 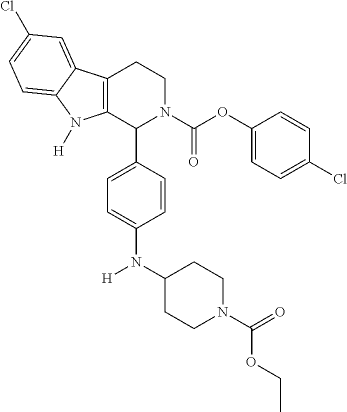 | 422 |
| 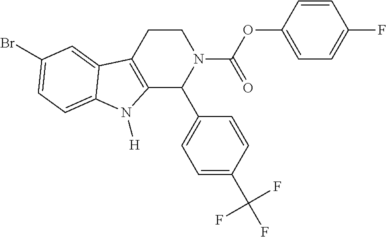 | 426 |
| 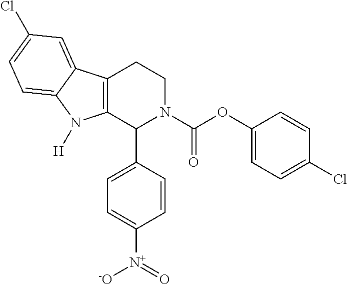 | 427 |
| 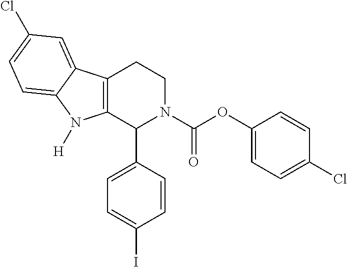 | 428 |
| 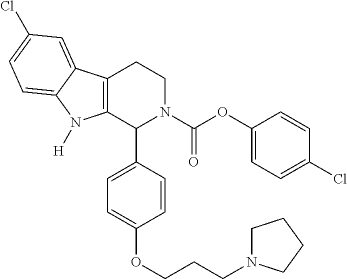 | 429 |
| 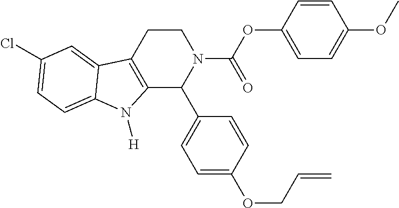 | 432 |
| 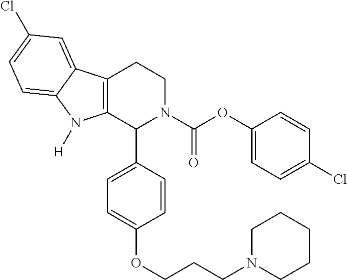 | 433 |
| 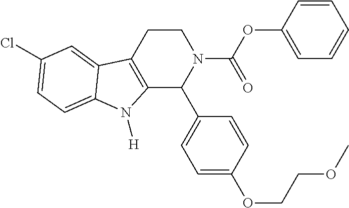 | 436 |
| 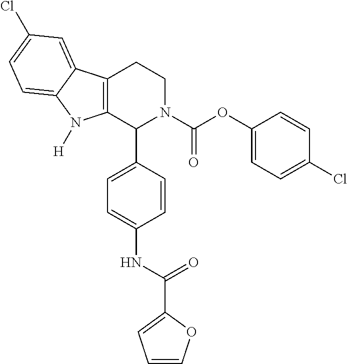 | 437 |
| 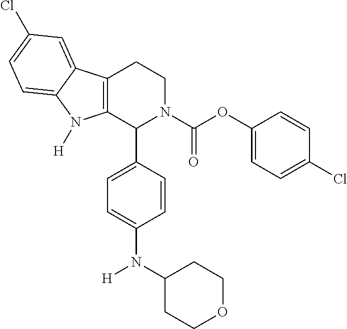 | 440 |
| 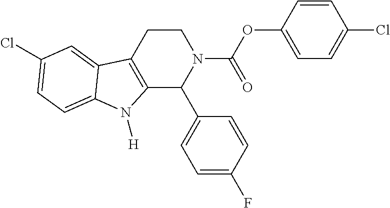 | 444 |
| 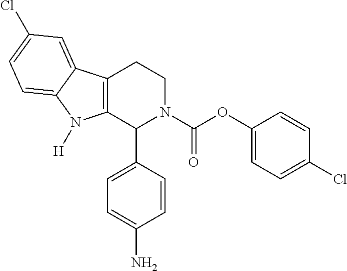 | 446 |
| 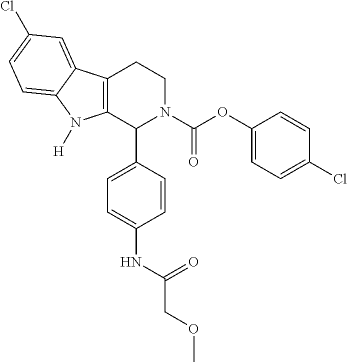 | 448 |
| 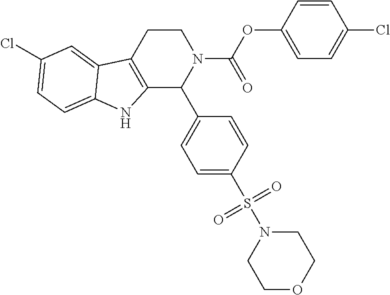 | 450 |
| 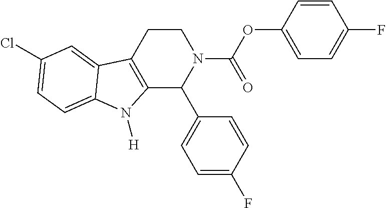 | 452 |
| 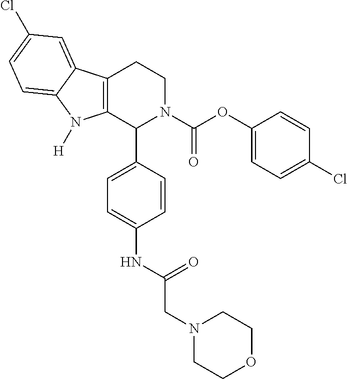 | 454 |
| 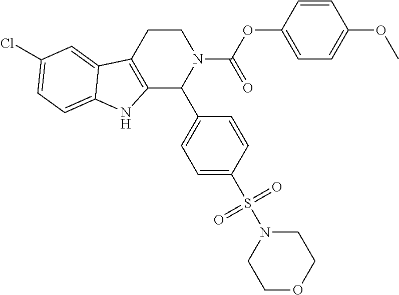 | 455 |
| 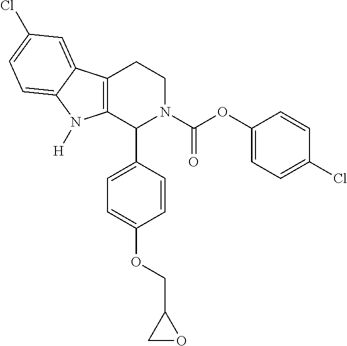 | 460 |
| 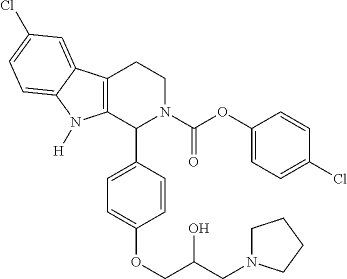 | 462 |
| 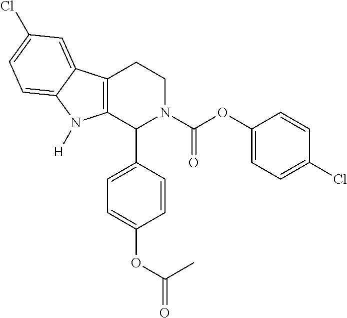 | 463 |
| 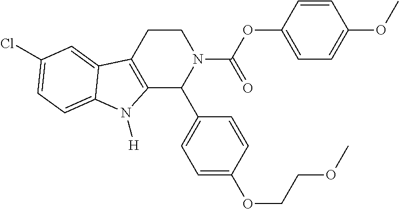 | 465 |
| 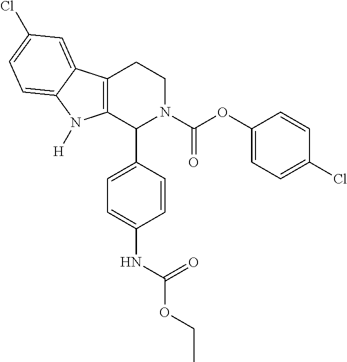 | 467 |
| 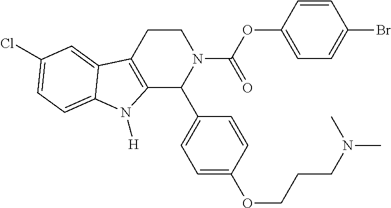 | 468 |
| 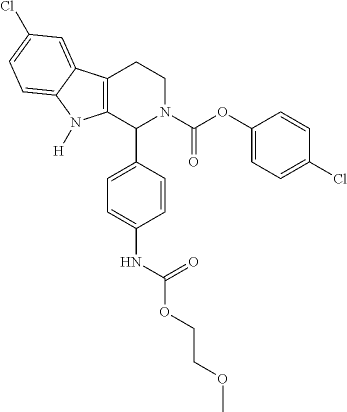 | 470 |
| 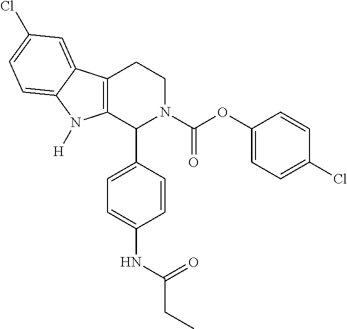 | 471 |
| 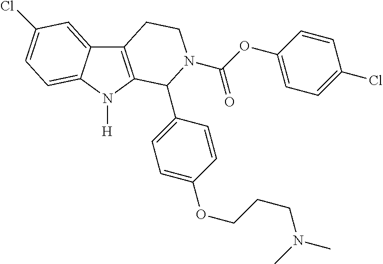 | 479 |
| 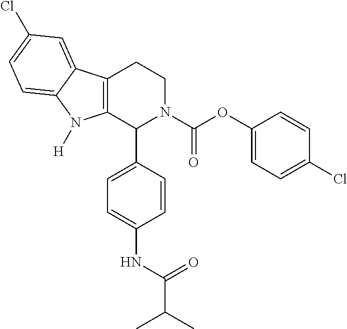 | 482 |
| 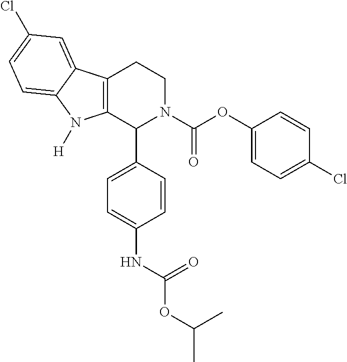 | 489 |
| 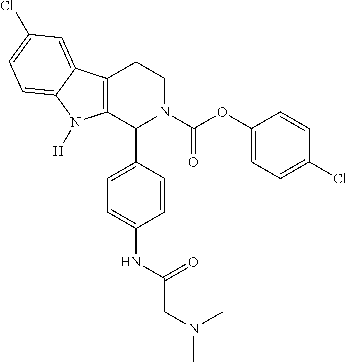 | 491 |
| 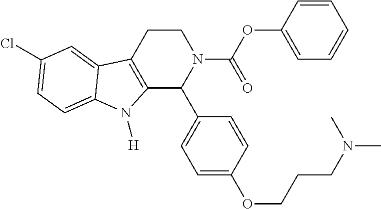 | 493 |
| 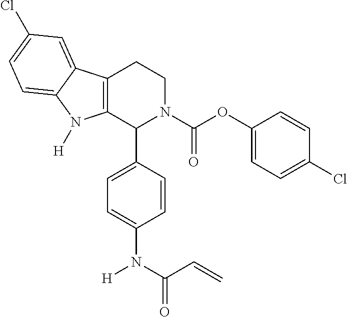 | 500 |
| 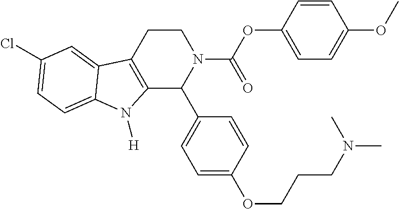 | 501 |
| 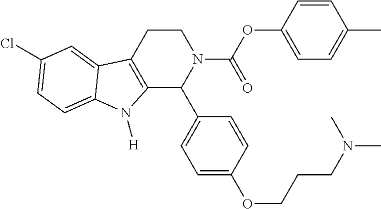 | 502 |
| 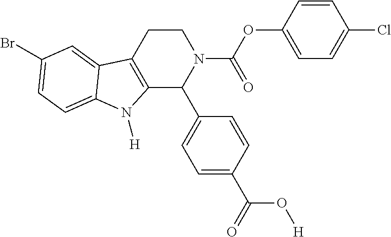 | 519 |
| 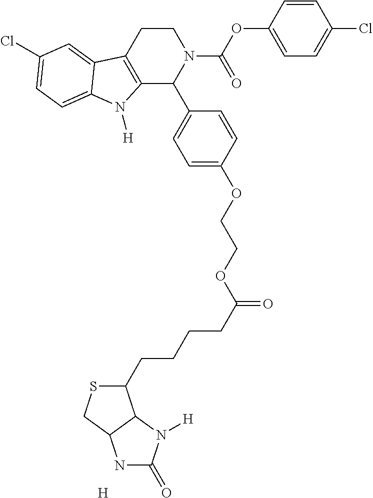 | 544 |
| 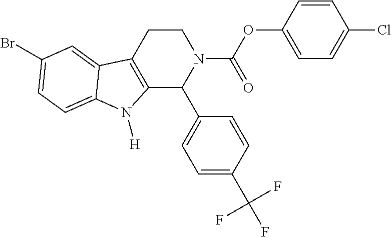 | 570 |
| 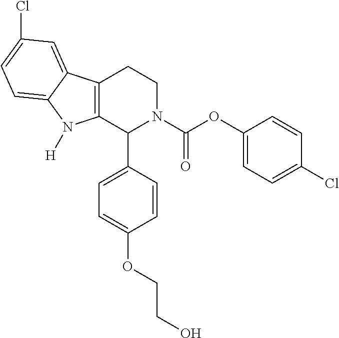 | 571 |
| 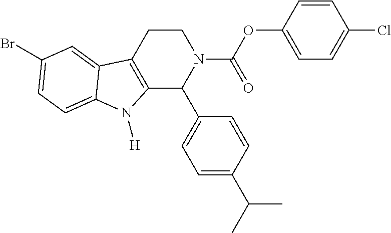 | 572 |
| 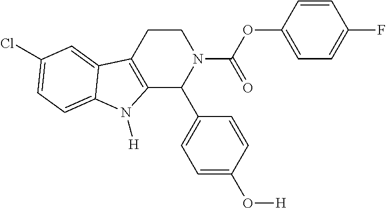 | 575 |
| 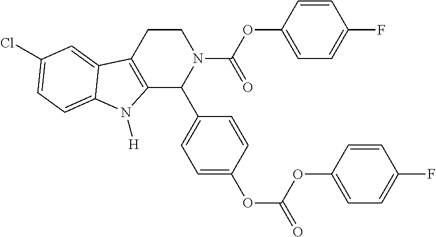 | 576 |
| 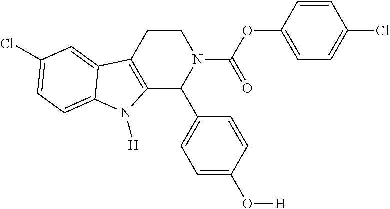 | 577 |
| 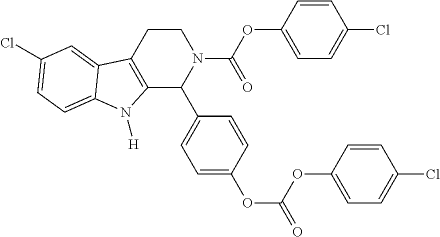 | 578 |
| 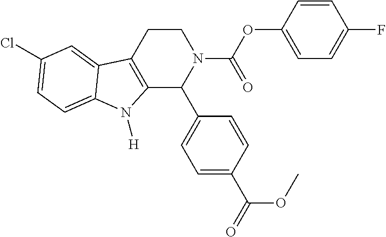 | 579 |
| 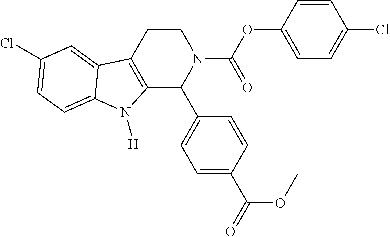 | 580 |
| 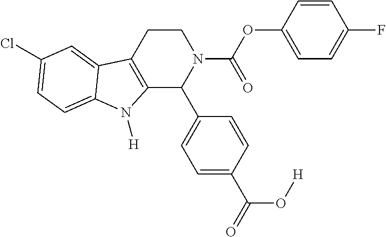 | 581 |
| 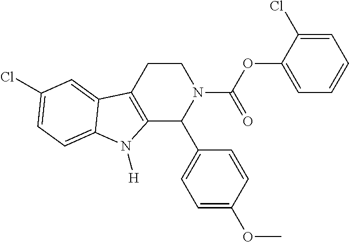 | 587 |
| 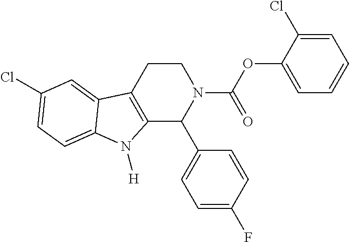 | 588 |
| 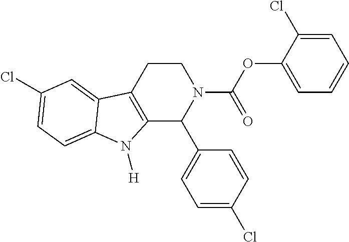 | 589 |
| 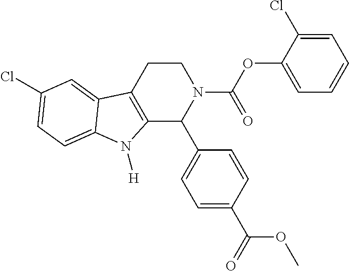 | 590 |
| 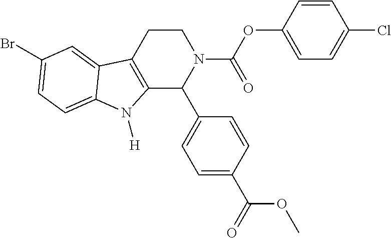 | 591 |
| 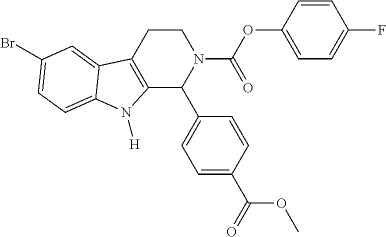 | 592 |
| 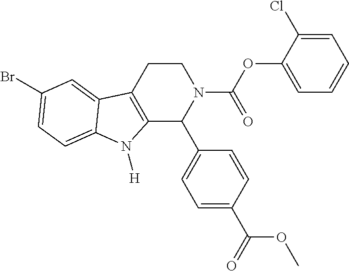 | 593 |
| 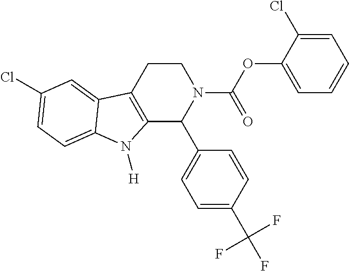 | 594 |
| 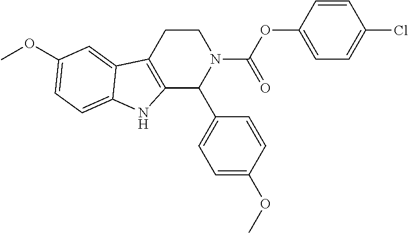 | 614 |
| 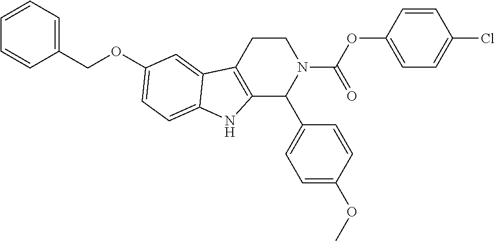 | 616 |
| 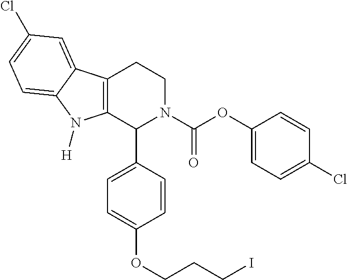 | 617 |
| 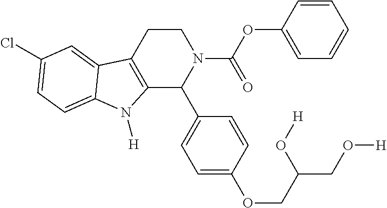 | 626 |
| 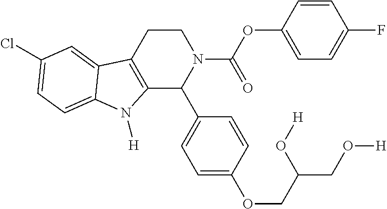 | 627 |
| 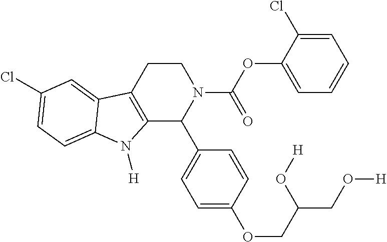 | 628 |
| 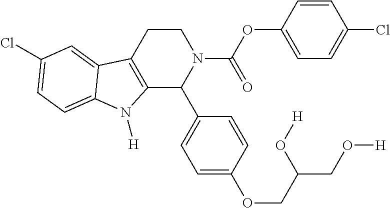 | 629 |
| 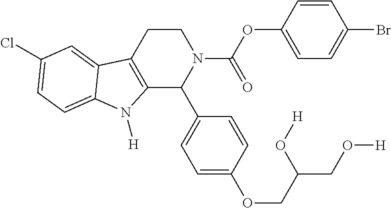 | 630 |
| 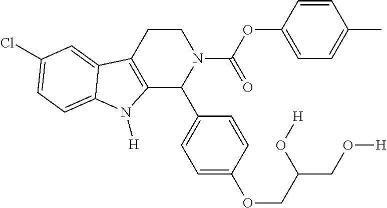 | 631 |
| 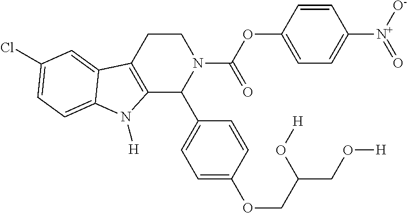 | 632 |
| 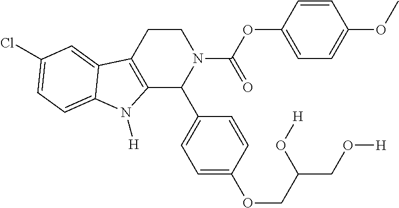 | 635 |
| 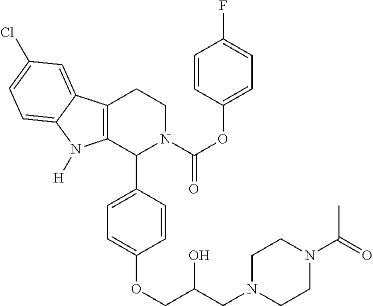 | 637 |
| 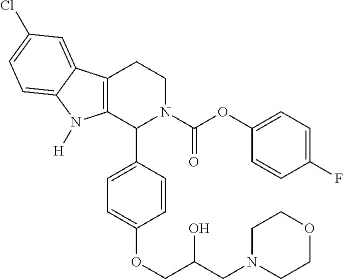 | 638 |
| 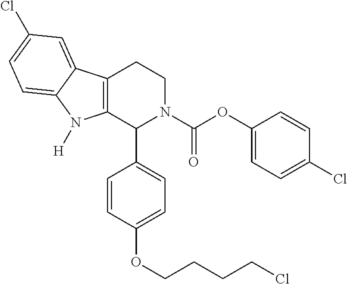 | 660 |
| 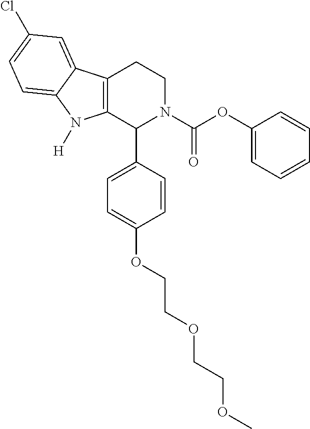 | 670 |
| 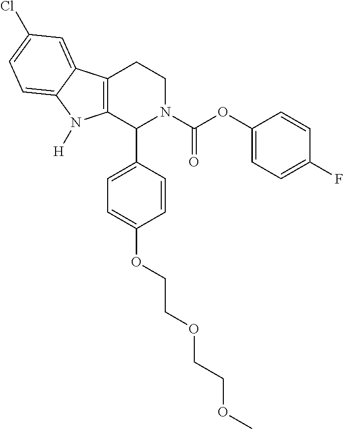 | 673 |
| 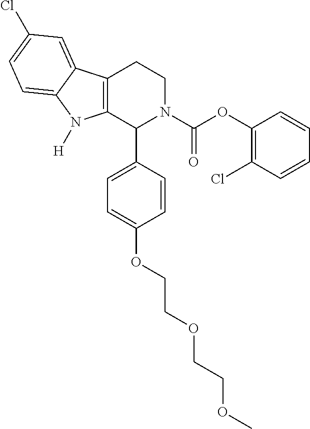 | 674 |
| 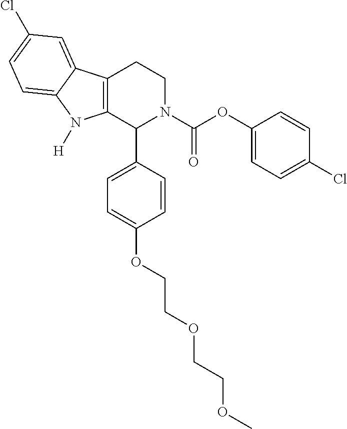 | 675 |
| 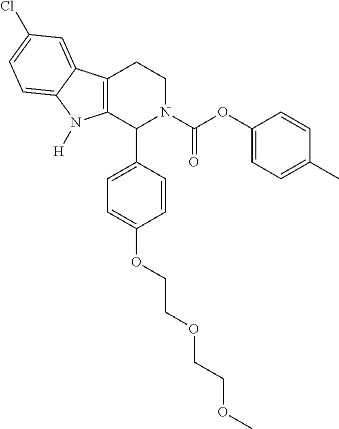 | 677 |
| 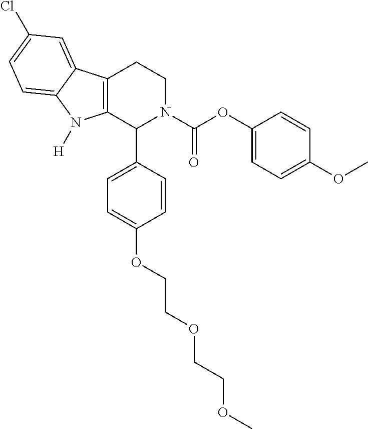 | 678 |
| 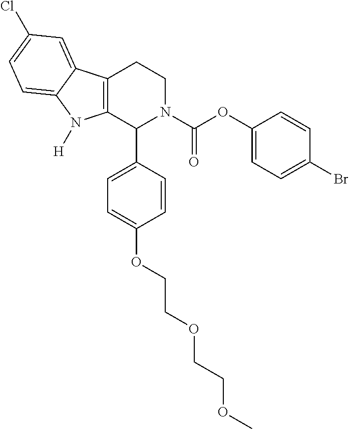 | 680 |
| 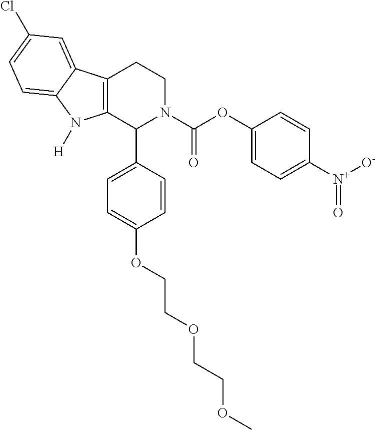 | 681 |
| 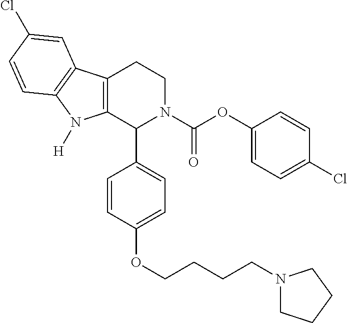 | 698 |
| 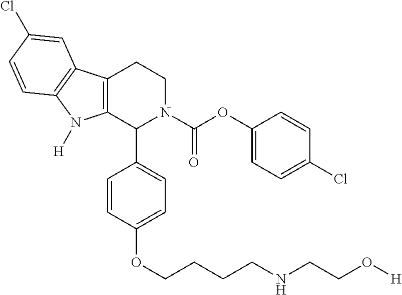 | 699 |
| 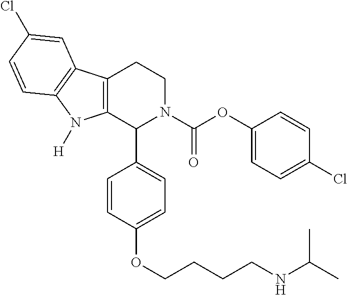 | 700 |
| 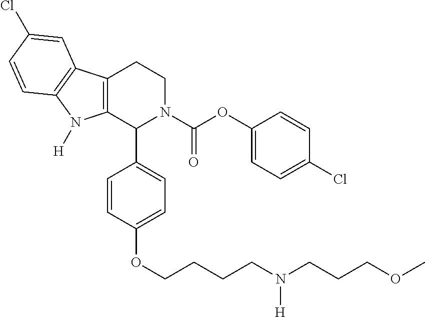 | 701 |
| 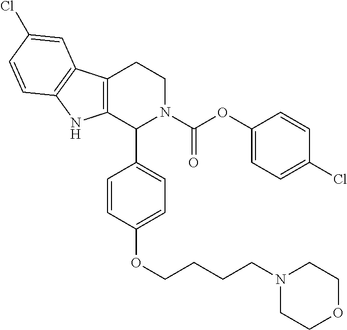 | 702 |
| 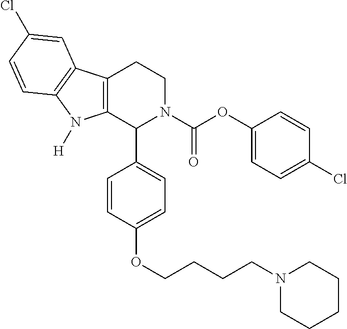 | 703 |
| 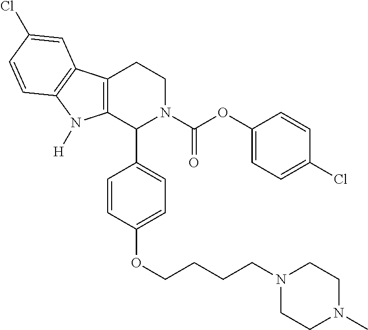 | 704 |
| 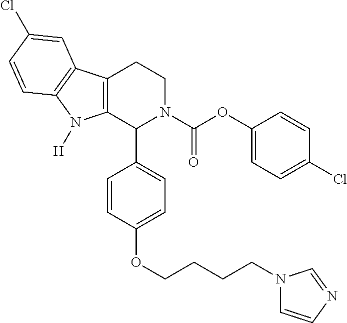 | 705 |
| 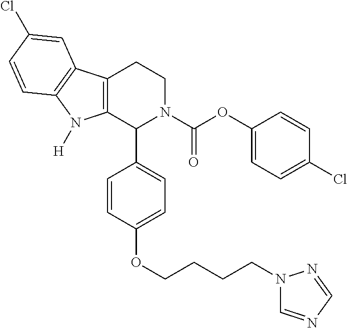 | 706 |
| 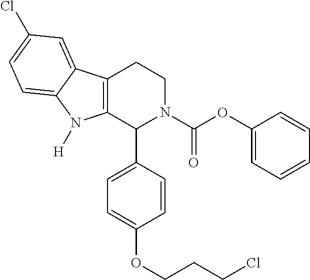 | 710 |
| 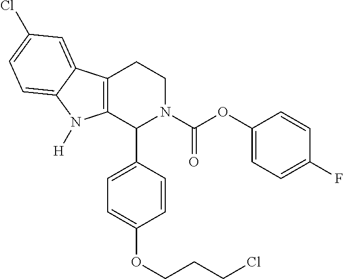 | 712 |
| 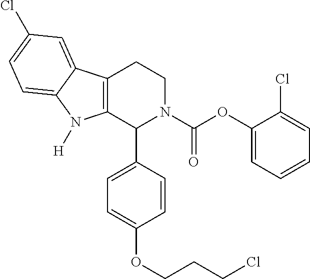 | 713 |
| 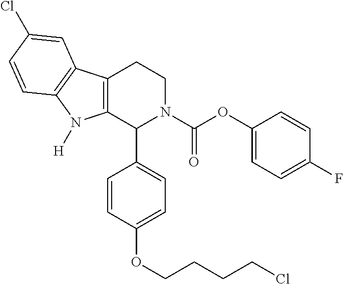 | 719 |
| 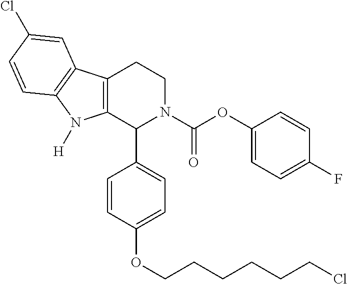 | 723 |
| 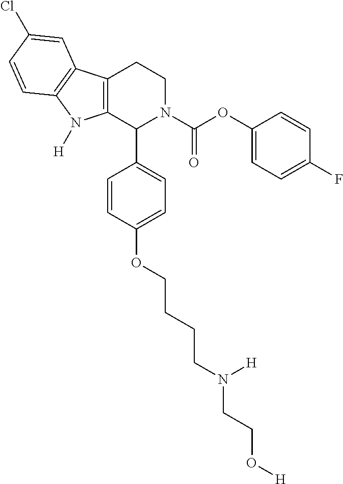 | 735 |
| 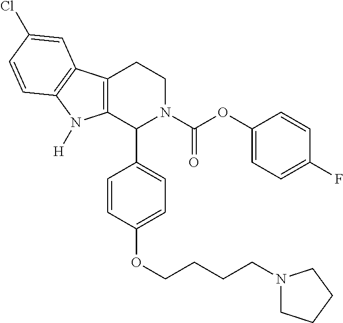 | 736 |
| 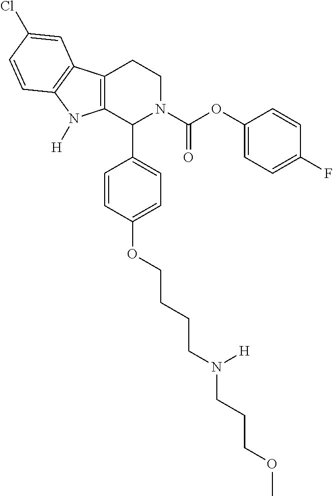 | 737 |
| 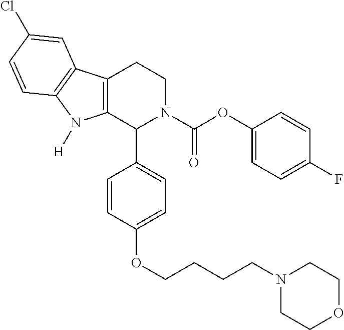 | 738 |
| 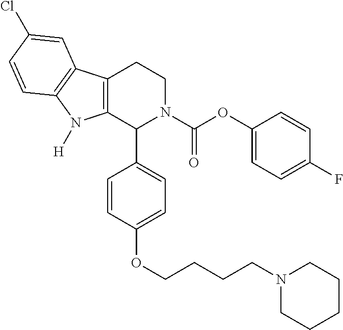 | 739 |
| 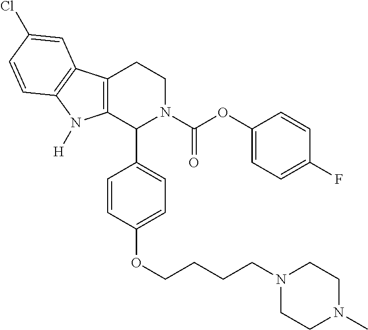 | 740 |
| 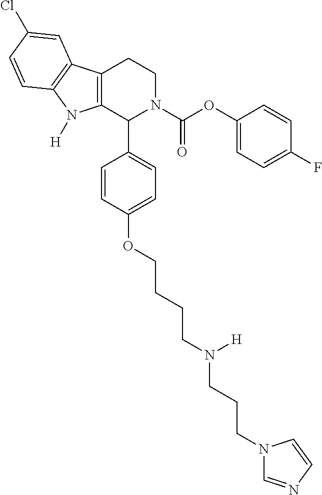 | 741 |
| 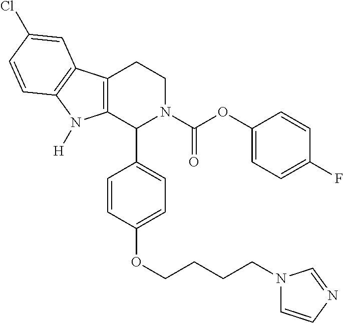 | 742 |
| 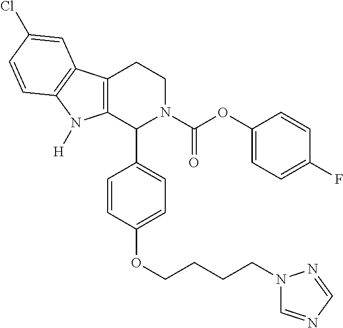 | 743 |
| 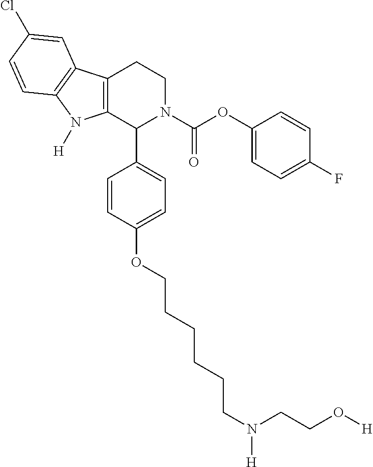 | 772 |
| 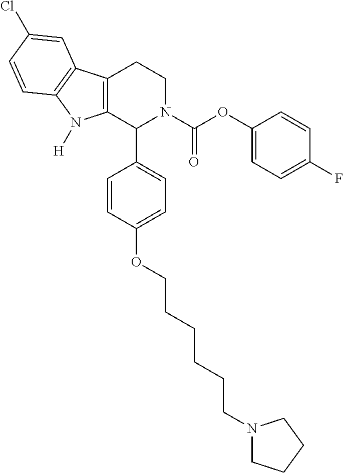 | 773 |
| 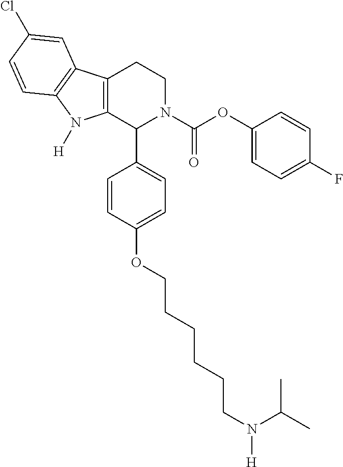 | 774 |
| 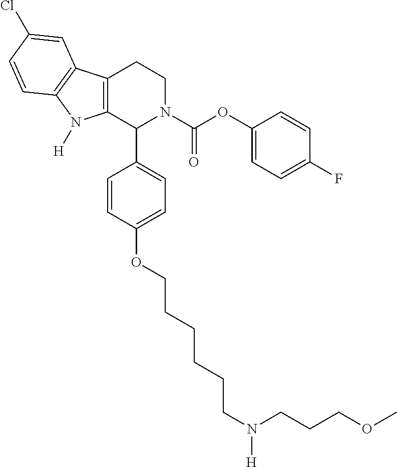 | 775 |
| 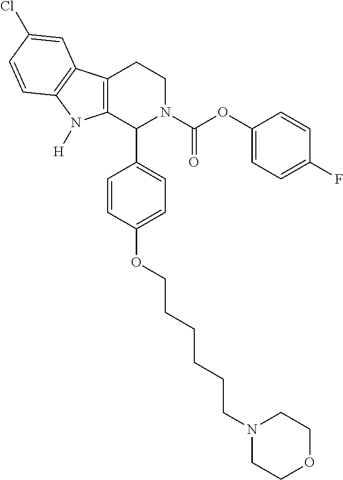 | 776 |
| 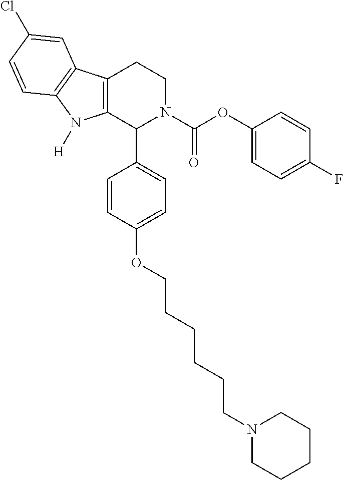 | 777 |
| 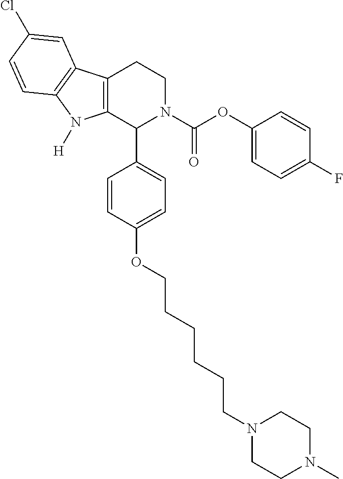 | 778 |
| 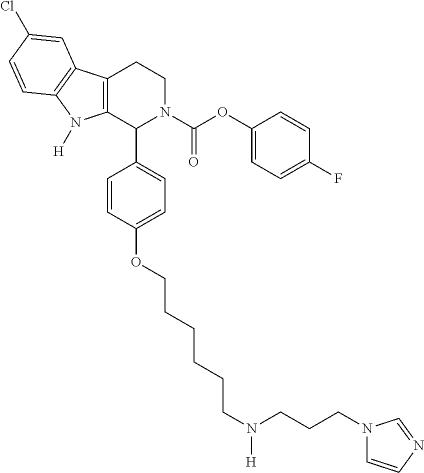 | 779 |
| 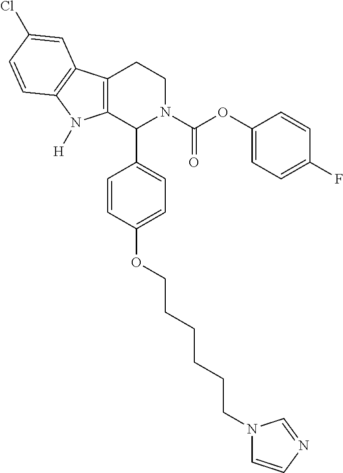 | 780 |
| 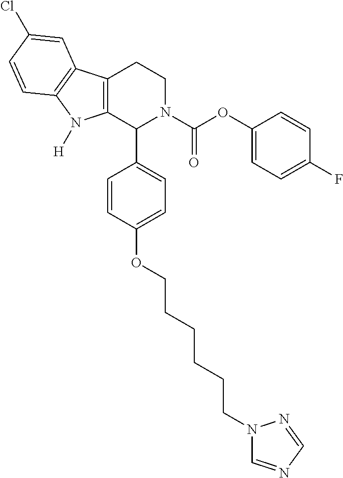 | 781 |
| 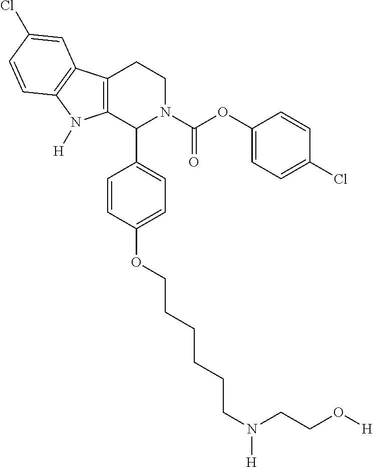 | 782 |
| 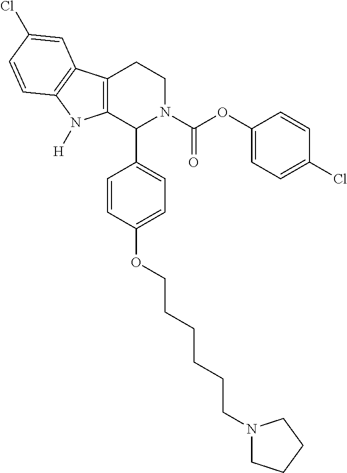 | 783 |
| 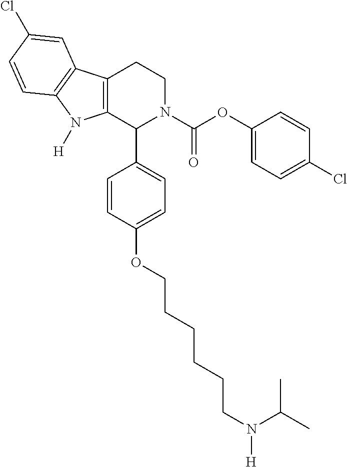 | 784 |
| 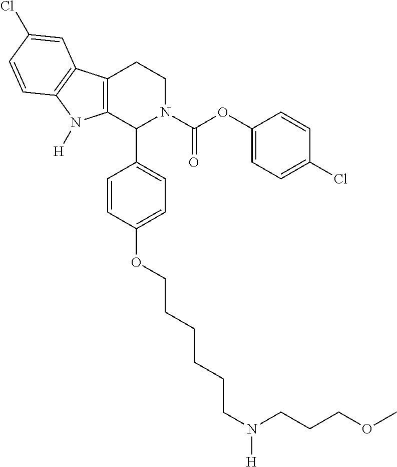 | 785 |
| 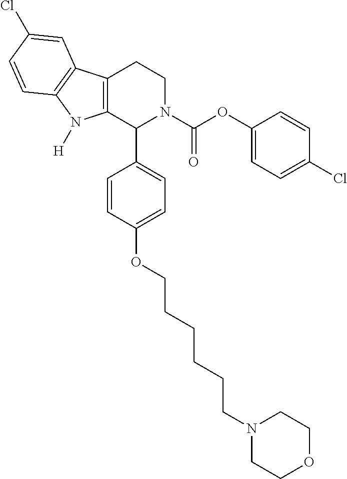 | 786 |
| 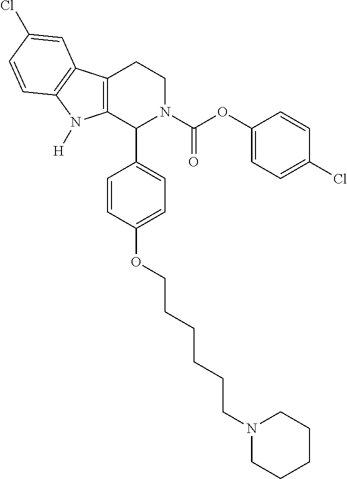 | 787 |
| 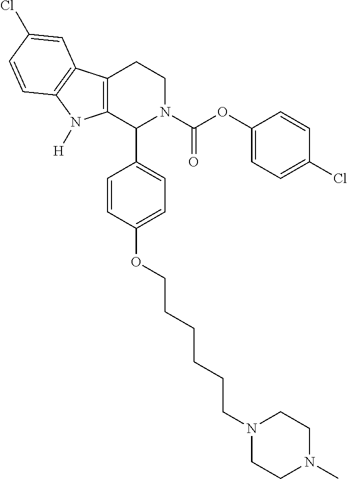 | 788 |
| 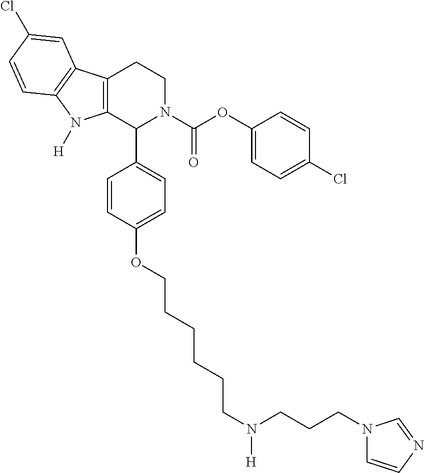 | 789 |
| 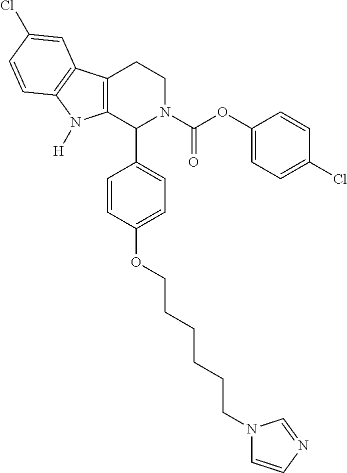 | 790 |
| 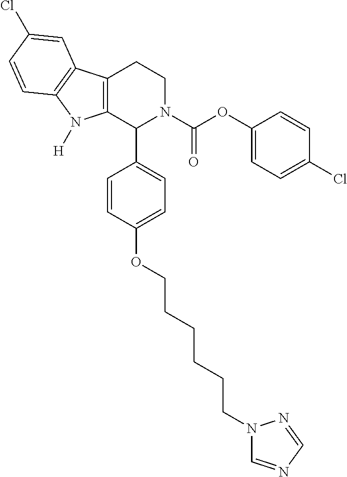 | 791 |
| 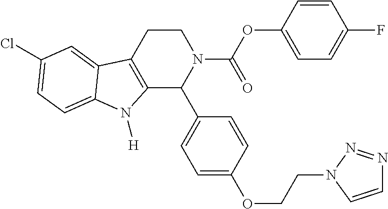 | 833 |
| 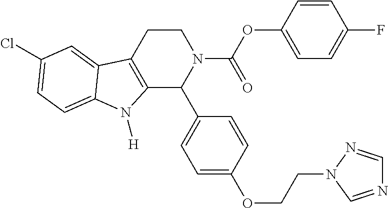 | 834 |
| 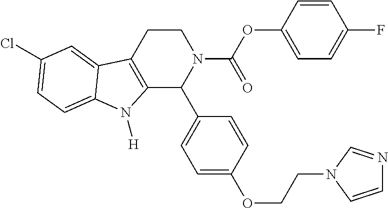 | 835 |
| 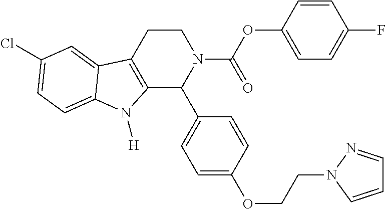 | 836 |
| 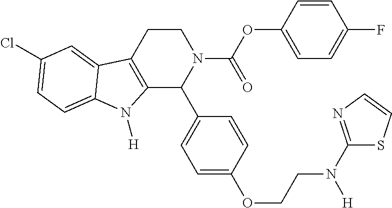 | 837 |
| 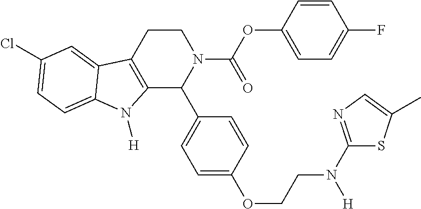 | 838 |
| 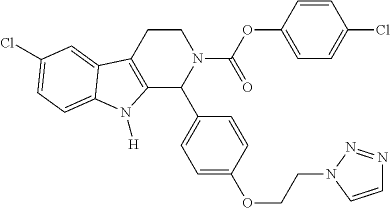 | 839 |
| 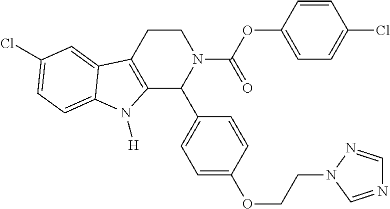 | 840 |
| 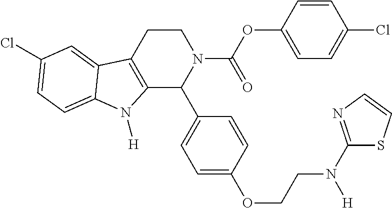 | 841 |
| 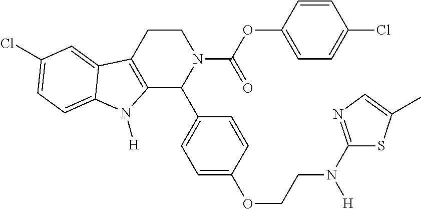 | 842 |
| 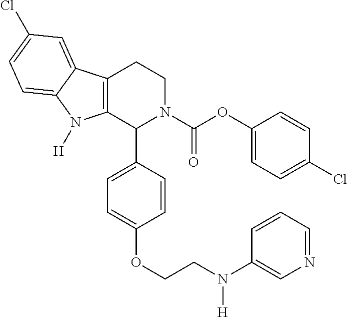 | 843 |
| 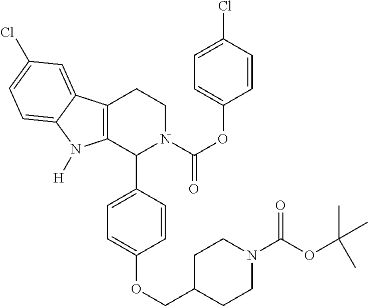 | 845 |
| 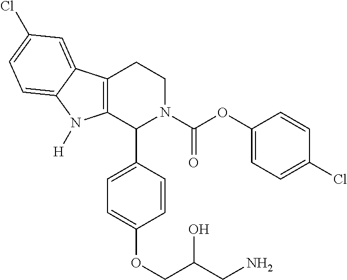 | 846 |
| 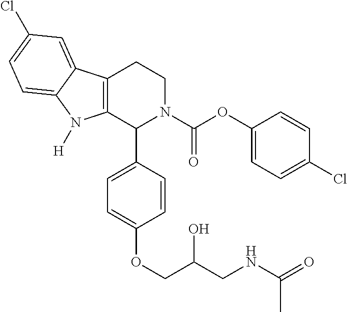 | 847 |
| 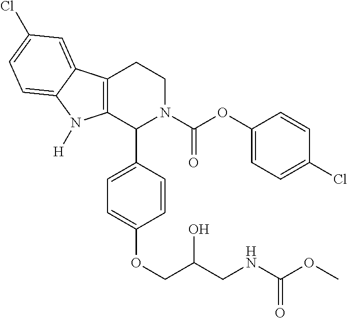 | 848 |
| 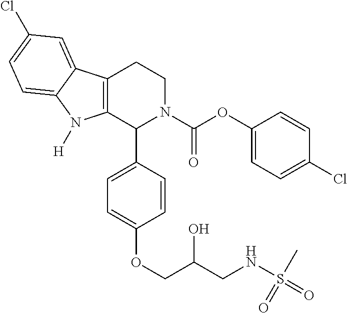 | 849 |
| 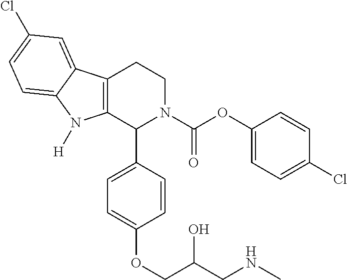 | 850 |
| 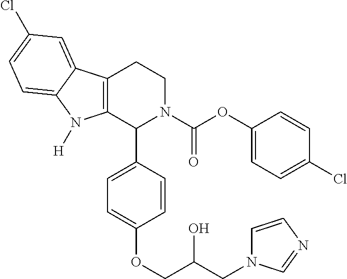 | 867 |
| 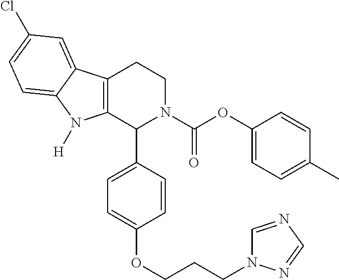 | 882 |
| 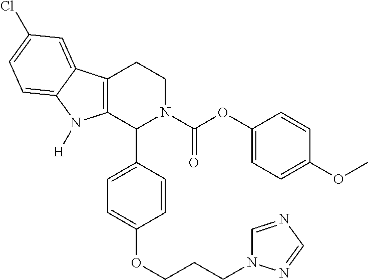 | 888 |
| 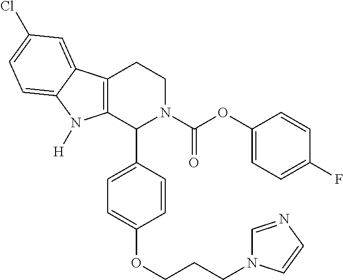 | 889 |
| 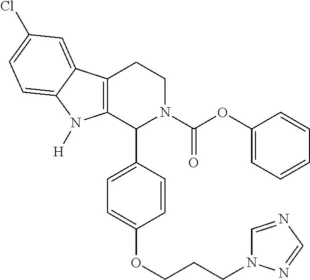 | 891 |
| 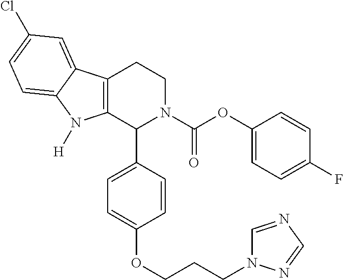 | 892 |
| 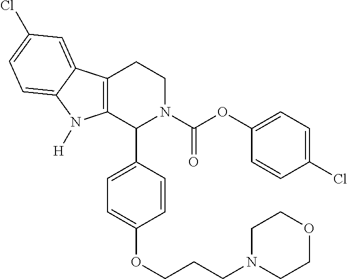 | 894 |
| 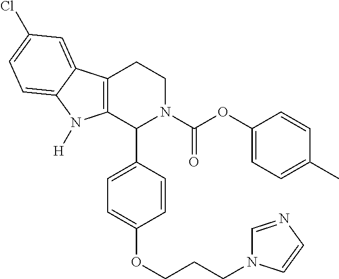 | 900 |
| 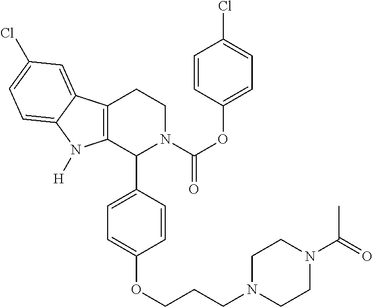 | 903 |
| 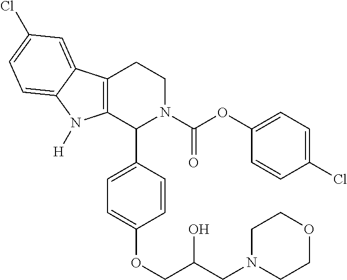 | 904 |
| 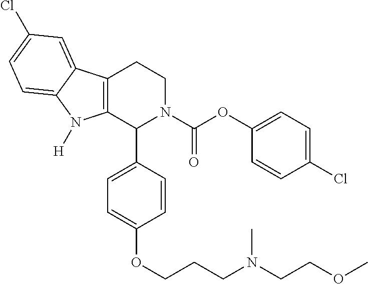 | 908 |
| 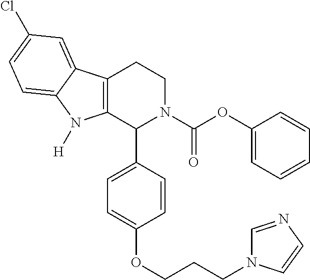 | 911 |
| 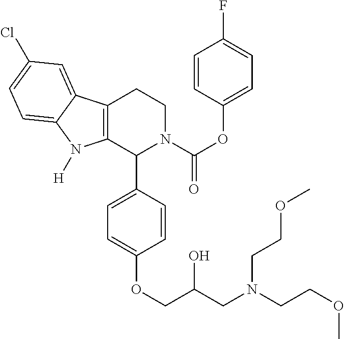 | 913 |
| 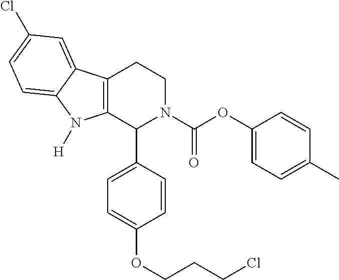 | 915 |
| 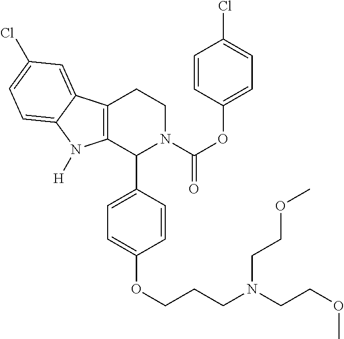 | 916 |
| 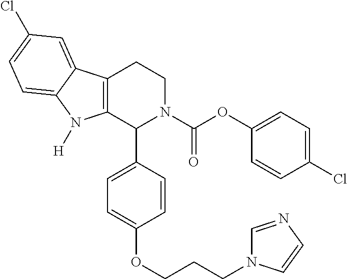 | 917 |
| 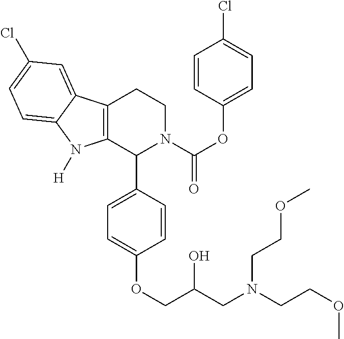 | 918 |
| 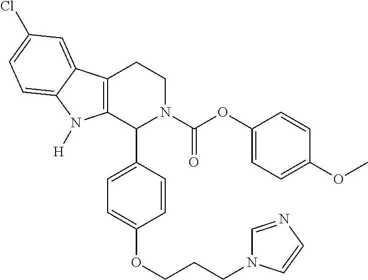 | 920 |
| 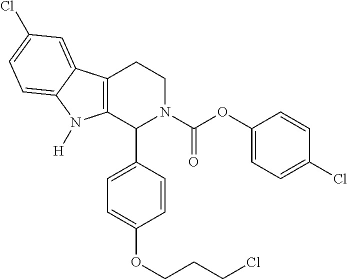 | 921 |
| 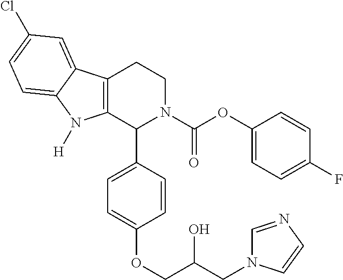 | 922 |
| 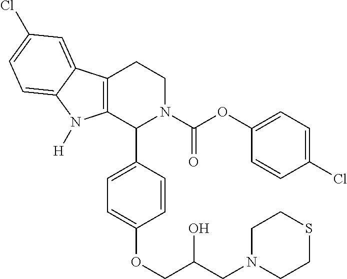 | 923 |
| 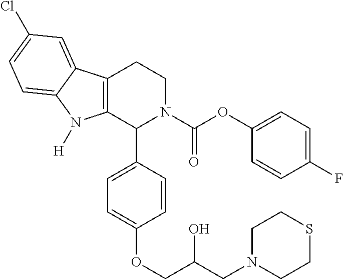 | 925 |
| 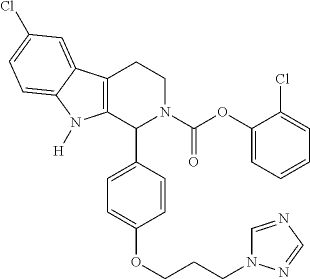 | 926 |
| 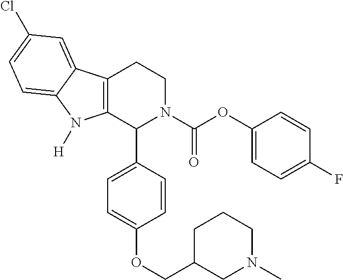 | 932 |
| 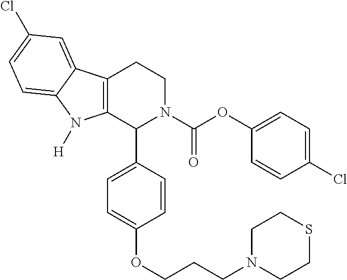 | 933 |
| 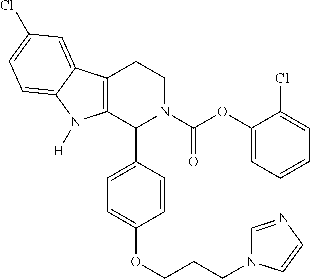 | 934 |
| 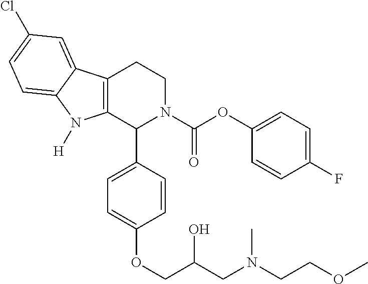 | 936 |
| 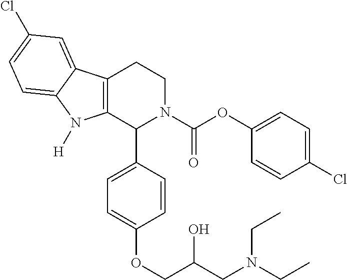 | 938 |
| 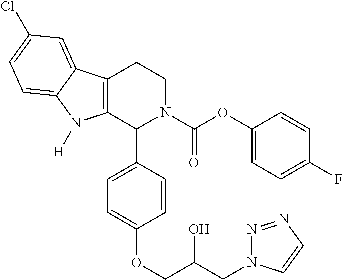 | 941 |
| 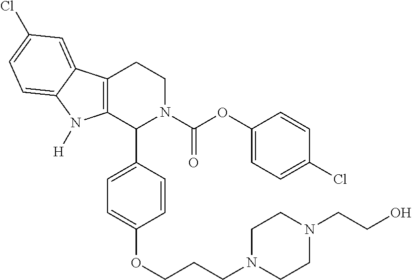 | 942 |
| 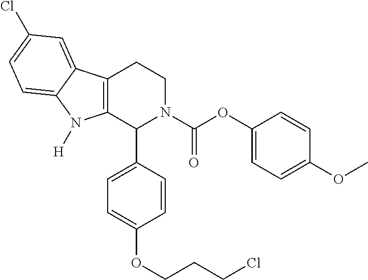 | 944 |
| 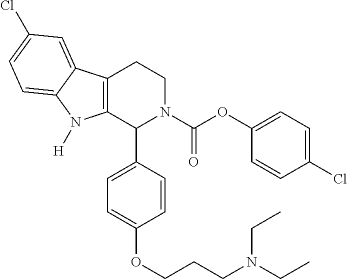 | 946 |
| 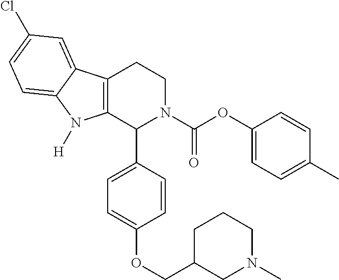 | 951 |
| 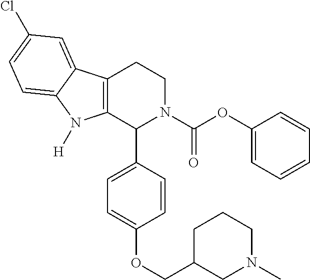 | 952 |
| 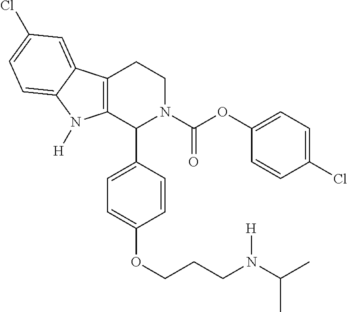 | 953 |
| 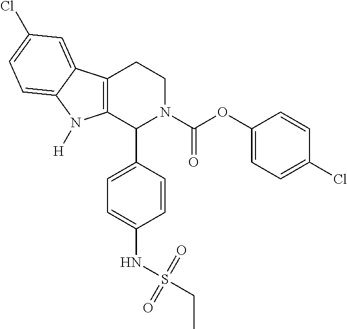 | 958 |
| 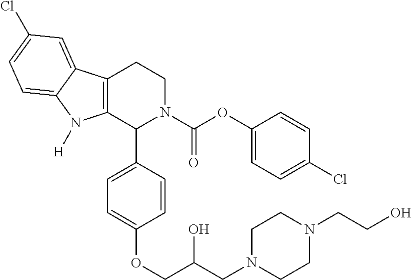 | 960 |
| 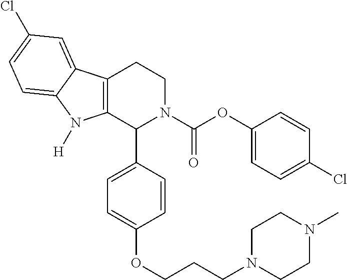 | 961 |
| 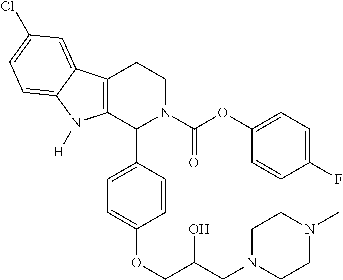 | 963 |
| 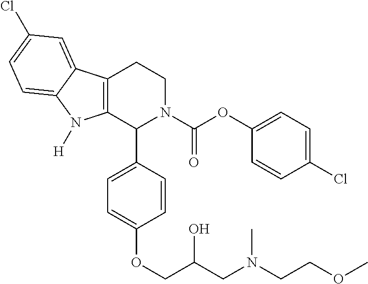 | 964 |
| 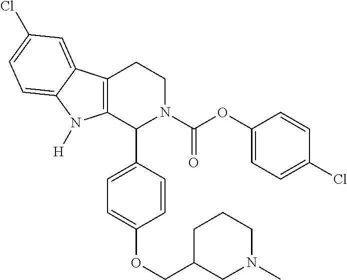 | 966 |
| 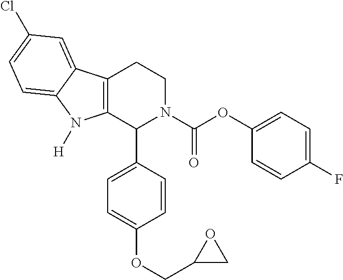 | 967 |
| 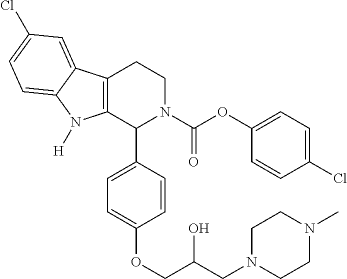 | 970 |
| 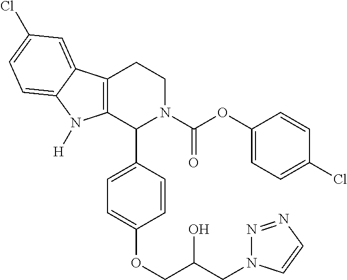 | 973 |
| 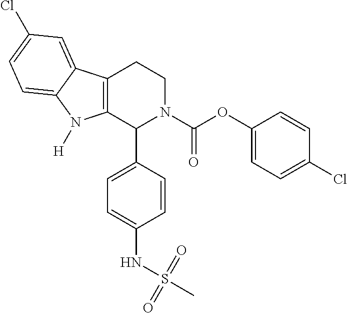 | 974 |
| 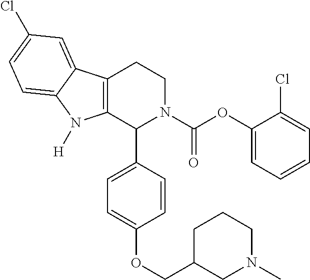 | 976 |
| 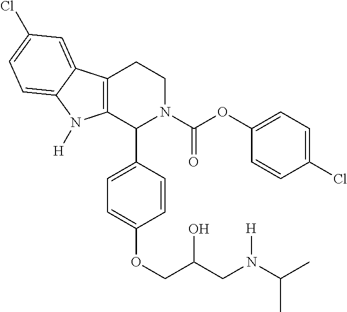 | 977 |
| 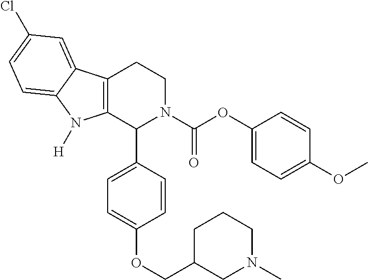 | 981 |
| 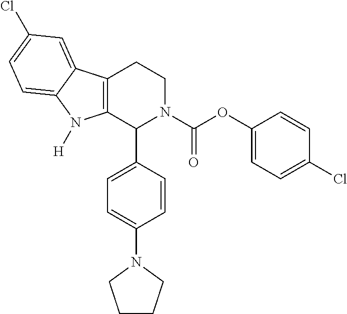 | 984 |
| 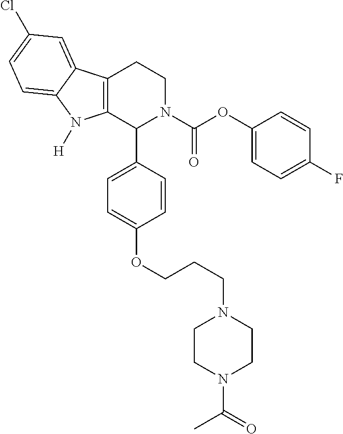 | 988 |
| 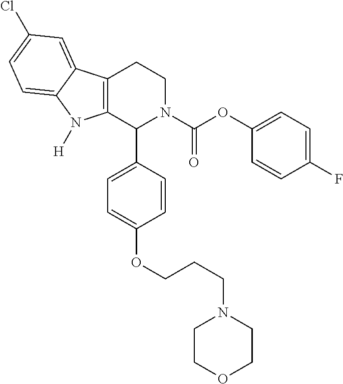 | 989 |
| 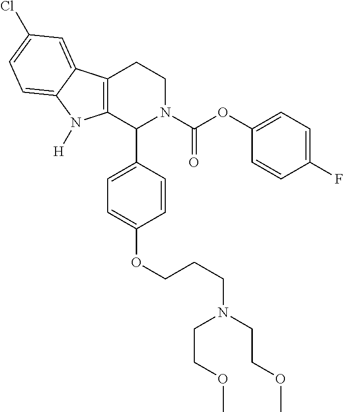 | 990 |
| 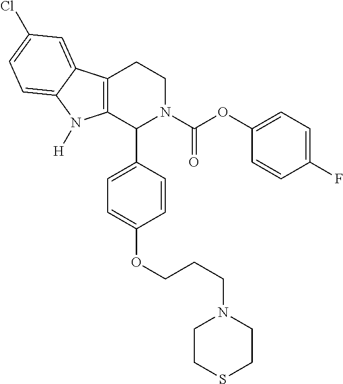 | 991 |
| 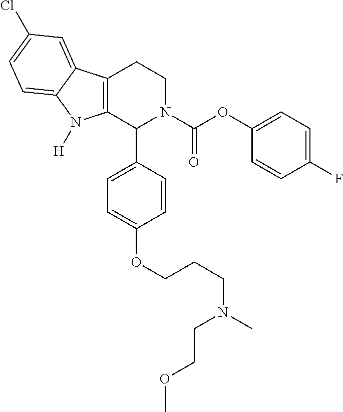 | 992 |
| 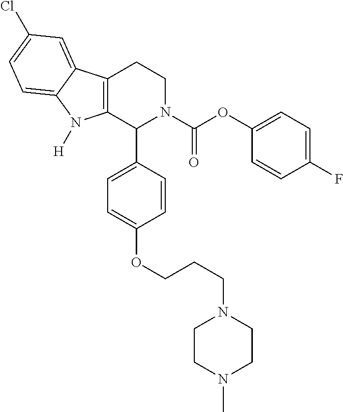 | 993 |
| 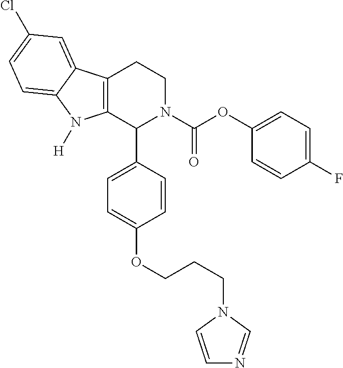 | 994 |
| 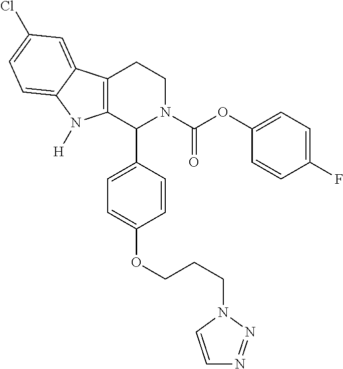 | 995 |
| 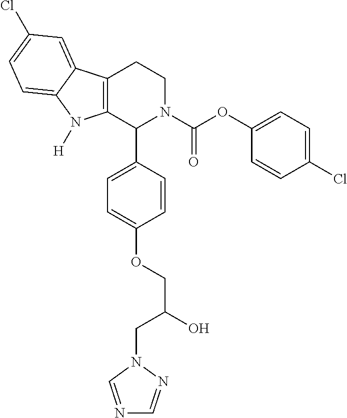 | 996 |
| 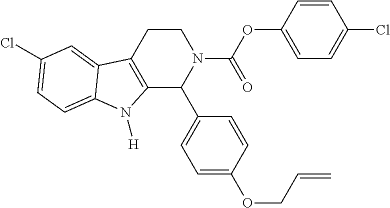 | 999 |
| 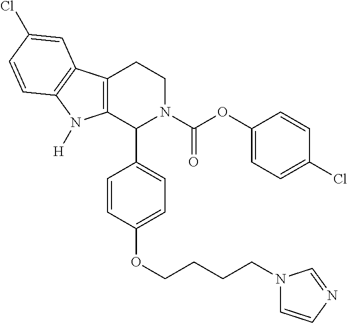 | 1001 |
| 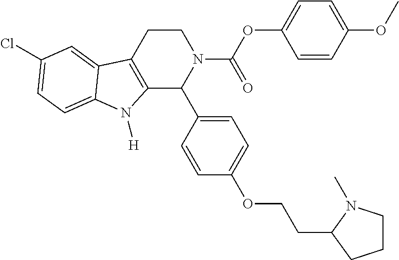 | 1005 |
| 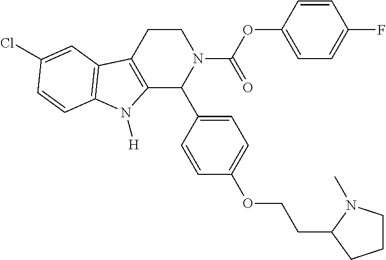 | 1008 |
| 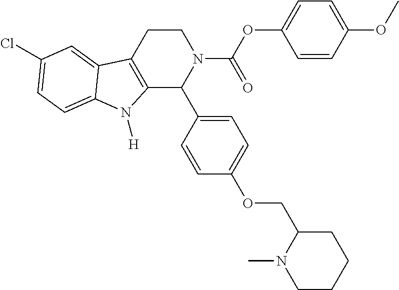 | 1009 |
| 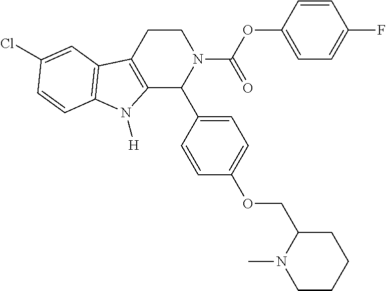 | 1011 |
| 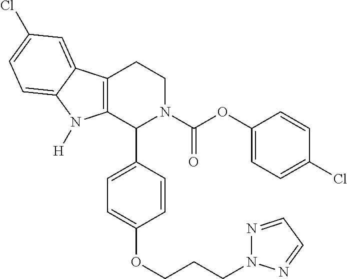 | 1016 |
| 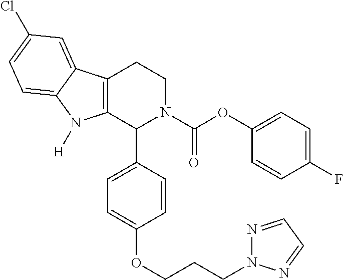 | 1017 |
| 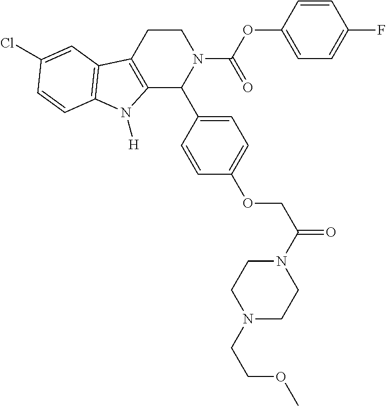 | 1021 |
| 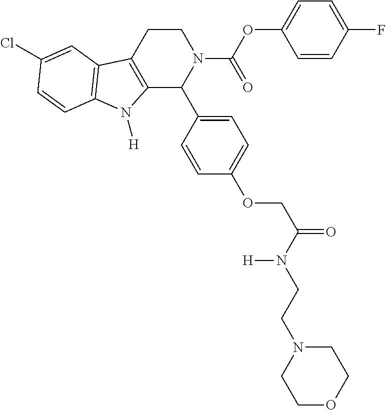 | 1022 |
| 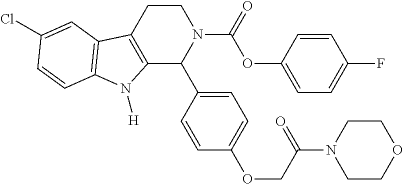 | 1023 |
| 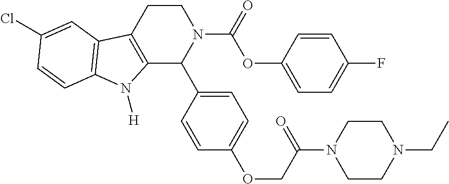 | 1024 |
| 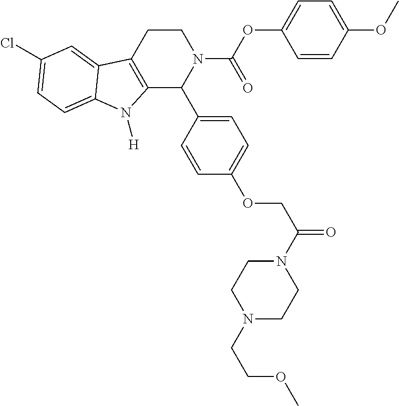 | 1025 |
| 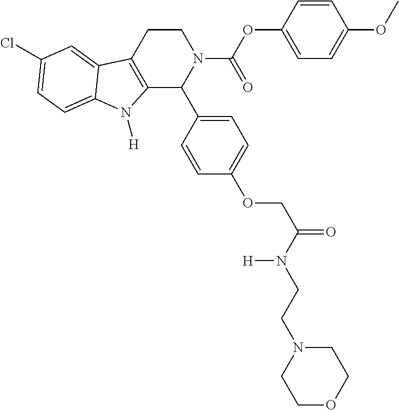 | 1026 |
| 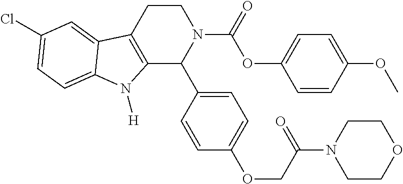 | 1027 |
| 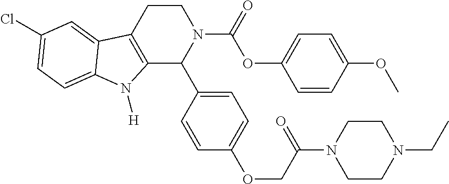 | 1028 |
| 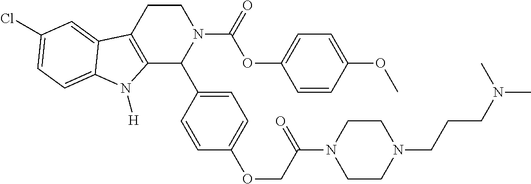 | 1029 |
| 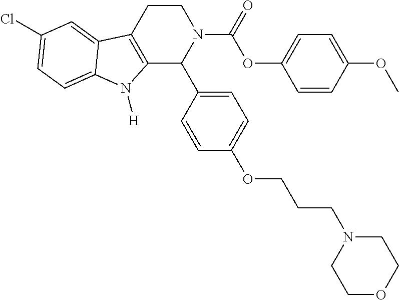 | 1030 |
| 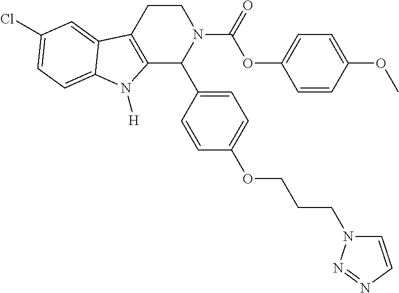 | 1031 |
| 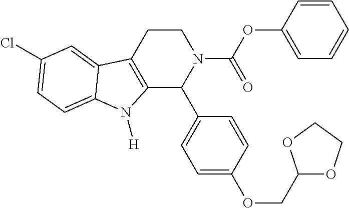 | 1050 |
| 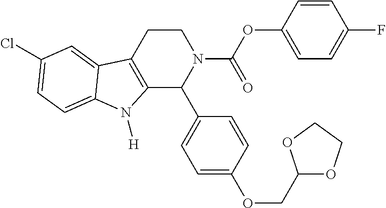 | 1051 |
| 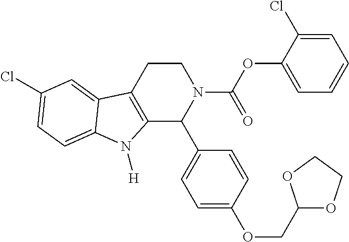 | 1052 |
| 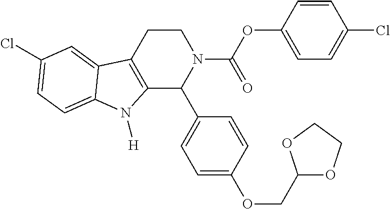 | 1053 |
| 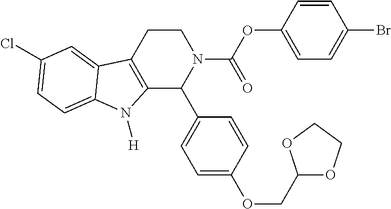 | 1054 |
| 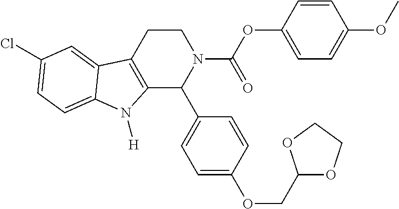 | 1055 |
| 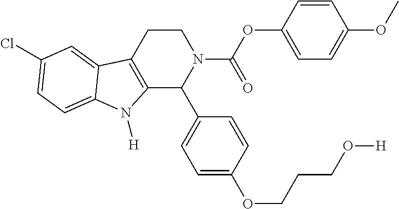 | 1058 |
| 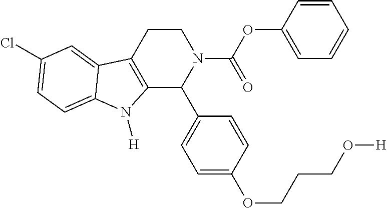 | 1062 |
| 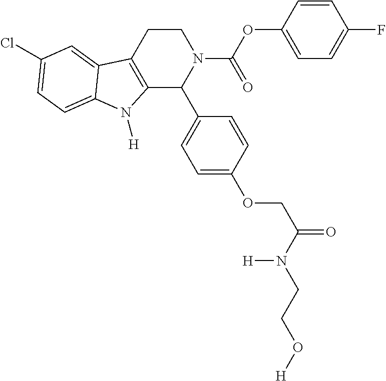 | 1063 |
| 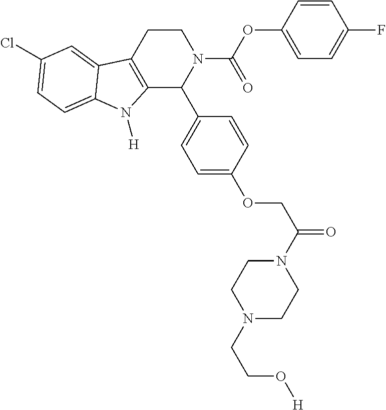 | 1064 |
| 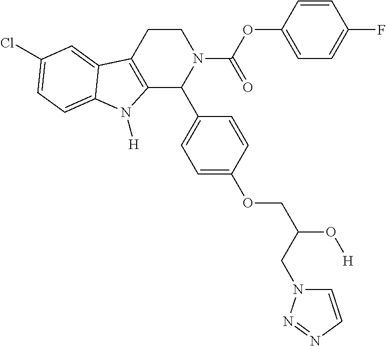 | 1066 |
| 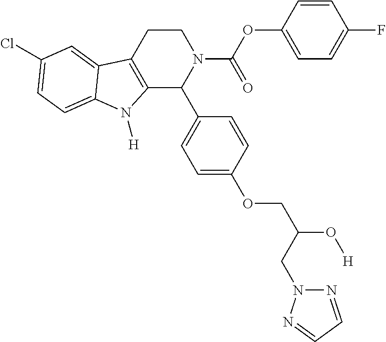 | 1067 |
| 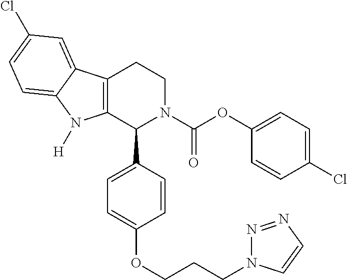 | 1068 |
| 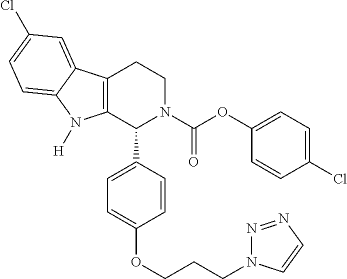 | 1069 |
| 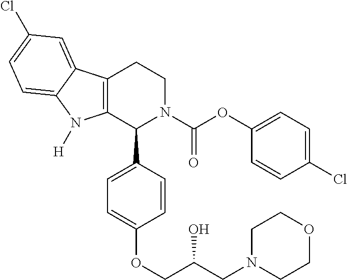 | 1070 |
| 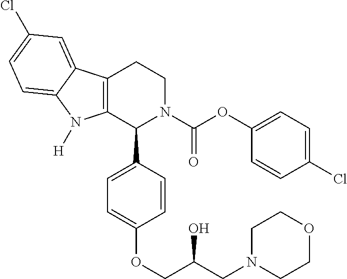 | 1071 |
| 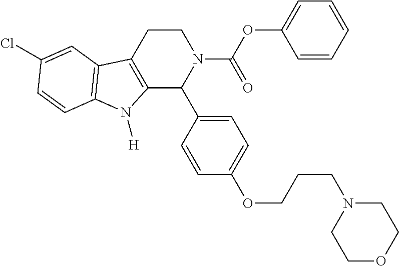 | 1075 |
| 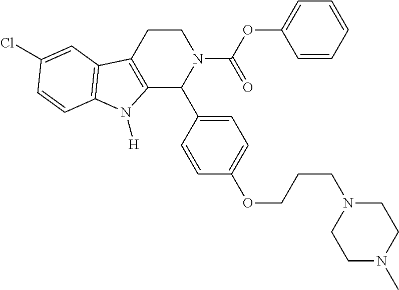 | 1076 |
| 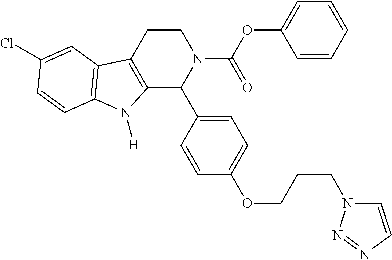 | 1077 |
| 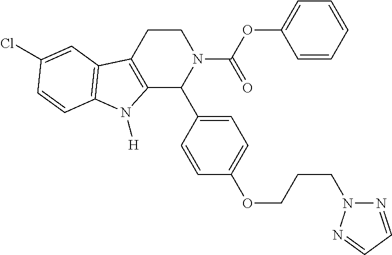 | 1078 |
| 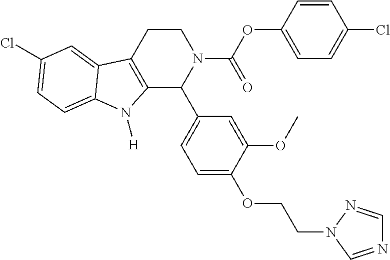 | 1086 |
| 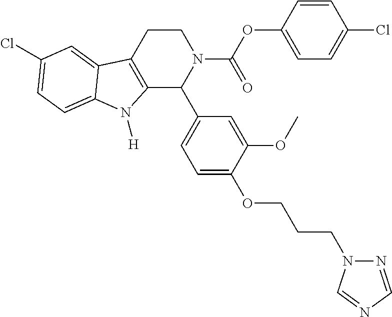 | 1087 |
| 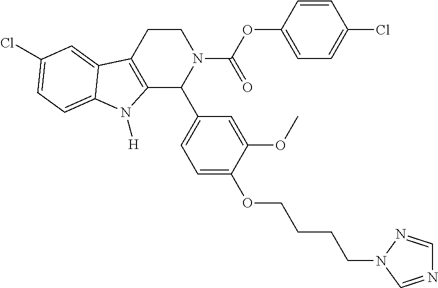 | 1088 |
| 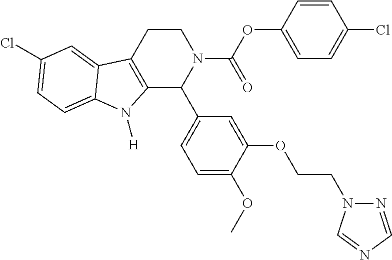 | 1089 |
| 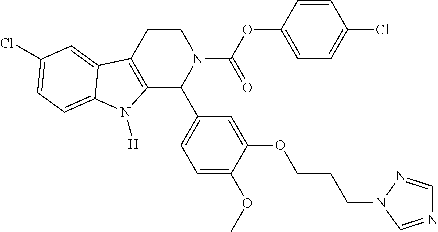 | 1090 |
| 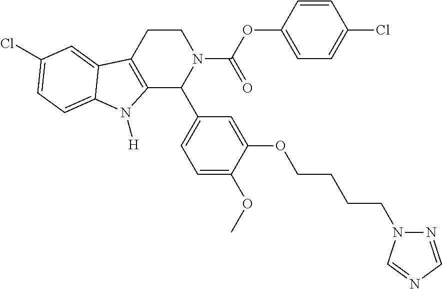 | 1091 |
| 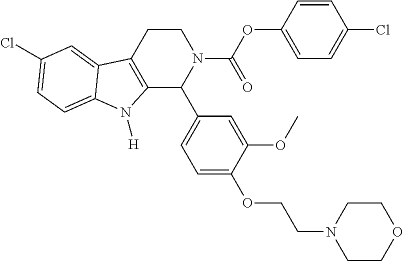 | 1092 |
| 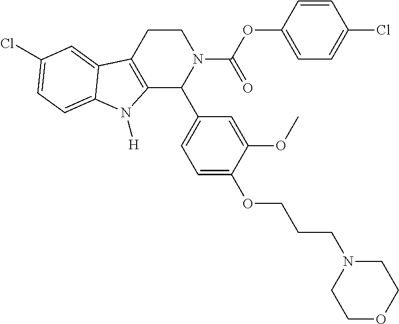 | 1093 |
| 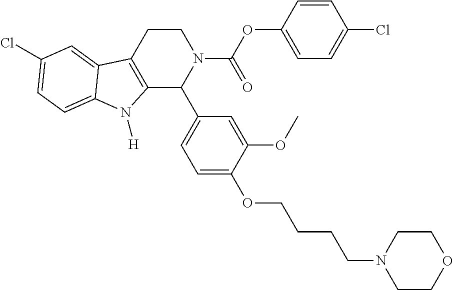 | 1094 |
| 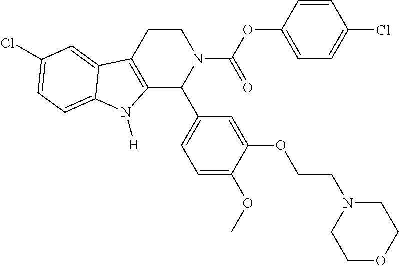 | 1095 |
| 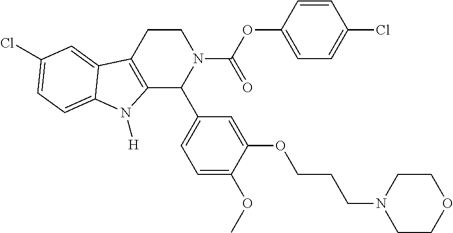 | 1096 |
| 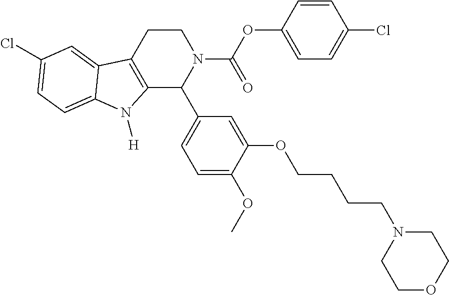 | 1097 |
| 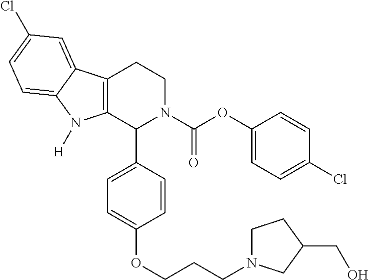 | 1098 |
| 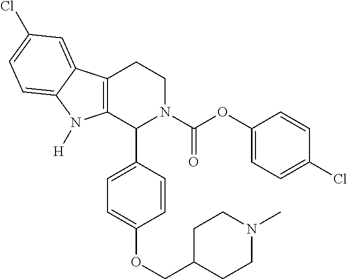 | 1099 |
| 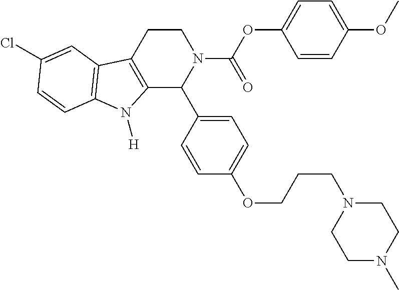 | 1108 |
| 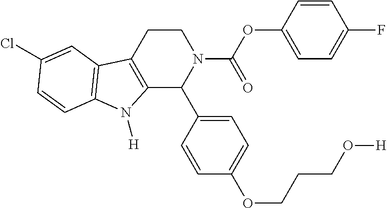 | 1110 |
| 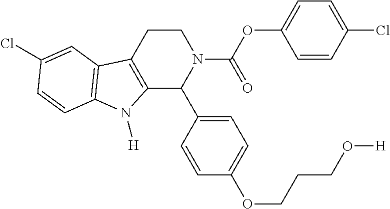 | 1111 |
| 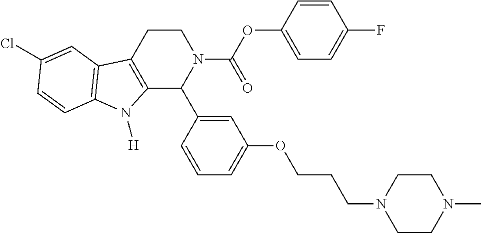 | 1113 |
| 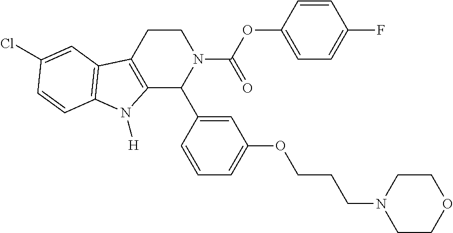 | 1115 |
| 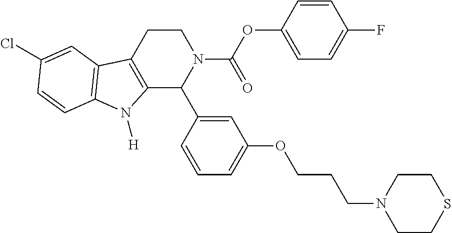 | 1117 |
| 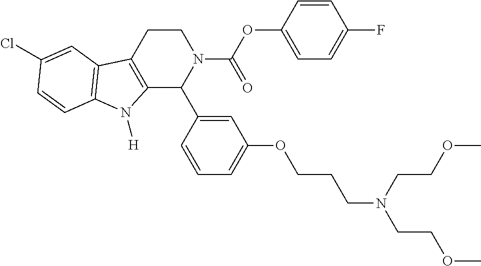 | 1119 |
| 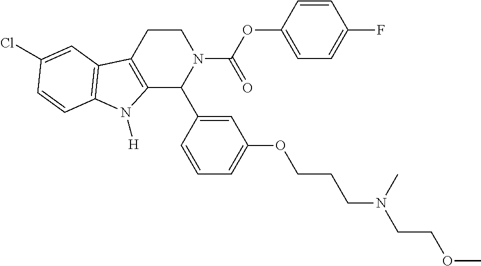 | 1121 |
| 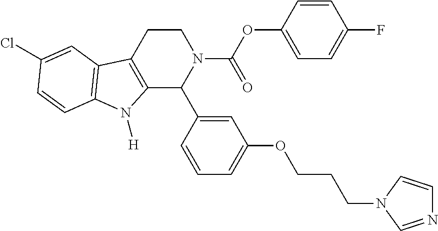 | 1123 |
| 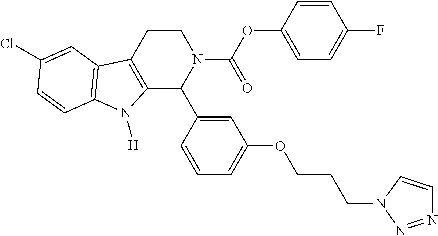 | 1125 |
| 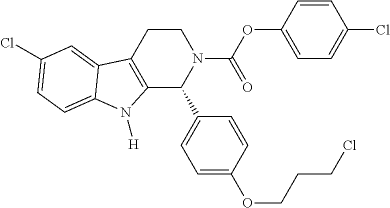 | 1126 |
| 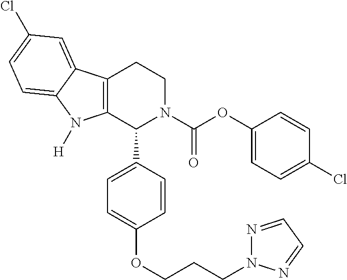 | 1127 |
| 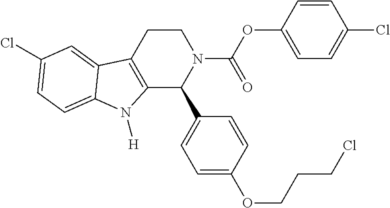 | 1128 |
| 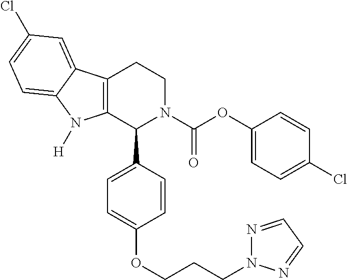 | 1129 |
| 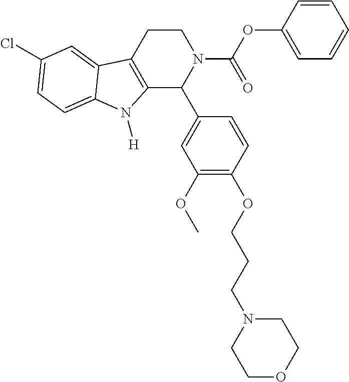 | 1130 |
| 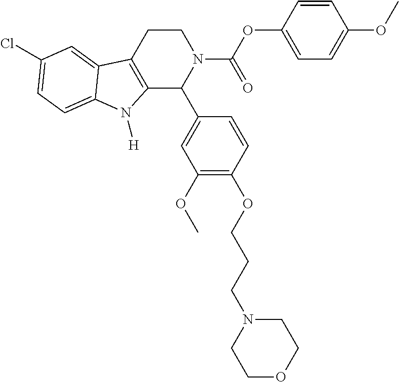 | 1131 |
| 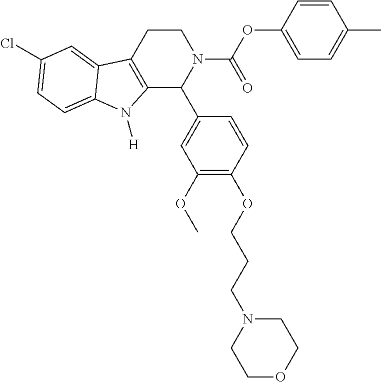 | 1132 |
| 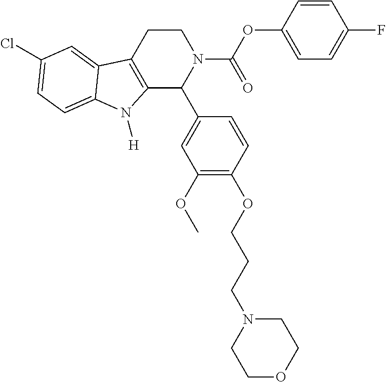 | 1133 |
| 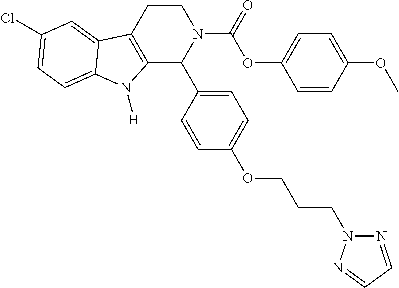 | 1134 |
| 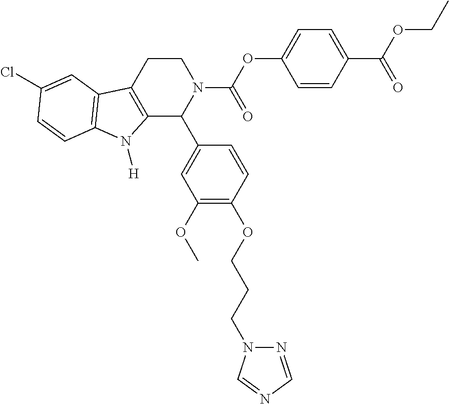 | 1043 |
| 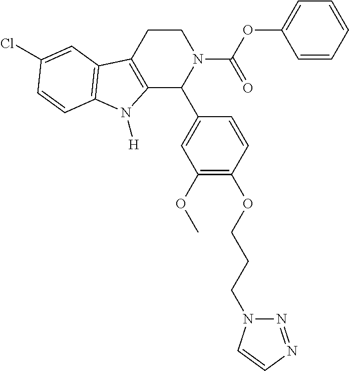 | 1144 |
| 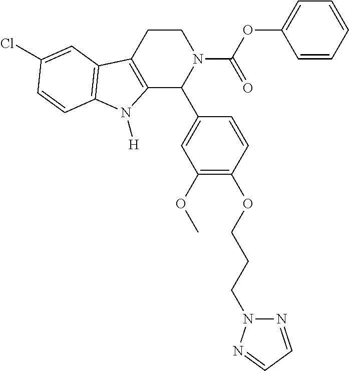 | 1145 |
| 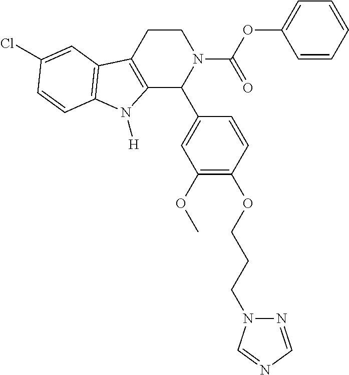 | 1150 |
| 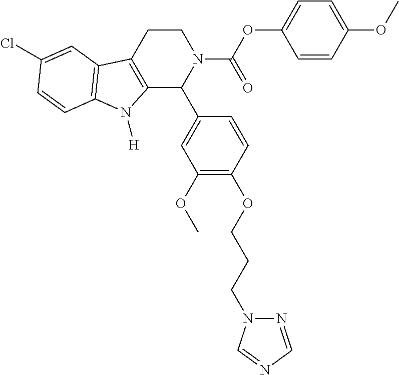 | 1151 |
| 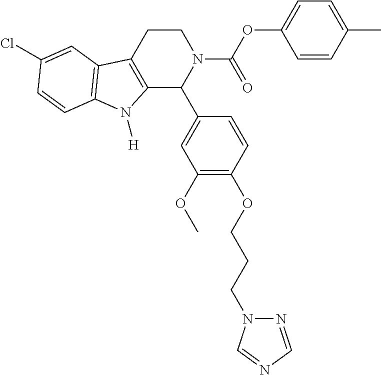 | 1152 |
| 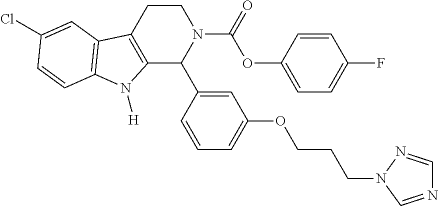 | 1155 |
| 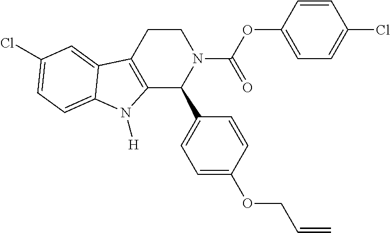 | 1159 |
| 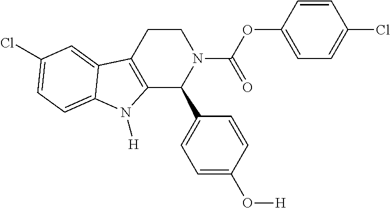 | 1160 |
| 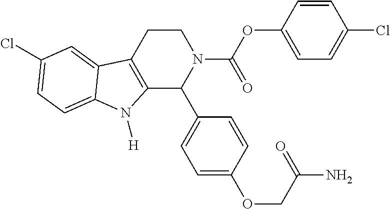 | 1161 |
| 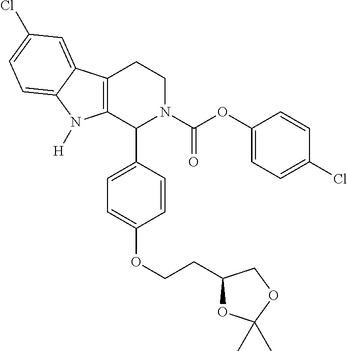 | 1162 |
| 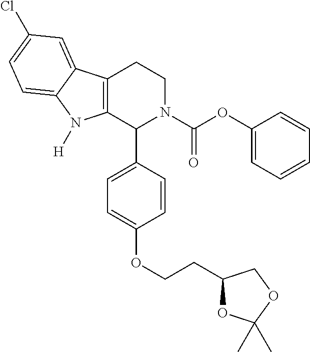 | 1168 |
| 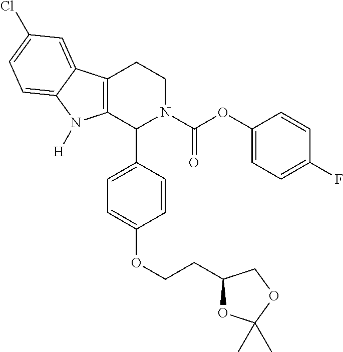 | 1169 |
| 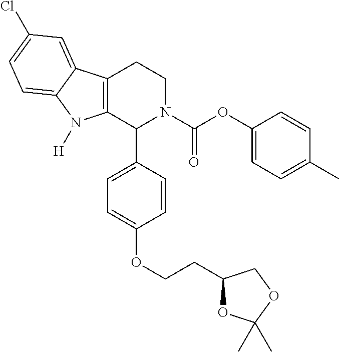 | 1170 |
| 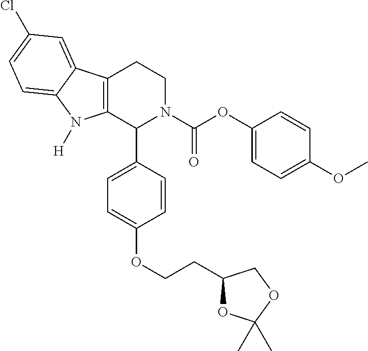 | 1171 |
| 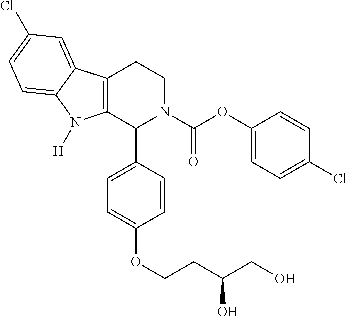 | 1172 |
| 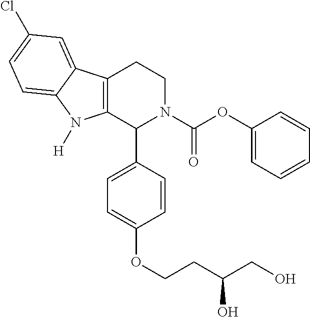 | 1178 |
| 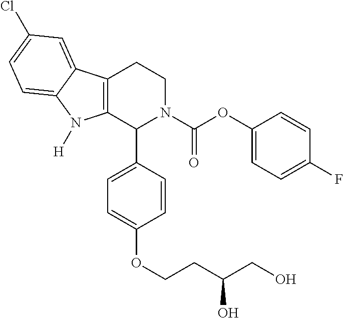 | 1179 |
| 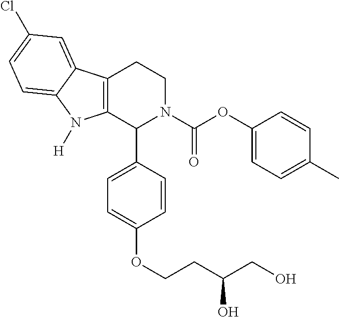 | 1180 |
| 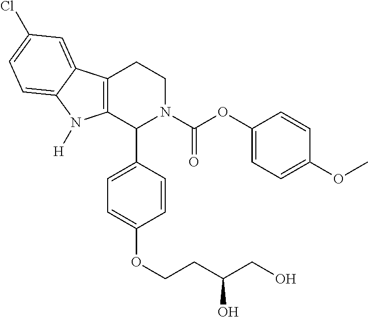 | 1181 |
| 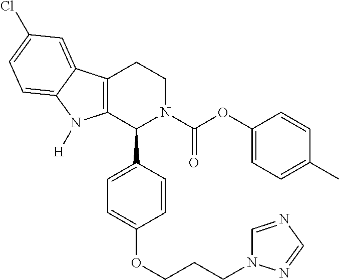 | 1182 |
| 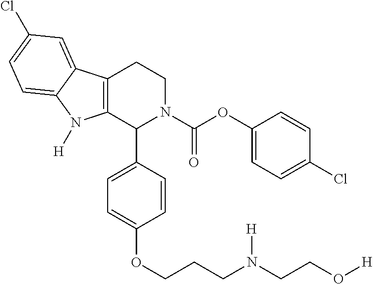 | 1183 |
| 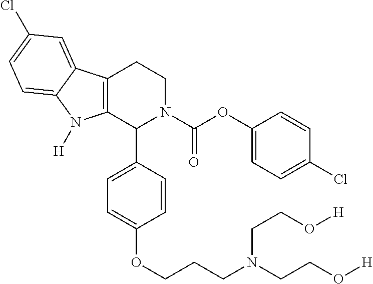 | 1184 |
| 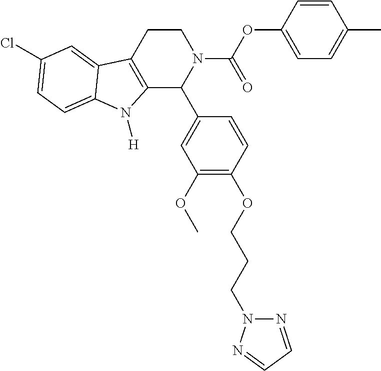 | 1194 |
| 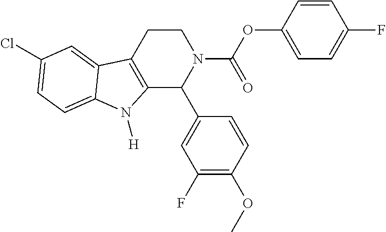 | 1195 |
| 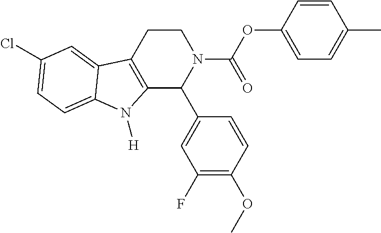 | 1196 |
| 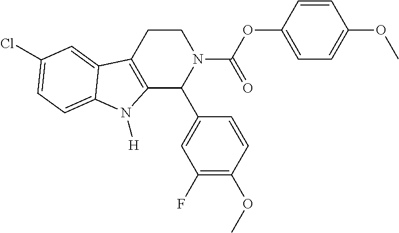 | 1197 |
| 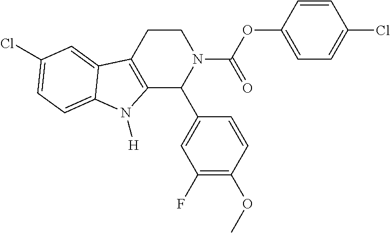 | 1199 |
| 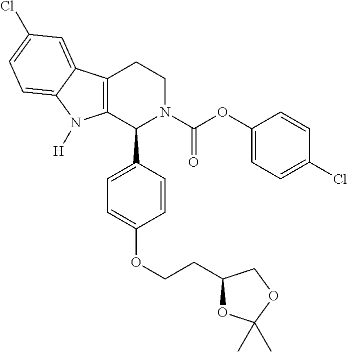 | 1203 |
| 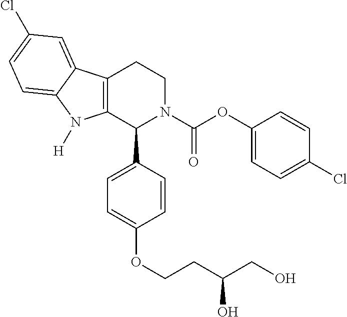 | 1205 |
| 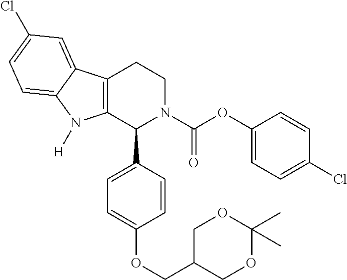 | 1207 |
| 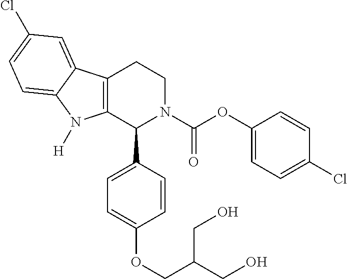 | 1209 |
| 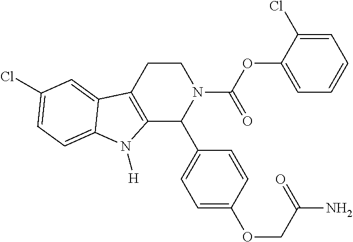 | 1213 |
| 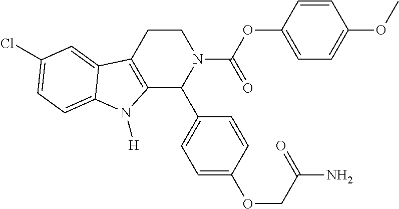 | 1216 |
| 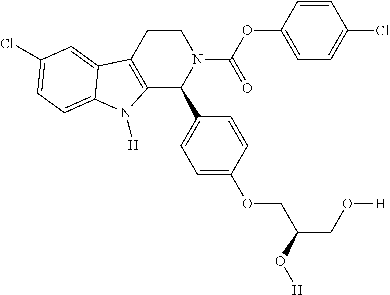 | 1223 |
| 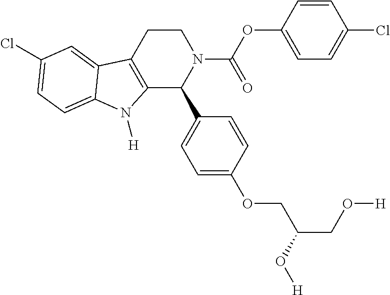 | 1224 |
| 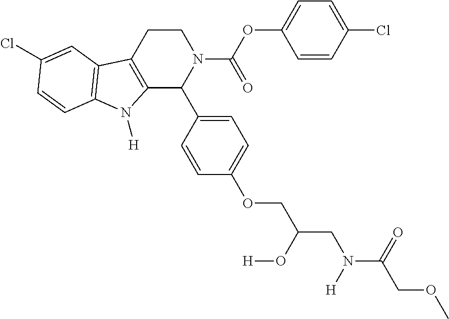 | 1225 |
| 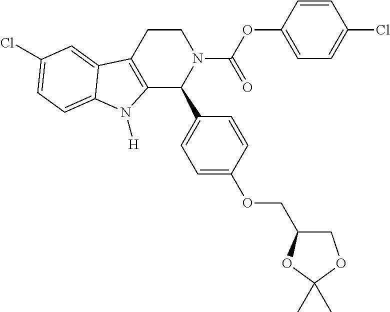 | 1227 |
| 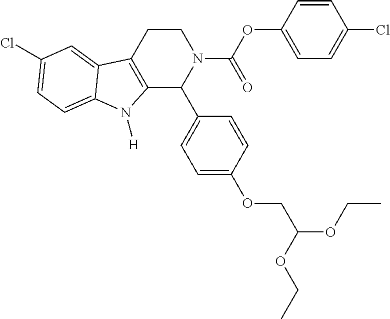 | 1228 |
| 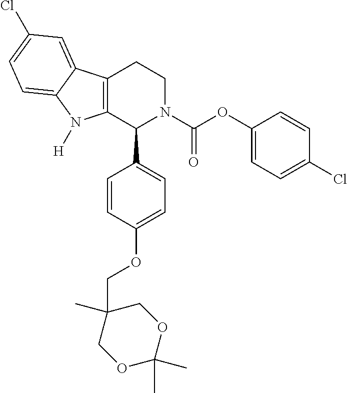 | 1229 |
| 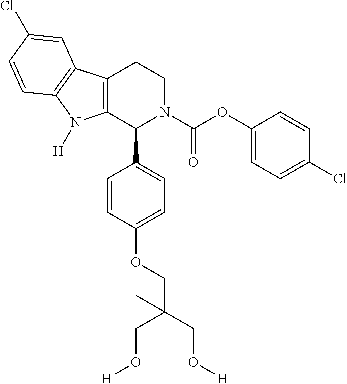 | 1230 |
| 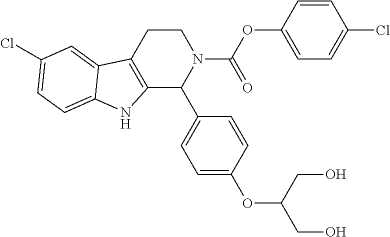 | 1231 |
| 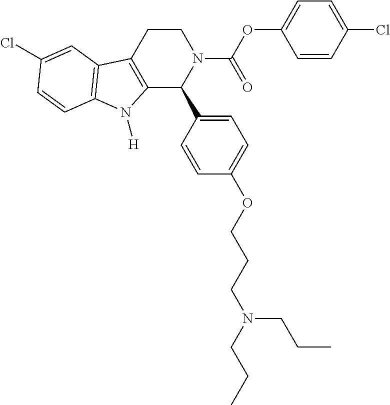 | 1234 |
| 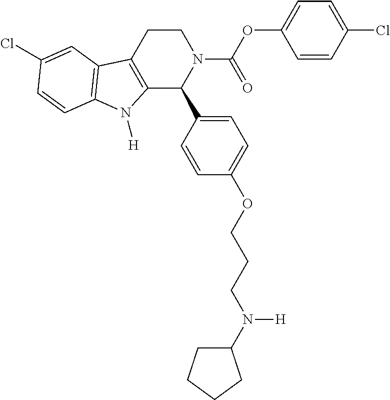 | 1235 |
| 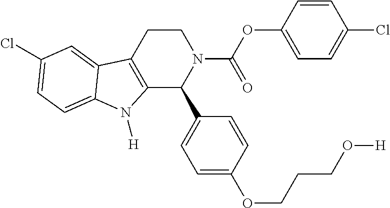 | 1250 |
| 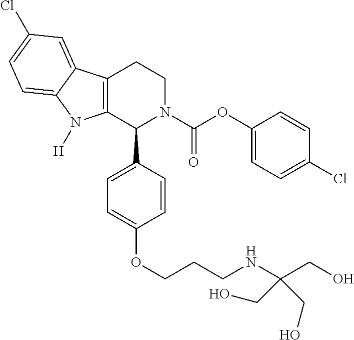 | 1255 |
| 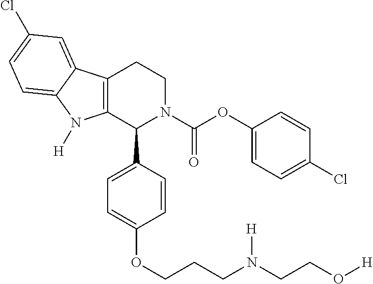 | 1257 |
| 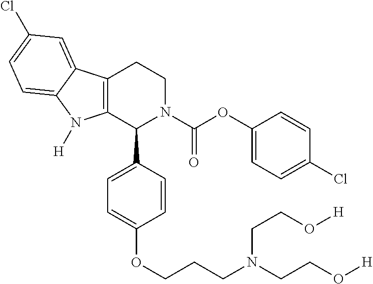 | 1258 |
| 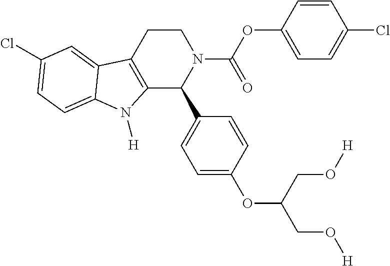 | 1259 |
| 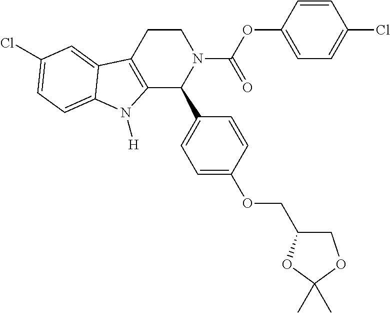 | 1260 |
| 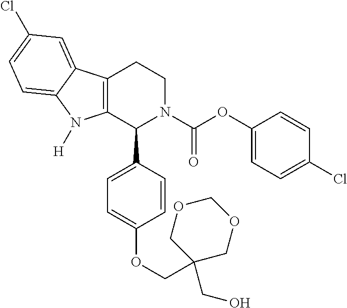 | 1263 |
| 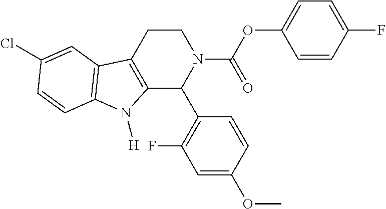 | 1265 |
| 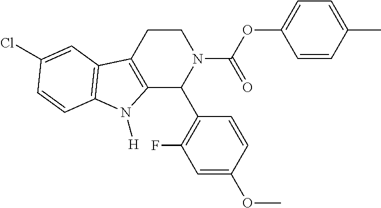 | 1266 |
| 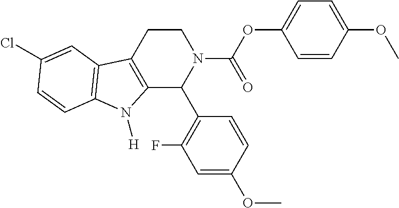 | 1267 |
| 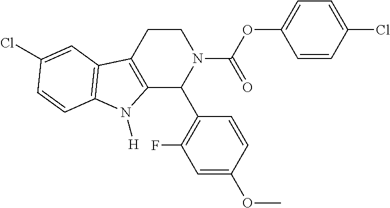 | 1269 |
| 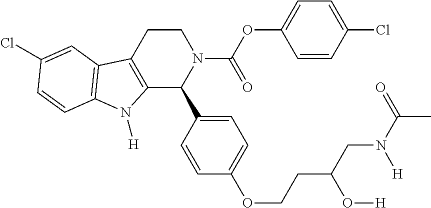 | 1276 |
| 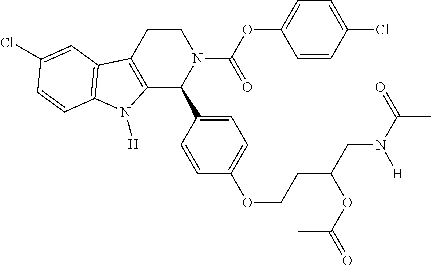 | 1277 |
| 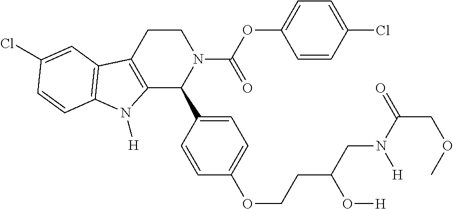 | 1278 |
| 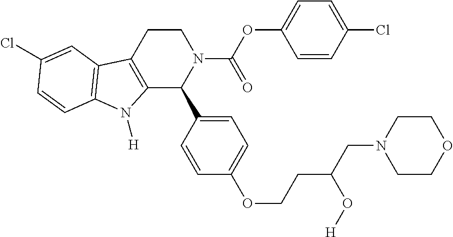 | 1279 |
| 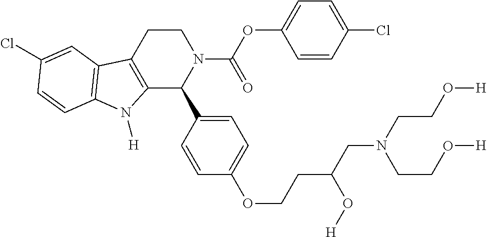 | 1280 |
| 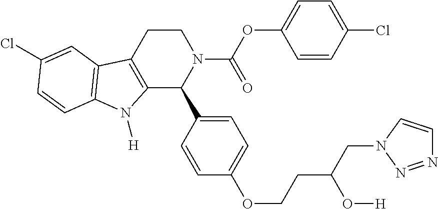 | 1281 |
| 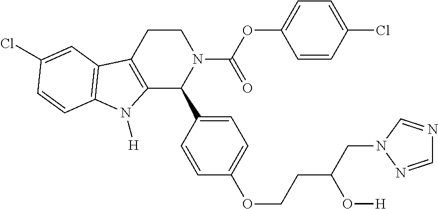 | 1282 |
| 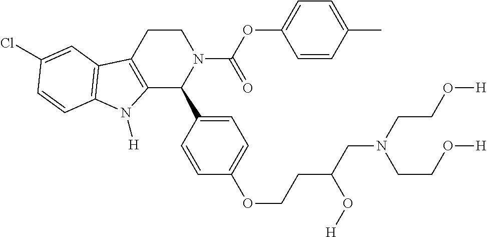 | 1288 |
| 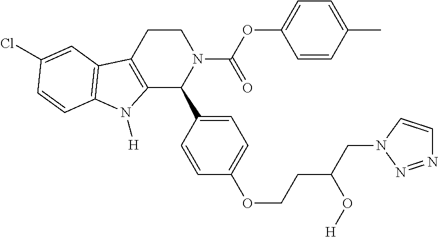 | 1289 |
| 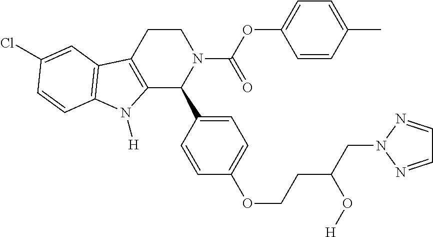 | 1290 |
| 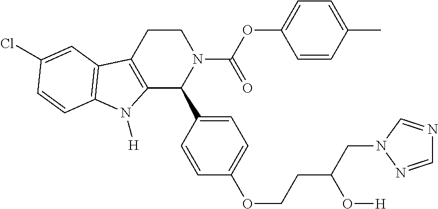 | 1291 |
| 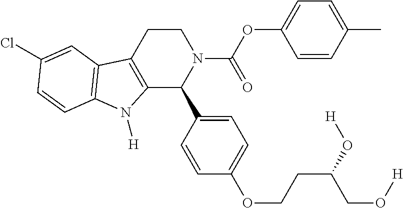 | 1292 |
| 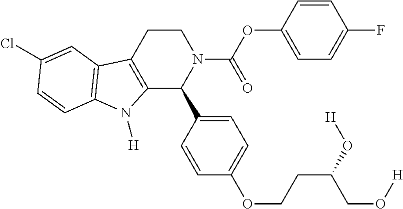 | 1293 |
| 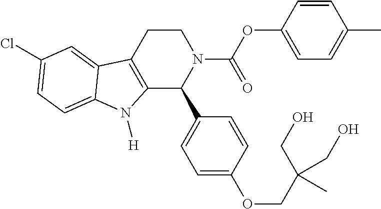 | 1297 |
| 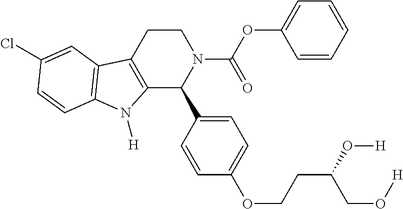 | 1299 |
| 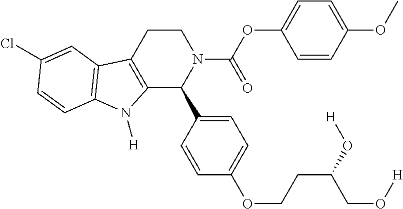 | 1300 |
| 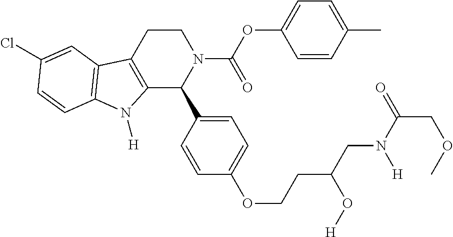 | 1301 |
| 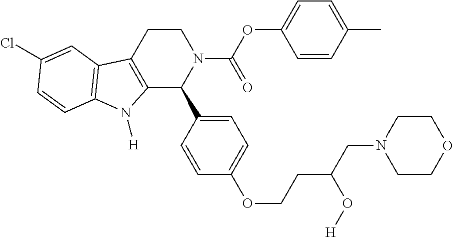 | 1302 |
| 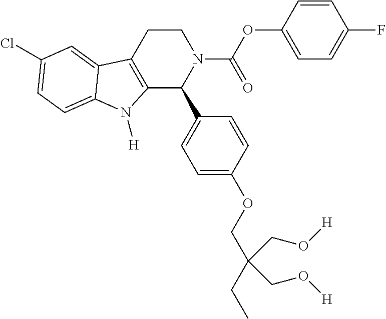 | 1328 |
| 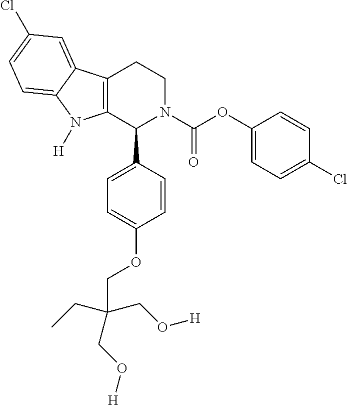 | 1329 |
| 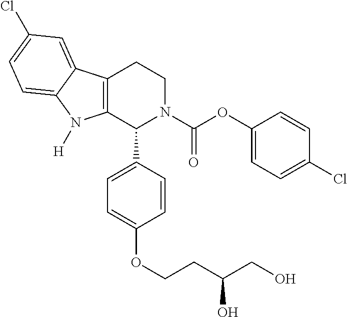 | 1330 |
| 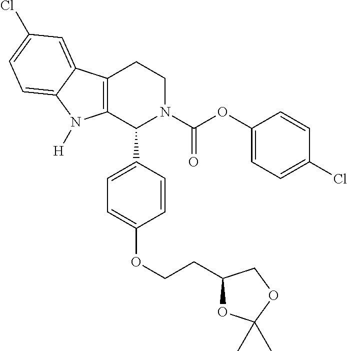 | 1331 |
| 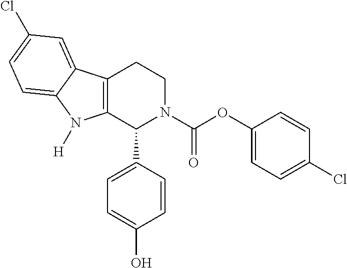 | 1332 |
| 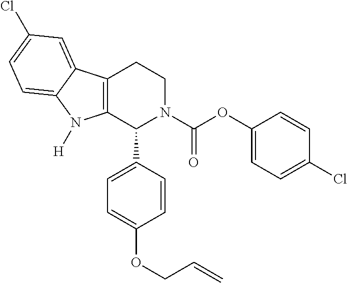 | 1333 |
| 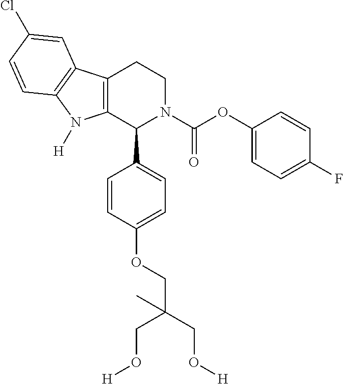 | 1335 |
| 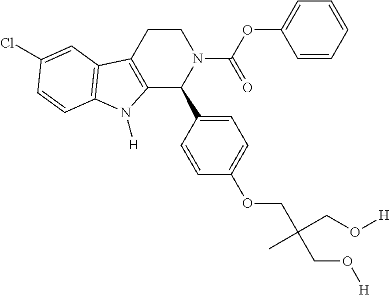 | 1336 |
| 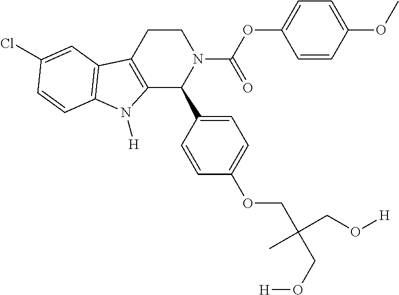 | 1337 |
| 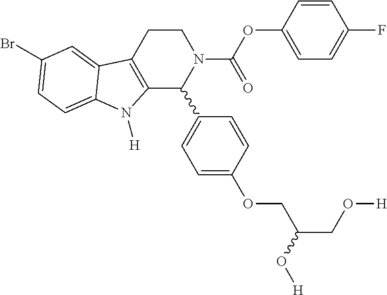 | 1343 |
| 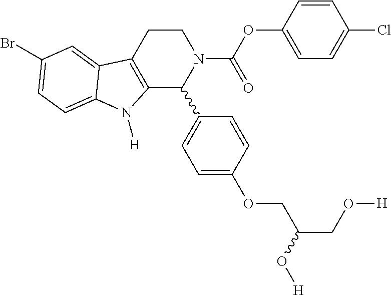 | 1344 |
| 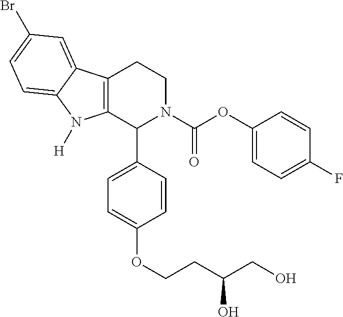 | 1348 |
| 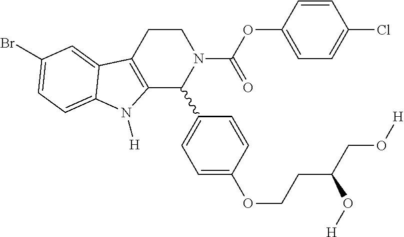 | 1349 |
| 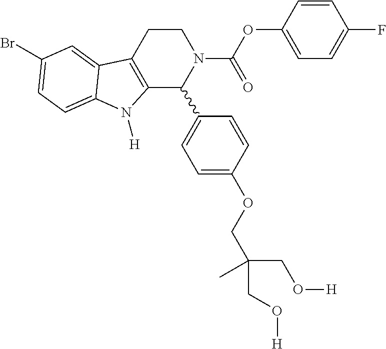 | 1352 |
| 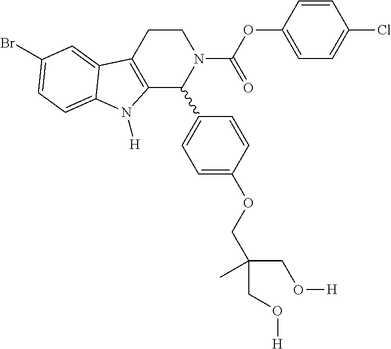 | 1353 |
| 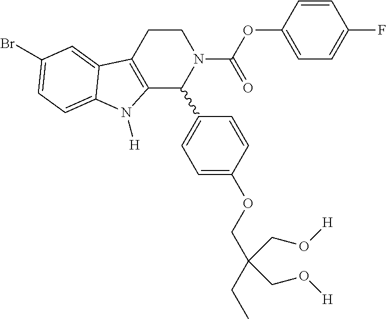 | 1357 |
| 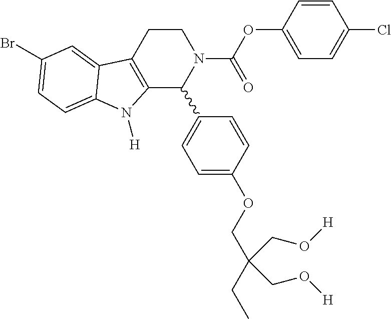 | 1358 |
| 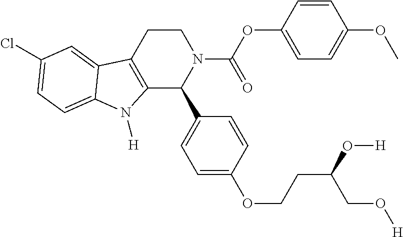 | 1361 |
| 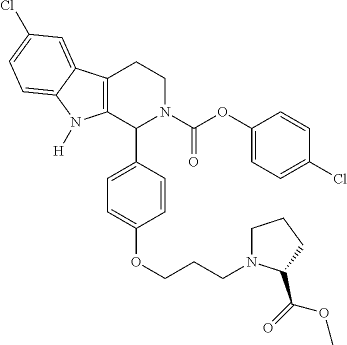 | 1362 |
| 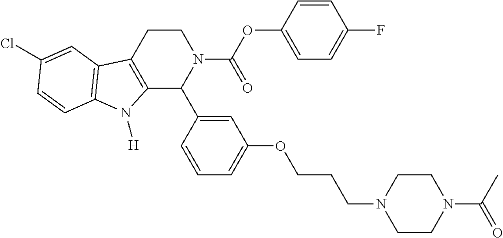 | 1364 |
| 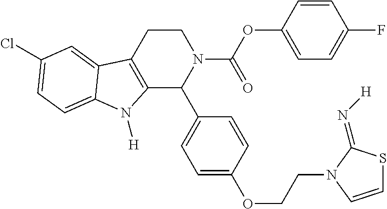 | 1391 |
| 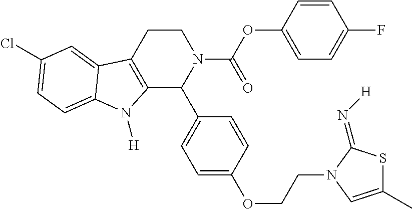 | 1392 |
| 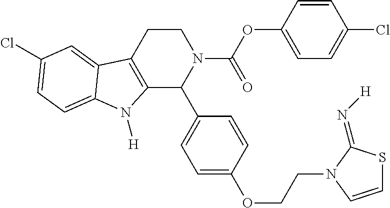 | 1393 |
| 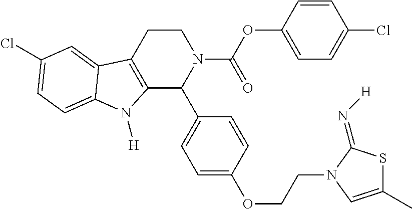 | 1394 |
| 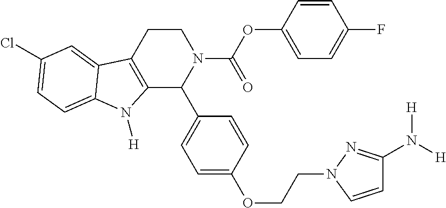 | 1413 |
| 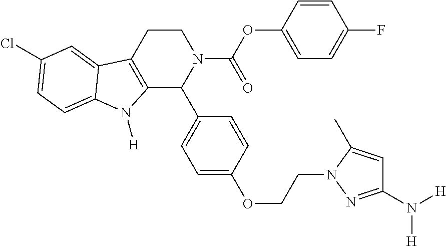 | 1414 |
| 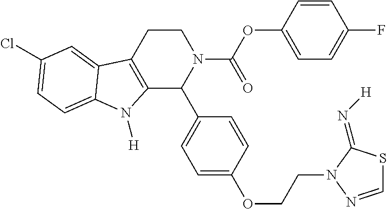 | 1415 |
| 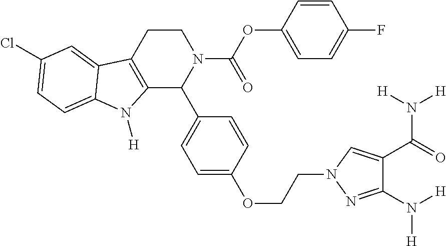 | 1416 |
| 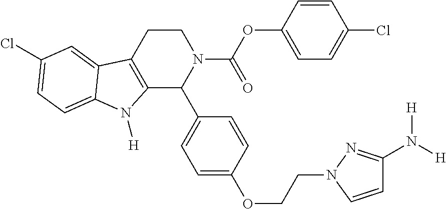 | 1417 |
| 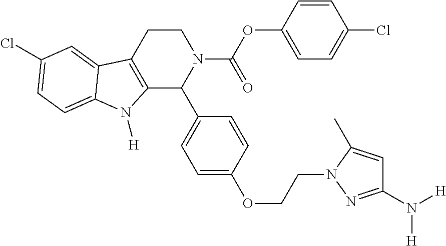 | 1418 |
| 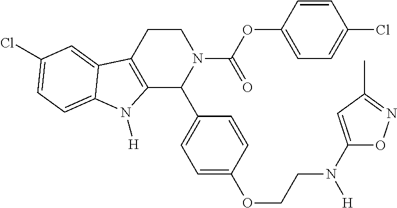 | 1419 |
| 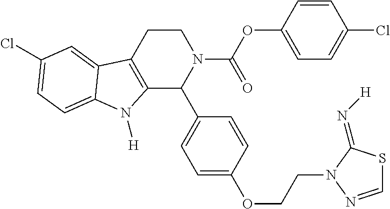 | 1420 |
| 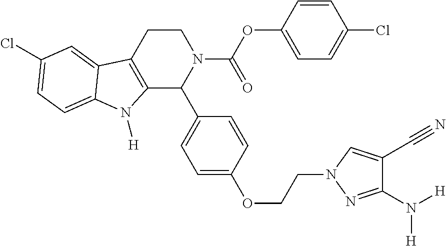 | 1421 |
| 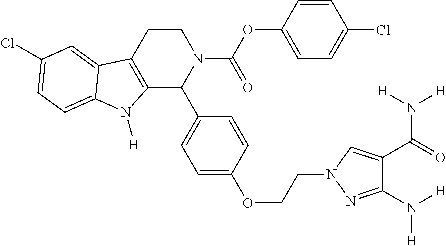 | 1422 |
| 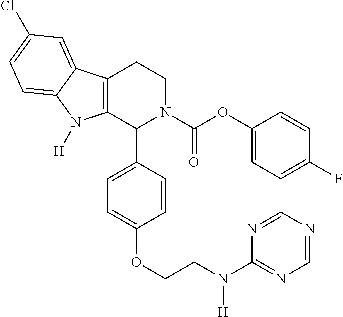 | 1440 |
| 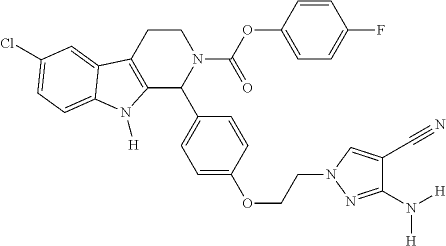 | 1441 |
| 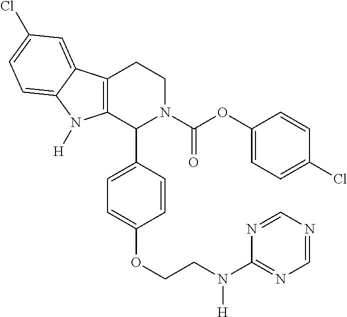 | 1442 |
| 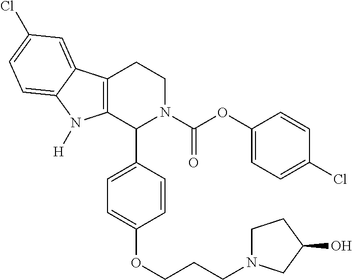 | 1476 |
| 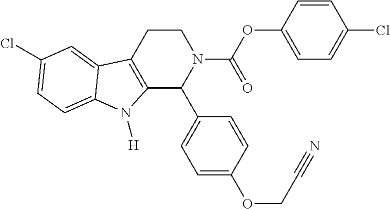 | 1520 |
| 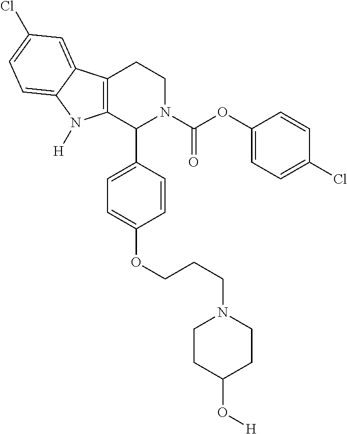 | 1537 |
| 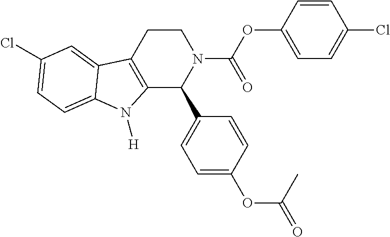 | 1538 |
| 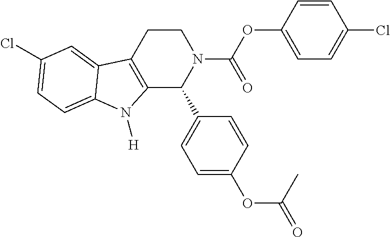 | 1539 |
| 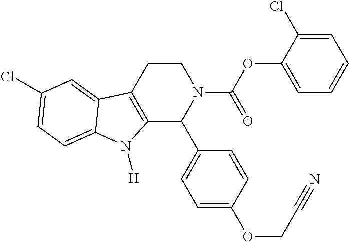 | 1546 |
| 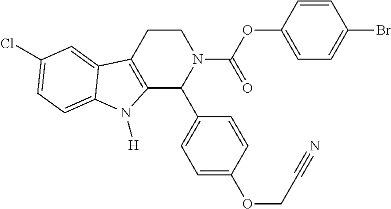 | 1547 |
| 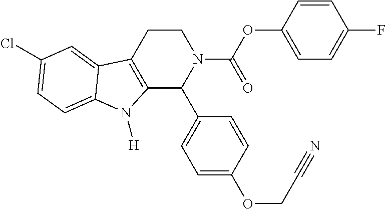 | 1548 |
| 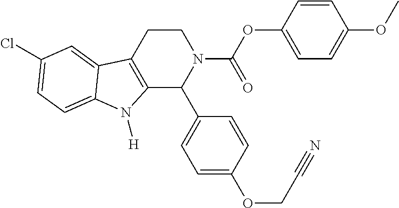 | 1549 |
| 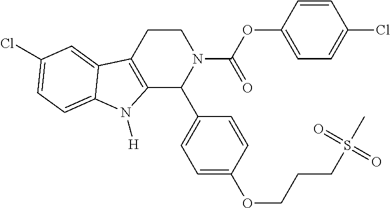 | 1551 |
| 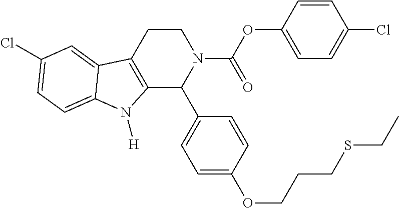 | 1552 |
| 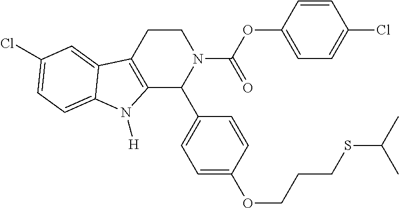 | 1553 |
| 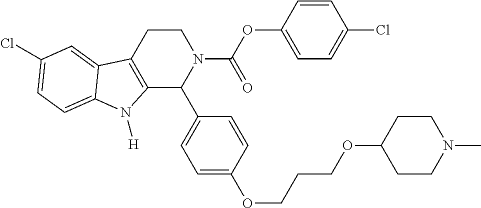 | 1554 |
| 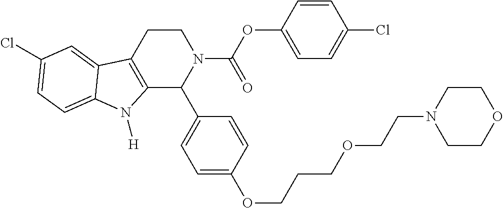 | 1555 |
| 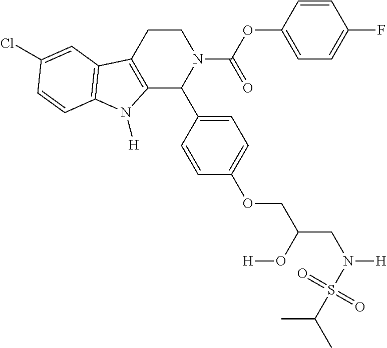 | 1557 |
| 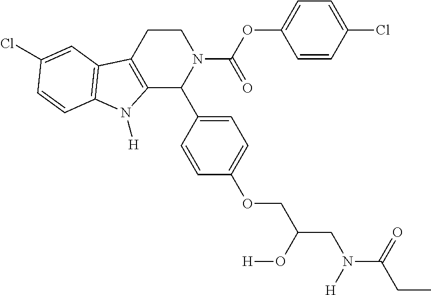 | 1558 |
| 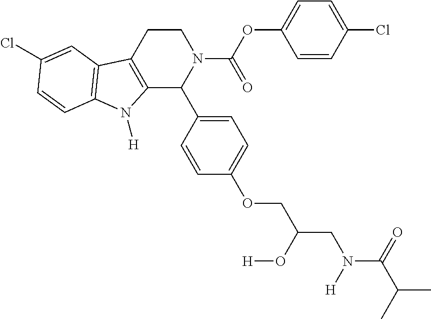 | 1559 |
| 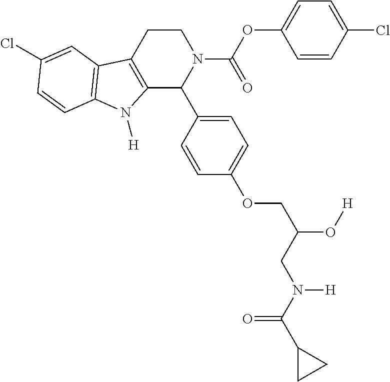 | 1560 |
| 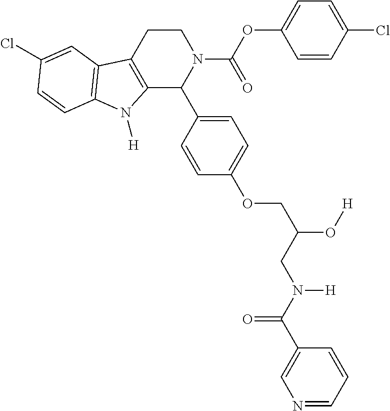 | 1561 |
| 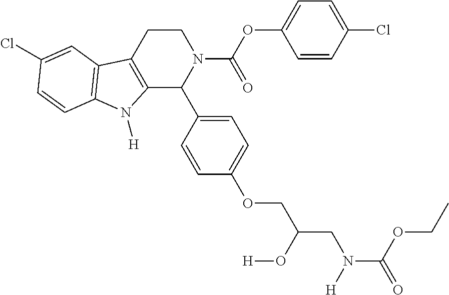 | 1562 |
| 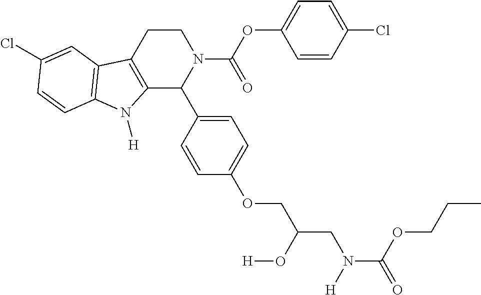 | 1563 |
| 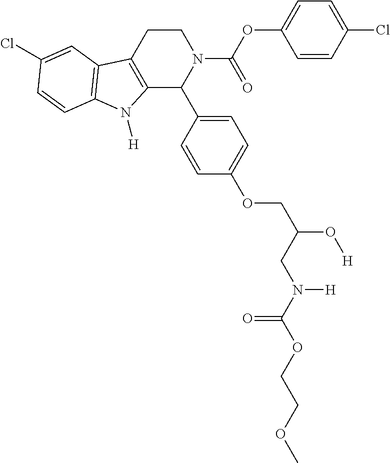 | 1564 |
| 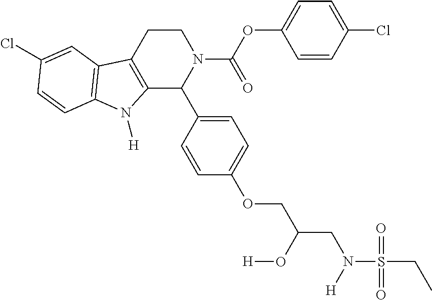 | 1565 |
| 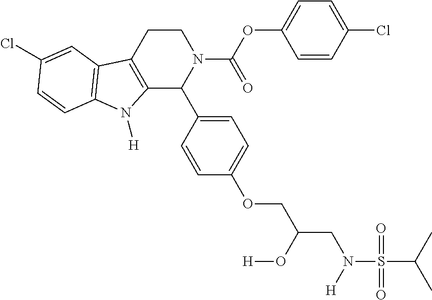 | 1566 |
| 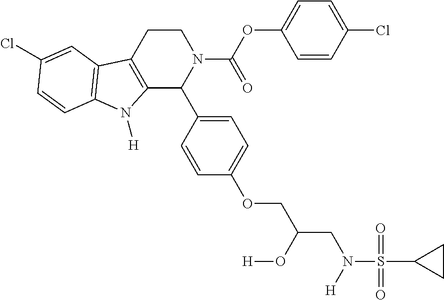 | 1567 |
| 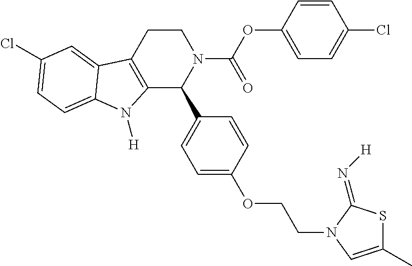 | 1568 |
| 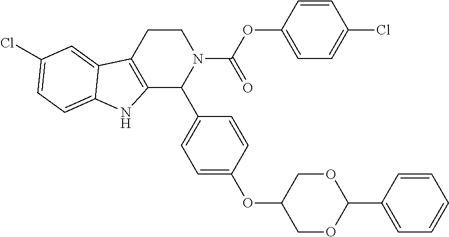 | 1569 |
| 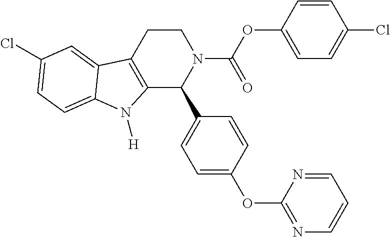 | 1570 |
| 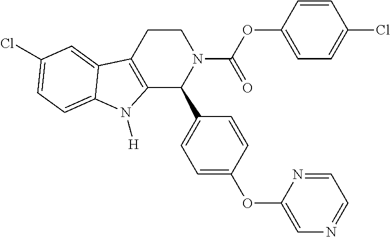 | 1571 |
| 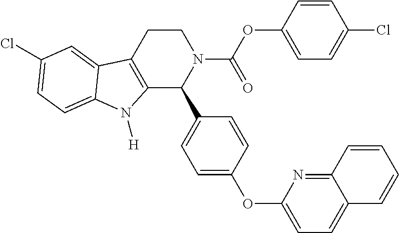 | 1572 |
| 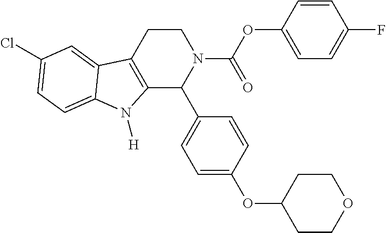 | 1577 |
| 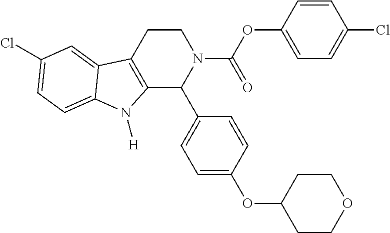 | 1578 |
| 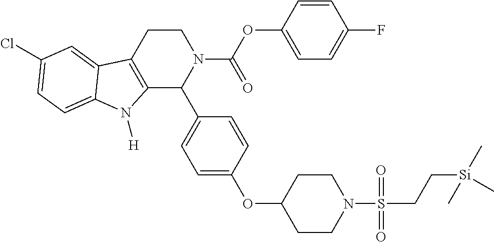 | 1580 |
| 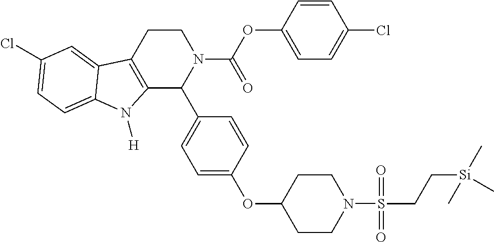 | 1581 |
| 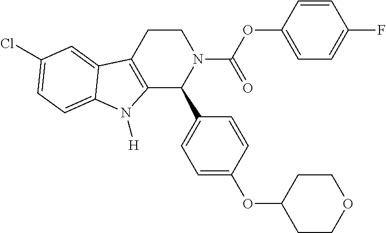 | 1604 |
| 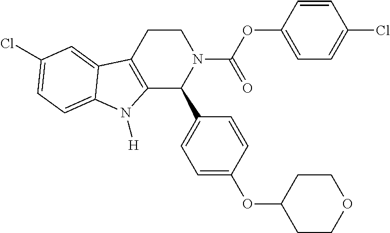 | 1605 |
| 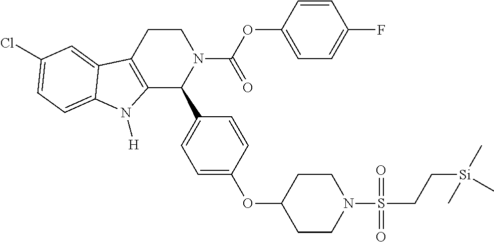 | 1607 |
| 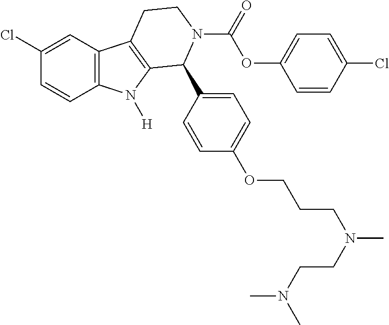 | 1611 |
| 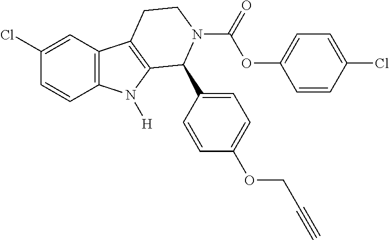 | 1612 |
| 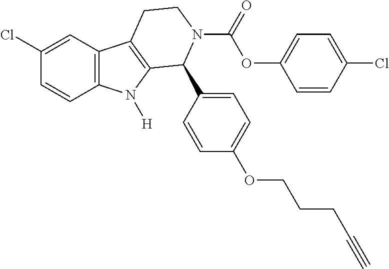 | 1613 |
| 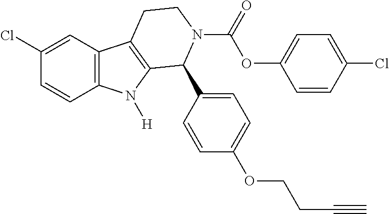 | 1614 |
| 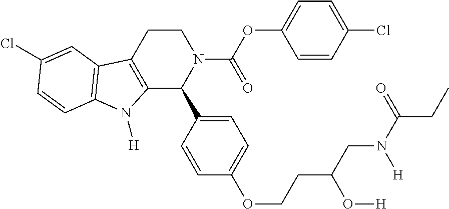 | 1625 |
| 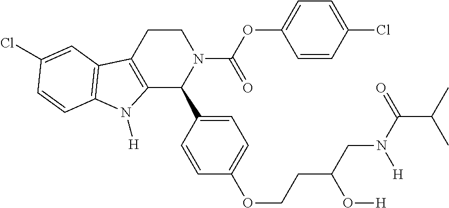 | 1626 |
| 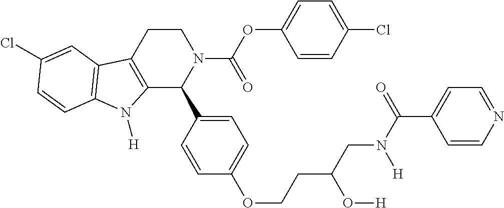 | 1627 |
| 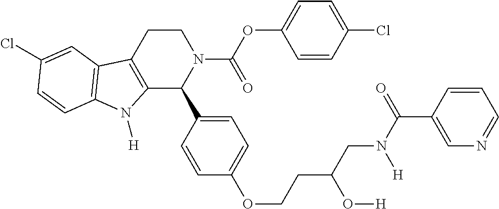 | 1628 |
| 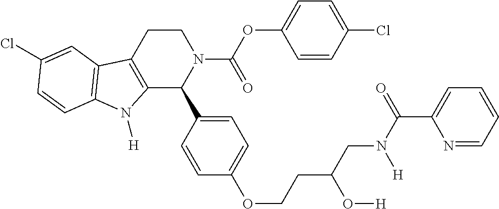 | 1629 |
| 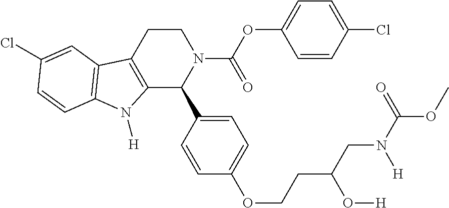 | 1635 |
| 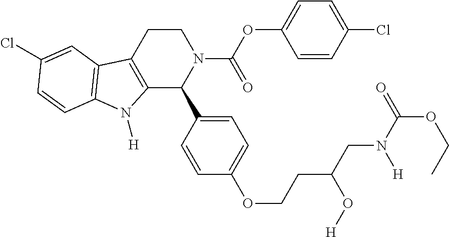 | 1636 |
| 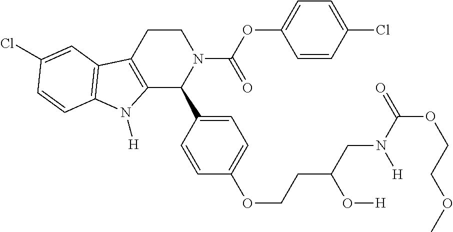 | 1637 |
| 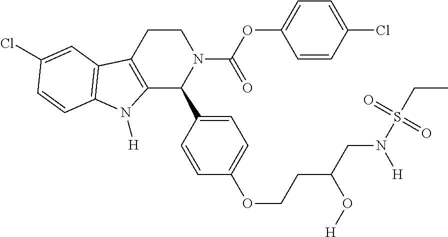 | 1638 |
| 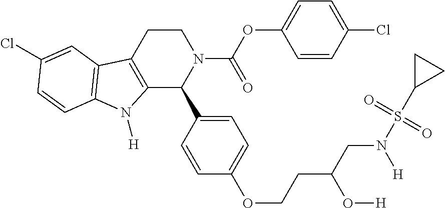 | 1639 |
| 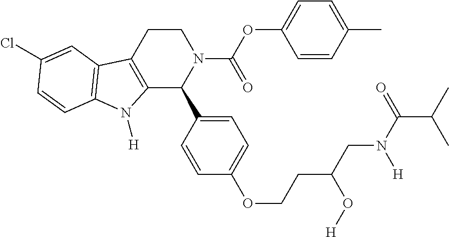 | 1640 |
| 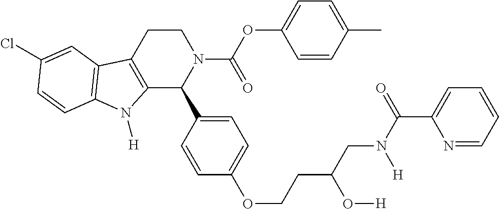 | 1641 |
| 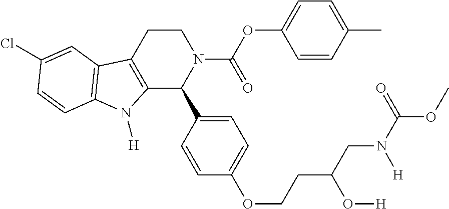 | 1642 |
| 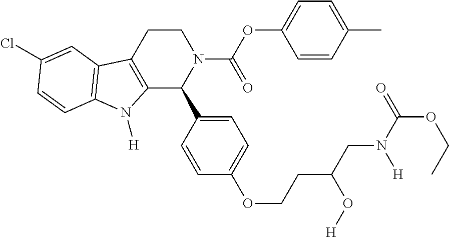 | 1643 |
| 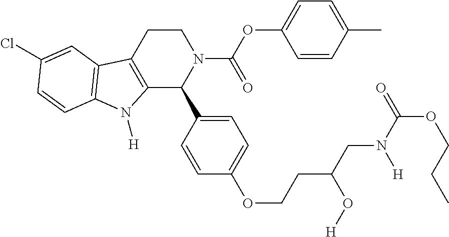 | 1644 |
| 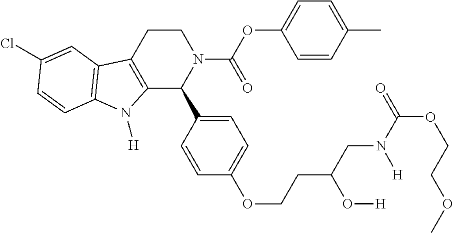 | 1645 |
| 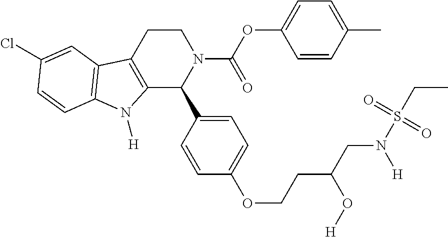 | 1646 |
| 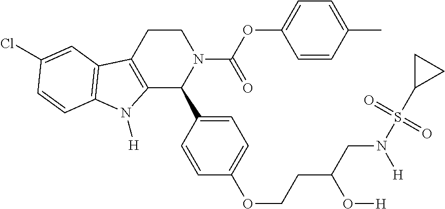 | 1647 |
| 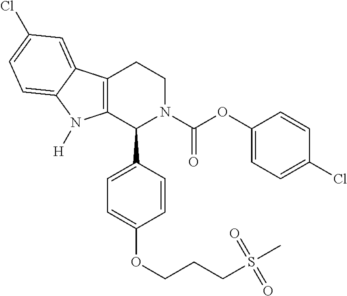 | 1648 |
| 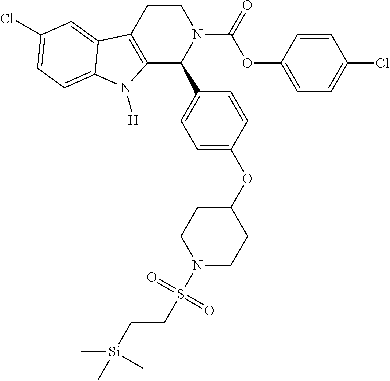 | 1652 |
| 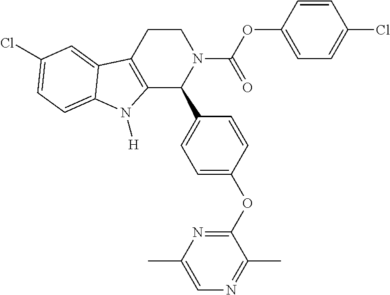 | 1658 |
| 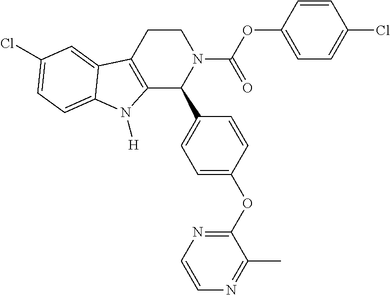 | 1659 |
| 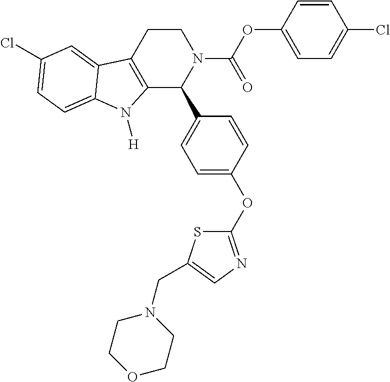 | 1660 |
| 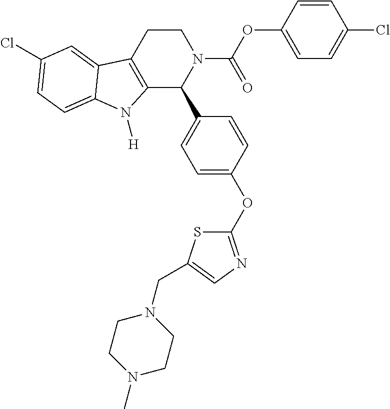 | 1661 |
| 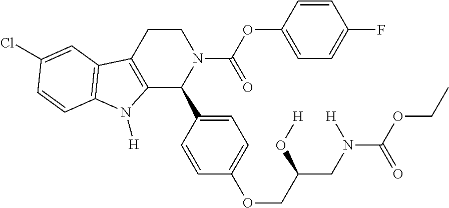 | 1663 |
| 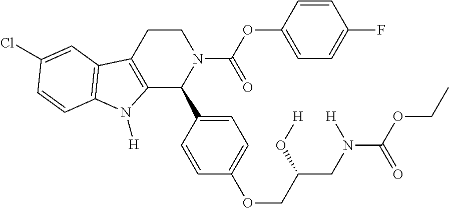 | 1664 |
| 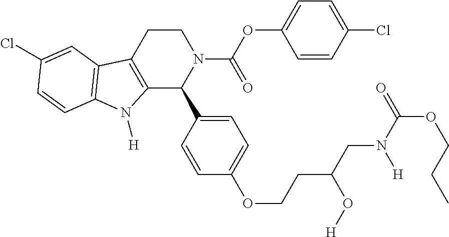 | 1666 |
| 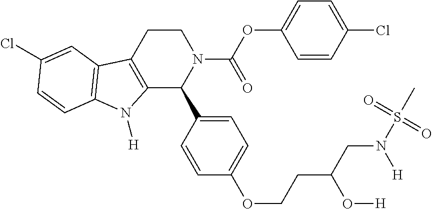 | 1667 |
| 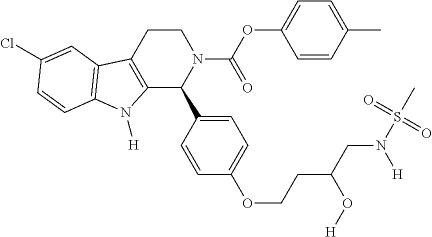 | 1668 |
| 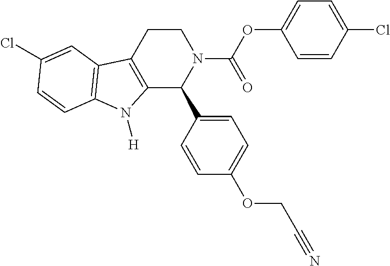 | 1669 |
| 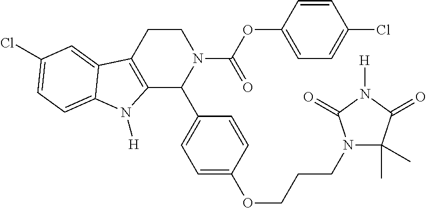 | 1671 |
| 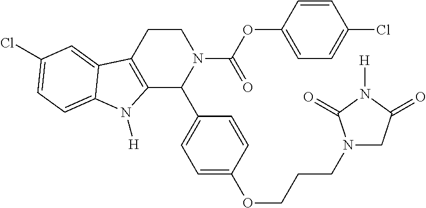 | 1672 |
| 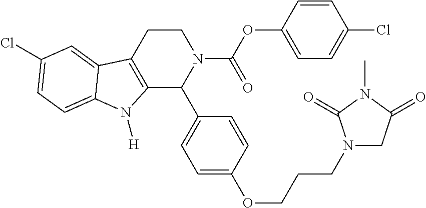 | 1673 |
| 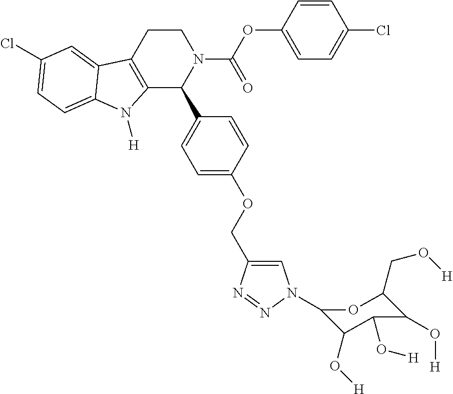 | 1674 |
| 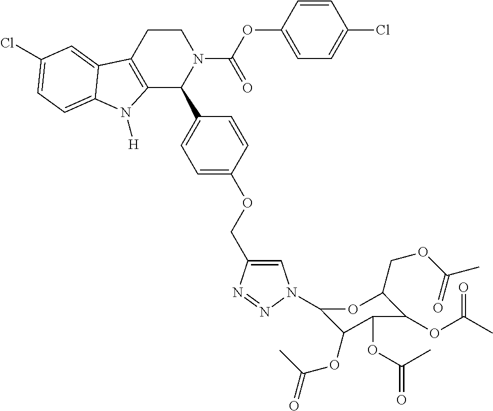 | 1675 |
| 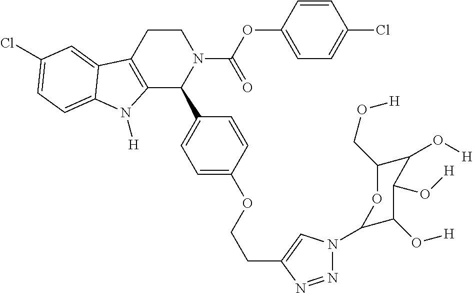 | 1676 |
| 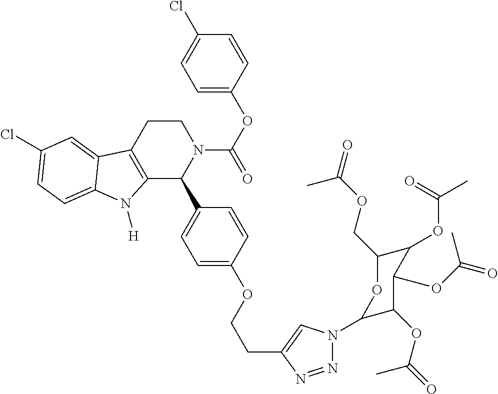 | 1677 |
| 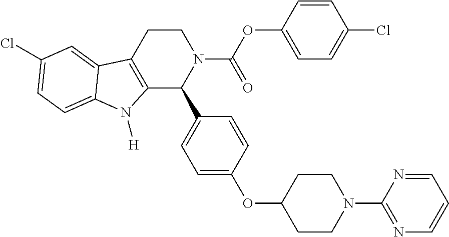 | 1681 |
| 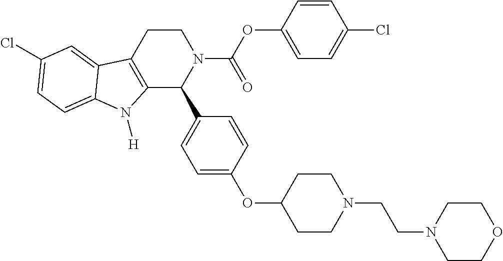 | 1682 |
| 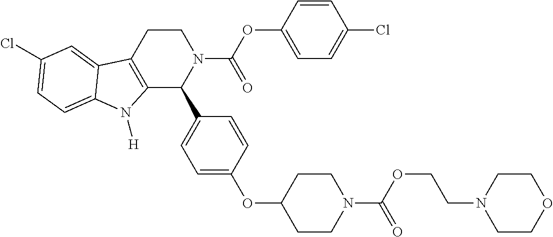 | 1693 |
| 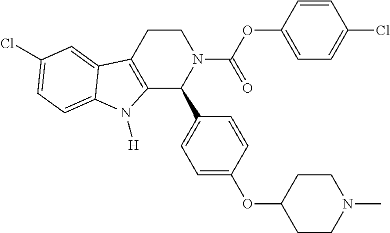 | 1694 |
| 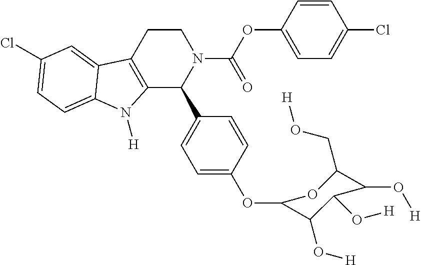 | 1695 |
| 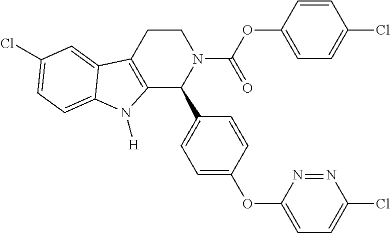 | 1698 |
| 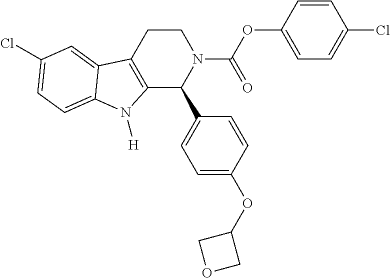 | 1701 |
| 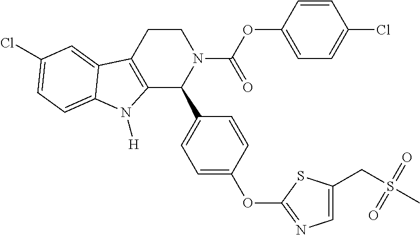 | 1702 |
| 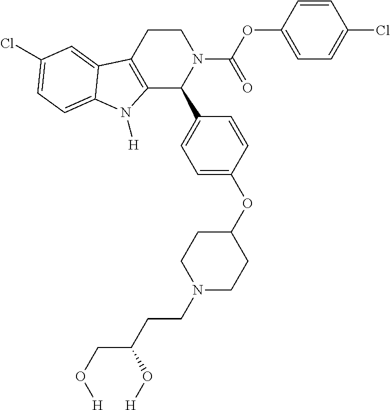 | 1703 |
| 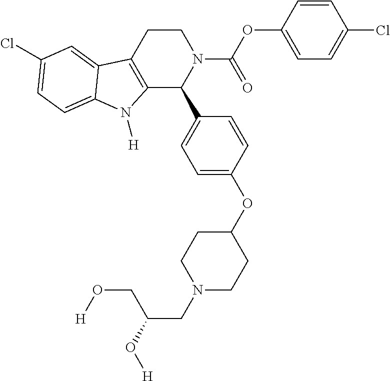 | 1704 |
| 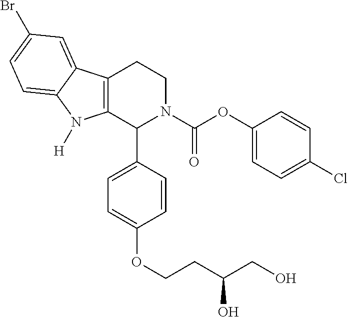 | 1725 |
| 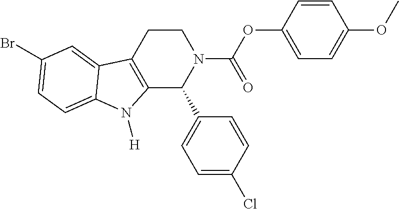 | 1726 |
| 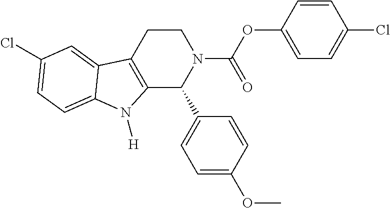 | 1727 |
| 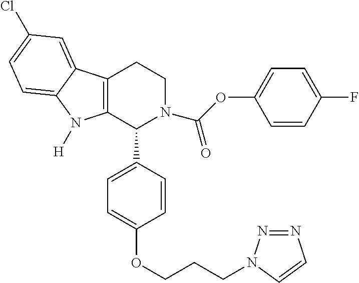 | 1728 |
| 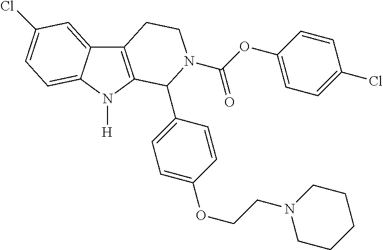 | 1729 |
| 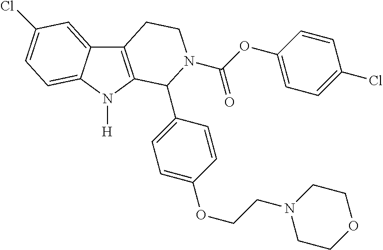 | 1730 |
| 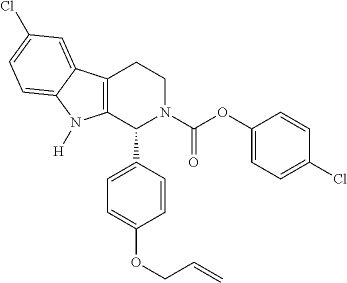 | 1731 |
| 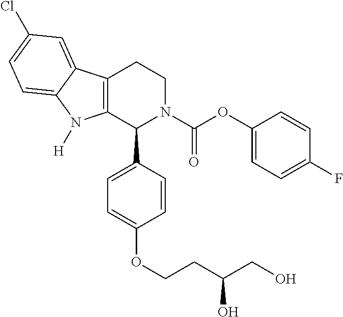 | 1732 |
| 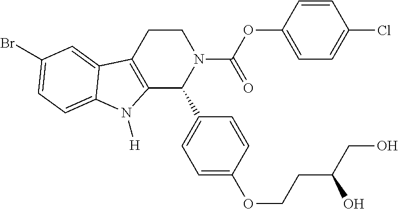 | 1733 |
| 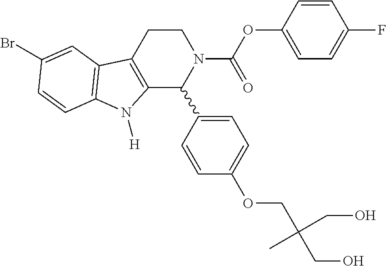 | 1734 |
| 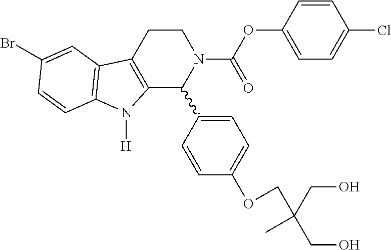 | 1735 |
| 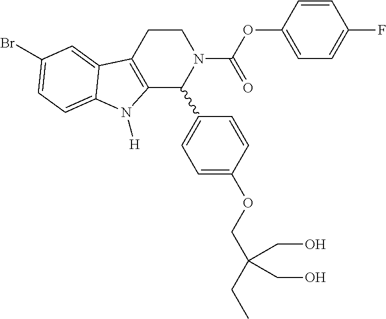 | 1736 |
| 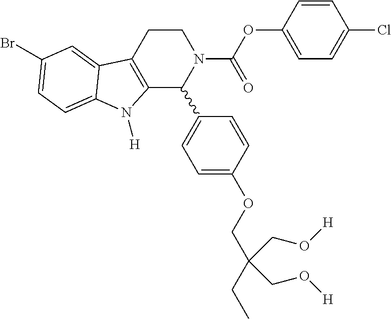 | 1737 |
| 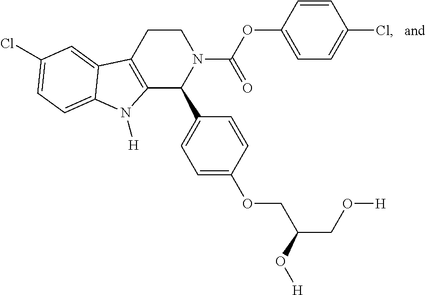 | 1738 |
| 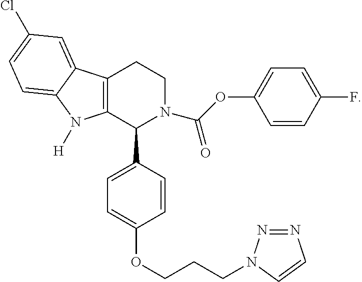 | 1739 |

In a further embodiment, additional examples of the Compounds provided herein are disclosed in International Patent Application Publication No. WO2005/089764 (“'764 publication”) on pages 26 to 98, and in copending U.S. Provisional Patent Application 61/181,653, entitled: METHODS FOR TREATING CANCER AND NON-NEOPLASTIC CONDITIONS, filed May 27, 2009, each of which are incorporated by reference herein in their entirety. Methods for preparing certain Compounds provided herein and the Compounds disclosed on pages 26 to 98 of the '764 publication are provided on pages 99 to 105 and 112 to 142 of the '764 publication and are incorporated by reference herein in their entirety and for all purposes. Methods for preparing certain Compounds provided herein and the Compounds disclosed in copending U.S. Provisional Patent Application 61/181,652, entitled: PROCESSES FOR THE PREPARATION OF SUBSTITUTED TETRAHYDRO BETA-CARBOLINES, filed May 27, 2009, are provided therein and are incorporated by reference herein in their entirety and for all purposes.

5.2 Pharmaceutical Properties and Formulations

5.2.1 Activity

Without being bound by any theory, Compounds described herein inhibit the translation of pathologically expressed human VEGF mRNA and, thus, inhibit the pathologic production of human VEGF protein. In particular, the Compounds act specifically through a mechanism dependent on the 5′ untranslated region (UTR) of the human VEGF mRNA to inhibit the pathologic production of human VEGF protein. The activity of the Compounds tested is post-transcriptional since quantitative real-time polymerase chain reaction (PCR) assessments of mRNA have shown that the Compounds do not alter the levels of human VEGF mRNA. Analyses of the effects of the Compounds tested on ribosome association with VEGF transcripts indicate that the Compounds do not impede initiation of VEGF translation or promote dissociation of ribosomes from human VEGF mRNA.

5.2.1.1 Inhibition of Pathological VEGF Production

Compounds are described that reduce or inhibit pathologic production of human VEGF (also known as VEGF-A and vascular permeability factor (VPF)). Exemplary Compounds have been shown to reduce or inhibit tumor production of VEGF as measured in cell culture and/or preclinical tumor models. Furthermore, the Compounds tested do not affect homeostatic, physiologically produced plasma VEGF levels in healthy humans.

By way of background, the human VEGF-A gene encodes a number of different products (isoforms) due to alternative splicing. The VEGF-A isoforms include VEGF₁₂₁, VEGF₁₆₅, VEGF₁₈₉ and VEGF₂₀₆ having 121, 165, 189 and 206 amino acids, respectively. VEGF₁₆₅ and VEGF₁₂₁ isoforms are soluble, whereas VEGF₁₈₉ and VEGF₂₀₆ isoforms are sequestered within the extracellular matrix. The activity of the Compounds tested was assessed by measuring the concentrations of soluble VEGF and/or extracellular matrix bound-VEGF in cell culture systems. In preclinical tumor models, the activity of the Compounds tested was assessed by measuring the concentrations of soluble VEGF. The data indicate that the Compounds tested inhibit the production of soluble as well as matrix associated forms of tumor derived VEGF.

In particular, a Compound provided herein has been shown to selectively inhibit stress (e.g., hypoxia) induced production of soluble human VEGF isoforms in cell culture without affecting soluble human VEGF production under normoxic conditions (see Sections 9.1.1.1 and 9.1.1.2). Thus, the Compound was shown to preferentially inhibit pathological production of soluble human VEGF isoforms resulting from hypoxia while sparing homeostatic production of soluble isoforms in unperturbed cells. Accordingly, in specific embodiments, a Compound selectively inhibits or reduces the pathological production of a soluble human VEGF isoform over inhibiting or reducing physiological production of a soluble human VEGF isoform.

A Compound provided herein has also shown to selectively inhibit pathological production of VEGF in tumor cells that constitutively overproduce VEGF even under normoxic conditions. See Section 9.1.1.3. In these studies, to better assess the Compound's activity, the inhibition of the pathological production of matrix-bound human VEGF was measured. Thus, in one embodiment, a Compound selectively inhibits or reduces the pathological production of a matrix-bound human VEGF isoform over inhibiting or reducing physiological production of a matrix-bound human VEGF isoform.

The ability of a Compound provided herein to inhibit pathologic production of human VEGF in cell culture has been demonstrated for multiple human tumor cells from a variety of different tissues. See Table 4 (Section 9.1.1.4).

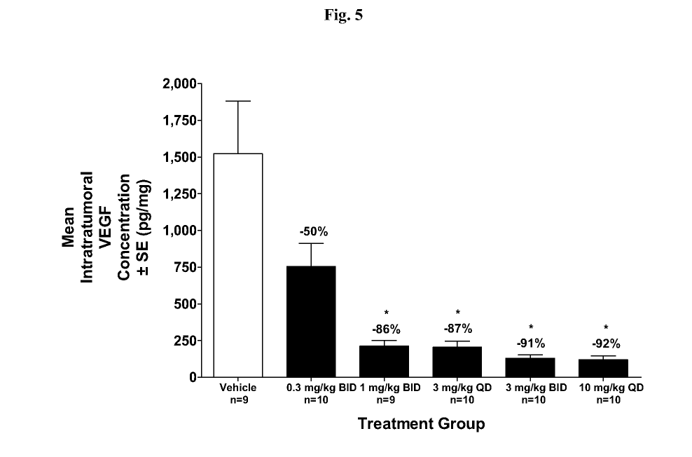

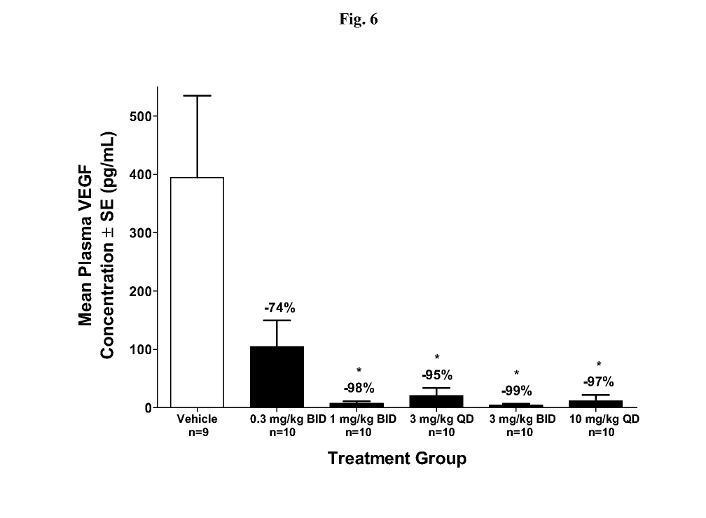

Exemplary Compounds inhibited intratumoral and pathologic plasma human VEGF production in animal models with pre-established human tumors. See Sections 9.1.2.1 to 9.1.2.3. In addition to reducing pathological induced human VEGF concentrations and edema, inflammation, pathological angiogenesis and tumor growth, a Compound provided herein has been shown to selectively reduce intratumoral levels of human growth factors and cytokines, such as IL-6, IL-8, osteopontin, MCP-1 and VEGF family members including human VEGF-C, VEGF-D and placental growth factor (P1GF). See Sections 9.1.2.1. In particular, the Compound shows a dose-dependent reduction in the concentration of intratumoral and pathologic plasma soluble human VEGF isoforms (see Section 9.1.2.2, in particular FIG. 5 and FIG. 6). Accordingly, in specific embodiments, a Compound provided herein, selectively inhibits or reduces the pathological production of one or more human VEGF family members. See Section 9.1.2.1.

5.2.1.2 Inhibition of Pathological Angiogenesis and Tumor Growth

Compounds are described that reduce or inhibit edema, inflammation, pathological angiogenesis and tumor growth. A Compound provided herein has been shown to have a profound effect on the architecture of the tumor vasculature in animal models with pre-established human tumors. The Compound reduced the total volume and diameter of blood vessels formed compared to vehicle treated subjects. See Section 9.2.1. The Compound also showed inhibition of tumor growth in the same model. A dose-response effect of the Compound that correlated with decreases in tumor and pathologic plasma VEGF concentrations was observed when tumor size was assessed. See Section 9.2.2. Thus, in one embodiment, the concentration of soluble pathologically produced VEGF in human plasma may be used to assess and monitor the pharmacodynamic effect of a Compound provided herein. In a specific embodiment, the concentration of either VEGF₁₂₁, VEGF₁₆₅, or both in human plasma may be used to assess and monitor the pharmacodynamic effect of a Compound provided herein.

In concert with a decrease in pathological tumor induced production of VEGF, a Compound provided herein demonstrated tumor regression or delay of tumor growth in various xenograft models, including models of breast cancer, neuroblastoma, and prostate cancer. See Section 9.2.5. Compounds that inhibit tumor growth in multiple preclinical models are more likely to have clinical efficacy. See Johnson et al., Br. J. Cancer 2001, 84(10):1424-31. Further, a Compound provided herein has shown activity in an orthotopic SY5Y neuroblastoma and SKNEP ewing sarcoma tumor model. In orthotopic tumor models, human tumor cells are implanted into the mouse in an organ that corresponds to the location of the human cells from which a tumor would arise. Such models may provide a better predictor of clinical efficacy than injection of tumors into the flanks of nude mice. See Hoffman, Invest. New Drugs 1999, 17(4):343-59. See Section 9.2.5.6.

An in vivo study in rats administered a ¹⁴C-radiolabeled Compound provided herein has been shown that the Compound penetrates all tissues investigated after oral administration. See Section 9.2.6 and Table 23. In one embodiment, a Compound provided herein is able to penetrate cells, tissues or organs that are surrounded by an endothelial cell barrier. In a specific embodiment, a Compound penetrates endothelial cell barriers, such as, but not limited to, the blood-brain barrier, the blood-eye barrier, the blood-testes barrier, blood-uterus barrier, or the blood-ovary barrier. The cells, tissues or organs surrounded by an endothelial cell barrier are, for example, cerebellum, cerebrum, ovary, testis, or the eye. The ability of a Compound to traverse such endothelial barriers makes it suited for the treatment of cancers, such as brain cancers, including but not limited to glioblastoma or neurofibromatosis.

5.2.1.3 Prolongation of Early G₁/Early S-Phase Cell Cycle Delay

Provided herein are Compounds that provoke a delay or prolongation of the cell cycle.

In addition to its effects on pathological VEGF production, a Compound provided herein induces a late G₁/early S-Phase cell cycle delay, i.e., between the late resting or pre-DNA synthesis phase, and the early in DNA synthesis phase in those tumor cell lines in which pathologic VEGF expression is decreased by the Compound. Further characterization indicates that this effect is concentration dependent, occurring at low nanomolar EC₅₀ values similar to those associated with reducing pathological VEGF production. See Section 9.3.1.1. The effect seen is reversible upon cessation of exposure to a Compound. See Section 9.3.1.2. The cell cycle delay and inhibition of pathological VEGF protein production occur in concert, linking these phenotypes in inflammation, pathological angiogenesis and tumor growth. Inhibition of pathological VEGF production in the same tumor cells used herein with small interfering RNA (siRNA) does not induce a delay or prolongation of the cell cycle (data not shown). Conversely, the use of mimosine, a DNA synthesis inhibitor that halts cell cycle progression at the G₁/S interface, does not delay or prolong the cell cycle or reduce VEGF production (data not shown). A Compound provided herein has demonstrated in an in vivo HT1080 xenograft model that the Compound delays cycling through the S-phase; an effect that is distinct from that of bevacizumab, which has no effect on tumor cell cycling. Thus, these experiments indicate that the effects of a Compound on the tumor cell cycle occur in parallel with its actions on pathological VEGF production in tumors.

5.2.2 Formulations

5.2.2.1 General Formulation Methods

The Compounds provided herein can be administered to a patient orally or parenterally in the conventional form of preparations, such as capsules, microcapsules, tablets, granules, powder, troches, pills, suppositories, injections, suspensions and syrups. Suitable formulations can be prepared by methods commonly employed using conventional, organic or inorganic additives, such as an excipient selected from fillers or diluents, binders, disintegrants, lubricants, flavoring agents, preservatives, stabilizers, suspending agents, dispersing agents, surfactants, antioxidants or solubilizers.

Excipients that may be selected are known to those skilled in the art and include, but are not limited to fillers or diluents (e.g., sucrose, starch, mannitol, sorbitol, lactose, glucose, cellulose, talc, calcium phosphate or calcium carbonate and the like), a binder (e.g., cellulose, carboxymethylcellulose, methylcellulose, hydroxymethylcellulose, hydroxypropylmethylcellulose, polypropylpyrrolidone, polyvinylpyrrolidone, gelatin, gum arabic, polyethyleneglycol or starch and the like), a disintegrant (e.g., sodium starch glycolate, croscarmellose sodium and the like), a lubricant (e.g., magnesium stearate, light anhydrous silicic acid, talc or sodium lauryl sulfate and the like), a flavoring agent (e.g., citric acid, or menthol and the like), a preservative (e.g., sodium benzoate, sodium bisulfite, methylparaben or propylparaben and the like), a stabilizer (e.g., citric acid, sodium citrate or acetic acid and the like), a suspending agent (e.g., methylcellulose, polyvinyl pyrrolidone or aluminum stearate and the like), a dispersing agent (e.g., hydroxypropylmethylcellulose and the like), surfactants (e.g., sodium lauryl sulfate, polaxamer, polysorbates and the like), antioxidants (e.g., ethylene diamine tetraacetic acid (EDTA), butylated hydroxyl toluene (BHT) and the like) and solubilizers (e.g., polyethylene glycols, SOLUTOL®, GELUCIRE® and the like). The effective amount of the Compound provided herein in the pharmaceutical composition may be at a level that will exercise the desired effect. Effective amounts contemplated are further discussed in Section 5.4.

The dose of a Compound provided herein to be administered to a patient is rather widely variable and can be subject to the judgment of a health-care practitioner. In general, a Compound provided herein can be administered one to four times a day. The dosage may be properly varied depending on the age, body weight and medical condition of the patient and the type of administration. In one embodiment, one dose is given per day. In any given case, the amount of the Compound provided herein administered will depend on such factors as the solubility of the active component, the formulation used and the route of administration.

A Compound provided herein can be administered orally, with or without food or liquid.

The Compound provided herein can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

In one embodiment, the Compound provided herein is administered orally using a capsule dosage form composition, wherein the capsule contains the Compound provided herein without an additional carrier, excipient or vehicle.

In another embodiment, provided herein are compositions comprising an effective amount of a Compound provided herein and a pharmaceutically acceptable carrier or vehicle, wherein a pharmaceutically acceptable carrier or vehicle can comprise one or more excipients, or a mixture thereof. In one embodiment, the composition is a pharmaceutical composition.

Compositions can be formulated to contain a daily dose, or a convenient fraction of a daily dose, in a dosage unit. In general, the composition is prepared according to known methods in pharmaceutical chemistry. Capsules can be prepared by mixing a Compound provided herein with one or more suitable carriers or excipients and filling the proper amount of the mixture in capsules.

5.2.2.2 Lipid-Based Formulation Methods

One embodiment, provided herein is a SEDDS or SMEDDS system comprising a Compound provided herein (e.g., an effective amount of a composition provided herein), and a carrier medium comprising a lipophilic component, a surfactant, and optionally a hydrophilic component. In certain embodiments, the present disclosure provides a SEDDS or SMEDDS system comprising a Compound provided herein, and a carrier medium comprising one or more surfactants and optionally one or more additives.

In certain embodiments, the SEDDS or SMEDDS system is suitable for oral administration.

One embodiment, provided herein is a SEDDS or SMEDDS system comprising a representative Compound provided herein and a carrier medium that comprises a lipophilic component, a surfactant, optionally a hydrophilic component and optionally an additive.

In one embodiment, the SEDDS or SMEDDS system forms an o/w (oil-in-water) microemulsion when diluted with water.

In one embodiment, of a SEDDS or SMEDDS system provided herein is a microemulsion comprising a Compound provided herein. In certain embodiments, the microemulsion is an o/w (oil-in-water) microemulsion. In one embodiment, the microemulsion comprises a Compound provided herein, a lipophilic component, a surfactant, water, and optionally a hydrophilic component and optionally an additive. In one embodiment, the microemulsion comprises a Compound provided herein, a lipophilic component, a surfactant, and water. In one embodiment, the microemulsion comprises a Compound provided herein, a surfactant, water, and optionally an additive.

The colloidal structures of the microemulsion form spontaneously or substantially spontaneously when the components of the SEDDS or SMEDDS system are brought into contact with an aqueous medium, e.g., by simple shaking by hand for a short period of time, for example for about 10 seconds. The SEDDS or SMEDDS system provided herein is thermodynamically stable, e.g., for at least 15 minutes or up to 4 hours, even to 24 hours. Typically, the system contains dispersed structures, i.e., droplets or liquid nanoparticles of a mean diameter less than about 200 nm (2,000 Å), e.g., less than about 150 nm (1,500 Å), typically less than about 100 nm (1,000 Å), generally greater than about 10 nm (100 Å) as measured by standard light scattering techniques, e.g., using a MALVERN ZETASIZER 300™ particle characterizing machine. Solid drug particles of mean diameter greater than 200 nm may also be present. The proportion of particles present may be temperature dependent.

In accordance with the present disclosure, Compounds provided herein may be present in an amount of up to about 20% by weight of the SEDDS or SMEDDS system provided herein, e.g., from about 0.05% by weight. In one embodiment, the Compound provided herein is present in an amount of from about 0.05 to about 15% by weight of the composition, or in an amount of from about 0.1 to about 5% by weight of the SEDDS or SMEDDS system.

In some embodiments, the SEDDS or SMEDDS system provided herein further comprises a carrier medium having a lipophilic component and a surfactant. In other embodiments, the carrier medium also comprises a lipophilic component, a hydrophilic component and a surfactant. In further embodiments, the carrier medium may comprise a surfactant. In some embodiments, the carrier medium also comprises a surfactant and an additive. In certain embodiments, the Compound provided herein can reside in the lipophilic component or phase.

In some embodiments, the SEDDS or SMEDDS system, the carrier medium, and the microemulsion comprise one or more lipophilic substances. In certain embodiments, the SEDDS or SMEDDS system, the carrier medium, and the microemulsion comprise one or more hydrophilic substances. In other embodiments, the SEDDS or SMEDDS system, the carrier medium, and the microemulsion comprise one or more surfactants. In further embodiments, the SEDDS or SMEDDS system, the carrier medium, and the microemulsion comprise one or more additives.

The compositions provided herein can include a variety of additives including antioxidants, antimicrobial agents, enzyme inhibitors, stabilizers, preservatives, flavors, sweeteners and further components known to those skilled in the art.

A. Lipophilic Components

Lipophilic components include, but are not limited to:

A1) Medium Chain Fatty Acid Triglyceride

These include, but are not limited to, triglycerides of saturated fatty acid having 6 to 12, e.g. 8 to 10, carbon atoms. In one embodiment, the medium chain fatty acid triglycerides include, but are not limited to, those known and commercially available under the trade names ACOMED®, LABRAFAC®, MYRITOL®, CAPTEX®, NEOBEE®M 5 F, MIGLYOL® 810, MIGLYOL®812, MIGLYOL®818, MAZOL®, SEFSOL®860, SEFSOL®870. In one embodiment, the lipophilic component is LABRAFAC®. In one embodiment, the lipophilic component is LABRAFAC®CC. In another embodiment, the lipophilic component is LABRAFAC®WL1349.

A2) Propylene Glycol Mono Fatty Acid Esters

The fatty acid constituent may include, but is not limited to, both saturated and unsaturated fatty acids having a chain length of from e.g. C₈-C₁₂. In one embodiment, the fatty acid is propylene glycol mono ester of caprylic and lauric acid as commercially available, e.g. under the trade names SEFSOL® 218, CAPRYOL®90 or LAUROGLYCOL®90, from e.g. Nikko Chemicals Co., Ltd. or Gattefossé or Capmul PG-8 from Abitec Corporation.

A3) Propylene Glycol Mono- and Di-Fatty Acid Esters

These include, but are not limited to, Laroglycol FCC and Capryol PGMC.

A4) Propylene Glycol Diesters

These include, but are not limited to, propylene glycol di-fatty acid esters such as propylene glycol dicaprylate (which is commercially available under the trade name MIGLYOL® 840 from e.g. sasol; Fiedler, H. P. “Lexikon der Hilfsstoffe für Pharmazie, Kosmetik and angrenzende Gebiete”, Edition Cantor, D-7960 Aulendorf, 4th revised and expanded edition (1996), volume 2, page 1008) or Captex 200 from Abitec Corporation.

A5) Propylene Glycol Monoacetate and Propylene Glycol

A6) Transesterified Ethoxylated Vegetable Oils

Transesterified ethoxylated vegetable oils are known and are commercially available under the trade name LABRAFIL® (H. Fiedler, loc. cit vol 2, page 880). Examples are LABRAFIL® M 2125 CS (obtained from corn oil and having an acid value of less than about 2, a saponification value of 155 to 175, an HLB value of 3 to 4, and an iodine value of 90 to 110), and LABRAFIL® M 1944 CS (obtained from kernel oil and having an acid value of about 2, a saponification value of 145 to 175 and an iodine value of 60 to 90). LABRAFIL® M 2130 CS (which is a transesterification product of a C₁₂-C₁₈ glyceride and polyethylene glycol and which has a melting point of about 35 to about 40° C., an acid value of less than about 2, a saponification value of 185 to 200 and an iodine value of less than about 3) may also be used. LABRAFIL® lipophilic components can be obtained, for example, from Gattefossé (Paramus, N.J., USA).

In one embodiment, the alkylene polyol ethers or esters include products obtainable by transesterification of glycerides, e.g. triglycerides, with poly-(C₂-C₄ alkylene) glycols, e.g. poly-ethylene glycols and, optionally, glycerol. Such transesterification products are generally obtained by alcoholysis of glycerides, e.g. triglycerides, in the presence of a poly-(C₂-C₄ alkylene)glycol, e.g. polyethylene glycol and, optionally, glycerol (i.e. to effect transesterification from the glyceride to the poly-alkylene glycol/glycerol component, i.e. via poly-alkylene glycolysis/glycerolysis). In general such reaction is effected by reacting the indicated components (glyceride, polyalkylene glycol and, optionally, glycerol) at elevated temperature under an inert atmosphere with continuous agitation.

In one embodiment, the glycerides are fatty acid triglycerides, e.g. (C₁₀-C₂₂ fatty acid) triglycerides, including natural and hydrogenated oils, in particular vegetable oils. In one embodiment, vegetable oils include, for example, olive, almond, peanut, coconut, palm, soybean and wheat germ oils and, in particular, natural or hydrogenated oils rich in (C₁₂-C₁₈ fatty acid) ester residues. In one embodiment, polyalkylene glycol materials are polyethylene glycols, in particular polyethylene glycols having a molecular weight of from ca. 500 to ca. 4,000, e.g. from ca. 1,000 to ca. 2,000.

In one embodiment, alkylene polyol ethers or esters include, but are not limited to, mixtures of C₃-C₅ alkylene triol esters, e.g. mono-, di- and tri-esters in variable relative amount, and poly (C₂-C₄ alkylene)glycol mono- and di-esters, together with minor amounts of free C₃-C₅ alkylene triol and free poly-(C₂-C₅ alkylene)glycol. As hereinabove set forth, in one embodiment, the alkylene triol moiety is glyceryl; in another embodiment, the polyalkylene glycol moieties include, but are not limited to, polyethylene glycol, in certain embodiments, having a molecular weight of from ca. 500 to ca. 4,000; and in another embodiment, the fatty acid moieties will be C₁₀-C₂₂ fatty acid ester residues, in certain embodiments, saturated C₁₀-C₂₂ fatty acid ester residues.

In one embodiment, the alkylene polyol ethers or esters include transesterification products of a natural or hydrogenated vegetable oil and a polyethylene glycol and, optionally, glycerol; or compositions comprising or consisting of glyceryl mono-, di- and tri-C₁₀-C₂₂ fatty acid esters and polyethylene glycol mono- and di-C₁₀-C₂₂ fatty esters (optionally together with, e.g. minor amounts of free glycerol and free polyethylene glycol).

In one embodiment, the alkylene polyol ethers or esters include, but are not limited, those commercially available under the trade name GELUCIRE® from e.g. Gattefossé, in particular the products:

a) GELUCIRE® 33/01, which has an m.p.=ca. 33-37° C. and a saponification value of about 230-255;

b) GELUCIRE® 39/01, m.p.=ca. 37.5-41.5° C., saponification value of about 225-245; and

c) GELUCIRE® 43/01, m.p.=ca. 42-46° C., saponification value of about 220-240.

Products (a) to (c) above all have an acid value of maximum of 3. The SEDDS or SMEDDS system provided herein may include mixtures of such ethers or esters.

B. Surfactants

The SEDDS or SMEDDS system provided herein can contain one or more surfactants to reduce the emulsion's interfacial tension thereby providing thermodynamic stability. Surfactants may be complex mixtures containing side products or unreacted starting products involved in the preparation thereof, e.g. surfactants made by polyoxyethylation may contain another side product, e.g. polyethylene glycol.

In one embodiment, surfactants include, but are not limited to:

B1) Polyoxyethylene Mono Esters of a Saturated C₁₀ to C₂₂ Polymer

These include, but are not limited to, C₁₁ substituted e.g. hydroxy fatty acid; e.g. 12 hydroxy stearic acid PEG ester, e.g. of PEG about e.g. 600-900, e.g. 660 Daltons MW, e.g. SOLUTOL®HS15 from BASF (Ludwigshafen, Germany). SOLUTOL®HS15, according to the BASF technical information (July 2003), comprises polyglycol mono- and di-esters of 12-hydroxystearic acid (=lipophilic part) and about 30% of free polyethylene glycol (=hydrophilic part). A small part of the 12-hydroxy group can be etherified with polyethylene glycol. SOLUTOL® HS15 has a hydrogenation value of 90 to 110, a saponification value of 53 to 63, an acid number of maximum 1, an iodine value of maximum 2, and a maximum water content of about 0.5% by weight. In one embodiment, the surfactant is SOLUTOL® HS15.

B2) Alkylene Polyol Ethers or Esters

In one embodiment, the alkylene polyol ethers or esters as described above for use in the pharmaceutical compositions provided herein include those commercially available under the trade name GELUCIRE® from e.g. Gattefossé (Paramus, N.J., USA), in particular the products:

a) GELUCIRE® 44/14, m.p.=ca. 42.5-47.5° C., saponification value of about 79-93;

b) GELUCIRE® 50/13, m.p.=ca. 46-51° C., saponification value of about 67-81;

Products (a) to (b) above both have an acid value of maximum of 2.

In one embodiment, the alkylene polyol ethers or esters have an iodine value of maximum 2. The SEDDS or SMEDDS system provided herein may further include mixtures of such ethers or esters.

GELUCIRE® products are inert semi-solid waxy materials with amphiphilic character. They are identified by their melting point and their HLB value. Most GELUCIRE® grades are saturated polyglycolised glycerides obtainable by polyglycolysis of natural hydrogenated vegetable oils with polyethylene glycols. They are composed of a mixture of mono-, di- and tri-glycerides and mono- and di-fatty acid esters of polyethylene glycol. In one embodiment, the C₁₀ glyceride is GELUCIRE® 44/14 which has a nominal melting point of 44° C. and an HLB of 14. GELUCIRE® 44/14 exhibits the following additional characterizing data: acid value of max. 2, iodine value of max. 2, saponification value of 79-93, hydroxyl value of 36-56, peroxide value of max. 6, alkaline impurities max. 80, water content max. 0.50, free glycerol content max. 3, monoglycerides content 3.0-8.0. (H. Fiedler, loc. cit., vol 1, page 676; manufacturer information).

In one embodiment, the surfactant is present in a range of from about 5 to about 99.9% by weight, or in a range of from about 30% to about 99.9% of the SEDDS or SMEDDS system provided herein.

In one embodiment, the surfactant comprises about 30% to about 70%, or about 40% to about 60% by weight of the carrier medium of the SEDDS or SMEDDS system provided herein.

In one embodiment, the SEDDS or SMEDDS system provided herein include additives e.g. antioxidants, flavors, sweeteners and other components known to those skilled in the art.

In one embodiment, the antioxidants include ascorbyl palmitate, butylated hydroxy anisole (BHA), 2,6-di-tert-butyl-4-methyl phenol (BHT) and tocopherols. In a further embodiment, the antioxidant is BHT.

In one embodiment, these additives may comprise about 0.005% to about 5% or about 0.01% to about 0.1% by weight of the total weight of the SEDDS or SMEDDS system. Antioxidants, or stabilizers typically provide up to about 0.005 to about 1% by weight based on the total weight of the composition. Sweetening or flavoring agents typically provide up to about 2.5% or 5% by weight based on the total weight of the composition.

The aforementioned additives can also include components that act as surfactants to solidify a liquid micro-emulsion pre-concentrate. These include solid polyethylene glycols (PEGs) and GELUCIRE® products, in one embodiment, the GELUCIRE® products include those such as GELUCIRE® 44/14 or GELUCIRE® 50/13.

When the SEDDS or SMEDDS system provided herein is combined with water or an aqueous solvent medium to obtain an emulsion, for example a microemulsion, the emulsion or microemulsion may be administered orally, for example in the form of a drinkable solution. The drinkable solution may comprise water or any other palatable aqueous system, such as fruit juice, milk and the like. In one embodiment, the relative proportion of the lipophilic component(s), the surfactant(s) and the hydrophilic component(s) lie within the “Microemulsion” region on a standard three way plot graph. The compositions will therefore be capable, on addition to an aqueous medium, of providing microemulsions, for example having a mean particle size of <200 nm.

In one embodiment, the carrier medium comprises about 30 to 70% by weight of one or more lipophilic components, wherein the one or more lipophilic components are a medium chain fatty acid triglyceride (A1), or a transesterified ethoxylated vegetable oil (A6). In a further embodiment, the medium chain fatty acid triglyceride (A1) is LABRAFAC® (Gattefossé, Paramus, N.J., USA). In another embodiment, the transesterified ethoxylated vegetable oil (A6) is LABRAFIL® (Gattefossé, Paramus, N.J., USA).

In one embodiment, the carrier medium comprises about 30 to 70% by weight of one or more surfactants, wherein the one or more surfactants are a polyoxyethylene mono ester (C₅), an alkylene polyol ether or ester (C₁₀), or a transesterified, polyoxyethylated caprylic-capric acid glyceride (C₁₃). In a further embodiment, the polyoxyethylene mono ester (C₅) is SOLUTOL® HS15 (BASF, Ludwigshafen, Germany). In another embodiment, the alkylene polyol ether or ester (C₁₀) is GELUCIRE®44/14 (Gattefosse, Paramus, N.J., USA). In yet another embodiment, the transesterified, polyoxyethylated caprylic-capric acid glyceride (C₁₃) is LABRASOL® (Gattefossé, Paramus, N.J., USA).

In one embodiment, the carrier medium comprises about 70% by weight LABRASOL®, about 18.3% by weight LABRAFAC® and about 11.7% by weight LABRAFIL®.

In one embodiment, the carrier medium comprises a range of about 65.1% to about 74.9% by weight LABRASOL®, a range of about 17.0% to about 19.6% by weight LABRAFAC® and a range of about 10.9% to about 12.5% by weight LABRAFIL®.

In one embodiment, the carrier medium comprises about 35% by weight LABRASOL®, about 35% by weight LABRAFAC® and about 30% by weight SOLUTOL® HS15.

In one embodiment, the carrier medium comprises a range of about 33.6% to about 37.4% by weight LABRASOL®, a range of about 33.6% to about 37.4% by weight LABRAFAC® and a range of about 27.9% to about 32.1% by weight SOLUTOL® HS15.

In one embodiment, the carrier medium comprises about 35% by weight LABRAFIL®, about 35% by weight LABRAFAC®, and about 30% by weight SOLUTOL®HS15.

In one embodiment, the carrier medium comprises a range of about 33.6% to about 37.4% by weight LABRAFIL®, a range of about 33.6% to about 37.4% by weight LABRAFAC®, and a range of about 27.9% to about 32.1% by weight SOLUTOL® HS15.

In one embodiment, the carrier medium comprises about 35% by weight GELUCIRE®44/14, about 35% by weight LABRAFAC®, and about 30% by weight SOLUTOL®HS15.

In one embodiment, the carrier medium comprises a range of about 33.6% to about 37.4% by weight GELUCIRE®44/14, a range of about 33.6% to about 37.4% by weight LABRAFAC®, and a range of about 27.9% to about 32.1% by weight SOLUTOL® HS15.

In one embodiment, provided herein is a SEDDS or SMEDDS system comprising a Compound provided herein, and a carrier medium comprising one or more surfactants. In one embodiment, the SEDDS or SMEDDS system additionally comprises an additive.

In one embodiment, the SEDDS or SMEDDS system comprises about 0.01% to about 5% by weight of a Compound provided herein.

In one embodiment, the dispersible pharmaceutical composition comprises about 95% to 99.09% by weight of one or more surfactants, wherein the one or more surfactants are selected from a group comprising an alkylene polyol ether or ester (C₁₀), and a polyoxyethylene mono ester (C₅). In a further embodiment, the alkylene polyol ether or ester (C₁₀) is GELUCIRE®44/14 (Gattefosse, Paramus, N.J., USA). In yet another embodiment, the polyoxyethylene mono ester (C₅) is SOLUTOL® HS15 (BASF, Ludwigshafen, Germany).

In one embodiment, the dispersible pharmaceutical composition comprises about 0.01% to about 0.1% by weight of an additive selected from a group comprising an antioxidant and a preservative. In a further embodiment, the additive is 2,6-di-tert-butyl-4-methylphenol (BHT).

In one embodiment, the SEDDS or SMEDDS system comprises about 0.28% by weight of a Compound provided herein, about 49.87% by weight of GELUCIRE®44/14, about 49.84% by weight of SOLUTOL®HS15 and about 0.01% by weight of BHT.

In one embodiment, the SEDDS or SMEDDS system comprises a range of about 0.26% to about 0.30% by weight of a Compound provided herein, a range of about 46.4% to about 53.4% by weight of GELUCIRE®44/14, a range of about 46.4% to about 53.3% by weight of SOLUTOL®HS15 and a range of about 0.009% to about 0.011% by weight of BHT.

In one embodiment, the SEDDS or SMEDDS system comprises about 1.43% by weight of a Compound provided herein, about 49.87% by weight of GELUCIRE®44/14, about 48.69% by weight of SOLUTOL®HS15 and about 0.01% by weight of BHT.

In one embodiment, the SEDDS or SMEDDS system comprises a range of about 1.33% to about 1.53% by weight of a Compound provided herein, a range of about 46.4% to about 53.4% by weight of GELUCIRE®44/14, a range of about 45.3% to about 52.1% by weight of SOLUTOL®HS15 and a range of about 0.009% to about 0.011% by weight of BHT.

In one embodiment, the SEDDS or SMEDDS system comprises about 2.67% by weight of a Compound provided herein, about 49.87% by weight of GELUCIRE®44/14, about 47.45% by weight of SOLUTOL®HS15 and about 0.01% by weight of BHT.

In one embodiment, the SEDDS or SMEDDS system comprises a range of about 2.48% to about 2.86% by weight of a Compound provided herein, a range of about 46.4% to about 53.4% by weight of GELUCIRE®44/14, a range of about 44.1% to about 50.8% by weight of SOLUTOL®HS15 and a range of about 0.009% to about 0.011% by weight of BHT.

In one embodiment, when the SEDDS or SMEDDS system provided herein is used to fill capsules for use in oral administration. The capsule may have a soft or hard capsule shell, for example, the capsule may be made of gelatine.

One group of SEDDS or SMEDDS systems provided herein may, on addition to water, provide aqueous microemulsions having an average particle size of about <200 nm (2,000 Å), about <150 nm (1,500 Å), or about <100 nm (1,000 Å).

In one embodiment, the SEDDS or SMEDDS systems provided herein exhibit advantageous properties when administered orally; for example in terms of consistency and high level of bioavailability obtained in standard bioavailability trials.

Pharmacokinetic parameters, for example, drug substance absorption and measured for example as blood levels, also can become more predictable and problems in administration with erratic absorption may be eliminated or reduced. Additionally pharmaceutical compositions provided herein are effective with biosurfactants or tenside materials, for example bile salts, being present in the gastro-intestinal tract. That is, pharmaceutical compositions provided herein are fully dispersible in aqueous systems comprising such natural tensides and thus capable of providing emulsion or microemulsion systems and/or particulate systems in situ which are stable. The function of pharmaceutical compositions provided herein upon oral administration remain substantially independent of and/or unimpaired by the relative presence or absence of bile salts at any particular time or for any given individual. Compositions provided herein may also reduce variability in inter- and intra-patient dose response.

In one embodiment, provided herein is a SEDDS or SMEDDS system comprising a Compound provided herein, and a carrier medium comprising one or more lipophilic components and one or more surfactants.

5.3 Patient Populations

In some embodiments, a subject treated for NF in accordance with the methods provided herein is a human who has or is diagnosed with NF (including NF1, NF2, and schwannomatosis). In other embodiments, a subject treated for NF in accordance with the methods provided herein is a human predisposed or susceptible to NF (including NF1, NF2, and schwannomatosis). In some embodiments, a subject treated for NF in accordance with the methods provided herein is a human at risk of developing NF. In specific embodiments, a subject treated for NF in accordance with the methods provided herein is human that meets one, two or more, or all of the criteria for subjects in the working examples in Section 11 et seq. In one embodiment, the subject is a male human. In another embodiment, the subject is a female human.

In specific embodiments, a subject diagnosed with NF that is treated in accordance with the method provided herein has one or more neurofibromas growing along nerves in the body, or on or under the skin. In particular embodiments, one or more of the neurofibromas is cancerous or metastatic. In other embodiments, one or more of the neurofibromas is benign. In certain embodiments, a subject diagnosed with NF has abnormalities such as skin changes and bone deformities. In particular embodiments, a subject treated for NF in accordance with the methods provided herein is a human diagnosed with NF that has neurologic, ophthalmologic, and/or cutaneous abnormalities.

In particular embodiments, a subject diagnosed with NF that is treated in accordance with the method provided herein has brown café au lait spots. Such spots may not hurt or itch and may never progress to anything more serious than spots. Such spots can be found anywhere on the body, though not usually on the face. In some embodiments, tiny spots—freckles—may be seen under the arms or in the groin area.

In some embodiments, a subject treated for NF in accordance with the methods provided herein inherited NF. In other embodiments, a subject treated for NF in accordance with the methods provided herein developed NF spontaneously through gene mutation.

In one embodiment, a subject treated for NF in accordance with the methods provided herein is a human infant. In one embodiment, a subject treated for NF in accordance with the methods provided herein is an elderly human. In another embodiment, a subject treated for NF in accordance with the methods provided herein is a human adult. In another embodiment, a subject treated for NF in accordance with the methods provided herein is a human child. In another embodiment, a subject treated for NF in accordance with the methods provided herein is a human toddler. In a specific embodiment, a subject treated for NF in accordance with the methods provided herein is a human that is 18 years old or is older than 18 years old. In certain embodiments, a subject treated for NF in accordance with the methods provided herein is a female human that is not pregnant or is not breastfeeding. In other embodiments, a subject treated for NF in accordance with the methods provided herein is a human that is pregnant or will become pregnant, or is breastfeeding.

In certain embodiments, a subject treated for NF in accordance with the methods provided herein is a human that is about 1 month to 12 months old, about 1 year to 10 years old, about 10 to 20 years old, about 12 to 18 years old, about 20 to 30 years old, about 30 to 40 years old, about 40 to 50 years old, about 50 to 60 years old, about 60 to 70 years old, about 70 to 80 years old, about 80 to 90 years old, about 90 to 100 years old, or any age in between. In a specific embodiment, a subject treated for NF in accordance with the methods provided herein is a human that is 18 years old or older. In a particular embodiment, a subject treated for NF in accordance with the methods provided herein is a human child that is between the age of 1 year old and 18 years old. In a certain embodiment, a subject treated for NF in accordance with the methods provided herein is a human that is between the age of 12 years old and 18 years old.

In particular embodiments, a subject treated for NF in accordance with the methods provided herein is a human that is in an immunocompromised state or immunosuppressed state. In certain embodiments, a subject treated for NF in accordance with the methods provided herein is a human receiving or recovering from immunosuppressive therapy. In certain embodiments, a subject treated for NF in accordance with the methods provided herein is a human who is, will or has undergone surgery, drug therapy such as chemotherapy and/or radiation therapy.

In specific embodiments, a subject treated for NF in accordance with the methods provided herein is suffering from a condition, e.g., stroke or cardiovascular conditions that may require VEGF therapy, wherein the administration of anti-angiogenic therapies other than a Compound may be contraindicated. For example, in certain embodiments, a subject treated for NF in accordance with the methods provided herein has suffered from a stroke or is suffering from a cardiovascular condition. In some embodiments, a subject treated for NF in accordance with the methods provided herein is a human experiencing circulatory problems. In certain embodiments, a subject treated for NF in accordance with the methods provided herein is a human with diabetic polyneuropathy or diabetic neuropathy. In some embodiments, a subject treated for NF in accordance with the methods provided herein is a human receiving VEGF protein or VEGF gene therapy. In other embodiments, a subject treated for NF in accordance with the methods provided herein is not a human receiving VEGF protein or VEGF gene therapy.

In some embodiments, a subject treated for NF in accordance with the methods provided herein is administered a Compound or a pharmaceutical composition thereof, or a combination therapy before any adverse effects or intolerance to therapies other than the Compound develops. In some embodiments, a subject treated for NF in accordance with the methods provided herein is a refractory patient. In a certain embodiment, a refractory patient is a patient with a tumor that is refractory to a standard therapy (e.g., surgery, radiation, and/or drug therapy such as chemotherapy). In certain embodiments, a patient with cancer associated with NF, is refractory to a therapy when the cancer has not significantly been eradicated and/or the symptoms have not been significantly alleviated. The determination of whether a patient is refractory can be made either in vivo or in vitro by any method known in the art for assaying the effectiveness of a treatment of NF, using art-accepted meanings of “refractory” in such a context. In various embodiments, a patient with NF is refractory when one or more tumors associated with NF have not decreased or have increased. In various embodiments, a patient with cancer associated with NF is refractory when one or more tumors metastasize and/or spreads to another organ. In some embodiments, a patient is in remission. In certain embodiments, a patient is experiencing recurrence of one or more tumors associated with NF.

In some embodiments, a subject treated for NF in accordance with the methods provided herein is a human that has proven refractory to therapies other than treatment with a Compound, but is no longer on these therapies. In certain embodiments, a subject treated for NF in accordance with the methods provided herein is a human already receiving one or more conventional anti-cancer therapies, such as surgery, drug therapy such as chemotherapy, or radiation. Among these patients are refractory patients, patients who are too young for conventional therapies, and patients with recurring tumors despite treatment with existing therapies.

In some embodiments, a subject treated for NF in accordance with the methods provided herein is a human susceptible to adverse reactions to conventional therapies. In some embodiments, a subject treated for NF in accordance with the methods provided herein is a human that has not received a therapy, e.g., drug therapy such as chemotherapy, surgery, or radiation therapy, prior to the administration of a Compound or a pharmaceutical composition thereof. In other embodiments, a subject treated for NF in accordance with the methods provided herein is a human that has received a therapy prior to administration of a Compound. In some embodiments, a subject treated for NF in accordance with the methods provided herein is a human that has experienced adverse side effects to the prior therapy or the prior therapy was discontinued due to unacceptable levels of toxicity to the human.

In some embodiments, a subject treated for NF in accordance with the methods provided herein has had no prior exposure to another anti-angiogenic therapy (e.g., an anti-VEGF monoclonal antibody, an anti-VEGFR monoclonal antibody, a tyrosine kinase inhibitor, or other angiogenesis pathway modulator). In particular embodiments, a subject treated for NF in accordance with the methods provided herein does not have uncontrolled hypertension, major bleeding, HIV infection or recent acute cardiovascular event. In some embodiments, a subject treated for NF in accordance with the methods provided herein is not, has not and/or will not receive a drug that is primarily metabolized by CYP2D6. In particular embodiments, a subject treated for NF in accordance with the methods provided herein has not and will not received a drug that is primarily metabolized by CYP2D6 1, 2, 3 or 4 weeks before receiving a Compound or a pharmaceutical composition thereof and 1, 2, 3 or 4 weeks after receiving the Compound or pharmaceutical composition. Examples of such drugs include, without limitation, some antidepressants (e.g., tricyclic antidepressants and selective serotonin uptake inhibitors), some antipsychotics, some beta-adrenergic receptor blockers, and certain anti-arrhythmics. In specific embodiments, a subject treated for NF in accordance with the methods provided herein is not, has not and/or will not receive tamoxifen. In particular embodiments, a subject treated for NF in accordance with the methods provided herein has not and will not received tamoxifen 1, 2, 3 or 4 weeks before receiving a Compound or a pharmaceutical composition thereof and 1, 2, 3 or 4 weeks after receiving the Compound or pharmaceutical composition. In specific embodiments, a subject treated for NF in accordance with the methods provided herein has received tamoxifen, e.g., for 1, 2, 3 or 4 weeks before receiving a Compound or a pharmaceutical composition thereof

5.3.1 NF1 Patients

In specific embodiments, a subject treated for NF in accordance with the methods provided herein is a human diagnosed with NF1 (also known as von Recklinghausen disease). In such embodiments, the subject contains a mutation of a gene on chromosome 17q11.2 called neurofibromin, a gene encoding a large protein called Neurofibromin. In certain embodiments, the gene mutation is inherited. In other embodiments, the gene mutation is a spontaneous mutation. In specific embodiments, a subject diagnosed with NF1 that is treated for NF in accordance with the methods provided herein has one or more of the following: light brown skin spots at birth or during childhood, neurofibromas (tumors that grow along nerves under the skin), plexiform neurofibromas (tumors involving multiple nerves), spinal cord and optic nerve tumors, and learning disabilities.

In particular embodiments, a subject diagnosed with NF1 that is treated for NF in accordance with the methods provided herein has two or more of the following: (a) six or more light brown spots on the skin (often called “cafe-au-lait” spots), e.g., measuring more than 5 millimeters in diameter in children, or more than 15 millimeters across in adolescents and adults; (b) two or more neurofibromas, or one plexiform neurofibroma (a neurofibroma that involves many nerves); (c) freckling in the area of the armpit or the groin; (d) two or more growths on the iris of the eye (known as Lisch nodules or iris hamartomas); (e) a tumor on the optic nerve (optic glioma); (f) abnormal development of the spine (scoliosis), the temple (sphenoid) bone of the skull, or the tibia (one of the long bones of the shin); and (g) a first degree relative (parent, sibling, or child) with NF1. In one embodiment, a subject treated for NF in accordance with the methods provided herein has 1, 2, 3, 4, 5, 6, 7, 8, 9, 10, or more cafe-au-lait spots. In another embodiment, a subject treated for NF in accordance with the methods provided herein has less than 6 cafe-au-lait spots.

In certain embodiments, a subject diagnosed with NF1 that is treated for NF in accordance with the methods provided herein displays one or more NF1 symptoms, e.g., larger than normal head circumference; shorter than average height; hydrocephalus (the abnormal buildup of fluid in the brain); headache and epilepsy; and cardiovascular complications associated with NF1, including congenital heart defects, high blood pressure (hypertension), and constricted, blocked, or damaged blood vessels (vasculopathy). In some embodiments, a subject treated for NF in accordance with the methods provided herein is a human child with NF1 that has poor linguistic and/or visual-spatial skills. In other embodiments, a subject treated for NF in accordance with the methods provided herein is a human child with NF1 that has low scores on academic achievement tests, including but not limited to those that measure reading, spelling, and/or math skills. In specific embodiments, a subject treated for NF in accordance with the methods provided herein is a human child or a human adult with NF1 that has a learning disability, such as ADHD. In particular embodiments, a subject treated for NF in accordance with the methods provided herein is a human with NF1 that has exhibited one or more symptoms of NF1 at birth and/or during early childhood. In other embodiments, a subject treated for NF in accordance with the methods provided herein is a human with NF1 that has exhibited one or more symptoms of NF1 during adolescence and/or adulthood.

In certain embodiments, a subject treated for NF in accordance with the methods provided herein is a human diagnosed with NF1 that has received one or more anti-cancer therapies such as surgery, radiation and/or drug therapies such as chemotherapy. In other specific embodiments, a subject treated for NF in accordance with the methods provided herein is a human diagnosed with NF1 that has not received one or more anti-cancer therapies such as surgery, radiation and/or drug therapy such as chemotherapy. In other embodiments, a subject treated for NF in accordance with the methods provided herein is a human diagnosed with NF1 that has received a therapy for NF1 or a condition associated therewith that is aimed at controlling or relieving one or more symptoms thereof, e.g., headache and epileptic seizures.

5.3.2 NF2 Patients

In specific embodiments, a subject treated for NF in accordance with the methods provided herein is a human diagnosed with NF2. In some embodiments, a subject treated for NF in accordance with the methods provided herein is a human diagnosed with NF2 that is not a good surgical candidate. In certain embodiments, the subject is at elevated risk for surgical complications (e.g., deafness, lower cranial nerve injury, or facial weakness) or who refuses surgery.

In certain embodiments, a subject treated for NF in accordance with the methods provided herein is a human diagnosed with NF2 that has progressive hearing loss related to VS. In specific embodiments, a subject treated for NF in accordance with the methods provided herein is diagnosed with NF2 that is not a good surgical candidate and has progressive hearing loss related to VS. In some embodiments, a subject treated for NF in accordance with the methods provided herein has VS. In particular embodiments, the subject possess evidence of a progressive increase in VS size or worsening hearing loss due to VS. In certain embodiments, the progressive VS growth is characterized as greater than or equal to 20% increase in either volume or greater than or equal to 2 mm increase in greatest linear dimension based on serial MRI studies.

In particular embodiments, a subject treated for NF in accordance with the methods provided herein is a human diagnosed with NF that has neurologic, ophthalmologic, and/or cutaneous abnormalities. In certain embodiments, a subject treated for NF in accordance with the methods provided herein is a human diagnosed with NF2 that has an inactivation of the tumor suppressor gene, NF2, which encodes the protein Merlin (a.k.a. schwannomin). In particular embodiments, a subject treated for NF in accordance with the methods provided herein is a human diagnosed with NF2 that presents/presented symptoms such as hearing loss, tinnitus, visual impairment (such as vision loss from cataracts), imbalance, or painful skin lesions or tumors. For example, such symptoms may present in a human between the ages of 17 and 21. In other embodiments, the subject exhibits symptoms such as weakness in an arm or leg, seizures, vertigo, and/or facial weakness/paralysis. In certain embodiment, a formal diagnosis of NF2 has, is or can be established by the presence of bilateral VS or unilateral VS in conjunction with the presence of NF2 associated tumors (e.g., meningiomas, schwannomas, ependymomas, glioma, neurofibroma), posterior cataracts, or a family history of other NF2-related tumors. In addition to the morbidity associated with auditory and vestibular deficits, a subject treated for NF in accordance with the methods provided herein may experience other neurologic dysfunction related to VS growth (e.g., due to compression of other cranial nerves). In specific embodiments, a subject treated for NF in accordance with the methods provided herein is a human diagnosed with NF2 that has skull-base tumors (including VS and meningiomas).

In certain embodiments, a subject treated for NF, e.g., NF2, in accordance with the methods provided herein is a human diagnosed with NF2 that has undergone surgery as the primary treatment option. In specific embodiments, a subject treated for NF, e.g., NF2, in accordance with the methods provided herein is a human diagnosed with NF2 that has not undergone surgery for removal of one or more tumors. In particular embodiments, surgical removal of all tumors is not possible or advisable. Surgery may not be an option for a patient because it may introduce significant post operative risks, including hearing loss, facial weakness, and dysphagia. In certain embodiments, a subject treated for NF, e.g., NF2, in accordance with the methods provided herein is a human diagnosed with NF2 that has undergone irradiation of tumors. In specific embodiments, a subject treated for NF, e.g., NF2, in accordance with the methods provided herein is a human diagnosed with NF2 that has not been treated with surgery, radiation or drug therapy such as chemotherapy for the NF.

In specific embodiments, a subject treated for NF, e.g., NF2, in accordance with the methods provided herein is a human having a diagnosis of NF2 by National Institutes of Health (NIH) criteria (see NIH. Neurofibromatosis. Conference statement. National Institutes of Health Consensus Development Conference. Arch Neurol. 1988 May 45(5): 575-8) with evidence of either: (i) Bilateral VS, or (ii) First-degree family relative with NF2 and either unilateral VS or any 2 of: meningioma, schwannoma, glioma, neurofibroma, and juvenile posterior subcapsular lens opacity. In some embodiments, a subject treated for N, e.g., NF2, in accordance with the methods provided herein is a human with evidence of disease progression defined by any of the following features: (i) progressive VS growth (≧20% increase in either volume [if volumetric measurement performed], or ≧2 mm increase in greatest linear dimension) based on serial MRI studies in subjects who are at elevated risk for surgical complications (e.g., deafness, lower cranial nerve injury, or facial weakness) or who refuse surgery; and (ii) progressive hearing loss related to VS (i.e., not due to surgery or radiation) with a word recognition score of <85% in at least 1 affected ear. In some embodiments, a subject's participation in the methods for treating NF2 provided herein, in the judgment of a physician, offers acceptable benefit:risk when considering current NF2 disease status, medical condition, and the potential benefits of and risks of surgery or irradiation. In certain embodiments, a subject treated for NF in accordance with the methods provided herein has discontinued all therapies (except corticosteroids) for the treatment of NF2 4 weeks or more before initiation of said method (e.g., administration of a Compound). In some embodiments, a subject treated for NF, e.g., NF2, in accordance with the methods provided herein meets one or more of the following conditions:
a. The condition that all acute toxic effects (excluding alopecia or neurotoxicity) of any prior therapy resolved to Common Terminology Criteria for Adverse Events (“CTCAE”) Grade≦1 before initiation of treatment with a Compound; and/or
b. Adequate functional status (Karnofsky Performance Score≧60).

In certain embodiments, a subject treated for NF, e.g., NF2, in accordance with the methods provided herein is not pregnant or will not become pregnant.

5.3.3 Schwannomatosis Patients

In one embodiment, a subject treated for NF in accordance with the methods provided herein is a human diagnosed with schwannomatosis. In a specific embodiment, a subject treated for NF in accordance with the methods provided herein is a human that has one or more mutations in the SMARCB1 (hSnf5/INI1) tumor suppressor gene. In particular embodiments, a subject treated for NF in accordance with the methods provided herein is a human that has multiple schwannomas or tumors of nerve sheaths. In specific embodiments, a subject treated for NF in accordance with the methods provided herein is a human diagnosed with schwannomatosis that does not have vestibular (ear nerve) tumors. In particular embodiments, a subject treated for NF in accordance with the methods provided herein is a human diagnosed with schwannomatosis that develops tumors on the sheaths, or coverings, of the nerves.

5.4 Dosage and Administration

In accordance with the methods for treating NF provided herein, a Compound or a pharmaceutical composition thereof can be administered to a subject in need thereof by a variety of routes in amounts which result in a beneficial or therapeutic effect. A Compound or pharmaceutical composition thereof may be orally administered to a subject in need thereof in accordance with the methods for treating NF provided herein. The oral administration of a Compound or a pharmaceutical composition thereof may facilitate subjects in need of such treatment complying with a regimen for taking the Compound or pharmaceutical composition. Thus, in a specific embodiment, a compound or pharmaceutical composition thereof is administered orally to a subject in thereof.

Other routes of administration include, but are not limited to, intravenous, intrathecal, intradermal, intramuscular, subcutaneous, intranasal, inhalation, transdermal, topical, transmucosal, intracranial, intratumoral, epidural and intra-synovial. In one embodiment, a Compound or a pharmaceutical composition thereof is administered systemically (e.g., parenterally) to a subject in need thereof. In another embodiment, a Compound or a pharmaceutical composition thereof is administered locally (e.g., intratumorally) to a subject in need thereof. In one embodiment, a Compound or a pharmaceutical composition thereof is administered intrathecally or via a route that permits the Compound to cross the blood-brain barrier (e.g., orally).

Evaluation has indicated that Compound #10 penetrates the blood-brain barrier. Table 33 provides brain tissue plasma concentration ratios determined by whole-body autoradiography at specified times after a single oral administration of ¹⁴C-Compound #10 to rats (50 mg/kg).

TABLE 33
Blood-Brain Barrier Penetration

| | 6 Hours | | 12 Hours | | 24 Hours | | 48 Hours | | 72 Hours | |
|---|---|---|---|---|---|---|---|---|---|---|
| Tissue | M | F | M | F | M | F | M | F | M | F |
| Cerebellum | 1.55 | 1.23 | 1.85 | 2.85 | 1.74 | 1.59 | 1.21 | 1.17 | NA | 2.04 |
| Cerebrum | 1.52 | 1.22 | 1.75 | 2.79 | 1.89 | 1.57 | 1.35 | 1.68 | NA | 1.56 |
| Medulla | 1.60 | 1.42 | 1.98 | 3.82 | 1.83 | 1.69 | 1.20 | 2.01 | NA | 1.88 |
| Olfactory lobe | 1.42 | 1.38 | 1.35 | 2.45 | 1.23 | 1.13 | 0.967 | NA | NA | 3.33 |
| Pituitary gland | 4.06 | 4.27 | 3.22 | 5.48 | 2.72 | 2.33 | 0.890 | 3.68 | NA | 1.58 |
| Spinal cord | 1.14 | 0.898 | 1.24 | 1.92 | 1.75 | 1.60 | 1.43 | 1.60 | 1.84 | 2.75 |

In accordance with the methods for treating NF provided herein that involve administration of a Compound in combination with one or more additional therapies, the Compound and one or more additional therapies may be administered by the same route or a different route of administration.

The dosage and frequency of administration of a Compound or a pharmaceutical composition thereof is administered to a subject in need thereof in accordance with the methods for treating NF provided herein will be efficacious while minimizing any side effects. The exact dosage and frequency of administration of a Compound or a pharmaceutical composition thereof can be determined by a practitioner, in light of factors related to the subject that requires treatment. Factors which may be taken into account include the severity of the disease state, general health of the subject, age, weight, and gender of the subject, diet, time and frequency of administration, drug combination(s), reaction sensitivities, and tolerance/response to therapy. The dosage and frequency of administration of a Compound or a pharmaceutical composition thereof may be adjusted over time to provide sufficient levels of the Compound or to maintain the desired effect.

In certain embodiments, a Compound or a pharmaceutical composition thereof is administered to a subject in need thereof in accordance with the methods for treating NF provided herein at a dosage and a frequency of administration that achieves one or more of the following: (i) decreases the production and/or concentration of VEGF or other angiogenic or inflammatory mediators or a change in tumor blood flow or metabolism, or peritumoral inflammation or edema in a subject with NF or an animal model with a pre-established human tumor; (ii) reduces or ameliorates the severity of NF and/or a symptom associated therewith in a subject with NF; (iii) reduces the number symptoms and/or the duration of a symptom(s) associated with NF in a subject with NF; (iv) prevents the onset, progression or recurrence of one or more symptoms associated with NF in a subject with NF; (v) prevents the recurrence of a tumor associated with NF; (vi) reduces hearing loss, tinnitus, visual impairment, imbalance, and/or painful skin lesions associated with NF in a subject with NF; (vii) improves hearing, hearing function and/or word recognition in a subject with NF; and/or (viii) enhances or improves the therapeutic effect of another therapy in a subject with NF or an animal model with a pre-established human tumor.

In certain embodiments, a Compound or a pharmaceutical composition thereof is administered to a subject in need thereof in accordance with the methods for treating NF provided herein at a dosage and a frequency of administration that results in one or more of the following: (i) regression of a tumor associated with NF and/or inhibition of the progression of a tumor associated with NF in a subject with NF or an animal model with a pre-established human tumor; (ii) reduction in the growth of a tumor or neoplasm associated with NF and/or decrease in the tumor size (e.g., volume or diameter) of a tumor associated with NF (e.g., neurofibromas, plexiform neurofibromas, meningiomas, schwannomas, gliomas, or ependymomas) in a subject with NF or an animal model with a pre-established human tumor; (iii) the size of a tumor associated with NF is maintained and/or the tumor does not increase or increases by less than the increase of a similar tumor after administration of a standard therapy as measured by conventional methods available to one of skill in the art, such as MRI, DCE-MRI, PET scan, X-ray, and CT scan; (iv) reduction in the formation of a tumor associated with NF (e.g., neurofibromas, plexiform neurofibromas, meningiomas, schwannomas, gliomas, or ependymomas) in a subject with NF or an animal model with a pre-established human tumor; (v) eradication, removal, or control of primary, regional and/or metastatic tumors associated with NF in a subject with NF or an animal model with a pre-established human tumor; (vi) a decrease in the number or size of metastases associated with NF in a subject with NF or an animal model with a pre-established human tumor; and/or (vii) reduction in the growth of a pre-established tumor (e.g., glioma, etc.) or neoplasm and/or decrease in the tumor size (e.g., volume or diameter) of a pre-established tumor (e.g., glioma, etc.) in a subject with NF or an animal model with a pre-established human tumor.

In certain embodiments, a Compound or a pharmaceutical composition thereof is administered to a subject in need thereof in accordance with the methods for treating NF provided herein at a dosage and a frequency of administration that achieve one or more of the following: (i) improvement in neural function, e.g., hearing, balance, tinnitus, or vision; (ii) inhibition or reduction in pathological production of VEGF; (iii) stabilization or reduction of peritumoral inflammation or edema in a subject; (iv) reduction of the concentration of VEGF or other angiogenic or inflammatory mediators (e.g., cytokines or interleukins) in biological specimens (e.g., plasma, serum, cerebral spinal fluid, urine, or any other biofluids); (v) reduction of the concentration of P1GF, VEGF-C, VEGF-D, VEGFR-1, VEGFR-2, IL-6, and/or IL-8 in biological specimens (e.g., plasma, serum, cerebral spinal fluid, urine, or any other biofluids); (vi) inhibition or decrease in tumor metabolism or perfusion; (vii) inhibition or decrease in angiogenesis or vascularization; and/or (viii) improvement in quality of life as assessed by methods well known in the art, e.g., tinnitus questionnaires.

In one aspect, a method for treating NF presented herein involves the administration of a unit dosage of a Compound or a pharmaceutical composition thereof. The unit dosage may be administered as often as determined effective (e.g., once, twice or three times per day, every other day, once or twice per week, biweekly or monthly). In certain embodiments, a method for treating NF presented herein involves the administration to a subject in need thereof of a unit dose of a Compound or a pharmaceutical composition thereof that ranges from about 0.1 milligram (mg) to about 1000 mg, from about 1 mg to about 1000 mg, from about 5 mg to about 1000 mg, from about 10 mg to about 500 mg, from about 100 mg to about 500 mg, from about 150 mg to about 500 mg, from about 150 mg to about 1000 mg, from about 250 mg to about 1000 mg, from about 300 mg to about 1000 mg, or from about 500 mg to about 1000 mg, or any range in between. In some embodiments, a method for treating NF presented herein involves the administration to a subject in need thereof of a unit dose of a Compound or a pharmaceutical composition thereof of about 15 mg, 16, mg, 17 mg, 18 mg, 19 mg, 20 mg, 21, mg, 22 mg, 23 mg, 24 mg, 25 mg, 26 mg, 27 mg, 28 mg, 29 mg, 30 mg or 40 mg. In certain embodiments, a method for treating NF presented herein involves the administration to a subject in need thereof of a unit dose of a Compound or a pharmaceutical composition thereof of about 50 mg, 60 mg, 70 mg, 80 mg, 90 mg, 100 mg, 110 mg, 120 mg, 125 mg, 130 mg, 140 mg, 150 mg, 175 mg, 200 mg, 250 mg, 300 mg, 350 mg, 400 mg, 450 mg, 500 mg, 550 mg, 600 mg, 650 mg, 700 mg, 750 mg, 800 mg, 850 mg, or 900 mg. In some embodiments, a method for treating NF presented herein involves the administration to a subject in need thereof of a unit dose of a Compound or a pharmaceutical composition thereof of at least about 0.1 mg, 1 mg, 5 mg, 10 mg, 20 mg, 30 mg, 40 mg, 50 mg, 60 mg, 70 mg, 80 mg, 90 mg, 100 mg, 110 mg, 120 mg, 125 mg, 130 mg, 140 mg, 150 mg, 175 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg or more. In certain embodiments, a method for treating NF presented herein involves the administration to a subject in need thereof of a unit dose of a Compound or a pharmaceutical composition thereof of less than about 35 mg, less than about 40 mg, less than about 45 mg, less than about 50 mg, less than about 60 mg, less than about 70 mg, or less than about 80 mg.

In specific embodiments, a method for treating NF presented herein involves the administration to a subject in need thereof of a unit dose of a Compound or a pharmaceutical composition thereof of about 40 mg to about 500 mg, about 40 mg to about 200 mg, about 40 mg to about 150 mg, about 75 mg to about 500 mg, about 75 mg to about 450 mg, about 75 mg to about 400 mg, about 75 mg to about 350 mg, about 75 mg to about 300 mg, about 75 mg to about 250 mg, about 75 mg to about 200 mg, about 100 mg to about 200 mg, or any range in between. In other specific embodiments, a method for treating NF presented herein involves the administration to a subject in need thereof of a unit dose of a Compound or a pharmaceutical composition thereof of about 35 mg, 40 mg, 50 mg, 60 mg, 75 mg, 100 mg, 125 mg, 150 mg, 175 mg, 200 mg, 225 mg, 250 mg or 300 mg. In some embodiments, a method for treating NF presented herein involves the administration to a subject in need thereof of a unit dose of a Compound or a pharmaceutical composition thereof of about 350 mg, 400 mg, 500 mg, 600 mg, 700 mg, 800 mg, 900 mg, or 1000 mg. In some embodiments, a unit dose of a Compound or a pharmaceutical composition thereof is administered to a subject once per day, twice per day, three times per day; once, twice or three times every other day (i.e., on alternate days); once, twice or three times every two days; once, twice or three times every three days; once, twice or three times every four days; once, twice or three times every five days; once, twice, or three times once a week, biweekly or monthly.

In certain embodiments, a method for treating NF presented herein involves the administration to a subject in need thereof of a unit dose of a Compound or a pharmaceutical composition thereof that ranges from about 50 mg to about 500 mg per day. In some embodiments, a method for treating NF presented herein involves the administration to a subject in need thereof of a unit dose of a Compound or a pharmaceutical composition thereof that ranges from about 80 mg to about 500 mg per day, about 100 mg to about 500 mg per day, about 80 mg to about 400 mg per day, about 80 mg to about 300 mg per day, about 80 mg to about 200 mg per day, about 200 mg to about 300 mg per day, about 200 mg to about 400 mg per day, or any range in between. In a specific embodiment, a method for treating NF presented herein involves the administration to a subject in need thereof of a unit dose of about 40 mg of a Compound or a pharmaceutical composition thereof twice per day. In another specific embodiment, a method for treating NF presented herein involves the administration to a subject in need thereof of a unit dose of about 80 mg of a Compound or a pharmaceutical composition thereof twice per day. In another specific embodiment, a method for treating NF presented herein involves the administration to a subject in need thereof of a unit dose of about 100 mg of a Compound or a pharmaceutical composition thereof twice per day. In another specific embodiment, a method for treating NF presented herein involves the administration to a subject in need thereof of a Compound or a pharmaceutical composition at the dosage, frequency of administration and route of administration set forth in the working examples infra in Section 11 et seq.

In some embodiments, a method for treating NF presented herein involves the administration of a dosage of a Compound or a pharmaceutical composition thereof that is expressed as mg per meter squared (mg/m²). The mg/m² for a Compound may be determined, for example, by multiplying a conversion factor for an animal by an animal dose in mg/kg to obtain the dose in mg/m² for human dose equivalent. For regulatory submissions the FDA may recommend the following conversion factors: Mouse=3, Hamster=4.1, Rat=6, Guinea Pig=7.7. (based on Freireich et al., Cancer Chemother. Rep. 50(4):219-244 (1966)). The height and weight of a human may be used to calculate a human body surface area applying Boyd's Formula of Body Surface Area. In specific embodiments, a method for treating NF presented herein involves the administration to a subject in need thereof of an amount of a Compound or a pharmaceutical composition thereof in the range of from about 0.1 mg/m² to about 1000 mg/m², or any range in between.

Other non-limiting exemplary doses of a Compound that may be used in the methods for treating NF provided herein include mg or microgram (m) amounts per kilogram (kg) of subject or sample weight per day such as from about 0.001 mg per kg to about 1500 mg per kg per day, from about 0.001 mg per kg to about 1400 mg per kg per day, from about 0.001 mg per kg to about 1300 mg per kg per day, from about 0.001 mg per kg to about 1200 mg per kg per day, from about 0.001 mg per kg to about 1100 mg per kg per day, from about 0.001 mg per kg to about 1000 mg per kg per day, from about 0.01 mg per kg to about 1500 mg per kg per day, from about 0.01 mg per kg to about 1000 mg per kg per day, from about 0.1 mg per kg to about 1500 mg per kg per day, from about 0.1 mg per kg to about 1000 mg per kg per day, from about 0.1 mg per kg to about 500 mg per kg per day, from about 0.1 mg per kg to about 100 mg per kg per day, or from about 1 mg per kg to about 100 mg per kg per day. In specific embodiments, oral doses for use in the methods provided herein are from about 0.01 mg to about 300 mg per kg body weight per day, from about 0.1 mg to about 75 mg per kg body weight per day, or from about 0.5 mg to 5 mg per kg body weight per day. In certain embodiments, In certain embodiments, oral doses for use in the methods provided herein involves the oral administration to a subject in need thereof of a dose of a Compound or a pharmaceutical composition thereof that ranges from about 80 mg to about 800 mg per kg per day, from about 100 mg to about 800 mg per kg per day, from about 80 mg to about 600 mg per kg per day, from about 80 mg to about 400 mg per kg per day, from about 80 mg to about 200 mg per kg per day, from about 200 mg to about 300 mg per kg per day, from about 200 mg to about 400 mg per kg per day, from about 200 mg to about 800 mg per kg per day, or any range in between. In certain embodiments, doses of a Compound that may be used in the methods provided herein include doses of about 0.1 mg/kg/day, 0.2 mg/kg/day, 0.3 mg/kg/day, 0.4 mg/kg/day, 0.5 mg/kg/day, 0.6 mg/kg/day, 0.7 mg/kg/day, 0.8 mg/kg/day, 0.9 mg/kg/day, 1 mg/kg/day, 1.5 mg/kg/day, 2 mg/kg/day, 2.5 mg/kg/day, 2.75 mg/kg/day, 3 mg/kg/day, 4 mg/kg/day, 5 mg/kg/day, 6 mg/kg/day, 6.5 mg/kg/day, 6.75 mg/kg/day, 7 mg/kg/day, 7.5 mg/kg/day, 8 mg/kg/day, 8.5 mg/kg/day, 9 mg/kg/day, 10 mg/kg/day, 11 mg/kg/day, 12 mg/kg/day, 13 mg/kg/day, 14 mg/kg/day or 15 mg/kg/day. In accordance with these embodiments, the dosage may be administered one, two or three times per day, every other day, or once or twice per week and the dosage may be administered orally.

In specific aspects, a method for treating NF presented herein involves the administration to a subject in need thereof of a Compound or a pharmaceutical composition thereof at a dosage that achieves a target plasma concentration of the Compound in a subject with NF or an animal model with a pre-established human tumor (e.g., tumor associated with NF). In a particular embodiment, a method for treating NF presented herein involves the administration to a subject in need thereof of a Compound or a pharmaceutical composition thereof at a dosage that achieves a plasma concentration of the Compound ranging from approximately 0.001 μg/mL to approximately 100 mg/mL, approximately 0.01 μg/mL to approximately 100 mg/mL, approximately 0.01 μg/mL to approximately 10 mg/mL, approximately 0.1 μg/mL to approximately 10 mg/mL, approximately 0.1 μg/mL to approximately 500 μg/mL, approximately 0.1 μg/mL to approximately 500 μg/mL, approximately 0.1 μg/mL to approximately 100 μg/mL, or approximately 0.5 μg/mL to approximately 10 μg/mL in a subject with NF or an animal model with a pre-established human tumors (e.g., tumors associated with NF). To achieve such plasma concentrations, a Compound or a pharmaceutical composition thereof may be administered at doses that vary from 0.001 μg to 100,000 mg, depending upon the route of administration. In certain embodiments, subsequent doses of a Compound may be adjusted accordingly based on the plasma concentrations of the Compound achieved with initial doses of the Compound or pharmaceutical composition thereof administered to the subject.

In specific aspects, a method for treating NF presented herein involves the administration to a subject in need thereof of a Compound or a pharmaceutical composition thereof at a dosage that achieves a target plasma concentration of VEGF, P1GF, VEGF-C, VEGF-D, IL-6, IL-8, VEGFR1 and/or VEGFR2 in a subject with NF or an animal model with a pre-established human tumor (e.g., tumor associated with NF). In a particular embodiment, a method for treating NF presented herein involves the administration to a subject in need thereof of a Compound or a pharmaceutical composition thereof at a dosage that achieves a plasma concentration of VEGF, P1GF, VEGF-C, and/or VEGF-D, IL-6, IL-8, VEGFR1 or VEGFR2 ranging from approximately 0.1 pg/mL to approximately 100 mg/mL, approximately 0.1 pg/mL to approximately 1 mg/mL, approximately 0.1 pg/mL to approximately 500 μg/mL, approximately 0.1 pg/mL to approximately 500 μg/mL, approximately 0.1 pg/mL to approximately 100 μg/mL, or approximately 4 pg/mL to approximately 10 μg/mL in a subject with NF or an animal model with a pre-established human tumor (e.g., tumor associated with NF). To achieve such plasma concentrations, a Compound or a pharmaceutical composition thereof may be administered at doses that vary from 0.1 pg to 100,000 mg, depending upon the route of administration. In certain embodiments, subsequent doses of a Compound or a pharmaceutical composition thereof may be adjusted accordingly based on the plasma concentrations of VEGF, P1GF, VEGF-C, VEGF-D, IL-6, IL-8, VEGFR1 or VEGFR2 achieved with initial doses of the Compound or pharmaceutical composition thereof administered to the subject.

In specific aspects, a method for treating NF presented herein involves the administration to a subject in need thereof of a Compound or a pharmaceutical composition thereof at a dosage and/or a frequency of administration that achieves an imaging outcome indicating inhibition, stability, and/or reduction in a monitoring parameter such as tumor size, tumor perfusion, tumor metabolism, or peritumoral inflammation or edema, as assessed, e.g., by MRI scan, DCE-MRI scan, PET scan, and/or CT scan. To achieve such imaging outcome, a Compound or a pharmaceutical composition thereof may be administered at doses that vary from 0.1 pg to 100,000 mg, depending upon the route and/or frequency of administration. In certain embodiments, subsequent doses of a Compound or a pharmaceutical composition thereof may be adjusted accordingly based on the imaging outcome achieved with initial doses of the Compound or pharmaceutical composition thereof administered to the subject, as assessed, e.g., by MRI scan, DCE-MRI scan, PET scan, and/or CT scan.

In particular embodiments, a method for treating NF presented herein involves the administration to a subject in need thereof of a Compound or a pharmaceutical composition thereof at a dosage that achieves the desired tissue to plasma concentration ratios of the Compound as determined, e.g., by any imaging techniques known in the art such as whole-body autoradiography, in a subject with NF or an animal model (such as an animal model with a pre-established human tumor, e.g., a tumor associated with NF). Table 23 lists exemplary tissue to plasma concentration ratios of a Compound as determined by whole-body autoradiography.

In some embodiments, a method for treating NF presented herein involves the administration to a subject in need thereof of one or more doses of an effective amount of a Compound or a pharmaceutical composition, wherein the effective amount may or may not be the same for each dose. In particular embodiments, a first dose of a Compound or pharmaceutical composition thereof is administered to a subject in need thereof for a first period of time, and subsequently, a second dose of a Compound is administered to the subject for a second period of time. The first dose may be more than the second dose, or the first dose may be less than the second dose. A third dose of a Compound also may be administered to a subject in need thereof for a third period of time.

In some embodiments, the dosage amounts described herein refer to total amounts administered; that is, if more than one Compound is administered, then, in some embodiments, the dosages correspond to the total amount administered. In a specific embodiment, oral compositions contain about 5% to about 95% of a Compound by weight.

The length of time that a subject in need thereof is administered a Compound or a pharmaceutical composition in accordance with the methods for treating NF presented herein will be the time period that is determined to be efficacious. In certain embodiments, a method for treating NF presented herein involves the administration of a Compound or a pharmaceutical composition thereof for a period of time until the severity and/or number of symptoms associated with NF decrease. In some embodiments, a method for treating NF presented herein involves the administration of a Compound or a pharmaceutical composition thereof for up to 48 weeks. In other embodiments, a method for treating NF presented herein involves the administration of a Compound or a pharmaceutical composition thereof for up to about 4 weeks, 8 weeks, 12 weeks, 16 week, 20 weeks, 24 weeks, 26 weeks (0.5 year), 52 weeks (1 year), 78 weeks (1.5 years), 104 weeks (2 years), or 130 weeks (2.5 years) or more. In certain embodiments, a method for treating NF presented herein involves the administration of a Compound or a pharmaceutical composition thereof for an indefinite period of time. In some embodiments, a method for treating NF presented herein involves the administration of a Compound or a pharmaceutical composition thereof for a period of time followed by a period of rest (i.e., a period wherein the Compound is not administered) before the administration of the Compound or pharmaceutical composition thereof is resumed. In specific embodiments, a method for treating NF presented herein involves the administration of a Compound or a pharmaceutical composition thereof in cycles, e.g., 1 week cycles, 2 week cycles, 3 week cycles, 4 week cycles, 5 week cycles, 6 week cycles, 8 week cycles, 9 week cycles, 10 week cycles, 11 week cycles, or 12 week cycles. In such cycles, the Compound or a pharmaceutical composition thereof may be administered once, twice, three times, or four times daily. In particular embodiments, a method for treating a NF presented herein involves the administration of a Compound or a pharmaceutical composition thereof twice daily in 4 week cycles.

In specific embodiments, the period of time of administration of a Compound or pharmaceutical composition thereof may be dictated by one or more monitoring parameters, e.g., concentration of VEGF or other angiogenic or inflammatory mediators (e.g., cytokines or interleukins such as IL-6 or IL-8); tumor size, blood flow, or metabolism; peritumoral inflammation or edema. In particular embodiments, the period of time of administration of a Compound or pharmaceutical composition thereof may be adjusted based on one or more monitoring parameters, e.g., concentration of VEGF or other angiogenic or inflammatory mediators (e.g., cytokines or interleukins such as IL-6 or IL-8); tumor size, blood flow, or metabolism; and/or peritumoral inflammation or edema.

In certain embodiments, in accordance with the methods for treating NF presented herein, a Compound or a pharmaceutical composition thereof is administered to a subject in need thereof prior to, concurrently with, or after a meal (e.g., breakfast, lunch, or dinner). In specific embodiments, in accordance with the methods for treating NF presented herein, a Compound or a pharmaceutical composition thereof is administered to a subject in need thereof in the morning (e.g., between 5 am and 12 pm). In certain embodiments, in accordance with the methods for treating NF presented herein, a Compound or a pharmaceutical composition thereof is administered to a subject in need thereof at noon (i.e., 12 pm). In particular embodiments, in accordance with the methods for treating NF presented herein, a Compound or a pharmaceutical composition thereof is administered to a subject in need thereof in the afternoon (e.g., between 12 pm and 5 pm), evening (e.g., between 5 pm and bedtime), and/or before bedtime.

In specific embodiments, a dose of a Compound or a pharmaceutical composition thereof is administered to a subject once per day, twice per day, three times per day; once, twice or three times every other day (i.e., on alternate days); once, twice or three times every two days; once, twice or three times every three days; once, twice or three times every four days; once, twice or three times every five days; once, twice, or three times once a week, biweekly or monthly.

5.5 Combination Therapy

Presented herein are combination therapies for the treatment of NF which involve the administration of a Compound in combination with one or more additional therapies to a subject in need thereof. In a specific embodiment, presented herein are combination therapies for the treatment of NF which involve the administration of an effective amount of a Compound in combination with an effective amount of another therapy to a subject in need thereof.

As used herein, the term “in combination,” refers, in the context of the administration of a Compound, to the administration of a Compound prior to, concurrently with, or subsequent to the administration of one or more additional therapies (e.g., agents, surgery, or radiation) for use in treating NF. The use of the term “in combination” does not restrict the order in which one or more Compounds and one or more additional therapies are administered to a subject. In specific embodiments, the interval of time between the administration of a Compound and the administration of one or more additional therapies may be about 1-5 minutes, 1-30 minutes, 30 minutes to 60 minutes, 1 hour, 1-2 hours, 2-6 hours, 2-12 hours, 12-24 hours, 1-2 days, 2 days, 3 days, 4 days, 5 days, 6 days, 7 days, 1 week, 2 weeks, 3 weeks, 4 weeks, 5 weeks, 6 weeks, 7 weeks, 8 weeks, 9 weeks, 10 weeks, 15 weeks, 20 weeks, 26 weeks, 52 weeks, 11-15 weeks, 15-20 weeks, 20-30 weeks, 30-40 weeks, 40-50 weeks, 1 month, 2 months, 3 months, 4 months 5 months, 6 months, 7 months, 8 months, 9 months, 10 months, 11 months, 12 months, 1 year, 2 years, or any period of time in between. In certain embodiments, a Compound and one or more additional therapies are administered less than 1 day, 1 week, 2 weeks, 3 weeks, 4 weeks, one month, 2 months, 3 months, 6 months, 1 year, 2 years, or 5 years apart.

In some embodiments, the combination therapies provided herein involve administering a Compound daily, and administering one or more additional therapies once a week, once every 2 weeks, once every 3 weeks, once every 4 weeks, once every month, once every 2 months (e.g., approximately 8 weeks), once every 3 months (e.g., approximately 12 weeks), or once every 4 months (e.g., approximately 16 weeks). In certain embodiments, a Compound and one or more additional therapies are cyclically administered to a subject. Cycling therapy involves the administration of the Compound for a period of time, followed by the administration of one or more additional therapies for a period of time, and repeating this sequential administration. In certain embodiments, cycling therapy may also include a period of rest where the Compound or the additional therapy is not administered for a period of time (e.g., 2 days, 3 days, 4 days, 5 days, 6 days, 7 days, 1 week, 2 weeks, 3 weeks, 4 weeks, 5 weeks, 10 weeks, 20 weeks, 1 month, 2 months, 3 months, 4 months, 5 months, 6 months, 7 months, 8 months, 9 months, 10 months, 11 months, 12 months, 2 years, or 3 years). In an embodiment, the number of cycles administered is from 1 to 12 cycles, from 2 to 10 cycles, or from 2 to 8 cycles.

In some embodiments, the methods for treating NF provided herein comprise administering a Compound as a single agent for a period of time prior to administering the Compound in combination with an additional therapy. In certain embodiments, the methods for treating NF provided herein comprise administering an additional therapy alone for a period of time prior to administering a Compound in combination with the additional therapy.

In some embodiments, the administration of a Compound and one or more additional therapies in accordance with the methods presented herein have an additive effect relative the administration of the Compound or said one or more additional therapies alone. In some embodiments, the administration of a Compound and one or more additional therapies in accordance with the methods presented herein have a synergistic effect relative to the administration of the Compound or said one or more additional therapies alone.

As used herein, the term “synergistic,” refers to the effect of the administration of a Compound in combination with one or more additional therapies (e.g., agents), which combination is more effective than the additive effects of any two or more single therapies (e.g., agents). In a specific embodiment, a synergistic effect of a combination therapy permits the use of lower dosages (e.g., sub-optimal doses) of a Compound or an additional therapy and/or less frequent administration of a Compound or an additional therapy to a subject. In certain embodiments, the ability to utilize lower dosages of a Compound or an additional therapy and/or to administer a Compound or said additional therapy less frequently reduces the toxicity associated with the administration of a Compound or said additional therapy, respectively, to a subject without reducing the efficacy of a Compound or said additional therapy, respectively, in the treatment of NF. In some embodiments, a synergistic effect results in improved efficacy of a Compound and each of said additional therapies in treating NF. In some embodiments, a synergistic effect of a combination of a Compound and one or more additional therapies avoids or reduces adverse or unwanted side effects associated with the use of any single therapy.

The combination of a Compound and one or more additional therapies can be administered to a subject in the same pharmaceutical composition. Alternatively, a Compound and one or more additional therapies can be administered concurrently to a subject in separate pharmaceutical compositions. A Compound and one or more additional therapies can be administered sequentially to a subject in separate pharmaceutical compositions. A Compound and one or more additional therapies may also be administered to a subject by the same or different routes of administration.

The combination therapies provided herein involve administrating to a subject to in need thereof a Compound in combination with conventional, or known, therapies for NF. Current therapies for NF, include surgery, and in some cases radiation or drug therapy such as chemotherapy. Other therapies for NF or a condition associated therewith are aimed at controlling or relieving symptoms, e.g., headaches and epileptic seizures. Accordingly, in some embodiments, the combination therapies provided herein involve administrating to a subject to in need thereof a pain reliever, a medication for epileptic seizures, or other therapy aimed at alleviating or controlling symptoms associated with NF or a condition associated therewith.

In specific embodiments, combination therapies provided herein involve administering to a subject in need thereof a Compound in combination with one or more behavioral therapies to treat learning disorders associated with NF1. In some embodiments, combination therapies provided herein involve administering to a subject in need thereof a Compound in combination with one or more therapies to treat ADHD related to NF1, e.g., RITALIN® (brand of methylphenidate).

Specific examples of anti-cancer agents that may be used in combination with a Compound include: a hormonal agent (e.g., aromatase inhibitor, selective estrogen receptor modulator (SERM), and estrogen receptor antagonist), chemotherapeutic agent (e.g., microtubule dissembly blocker, antimetabolite, topisomerase inhibitor, and DNA crosslinker or damaging agent), anti-angiogenic agent (e.g., VEGF antagonist, receptor antagonist, integrin antagonist, vascular targeting agent (VTA)/vascular disrupting agent (VDA)), radiation therapy, and conventional surgery.

Non-limiting examples of hormonal agents that may be used in combination with a Compound include aromatase inhibitors, SERMs, and estrogen receptor antagonists. Hormonal agents that are aromatase inhibitors may be steroidal or nonsteroidal. Non-limiting examples of nonsteroidal hormonal agents include letrozole, anastrozole, aminoglutethimide, fadrozole, and vorozole. Non-limiting examples of steroidal hormonal agents include aromasin (exemestane), formestane, and testolactone. Non-limiting examples of hormonal agents that are SERMs include tamoxifen (branded/marketed as NOLVADEX®), afimoxifene, arzoxifene, bazedoxifene, clomifene, femarelle, lasofoxifene, ormeloxifene, raloxifene, and toremifene. Non-limiting examples of hormonal agents that are estrogen receptor antagonists include fulvestrant. Other hormonal agents include but are not limited to abiraterone and lonaprisan.

Non-limiting examples of chemotherapeutic agents that may be used in combination with a Compound include microtubule disassembly blocker, antimetabolite, topisomerase inhibitor, and DNA crosslinker or damaging agent. Chemotherapeutic agents that are microtubule dissemby blockers include, but are not limited to, taxenes (e.g., paclitaxel (branded/marketed as TAXOL®), docetaxel, abraxane, larotaxel, ortataxel, and tesetaxel); epothilones (e.g., ixabepilone); and vinca alkaloids (e.g., vinorelbine, vinblastine, vindesine, and vincristine (branded/marketed as ONCOVIN®)).

Chemotherapeutic agents that are antimetabolites include, but are not limited to, folate antimetabolites (e.g., methotrexate, aminopterin, pemetrexed, raltitrexed); purine antimetabolites (e.g., cladribine, clofarabine, fludarabine, mercaptopurine, pentostatin, thioguanine); pyrimidine antimetabolites (e.g., 5-fluorouracil, capcitabine, gemcitabine (branded/marketed as GEMZAR®), cytarabine, decitabine, floxuridine, tegafur); and deoxyribonucleotide antimetabolites (e.g., hydroxyurea).

Chemotherapeutic agents that are topoisomerase inhibitors include, but are not limited to, class I (camptotheca) topoisomerase inhibitors (e.g., topotecan (branded/marketed as HYCAMTIN®) irinotecan, rubitecan, and belotecan); class II (podophyllum) topoisomerase inhibitors (e.g., etoposide or VP-16, and teniposide); anthracyclines (e.g., doxorubicin, epirubicin, Doxil, aclarubicin, amrubicin, daunorubicin, idarubicin, pirarubicin, valrubicin, and zorubicin); and anthracenediones (e.g., mitoxantrone, and pixantrone).

Chemotherapeutic agents that are DNA crosslinkers (or DNA damaging agents) include, but are not limited to, alkylating agents (e.g., cyclophosphamide, mechlorethamine, ifosfamide (branded/marketed as IFEX®), trofosfamide, chlorambucil, melphalan, prednimustine, bendamustine, uramustine, estramustine, carmustine (branded/marketed as BiCNU®), lomustine, semustine, fotemustine, nimustine, ranimustine, streptozocin, busulfan, mannosulfan, treosulfan, carboquone, N,N′N′-triethylenethiophosphoramide, triaziquone, triethylenemelamine); alkylating-like agents (e.g., carboplatin (branded/marketed as PARAPLATIN®), cisplatin, oxaliplatin, nedaplatin, triplatin tetranitrate, satraplatin, picoplatin); nonclassical DNA crosslinkers (e.g., procarbazine, dacarbazine, temozolomide (branded/marketed as TEMODAR®), altretamine, mitobronitol); and intercalating agents (e.g., actinomycin, bleomycin, mitomycin, and plicamycin).

Non-limiting examples of anti-angiogenic agents that may be used in combination with a Compound include VEGF antagonists, receptor antagonists, integrin antagonists (e.g., vitaxin, cilengitide, and S247), and VTAs/VDAs (e.g., fosbretabulin). VEGF antagonists include, but are not to, anti-VEGF antibodies (e.g., bevacizumab (branded/marketed as AVASTIN®) and ranibizumab (branded/marketed as LUCENTIS®)), VEGF traps (e.g., aflibercept), VEGF antisense or siRNA or miRNA, and aptamers (e.g., pegaptanib (branded/marketed as MACUGEN®)). Anti-angiogenic agents that are receptor antagonists include, but are not limited to, antibodies (e.g., ramucirumab) and kinase inhibitors (e.g., sunitinib, sorafenib, cediranib, panzopanib, vandetanib, axitinib, and AG-013958) such as tyrosine kinase inhibitors. Other non-limiting examples of anti-angiogenic agents include ATN-224, anecortave acetate (branded/marketed as RETAANE®), microtubule depolymerization inhibitor such as combretastatin A4 prodrug, and recombinant protein or protein fragment such as collagen 18 (endostatin).

Non-limiting examples of other therapies that may be administered to a subject in combination with a Compound include:
(1) a statin such as lovostatin (e.g., branded/marketed as MEVACOR®);
(2) an mTOR inhibitor such as sirolimus which is also known as Rapamycin (e.g., branded/marketed as RAPAMUNE®), temsirolimus (e.g., branded/marketed as TORISEL®), evorolimus (e.g., branded/marketed as AFINITOR®), and deforolimus;
(3) a farnesyltransferase inhibitor agent such as tipifarnib (e.g., branded/marketed as ZARNESTRA®);
(4) an antifibrotic agent such as pirfenidone;
(5) a pegylated interferon such as PEG-interferon alpha-2b;
(6) a CNS stimulant such as methylphenidate (branded/marketed as)RITALIN®);
(7) a HER-2 antagonist such as anti-HER-2 antibody (e.g., trastuzumab) or kinase inhibitor (e.g., lapatinib);
(8) an IGF-1 antagonist such as an anti-IGF-1 antibody (e.g., AVE1642 and IMC-A11) or an IGF-1 kinase inhibitor;
(9) EGFR/HER-1 antagonist such as an anti-EGFR antibody (e.g., cetuximab, panitumamab) or EGFR kinase inhibitor (e.g., erlotinib (e.g., branded/marketed as TARCEVA®), gefitinib);
(10) SRC antagonist such as bosutinib;
(11) cyclin dependent kinase (CDK) inhibitor such as seliciclib;
(12) Janus kinase 2 inhibitor such as lestaurtinib;
(13) proteasome inhibitor such as bortezomib;
(14) phosphodiesterase inhibitor such as anagrelide;
(15) inosine monophosphate dehydrogenase inhibitor such as tiazofurine;
(16) lipoxygenase inhibitor such as masoprocol;
(17) endothelin antagonist;
(18) retinoid receptor antagonist such as tretinoin or alitretinoin;
(19) immune modulator such as lenalidomide, pomalidomide, or thalidomide (e.g., branded/marketed as THALIDOMID®);
(20) kinase (eg, tyrosine kinase) inhibitor such as imatinib (e.g., branded/marketed as GLEEVEC®, dasatinib, erlotinib, nilotinib, gefitinib, sorafenib, sunitinib (e.g., branded/marketed as SUTENT®), lapatinib, AEE788, or TG100801;
(21) non-steroidal anti-inflammatory agent such as celecoxib (branded/marketed as CELEBREX®);
(22) human granulocyte colony-stimulating factor (G-CSF) such as filgrastim (branded/marketed as NEUPOGEN®);
(23) folinic acid or leucovorin calcium;
(24) integrin antagonist such as an integrin α5β1-antagonist (e.g., JSM6427); (25) nuclear factor kappa beta (NF-κβ) antagonist such as OT-551, which is also an anti-oxidant;
(26) hedgehog inhibitor such as CUR61414, cyclopamine, GDC-0449, or anti-hedgehog antibody;
(27) histone deacetylase (HDAC) inhibitor such as SAHA (also known as vorinostat (branded/marketed as ZOLINZA®, PCI-24781, SB939, CHR-3996, CRA-024781, ITF2357, JNJ-26481585, or PCI-24781;
(28) retinoid such as isotretinoin (e.g., branded/marketed as ACCUTANE®);
(29) hepatocyte growth factor/scatter factor (HGF/SF) antagonist such as HGF/SF monoclonal antibody (e.g., AMG 102);
(30) synthetic chemical such as antineoplaston;
(31) anti-diabetic such as rosiglitazone maleate (e.g., branded/marketed as AVANDIA®);
(32) antimalarial and amebicidal drug such as chloroquine (e.g., branded/marketed as ARALEN®);
(33) synthetic bradykinin such as RMP-7;
(34) platelet-derived growth factor receptor inhibitor such as SU-101;
(35) receptor tyrosine kinase inhibitors of Flk-1/KDR/VEGFR2, FGFR1 and PDGFR beta such as SU5416 and SU6668;
(36) anti-inflammatory agent such as sulfasalazine (e.g., branded/marketed as AZULFIDINE®); and
(37) TGF-beta antisense therapy.

Non-limiting examples of other therapies that may be administered to a subject in combination with a Compound include: a synthetic nonapeptide analog of naturally occurring gonadotropin releasing hormone such as leuprolide acetate (branded/marketed as LUPRON®); a nonsteroidal, anti-androgen such as flutamide (branded/marketed as EULEXIN®) or nilutamide (branded/marketed as NILANDRON®); a non-steroidal androgen receptor inhibitor such as bicalutamide (branded/marketed as CASODEX®); steroid hormone such as progesterone; anti-fungal agent such as Ketoconazole (branded/marketed as NIZORAL®); glucocorticoid such as prednisone; estramustine phosphate sodium (branded/marketed as EMCYT®); and bisphosphonate such as pamidronate, alendronate, and risedronate.

In particular embodiments, combination therapies provided herein comprise administering a Compound in combination with one or more auditory therapies, e.g., hearing/auditory implants in NF2 subjects with hearing impairment. In certain embodiments, combination therapies provided herein comprise administering a Compound in combination with a seizure medication.

Other specific examples of therapies that may be used in combination with a Compound include, but are not limited to, antibodies that specifically bind to a tumor specific antigen or tumor associated antigen, e.g., anti-EGFR/HER-1 antibodies.

Additional specific examples of therapies that may be used in combination with a Compound include, but are not limited to, agents associated with cancer immunotherapy, e.g., cytokines, interleukins, and cancer vaccines.

Specific examples of agents alleviating side-effects associated with NF, that can be used as therapies in combination with a Compound, include, but are not limited to: antiemetics, e.g., Ondansetron hydrochloride (branded/marketed as ZOFRAN®), Granisetron hydrochloride (branded/marketed as KYTRIL®), Lorazepam (branded/marketed as ATIVAN®) and Dexamethasone (branded/marketed as DECADRON®).

In certain embodiments, combination therapies provided herein for treating NF comprise administering a Compound in combination with one or more agents used to treat and/or manage one or more of the following conditions: bleeding, arterial and venous thrombosis, hypertension, delayed wound healing, asymptomatic proteinuria, nasal septal perforation, reversible posterior leukoencephalopathy syndrome in association with hypertension, light-headedness, ataxia, headache, hoarseness, nausea, vomiting, diarrhea, rash, subungual hemorrhage, myelosuppression, fatigue, hypothyroidism, QT interval prolongation, and heart failure.

In certain embodiments, combination therapies provided herein for treating NF comprise administering a Compound in combination with one or more current anti-angiogenesis agents and one or more agents used to treat and/or manage a side effect observed with one or more of the current anti-angiogenesis agents, such as, bleeding, arterial and venous thrombosis, hypertension, delayed wound healing, asymptomatic proteinuria, nasal septal perforation, reversible posterior leukoencephalopathy syndrome in association with hypertension, light-headedness, ataxia, headache, hoarseness, nausea, vomiting, diarrhea, rash, subungual hemorrhage, myelosuppression, fatigue, hypothyroidism, QT interval prolongation, or heart failure.

In certain embodiments, a Compound is not used in combination with a drug that is primarily metabolized by CYP2D6 (such as an antidepressant (e.g. a tricyclic antidepressant, a selective serotonin reuptake inhibitor, and the like), an antipsychotic, a beta-adrenergic receptor blocker, or certain types of anti-arrhythmics) to treat NF.

### 6. EXAMPLE

### Preparation of Compounds Provided Herein

The following examples are presented by way of illustration not limitation.

Methods for preparing certain Compounds provided herein and the Compounds disclosed on pages 26 to 98 of the '764 publication are provided on pages 112 to 142 of the '764 publication and are incorporated by reference herein on pages 99 to 105 and in their entireties and for all purposes. Methods for preparing certain Compounds provided herein and the Compounds disclosed in copending U.S. Provisional Patent Application 61/181,652, entitled: PROCESSES FOR THE PREPARATION OF SUBSTITUTED TETRAHYDRO BETA-CARBOLINES, filed May 27, 2009, are provided therein and are incorporated by reference herein in their entirety and for all purposes.

### 7. EXAMPLE

### Formulation of Compound #10

The following example illustrates how Compound #10 may be formulated for oral administration.

For clinical use, Compound #10 has been formulated using cGMPs. Compound #10 is intended for oral administration and is provided in size 00 color coded, hard gelatin capsules. As shown in Table 2, each capsule contains 2 mg (white), 10 mg (gray), or 20 mg (orange) of the Compound formulated by w/w % (weight/weight %) in a SEDDS or SMEDDS system. The formulated product in the capsules appears as an opaque, off white soft solid at room temperature. If warmed, the encapsulated system begins to soften at temperatures of 38 to 40° C. and eventually becomes a clear, yellow liquid at ≧44° C.

TABLE 2
Composition of Compound #10 Capsules

| | 2 mg Capsule | 10 mg Capsule | 20 mg Capsule |
|---|---|---|---|
| Component | (w/w %) | (w/w %) | (w/w %) |
| Compound #10 | 0.28 [0.26-0.30] | 1.43 [1.33-1.53] | 2.67 [2.48-2.86] |
| GELUCIRE ® 44/14 | 49.87 [46.4-53.4] | 49.87 [46.4-53.4] | 49.87 [46.4-53.4] |
| SOLUTOL ® HS15 | 49.84 [46.4-53.3] | 48.69 [45.3-52.1] | 47.45 [44.1-50.8] |
| BHT | 0.01 [0.009-0.011] | 0.01 [0.009-0.011] | 0.01 [0.009-0.011] |
| Total Weight (100%) (mg) | 700 | 700 | 750 |

### 8. EXAMPLE

### Assay to Evaluate Effect on Hypoxia-Inducible Endogenous VEGF Expression

The ability of the Compounds to modulate hypoxia-inducible endogenous VEGF expression may be analyzed as follows. VEGF protein levels may be monitored by an ELISA assay (R&D Systems). Briefly, HeLa cells may be cultured for 24-48 hours under hypoxic conditions (1% O₂, 5% CO₂, balanced with nitrogen) in the presence or absence of a Compound. The conditioned media may then be assayed by ELISA, and the concentration of VEGF calculated from the standard ELISA curve of each assay.

A dose-response analysis may be performed using the ELISA assay and conditions described above. The conditions for the dose-response ELISA are analogous to those described above. A series of, e.g., seven different concentrations may be analyzed. In parallel, a dose-response cytotoxicity assay may be performed using Cell Titer Glo (Promega) under the same conditions as the ELISA to ensure that the inhibition of VEGF expression was not due to the cytotoxicity. Dose-response curves may be plotted using percentage inhibition versus concentration of the Compound, and EC₅₀ and CC₅₀ values may be generated for each Compound with the maximal inhibition set as 100% and the minimal inhibition as 0%. In one embodiment, Compounds will have an EC₅₀ of less than 50, less than 10, less than 2, less than 0.5, or less than 0.01.

The EC₅₀ for a series of Compounds is provided in Table 3.

TABLE 3

| | | LC/MS | |
|---|---|---|---|
| | LC/MS | Retention Time | ELISA |
| Compound | [M + H] | (min) | EC₅₀ μM |
| #10 | 467.15 | 4.51 | ***** |
| 17 | 447.14 | 4.44 | ***** |
| 60 | 433.17 | 4.27 | **** |
| 76 | 449.26 | 4.3 | **** |
| 121 | 403.32 | 4.27 | **** |
| 142 | 462.15 | 4.11 | *** |
| 160 | 450.15 | 3.95 | *** |
| 186 | 462.19 | 3.81 | ** |
| 192 | 495.28 | 4.89 | ** |
| 331 | ~0.010 | 2.94 | * |
| #332 | ~0.010 | 4 | * |
| 341 | 447.26 | 4.25 | *** |
| 344 | 459.31 | 4.91 | **** |
| 346 | 587 | 4.04 | **** |
| 347 | 451.16 | 3.93 | ***** |
| 348 | 479.28 | 4.13 | ***** |
| 350 | 462.17 | 3.66 | ***** |
| 351 | 471.17 | 3.93 | **** |
| 353 | 497.16 | 3.94 | ***** |
| 354 | 525.2 | 4.19 | ***** |
| 355 | 511.21 | 3.81 | ***** |
| 359 | 511.25 | 3.64 | *** |
| 360 | 516 | 3.82 | **** |
| 366 | 553.3 | 4.42 | * |
| 372 | 486.9 | 4.96 | * |
| 388 | 495.4 | 3.94 | ***** |
| 391 | 562.55 | 3.63 | ***** |
| 395 | 481.32 | 3.51 | ***** |
| 397 | 535.3 | 4.29 | ***** |
| 398 | 481.3 | 4.23 | ***** |
| 400 | 493.3 | 4.43 | ***** |
| 401 | 451.3 | 3.99 | ***** |
| 403 | 479.3 | 4.23 | ***** |
| 405 | 551.17 | 4.58 | ***** |
| 409 | 477.4 | 4.18 | ***** |
| 410 | 451.3 | 3.99 | ***** |
| 413 | 459.3 | 4.16 | ***** |
| 415 | 637.64 | 2.82 | ***** |
| 417 | 562.47 | 4.15 | ***** |
| 418 | 511.3 | 4.13 | ***** |
| 421 | 553.30 | 4.05 | ***** |
| 422 | 359.29 | 4.17 | ***** |
| 426 | 535.27 | 4.29 | ***** |
| 427 | 554.3 | 4.45 | ***** |
| 428 | 563.55 | 4.64 | ***** |
| 429 | 564.42 | 2.77 | ***** |
| 432 | 489.4 | 4.14 | ***** |
| 433 | 578.44 | 2.82 | ***** |
| 436 | 477.4 | 3.93 | ***** |
| 437 | 543.4 | 3.92 | ***** |
| 440 | 536.43 | 3.95 | ***** |
| 444 | 455.28 | 3.73 | **** |
| 446 | 383.3 | 4.10 | **** |
| 448 | 501.27 | 3.65 | **** |
| 450 | 587 | 4.04 | **** |
| 452 | 439.3 | 3.56 | **** |
| 454 | 579.3 | 2.75 | **** |
| 455 | 583 | 3.84 | **** |
| 460 | 509.30 | 4.16 | **** |
| 462 | 580.56 | 2.85 | **** |
| 463 | 495.44 | 4.13 | **** |
| 465 | 507.4 | 3.98 | **** |
| 467 | 524.2 | 4.02 | **** |
| 468 | 582.2 | 2.81 | **** |
| 470 | 554.3 | 3.90 | **** |
| 471 | 620.18 | 3.85 | **** |
| 479 | 538.3 | 2.76 | *** |
| 482 | 522.3 | 3.95 | *** |
| 489 | 538.3 | 4.15 | *** |
| 491 | 537.31 | 2.64 | *** |
| 493 | 504.3 | 2.68 | *** |
| 500 | 506.29 | 3.85 | *** |
| 501 | 534.3 | 2.68 | *** |
| 502 | 518.3 | 2.76 | *** |
| 519 | 527.2 | 3.88 | ** |
| 530 | 466.28 | 3.21 | ** |
| 536 | 482.29 | 3.29 | ** |
| 540 | 428.28 | 3.43 | ** |
| 543 | 466.34 | 3.29 | ** |
| 544 | 723.58 | 3.92 | ***** |
| 545 | 466.31 | 3.28 | ** |
| 554 | 482.32 | 3.41 | ** |
| 570 | 549.22 | 4.59 | ***** |
| 571 | 497.13 | 3.50 | ** |
| 572 | 525.29 | 4.14 | ***** |
| 575 | 437.33 | 3.16 | ** |
| 576 | 575.43 | 3.71 | *** |
| 577 | 453.28 | 3.34 | *** |
| 578 | 610.45 | 3.94 | *** |
| 579 | 481.32 | 3.51 | ***** |
| 580 | 495.29 | 3.64 | ***** |
| 581 | 465.43 | 3.64 | * |
| 583 | 512.26 | 3.39 | *** |
| 584 | 466.37 | 3.34 | *** |
| 587 | 467.29 | 3.66 | *** |
| 588 | 455.26 | 3.69 | *** |
| 589 | 471.3 | 3.83 | *** |
| 590 | 495.31 | 3.64 | **** |
| 591 | 541.35 | 3.73 | ***** |
| 592 | 523.42 | 3.58 | ***** |
| 593 | 541.38 | 3.69 | **** |
| 594 | 505.38 | 3.83 | *** |
| 614 | 463 | 3.88 | ** |
| 616 | 540 | 4.17 | ** |
| 617 | 621.57 | 4.13 | **** |
| 626 | 493.6 | 3.48 | **** |
| 627 | 511.6 | 3.53 | ***** |
| 628 | 527.4 | 3.62 | *** |
| 629 | 527.5 | 3.72 | ***** |
| 630 | 573.5 | 3.75 | ***** |
| 631 | 507.6 | 3.65 | ***** |
| 632 | 538.6 | 3.53 | **** |
| 635 | 523.6 | 3.47 | **** |
| 637 | 621.62 | 2.77 | ***** |
| 638 | 580.56 | 2.80 | ***** |
| 660 | 543.7 | 4.92 | ***** |
| 670 | 521.6 | 4.02 | ***** |
| 673 | 539.6 | 4.02 | **** |
| 674 | 555.6 | 4.13 | **** |
| 675 | 555.6 | 4.22 | **** |
| 677 | 535.6 | 4.15 | **** |
| 678 | 551.6 | 3.98 | *** |
| 680 | 599.5 | 4.27 | ***** |
| 681 | 566.6 | 4.02 | **** |
| 698 | 578.5 | 2.43 | **** |
| 699 | 568.5 | 2.35 | **** |
| 700 | 566.6 | 2.45 | **** |
| 701 | 596.6 | 2.47 | **** |
| 702 | 594.6 | 2.43 | **** |
| 703 | 592.6 | 2.48 | **** |
| 704 | 607.6 | 2.20 | *** |
| 705 | 575.5 | 2.47 | **** |
| 706 | 576.5 | 3.58 | ***** |
| 710 | 495.45 | 4.42 | ***** |
| 712 | 513.50 | 4.42 | ***** |
| 713 | 529.46 | 4.62 | **** |
| 719 | 527.5 | 4.47 | ***** |
| 723 | 555.4 | 4.09 (non polar) | ***** |
| 735 | 552.5 | 2.98 | ***** |
| 736 | 562.5 | 3.15 | ***** |
| 737 | 580.6 | 3.17 | **** |
| 738 | 578.5 | 3.02 | ***** |
| 739 | 576.6 | 3.17 | ***** |
| 740 | 591.6 | 2.75 | *** |
| 741 | 616.5 | 2.62 | *** |
| 742 | 559.5 | 3.13 | ***** |
| 743 | 560.5 | 3.83 | ***** |
| 772 | 580.5 | 3.03 | ***** |
| 773 | 590.6 | 3.12 | ***** |
| 774 | 578.5 | 3.12 | **** |
| 775 | 608.6 | 3.05 | ***** |
| 776 | 606.5 | 3.05 | ***** |
| 777 | 604.6 | 3.12 | ***** |
| 778 | 619.6 | 2.77 | ***** |
| 779 | 644.5 | 2.63 | *** |
| 780 | 587.5 | 3.10 | ***** |
| 781 | 588.5 | 4.05 | ***** |
| 782 | 596.5 | 3.10 | ***** |
| 783 | 606.5 | 3.18 | ***** |
| 784 | 594.5 | 3.27 | ***** |
| 785 | 624.5 | 3.22 | ***** |
| 786 | 622.5 | 3.12 | ***** |
| 787 | 620.5 | 3.20 | ***** |
| 788 | 635.6 | 2.85 | **** |
| 789 | 660.5 | 2.68 | *** |
| 790 | 603.5 | 3.22 | ***** |
| 791 | 604.5 | 4.25 | ***** |
| 833 | 532.4 | 3.50 | *** |
| 834 | 532.4 | 3.42 | **** |
| 835 | 531.4 | 2.57 | *** |
| 836 | 531.4 | 3.67 | **** |
| #837 | 563.4 | 2.93 | ***** |
| #838 | 577.4 | 2.82 | ***** |
| 839 | 548.3 | 3.63 | **** |
| 840 | 548.3 | 3.58 | **** |
| #841 | 579.3 | 3.08 | ***** |
| #842 | 593.3 | 2.95 | ***** |
| #843 | 573.4 | 2.75 | ***** |
| 845 | 648.48 | 4.45 | *** |
| 846 | 526.45 | 2.57 | *** |
| 847 | 568.37 | 3.40 | **** |
| 848 | 585.30 | 3.57 | ***** |
| 849 | 604.37 | 3.52 | **** |
| 850 | 540.39 | 2.60 | *** |
| 851 | 495.06 | 4.37 | ***** |
| 853 | 549.09 | 4.38 | ***** |
| 854 | 523.17 | 4.73 | ***** |
| 855 | 455.19 | 4.15 | **** |
| 857 | 505.16 | 4.30 | ***** |
| 860 | 467.2 | 4.13 | ***** |
| 861 | 451.12 | 4.10 | **** |
| 862 | 471.17 | 4.32 | ***** |
| 863 | 514.55 | 4.38 | ***** |
| 867 | 577.43 | 2.85 | **** |
| 882 | 542.51 | 3.87 | ***** |
| 888 | 558.54 | 3.70 | ***** |
| 889 | 545.55 | 2.93 | ***** |
| 891 | 528.49 | 3.69 | ***** |
| 892 | 546.50 | 3.75 | ***** |
| 894 | 580.47 | 2.72 | ***** |
| 900 | 541.55 | 3.00 | ***** |
| 903 | 621.39 | 2.72 | ***** |
| 904 | 596.54 | 2.85 | ***** |
| 908 | 582.43 | 2.79 | ***** |
| 911 | 527.54 | 2.88 | ***** |
| 913 | 626.6 | 2.88 | ***** |
| 915 | 509.56 | 4.63 | ***** |
| 916 | 626.40 | 2.82 | ***** |
| 917 | 561.46 | 2.95 | ***** |
| 918 | 642.56 | 2.85 | ***** |
| 920 | 557.57 | 2.87 | ***** |
| 921 | 527.39 | 4.52 | ***** |
| 922 | 561.53 | 2.85 | ***** |
| 923 | 612.51 | 2.92 | ***** |
| 925 | 596.54 | 2.88 | ***** |
| 926 | 5.62 | 3.85 | ***** |
| 932 | 548.49 | 3.17 | ***** |
| 933 | 596.37 | 2.79 | ***** |
| 934 | 561.53 | 2.95 | ***** |
| 936 | 582.6 | 2.83 | ***** |
| 938 | 582.53 | 2.85 | ***** |
| 941 | 562.55 | 3.63 | ***** |
| 942 | 623.35 | 2.73 | **** |
| 944 | 525.56 | 4.36 | **** |
| 946 | 566.53 | 2.77 | **** |
| 951 | 544.53 | 3.27 | **** |
| 952 | 530.53 | 3.12 | **** |
| 953 | 552.46 | 2.90 | **** |
| 958 | 542.36 | 3.84 | **** |
| 960 | 639.57 | 2.70 | **** |
| 961 | 593.52 | 2.64 | **** |
| 963 | 593.61 | 2.72 | **** |
| 964 | 598.55 | 2.83 | **** |
| 966 | 564.45 | 3.32 | **** |
| 967 | 491.57 | 4.00 | **** |
| 970 | 609.54 | 2.72 | **** |
| 973 | 578.47 | 3.80 | **** |
| 974 | 528.34 | 3.79 | *** |
| 976 | 564.46 | 3.23 | *** |
| 977 | 568.53 | 2.85 | *** |
| 981 | 560.51 | 3.12 | *** |
| 984 | 5.06.19 | 3.97 | ** |
| 988 | 605.62 | 2.52 | ***** |
| 989 | 564.61 | 2.55 | ***** |
| 990 | 610.62 | 2.67 | ***** |
| 991 | 580.58 | 2.60 | *** |
| 992 | 566.61 | 2.60 | *** |
| 993 | 577.61 | 2.45 | ***** |
| 994 | 545.54 | 2.57 | ***** |
| 995 | 546.57 | 3.53 | ***** |
| 996 | 578.46 | 3.71 | ***** |
| 999 | 493.3 | 4.43 | ***** |
| 1001 | 575.5 | 2.98 | **** |
| 1005 | 560.3 | 4.55 | ** |
| 1008 | 548.2 | 4.79 | *** |
| 1009 | 468.1 | 3.90 | *** |
| 1011 | 560.2 | 5.54 | *** |
| 1016 | 560.51 | 4.23 | * |
| 1017 | 544.39 | 4.08 | ***** |
| 1021 | 621.2 | 4.35 | *** |
| 1022 | 607.2 | 5.05 | *** |
| 1023 | 586.1 | 5.93 | **** |
| 1024 | 591.2 | 5.01 | *** |
| 1025 | 633.2 | 4.29 | *** |
| 1026 | 619.2 | 4.24 | **** |
| 1027 | M − 1: 574.1 | 5.03 | *** |
| 1028 | 603.2 | 4.23 | *** |
| 1029 | 660.2 | 3.87 | * |
| 1030 | 576.2 | 5.29 | **** |
| 1031 | 558.0 | 4.69 | ***** |
| 1050 | 505.33 | 3.85 | ***** |
| 1051 | 523.4 | 3.88 | ***** |
| 1052 | 539.3 | 3.97 | **** |
| 1053 | 537.5 | 4.00 | ***** |
| 1054 | 583.4 | 4.07 | ***** |
| 1055 | 535.4 | 3.82 | **** |
| 1058 | 507.0 | 5.88 | ***** |
| 1062 | 477.1 | 5.53 | ***** |
| 1063 | 560.1 | 5.47 | **** |
| 1064 | 607.1 | 4.84 | **** |
| 1066 | 562.55 | 3.63 | ***** |
| 1067 | 562.1 | 5.33 | **** |
| 1068 | 562.1 | 5.70 | ***** |
| 1069 | 562.27 | 3.9 | ***** |
| 1070 | 596.24 | 2.40 | ***** |
| 1071 | 598.21 | 2.48 | ***** |
| 1075 | 546.3 | 4.55 | **** |
| 1076 | 559.3 | 4.08 | *** |
| 1077 | 528.1 | 5.51 | **** |
| 1078 | 528.1 | 4.74 | **** |
| 1086 | 577.9 | 3.73 | **** |
| 1087 | 591.9 | 3.78 | **** |
| 1088 | 605.9 | 3.87 | **** |
| 1089 | 577.9 | 3.75 | ** |
| 1090 | 591.9 | 3.80 | ** |
| 1091 | 605.9 | 3.85 | ** |
| 1092 | 595.9 | 2.45 | **** |
| 1093 | 610.0 | 2.47 | **** |
| 1094 | 624.0 | 2.48 | **** |
| 1095 | 596.0 | 2.47 | ** |
| 1096 | 610.0 | 2.47 | ** |
| 1097 | 624.0 | 2.50 | *** |
| 1098 | 594.57 | 2.47 | **** |
| 1099 | 564.52 | 2.45 | **** |
| 1108 | 589.4 | 3.97 | **** |
| 1110 | M − 1: 493.1 | 5.48 | ***** |
| 1111 | 509.1 | 4.84 | ***** |
| 1113 | 577.4 | 34.06 | ** |
| 1115 | 564.3 | 4.61 | **** |
| 1117 | 580.3 | 4.79 | **** |
| 1119 | 610.3 | 4.85 | *** |
| 1121 | 566.3 | 4.74 | * |
| 1123 | 545.2 | 4.65 | *** |
| 1125 | 546.1 | 5.84 | ** |
| 1126 | 530.8 | 4.3 | ** |
| 1127 | 562.24 | 4.20 | *** |
| 1128 | 530.8 | 4.32 | ***** |
| 1129 | 562.26 | 4.13 | ***** |
| 1130 | 576.3 | 4.668 | **** |
| 1131 | 606.0 | 4.646 | **** |
| 1132 | 590.5 | 4.826 | **** |
| 1134 | 558.1 | 3.68 | ***** |
| 1143 | 510 | 4.300 | **** |
| 1144 | 558.5 | 4.711 | *** |
| 1145 | 558.5 | 5.05 | **** |
| 1150 | 558.5 | 4.664 | **** |
| 1151 | 588.5 | 4.616 | *** |
| 1152 | 572.5 | 4.891 | **** |
| 1155 | 546.3 | 5.54 | *** |
| 1159 | 493 | 4.22 | ***** |
| 1160 | 453 | 3.73 | ***** |
| 1161 | 492 | 3.65 | ***** |
| 1162 | 579.17 | 4.28 | ***** |
| 1168 | 547.27 | 4.18 | ***** |
| 1169 | 565.24 | 4.17 | ***** |
| 1170 | 561.28 | 4.37 | ***** |
| 1171 | 577.28 | 4.13 | ***** |
| 1172 | 539.20 | 3.58 | ***** |
| 1178 | 507.19 | 3.37 | ***** |
| 1179 | 525.25 | 3.38 | ***** |
| 1180 | 521.23 | 3.52 | ***** |
| 1181 | 537.20 | 3.35 | ***** |
| 1182 | 542.27 | 3.70 | ***** |
| 1183 | 556.26 | 2.45 | ***** |
| 1184 | 600.38 | 2.43 | ***** |
| 1194 | 572.5 | 5.237 | ***** |
| 1195 | 469.5 | 5.192 | **** |
| 1196 | 465 | 5.373 | **** |
| 1197 | 481 | 5.156 | **** |
| 1199 | 485 | 5.407 | **** |
| 1203 | 581.24 | 4.40 | ***** |
| 1205 | 539.29 | 3.58 | ***** |
| 1207 | 581.24 | 4.35 | ***** |
| 1209 | 539.26 | 3.67 | ***** |
| 1213 | 510 | 3.45 | *** |
| 1216 | 506 | 3.37 | **** |
| 1223 | 527.2 | 3.52 | ***** |
| 1224 | 527.0 | 3.53 | ***** |
| 1225 | 597.9 | 4.69 | **** |
| 1227 | 565.2 | 4.18 | ***** |
| 1228 | 567.2 | 4.37 | ***** |
| 1229 | 595.39 | 4.47 | ***** |
| 1230 | 555.24 | 3.73 | ***** |
| 1231 | 528 | 3.48 | **** |
| 1234 | 594.00 | 5.135 | ***** |
| 1235 | 578.0 | 4.785 | **** |
| 1250 | 511.07 | 3.93 | ***** |
| 1255 | 614.35 | 2.35 | *** |
| 1257 | 554.26 | 2.42 | **** |
| 1258 | 600.14 | 2.43 | ***** |
| 1259 | 527.2 | 3.50 | **** |
| 1260 | 565.2 | 4.18 | ***** |
| 1263 | 583.00 | 3.85 | ***** |
| 1265 | 469.0 | 5.478 | **** |
| 1266 | 465.0 | 5.667 | ***** |
| 1267 | 481.0 | 5.426 | **** |
| 1269 | 485.0 | 5.723 | ***** |
| 1276 | M + 23: 604.2 | 4.47 | ***** |
| 1277 | M + 23: 646.2 | 4.83 | ***** |
| 1278 | M + 23: 634.2 | 4.60 | ***** |
| 1279 | 610.2 | 5.28 | ***** |
| 1280 | 628.2 | 5.22 | **** |
| 1281 | M + 23: 614.1 | 4.65 | ***** |
| 1282 | 592.0 | 5.90 | ***** |
| 1288 | 608.2 | 4.51 | **** |
| 1289 | M + 23: 594.2 | 4.80 | ***** |
| 1290 | M + 23: 594.2 | 5.18 | ***** |
| 1291 | M + 23: 594.2 | 4.88 | **** |
| 1292 | M − 1: 519.2 | 5.53 | ***** |
| 1293 | M − 1: 523.2 | 5.34 | ***** |
| 1297 | 535.31 | 3.67 | **** |
| 1299 | M − 1: 505.2 | 5.28 | ***** |
| 1300 | M − 1: 535.2 | 4.55 | ***** |
| 1301 | M + 23: 614.2 | 5.96 | **** |
| 1302 | 590.2 | 5.37 | *** |
| 1328 | 553.4 | 3.65 | ***** |
| 1329 | 569.3 | 3.83 | ***** |
| 1330 | 539.28 | 3.60 | * |
| 1331 | 581.25 | 4.50 | * |
| 1332 | 451.27 | 3.75 | * |
| 1333 | 499.40 | 3.90 | * |
| 1335 | M − 1: 573.0 | 4.82 | **** |
| 1336 | M − 1: 519.1 | 5.76 | **** |
| 1337 | M − 1: 549.2 | 4.33 | **** |
| 1343 | 555.1 | 3.53 | ***** |
| 1344 | 571.0 | 3.70 | ***** |
| 1348 | 569.1 | 3.60 | ***** |
| 1349 | 585.0 | 3.77 | ***** |
| 1352 | 583.1 | 3.72 | ***** |
| 1353 | 599.0 | 3.88 | ***** |
| 1357 | 597.2 | 3.77 | ***** |
| 1358 | 613.2 | 3.93 | ***** |
| 1361 | M − 1: 535.2 | 5.42 | **** |
| 1362 | 622.57 | 2.53 | ***** |
| 1364 | 605.3 | 4.41 | *** |
| 1391 | 563.4 | 2.93 | ***** |
| 1392 | 577.4 | 2.82 | ***** |
| 1393 | 579.4 | 3.08 | ***** |
| 1394 | 593.3 | 2.95 | ***** |
| 1413 | 546.4 | 3.23 | ***** |
| 1414 | 560.4 | 2.83 | ***** |
| 1415 | 564.4 | 3.65 | ***** |
| 1416 | 589.5 | 3.40 | *** |
| 1417 | 562.4 | 3.42 | ***** |
| 1418 | 576.41 | 2.95 | **** |
| 1419 | 577.4 | 4.05 | **** |
| 1420 | 580.3 | 3.83 | ***** |
| 1421 | 587.4 | 3.88 | ***** |
| 1422 | 605.4 | 3.55 | **** |
| 1440 | 558.9 | 3.65 | ***** |
| 1441 | 571.5 | 3.75 | **** |
| 1442 | 574.9 | 3.85 | ***** |
| 1476 | 580.56 | 2.43 | *** |
| 1520 | 492 | 3.87 | ***** |
| 1537 | 594.23 | 2.40 | ***** |
| 1538 | 495.2 | 3.95 | ***** |
| 1539 | 495.08 | 3.95 | *** |
| 1546 | 492 | 3.85 | *** |
| 1547 | 534, 536 | 3.93 | ***** |
| 1548 | 474 | 3.75 | **** |
| 1549 | 488 | 3.77 | **** |
| 1551 | 573 | 3.83 | ***** |
| 1552 | 555 | 4.68 | ***** |
| 1553 | 569 | 4.88 | ***** |
| 1554 | 608 | 2.40 | * |
| 1555 | 624 | 3.80 | ***** |
| 1557 | M − 1: 614.2 | 4.52 | ** |
| 1558 | M + 23: 604.2 | 4.57 | **** |
| 1559 | 596.1 | 4.88 | **** |
| 1560 | M + 23: 616.2 | 4.82 | **** |
| 1561 | 631.1 | 4.15 | **** |
| 1562 | M − 1: | 4.98 | **** |
| | 596.0 (cal: 597.1) | | |
| 1563 | M − 1: 610.0 | 5.25 | **** |
| 1564 | M + 23: 650.2 | 4.83 | ***** |
| 1565 | M − 1: 616.1 | 4.83 | **** |
| 1566 | M − 1: 630.1 | 4.85 | *** |
| 1567 | M + 23: 652.1 | 4.93 | *** |
| 1568 | 593.2 | 2.43 | **** |
| 1569 | 615 | 4.52 | ***** |
| 1570 | 531 | 3.90 | ***** |
| 1571 | 531 | 4.00 | ***** |
| 1572 | 580 | 4.53 | ***** |
| 1577 | 521 | 3.93 | ***** |
| 1578 | 537 | 4.12 | ***** |
| 1580 | 684 | 4.32 | ***** |
| 1581 | 700 | 4.60 | ***** |
| 1604 | 521 | 3.95 | ***** |
| 1605 | 537 | 4.13 | ***** |
| 1607 | 684 | 4.30 | ***** |
| 1611 | 595.2 | 24.453 | ***** |
| 1612 | 491.365 | 5.676 | ***** |
| 1613 | 519.5 | 5.932 | ***** |
| 1614 | 505.5 | 5.775 | ***** |
| 1625 | M + 23: 618.2 | 4.61 | ***** |
| 1626 | M + 23: 632.2 | 4.76 | ***** |
| 1627 | M + 23: 667.2 | 3.96 | ***** |
| 1628 | M + 23: 667.1 | 4.03 | ***** |
| 1629 | M + 23: 667.1 | 4.92 | ***** |
| 1635 | M + 23: 620.1 | 4.73 | ***** |
| 1636 | M + 23: 634.1 | 4.92 | ***** |
| 1637 | M + 23: 664.1 | 5.03 | ***** |
| 1638 | M + 23: 654.1 | 5.03 | ***** |
| 1639 | M + 23: 666.1 | 5.10 | ***** |
| 1640 | M + 23: 612.2 | 4.93 | ***** |
| 1641 | M + 23: 647.2 | 5.13 | ***** |
| 1642 | M + 23: 600.1 | 4.92 | ***** |
| 1643 | M + 23: 614.2 | 5.12 | ***** |
| 1644 | M + 23: 628.2 | 5.35 | ***** |
| 1645 | M + 23: 644.2 | 4.91 | ***** |
| 1646 | M + 23: 634.2 | 4.88 | ***** |
| 1647 | M + 23: 646.2 | 4.99 | ***** |
| 1648 | 571 | 3.80 | ***** |
| 1652 | 700 | 4.53 | ***** |
| 1658 | 559 | 4.25 | ***** |
| 1659 | 545 | 4.12 | ***** |
| 1660 | 635 | 2.80 | ***** |
| 1661 | 650 | 2.47 | ***** |
| 1663 | 580.0 | 4.59 | ***** |
| 1664 | 579.9 | 4.84 | ***** |
| 1666 | M + 23: 648.1 | 5.44 | ***** |
| 1667 | M + 23: 640.1 | 4.55 | ***** |
| 1668 | M + 23: 620.1 | 5.45 | **** |
| 1669 | 492.1 | 13.380 | ***** |
| 1671 | 623.3 | 3.85 | ***** |
| 1672 | 593.34 | 3.70 | ***** |
| 1673 | 605.18 | 3.82 | ***** |
| 1674 | 696 | 3.33 | ** |
| 1675 | 864 | 3.88 | *** |
| 1676 | 710 | 3.33 | * |
| 1677 | 878 | 3.90 | *** |
| 1681 | 614 | 4.42 | ***** |
| 1682 | 649 | 2.33 | ***** |
| 1693 | 693 | 2.53 | ***** |
| 1694 | 550 | 2.40 | ***** |
| 1695 | 615 | 3.13 | ** |
| 1698 | 567.19 | 4.02 | ***** |
| 1701 | 509 | 3.87 | ***** |
| 1702 | 628 | 3.80 | ***** |
| 1703 | 624 | 2.35 | ** |
| 1704 | 610 | 2.40 | **** |

#(S) Isomer prepared and tested.
Wherein:
1 star, >1uM (1000 nM)
2 stars, 0.2 to 1 uM (200 nM to 1000 nM)
3 stars, 0.04 uM to 0.2 uM (40 nM to 200 nM)
4 stars, 0.008 uM to 0.04 uM (8 nM to 40 nM)
5 stars, <0.008 uM (<8 nM)

LC/MS for certain Compounds was performed on either a Waters 2795 or 2690 model separations module coupled with a Waters Micromass ZQ mass spectrometer using a Waters Xterra MS C18 4.6×50 mm reverse phase column (detection at 254 nM). The methods employed a gradient of acetonitrile (ACN) in water at 2 mL/min at ambient temperature as shown in Table 3a. The mobile phase was buffered with a 0.1 N formic acid.

The standard 6 minute method maintains a constant 85/5/10 ratio of water/ACN/1% aqueous formic acid from 0 minutes to 0.5 minutes. The method runs a linear gradient from 85/5/10 at 0.5 minutes to 0/90/10 at 3.5 minutes. The method holds at 0/90/10 until 4.5 minutes then immediately drops back down to 85/5/10 and holds there until 6 minutes.

The non-polar 6 minute method maintains a constant 60/30/10 ratio of water/ACN/1% aqueous formic acid from 0 minutes to 0.5 minutes. The method runs a linear gradient from 60/30/10 at 0.5 minutes to 0/90/10 at 3.5 minutes. The method holds at 0/90/10 until 4.5 minutes then immediately drops back down to 60/30/10 and holds there until 6 minutes.

The polar 6 minute method maintains a constant 90/0/10 ratio of water/ACN/1% aqueous formic acid from 0 minutes to 0.5 minutes. The method runs a linear gradient from 90/0/10 at 0.5 minutes to 20/70/10 at 3.5 minutes. The method holds at 20/70/10 until 4.5 minutes then immediately drops back down to 90/0/10 and holds there until 6 minutes.

TABLE 3a

| | | | % 1% Aq. | |
|---|---|---|---|---|
| Time | % Acetonitrile | % Water | Formic Acid | Gradient |
| Standard | | | | |
| 0.00 | 5 | 85 | 10 | |
| 0.50 | 5 | 85 | 10 | hold |
| 3.50 | 90 | 0 | 10 | linear |
| | | | | hold |
| 4.50 | 5 | 85 | 10 | instant |
| 6.00 | 5 | 85 | 10 | hold |
| Non-Polar | | | | |
| 0.00 | 30 | 60 | 10 | |
| 0.50 | 30 | 60 | 10 | hold |
| 3.50 | 90 | 0 | 10 | linear |
| | | | | hold |
| 4.50 | 30 | 60 | 10 | instant |
| 6.00 | 30 | 60 | 10 | hold |
| Polar | | | | |
| 0.00 | 0 | 90 | 10 | |
| 0.50 | 0 | 90 | 10 | hold |
| 3.50 | 70 | 20 | 10 | linear |
| | | | | hold |
| 4.50 | 0 | 90 | 10 | instant |
| 6.00 | 0 | 90 | 10 | hold |

LC/MS for Compounds 1611 and 1669 was performed using a C18-BDS 5 (250×4.6 mm) column with a 0.7 mL/min flow rate. The following solvent gradient was employed using 0.1% TFA/water as solvent A and acetonitrile as solvent B: 20% B for 0-20 minutes, 70% B for 20-30 minutes, 100% B for 30-40 minutes, 20% B for 40-50 minutes.

### 9. EXAMPLE

### Compound Pharmacodynamics

The examples that follow demonstrate that the Compounds tested can inhibit the pathological production of human VEGF, and suppress edema, inflammation, pathological angiogenesis and tumor growth tumor growth. Compounds tested have been shown to inhibit the pathological production of human VEGF by multiple human tumor cells and/or human tumors in animal models with pre-established human tumors.

9.1 Inhibition of Pathological Production of VEGF

9.1.1 Cell Based Assays

9.1.1.1 Compound #10 and Compound 1205 Inhibit Pathological VEGF Production in Transformed Cells Grown Under Hypoxic Conditions

This example demonstrates the selective inhibition of Compound #10 and Compound 1205 on pathological VEGF production in transformed HeLa cells grown under stressed conditions while sparing VEGF production in HeLa cells grown under non-stressed conditions.

Experimental Design. HeLa (human cervical carcinoma) cell cultures were established under normoxic conditions (21% oxygen). HeLa cells increase VEGF production 4- to 5-fold in response to hypoxia. In one experimental design, vehicle (0.5% DMSO) alone, or a range of concentrations of Compound #10 was added to the HeLa cell cultures and the cells were incubated for 48 hours under either hypoxic (1% oxygen) or normoxic conditions. In another experimental design, vehicle (0.5% DMSO) alone, or a range of concentrations of Compound #10, Compound 1205, or Compound 1330 was added to the culture medium and the cells were incubated for 48 hours. At the completion of treatment, the conditioned media were collected and the VEGF protein levels were assayed in an enzyme-linked immunosorbent assay (ELISA) with primary antibodies that recognize the soluble VEGF₁₂₁ and VEGF₁₆₅ isoforms (R & D Systems, Minneapolis, Minn., USA). To ensure that decreases in VEGF concentration were not due to cytotoxicity, cultures were assayed using a standard assay (CELLTITER-GLO® Luminescent Cell Viability Assay; Promega, Madison, Wis., USA) that measures total cellular adenosine triphosphate (ATP) concentrations as an indicator of cell viability.

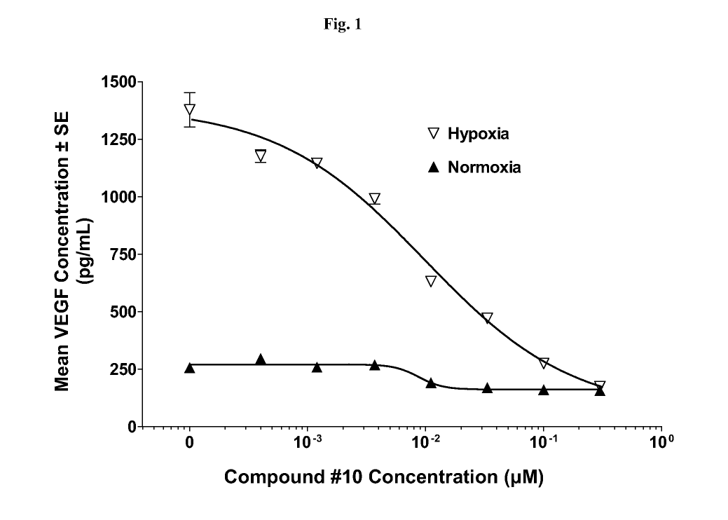

Results: FIG. 1 shows the concentrations of VEGF in conditioned media across the Compound #10 dose range tested. In the absence of Compound #10, media from hypoxic cells had substantial concentrations of VEGF (mean 1379 pg/mL). Compound #10 treatment induced dose dependent reductions in VEGF concentrations in the media, resulting in a maximal 87% decrease in VEGF concentration (to a mean of 175 pg/mL). By contrast, media from normoxic cells had relatively low concentrations of VEGF (mean 257 pg/mL) in the absence of Compound #10, and showed only a 39% decrease in VEGF concentrations (to a mean of 157 pg/mL) in the presence of Compound #10. No cytotoxicity was observed at any concentration tested. The data indicate that under stress conditions (with hypoxia), VEGF production was more sensitive to Compound #10 inhibition than under non-stress conditions (with normoxia). This data indicates that Compound #10 selectively inhibits or reduces pathological tumor-related production of soluble VEGF isoforms while sparing physiological VEGF production of the same isoforms. The (R)-enantiomer of Compound #10 showed lower activity (data not shown).

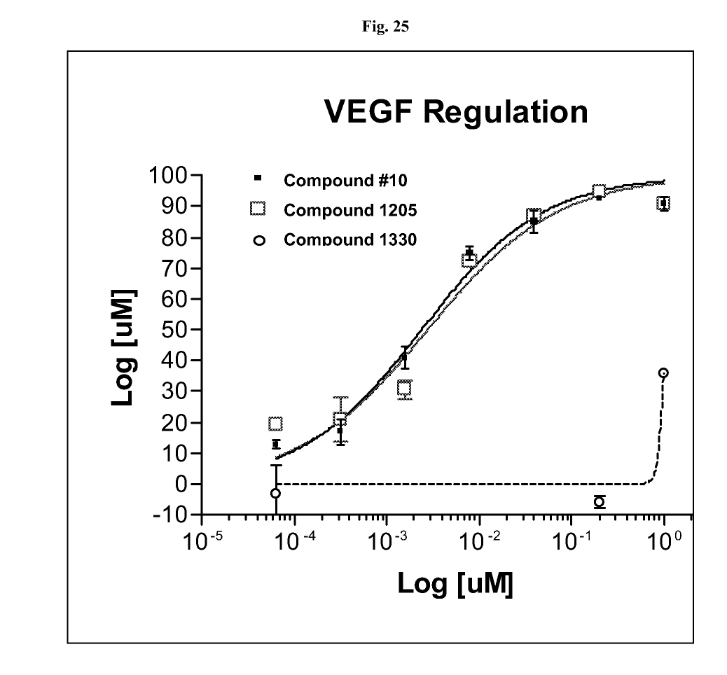

FIG. 25 shows the concentrations of VEGF in conditioned media across the dose range tested for Compound #10, Compound 1205 and Compound 1330. The data indicate that Compound #10 and Compound 1205 inhibit stress-induced VEGF production.

9.1.1.2 Compound #10 Inhibits Pathological VEGF Production in Nontransformed Cells Grown Under Hypoxic Conditions

This example demonstrates the inhibition of Compound #10 is selective for the pathological production of soluble VEGF isoforms in nontransformed keratinocytes grown under stressed conditions and does not affect the production of soluble VEGF isoforms in nontransformed keratinocytes grown under non-stressed conditions.

Experimental Design. Nontransformed normal human keratinocyte cell cultures were established under normoxic conditions (21% oxygen). Vehicle (0.5% DMSO) alone, or a range of concentrations of Compound #10 was added to the cultures and the cells were incubated for 72 hours under either under hypoxic (1% oxygen) or normoxic conditions. At the completion of treatment, cells were assessed for viability with an ATP assay and conditioned media were evaluated for VEGF protein levels by ELISA (as described in Section 9.1.1.1).

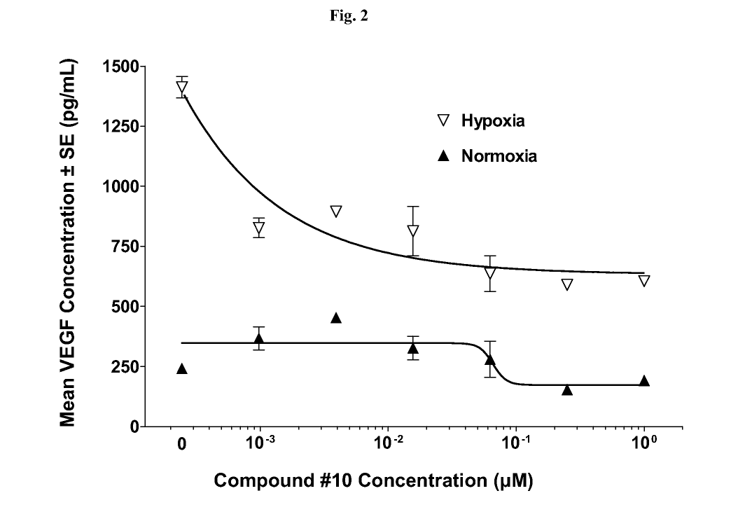

Results: FIG. 2 shows the concentrations of VEGF in conditioned media across the Compound #10 dose range tested. In the absence of Compound #10, media from hypoxic keratinocytes had substantial concentrations of VEGF (mean 1413 pg/mL). Compound #10 treatment induced dose dependent reductions in VEGF concentrations in the media, resulting in a maximal 57% decrease in VEGF concentration (to a mean of 606 pg/mL). By contrast, media from normoxic cells had relatively low concentrations of VEGF (mean 242 pg/mL) in the absence of Compound #10 and showed only a 21% decrease in VEGF concentrations (to a mean of 192 pg/mL) in the presence of Compound #10. No toxicity was observed at any concentration tested.

This data indicates that Compound #10 selectively inhibits or reduces pathological production of soluble VEGF isoforms in nontransformed keratinocytes grown under stressed hypoxic conditions while sparing physiological VEGF production of the same isoforms in unperturbed cells.

9.1.1.3 Compound #10 Inhibits Matrix-Bound Tumor VEGF Production

This example demonstrates that Compound #10 inhibits the pathological production of matrix bound/cell associated VEGF₁₈₉ and VEGF₂₀₆ isoforms resulting from oncogenic transformation.

Experimental Design. HT1080 (human fibrosacoma) cell cultures were established under normoxic conditions (21% oxygen). HT1080 cells constitutively overproduce VEGF even under normoxic conditions. Vehicle (0.5% DMSO) alone or a range of concentrations of Compound #10 was added to the cultures, and the cells were incubated for 48 hours under normoxic conditions. At the completion of treatment, the cells were washed and harvested. Cells were incubated with a primary antibody that recognizes the VEGF₁₈₉ and VEGF₂₀₆ isoforms. Infrared-dye labeled antibodies were applied secondarily, and the amounts of VEGF₁₈₉ and VEGF₂₀₆ were determined using the IN-CELL WESTERN® assay and ODYSSEY® infrared imaging system (Li-Cor, Lincoln, Nebr., USA); results are expressed as percentage inhibition relative to vehicle treated controls. Conventional Western blotting using the same primary antibody was also performed to confirm the presence of the matrix associated isoforms; for these experiments actin was used as a loading control. Actin is a ubiquitous housekeeping protein that is not known to be post transcriptionally regulated.

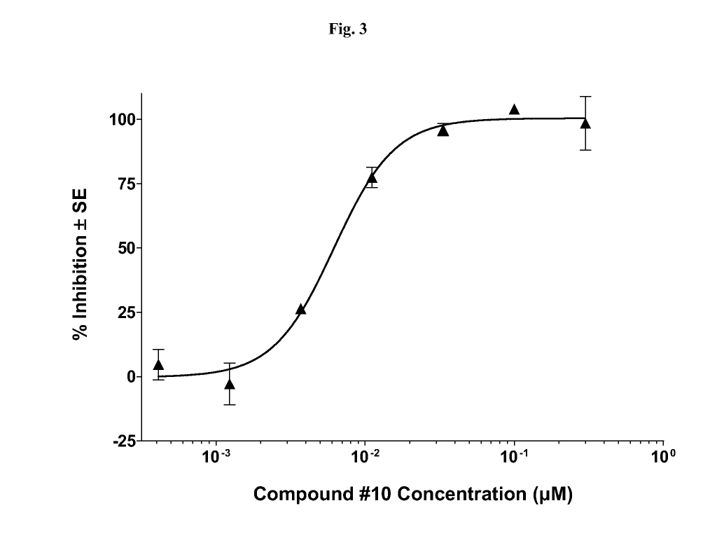

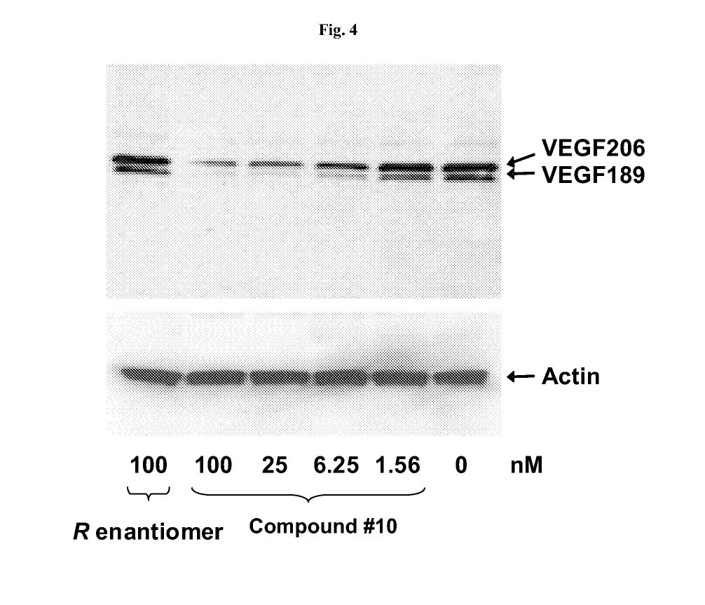

Results. As shown in FIG. 3, Compound #10 induced a potent concentration-dependent inhibition of VEGF₁₈₉ and VEGF₂₀₆ isoforms. These results demonstrate that Compound #10 inhibits the production of matrix-associated as well as soluble forms of tumor-derived VEGF. As shown in FIG. 4, immunoblotting documented the presence of 2 bands at the expected location for VEGF₁₈₉ and VEGF₂₀₆, and confirmed a concentration-dependent Compound #10 effect in reducing the amounts of these isoforms. The activity of the (R) enantiomer was relatively lower.

This data shows that Compound #10 inhibits pathological production of the matrix bound/cell associated VEGF isoforms resulting from oncogene transformation.

9.1.1.4 Compound #10 Inhibits Soluble VEGF Production in Multiple Human Tumor Cell Lines

This example demonstrates that Compound #10 inhibits soluble VEGF production in multiple human tumor cell lines.

Study Design. The activity of Compound #10 in suppressing VEGF production in a number of other human tumor cell lines has been assessed. These evaluations focused on cell lines that produce sufficient soluble VEGF (>200 pg/mL in conditioned media, either constitutively or under hypoxic stress) to allow assessment of Compound #10 activity by ELISA. In these experiments, cultures were established under normoxic conditions (21% oxygen). Cultures were then incubated for 48 hours with vehicle (0.5% DMSO) alone or with Compound #10 over a range of concentrations from 0.1 nM to 10 μM. Cells requiring induction of VEGF production were incubated under hypoxic conditions (1% oxygen). At the completion of treatment, the conditioned media were collected and assayed by ELISA (as described in Section 9.1.1.1) for soluble VEGF₁₂₁ and VEGF₁₆₅ isoforms; results were calculated as percentage inhibition relative to vehicle treated controls. EC₅₀ values were calculated from the dose concentration response curves.

Results. Compound #10 potently inhibited the production of soluble VEGF in 18 of the human tumor cell lines tested to date. The EC₅₀ values for cell lines showing VEGF inhibition are generally in the low nanomolar range, as presented in Table 4. Compound #10 did not show activity in several cell lines in which there was insufficient basal or inducible production of soluble VEGF. Other human cell lines that produce sufficient soluble VEGF in vitro or in vivo may be used, with appropriate adaptations, by those skilled in the art to measure inhibition of pathologically produced soluble human VEGF.

TABLE 4
Inhibition of Soluble VEGF Production by Compound #10 in
Human Cell Lines—EC₅₀ Values by Cell Line

| | | VEGF Inhibition |
|---|---|---|
| Tumor Type | Cell Line | EC₅₀ (nM) |
| Breast | MDA-MB-231^{a} | 5 |
| | MDA-MB-468^{a} | 5 |
| Cervical | HeLa^{a} | 2 |
| Colorectal | HCT-116 | 10 |
| Epidermoid | A431 | 10 |
| Fibrosarcoma | HT1080 | 10 |
| Gastric | SNU-1 | 0.1 |
| | AGS | 0.1 |
| | Kato III^{a} | 10 |
| Lung | NCI H460 | 10 |
| | A549 | 50 |
| | Calu-6^{a} | 7 |
| Melanoma | A375^{a} | 50 |
| Neuroblastoma | SY5Y^{a} | 5 |
| Ovarian | SKOV3^{a} | 10 |
| Pancreas | Capan-1^{a} | 5 |
| Prostate | LNCaP^{a} | 15 |
| Renal cell | HEK293 | 10 |

^{a}Cell lines requiring incubation under hypoxic conditions (1% oxygen) to induce VEGF production.
Abbreviations:
EC₅₀ = effective concentration achieving 50% of peak activity;
VEGF = vascular endothelial growth factor

9.1.2 Animal Model Systems

9.1.2.1 Compound #10 Selectively Inhibits Pathological VEGF Production Relative to Other Human Angiogenic Factors

This example demonstrates that Compound #10 selectively inhibits pathological VEGF production relative to other human angiogenic factors.

Experimental Design. In a series of experiments evaluating the effects of Compound #10 on intratumoral VEGF and tumor growth, intratumoral levels of VEGF-C, P1GF (Placental Growth Factor), FGF-2 (Fibroblast growth factor 2), survivin, PDGF (Platelet derived growth factor), and endostatin were also measured to assess the selectivity of Compound #10. VEGF-C and P1GF were analyzed to determine the in vivo effects of Compound #10 on other members of the VEGF family of angiogenic factors. All of these factors can be produced at tumor sites, and all may support tumor growth and metastases. See Yoon et al., Circ Res. 2003, 93(2):87 90; Ferrara et al., Nat. Rev. Drug Discov. 2004, 3(5):391 400; Luttun et al., Biochim. Biophys. Acta 2004,1654(1):79 94; Saharinen et al., Trends Immunol. 2004, 25(7):387 95. There is also evidence that VEGF-A may stimulate production of P1GF by a post transcriptional mechanism. See Yao et al., FEBS Lett. 2005, 579(5):1227 34. VEGF-B was not assessed. The angiogenic growth factor FGF-2 was analyzed because it promotes tumor survival (see Bikfalvi et al., Angiogenesis 1998, 1(2):155 73), and has a 5′-UTR IRES. See Vagner at al., Mol. Cell. Biol. 1995, 15(1):35 44; Hellen et al., Genes Dev. 2001, 15(13):1593 612. The survivin protein was similarly evaluated because the survivin mRNA has an IRES. PDGF was assessed because this protein has angiogenic activity and its mRNA contains an IRES. See Sella et al., Mol. Cell. Biol. 1999, 19(8):5429 40; Hellen et al., supra. Endostatin was included because antiangiogenic treatment in vivo has shown that compensatory decreases in endogenous angiogenic inhibitors such as endostatin, thrombospondin, and angiostatin, results in a more pro angiogenic environment. See Sim, Angiogenesis, 1998, 2(1):37-48.

In all of these experiments, HT1080 cells (5×10⁶ cells/mouse) were implanted subcutaneously in male athymic nude mice. When tumors had become established, mice were divided into groups (10 mice/group). Treatments comprised Compound #10 (either alone or as the racemic mixture) or the corresponding vehicle alone, administered by oral gavage BID (“bis in die”; twice a day) on Monday through Friday and QD (“quaque die”; daily) on Saturday and Sunday over periods of 7 to 21 days (Table 1). Tumor size was measured by calipers at the beginning and end of treatment. At the completion of Compound administration, the mice were sacrificed, and excised tumors were assayed by ELISA for intratumoral VEGF or other angiogenic factors using methods analogous to those described in Section 9.1.1.1.

Results. As summarized in the studies shown in Table 5, Compound #10 universally inhibited the production of intratumoral VEGF A and tumor size. Compound #10 also reduced intratumoral P1GF in the experiments where this factor was measured; the results show a variable effect on VEGF-C. Compound #10 did not have statistically significant effects on levels of the other proteins tested, except for FGF 2 levels (as shown in Study 5). In Study 5, treatment was initiated when the tumors were quite large (˜600 mm³). The study was continued for 15-days, and the tumors had become quite bulky by the time intratumoral protein levels were analyzed. However, Compound #10 still decreased intratumoral VEGF levels by 78%, although FGF-2 levels were noted to be significantly elevated at the time of study termination. In Studies 2 and 3, endostatin levels were depressed by 22 to 30%, although these changes were not statistically significant. Collectively, these data indicate that Compound #10 is selective for suppression of VEGF family proteins.

TABLE 5
Study Design and Efficacy Information for Assessments of Selectivity for
VEGF Inhibition by Compound #10 in Nude Mice Bearing HT1080 Xenografts.

| | | Study Number | | | | | | |
|---|---|---|---|---|---|---|---|---|
| Parameter | | 1 | 2 | 3 | 4 | 5 | 6 | 7 |
| Animal number per group | | 10 | 10 | 10 | 10 | 10 | 7 | 10 |
| Compound #10 dose (mg/kg)^{a} | | 1 | 5 | 5 | 5 | 5/50^{b} | 5 | 10 |
| Administration | Route | Oral | Oral | Oral | Oral | Oral | Oral | Oral |
| | Schedule | BID^{c} | BID^{c} | BID^{c} | BID^{c} | BID^{c} | QD^{d} | QD^{d} |

| | | | | | | | |
|---|---|---|---|---|---|---|---|
| Vehicle | DMSO/PEG | DMSO/PEG | DMSO/PEG | DMSO/PEG | DMSO/PEG | L21^{e} | L21^{e} |
| Compound #10 Treatment | 28 | 7 | 10 | 9 | 15 | 21 | 42 |
| duration (days) | | | | | | | |
| Vehicle-treatment duration (days) | 14 | 7 | 10 | 9 | 10 | 21 | 10 |
| Initial mean tumor size (mm³) | 85 | 390 | 285 | 610 | 610 | 180 | 160 |
| Final mean Compound | 450 | 595 | 735 | 953 | 1922 | 750 | 1770 |
| #10-treated tumor size (mm³) | | | | | | | |

Mean % difference relative to vehicle-treated animals^{f} in:

| | | | | | | | |
|---|---|---|---|---|---|---|---|
| Tumor size | −58*^{g} | −32* | −40* | −44* | −34*^{h} | −51* | −63*^{h} |
| Human VEGF-A (%) | −57* | −81* | −95* | −85* | −78* | −95* | −42 |
| Human VEGF-C (%)ⁱ | ND | −19 | −26 | ND | ND | −38* | +10 |
| Human PlGF (%)ⁱ | ND | −67* | −59* | ND | ND | −73 | −65* |
| FGF-2 | +3 | +3 | +5 | +15 | +31* | ND | ND |
| Survivin | +7 | ND | ND | −9 | ND | ND | ND |
| PDGF | +12 | ND | −30 | +23 | +20 | ND | ND |
| Endostatin | ND | −30 | −22 | ND | ND | ND | ND |

*p < 0.05 (Student's t-test relative to vehicle)
^{a}Some animals received racemic mixture; the dose is expressed as amount of Compound #10 in the mixture.
^{b}Mice were treated with 5 mg/kg for the first 9 days and with 50 mg/kg for the last 6 days.
^{c}Treatments were administered by oral gavage BID on Monday through Friday and QD on Saturday and Sunday for the number of days shown. All morning doses were given before 0830 hours. Evening doses were administered after 1630 hours (i.e., ≧8 hours after the morning dose).
^{d}Treatments were administered by oral gavage QD in the morning before 0830 hours on Monday through Friday for the number of days shown.
^{e}Vehicle was 35% Labrasol, 35% Labrafac and 30% Solutol).
^{f}Calculated as [1 − (treated/control)] × 100%
^{g}Difference in tumor size is shown for Day 14, the day the vehicle-treated mice were taken off study.
^{h}Difference in tumor size is shown for Day 10, the day the vehicle-treated mice were taken off study.
ⁱSix mice per group in Compound #10-treated and vehicle-treated groups were analyzed
Abbreviations:
BID = 2 times per day;
QD = 1 time per day;
DMSO = dimethyl sulfoxide;
PEG-300 = polyethylene glycol (molecular weight 300);
FGF-2 = basic fibroblast growth factor-2;
PDGF = platelet-derived growth factor;
PlGF = placental growth factor;
VEGF = vascular endothelial growth factor;
ND = not done

9.1.2.2 Compound #10 Dose-Dependently Reduces Tumor and Pathologically Produced Plasma Human VEGF Concentrations

This example demonstrates that Compound #10 dose-dependently reduces intratumoral and pathologically produced plasma human VEGF concentrations in vivo.

Experimental Study Design. HT1080 cells (5×10⁶ cells/mouse) were implanted subcutaneously in male athymic nude mice. When tumors had become established (i.e., the mean tumor size had reached 180±75 mm³), mice were divided into 6 groups and treatment was assigned as shown in Table 6.

TABLE 6
Study Design for Dose Response Assessment in Nude Mice
Bearing HT1080 Xenografts.

| | Number | | | | | | Dose |
|---|---|---|---|---|---|---|---|
| | of | | | | | Dose | Concen- |
| Test | Animals | | Dose | Administration^{a} | | Volume | tration |
| Compound | M | F | (mg/kg) | Route | Schedule | (mL/kg) | (mg/mL) |
| Vehicle^{b} | 10 | 0 | 0 | Oral | BID | 4 | 0 |
| Com- | 10 | 0 | 0.3 | Oral | BID | 4 | 0.075 |
| pound #10 | | | | | | | |
| Com- | 10 | 0 | 1 | Oral | BID | 4 | 0.25 |
| pound #10 | | | | | | | |
| Com- | 10 | 0 | 3 | Oral | QD | 4 | 0.75 |
| pound #10 | | | | | | | |
| Com- | 10 | 0 | 3 | Oral | BID | 4 | 0.75 |
| pound #10 | | | | | | | |
| Com- | 10 | 0 | 10 | Oral | QD | 4 | 2.5 |
| pound #10 | | | | | | | |

^{a}Treatments were administered by oral gavage 7-days per week (except the 10-mg-QD regimen, which was administered daily on Monday through Friday) for a total of 18 days. All morning doses were given before 0830 hours. For BID schedules, evening doses were administered after 1630 hours (i.e., ≧8 hours after the morning dose).
^{b}Vehicle was L21 (35% Labrasol, 35% Labrafac, and 30% Solutol).
Abbreviations:
BID = 2 times per day;
QD = 1 time per day

Tumor size was measured using calipers at periodic intervals during the study (data shown in Section 9.2.2). Retro-orbital blood collection was performed to assess Compound #10 trough plasma concentrations after the first dose (just prior to the second dose) on Day 1, Day 4, and Day 9, and at study termination. The study was ended after 18 days, when the vehicle treated tumors reached a mean volume of ˜1755 mm³. Retro-orbital terminal bleeding was performed at ˜8 to 16 hours (depending upon the schedule of Compound administration) after the last dose to assess pathologic plasma human VEGF concentrations and trough Compound #10 plasma concentrations. Mice were sacrificed, and excised tumors were homogenized in buffer containing protease inhibitors. Both terminal intratumoral and pathologic plasma human VEGF levels were measured using an ELISA that recognizes human VEGF₁₂₁ and VEGF₁₆₅ (as described in Section 9.1.1.1). Intratumoral VEGF levels were normalized to the total tumor protein concentration, while pathologic plasma human VEGF levels were expressed in pg/mL of plasma. Plasma Compound #10 concentrations were evaluated by high performance liquid chromatography and with tandem mass spectroscopy (HPLC-MS/MS).

Results. As shown in FIG. 5 and FIG. 6, Compound #10 significantly suppressed pathologic human VEGF levels in tumors and in plasma in all Compound #10 dose groups. At the suboptimal Compound #10 dose of 0.3 mg/kg BID, partial reductions in both tumor and pathologic plasma human VEGF concentrations were observed, while human VEGF reductions were essentially maximal at all Compound #10 dose levels of ≧1 mg/kg BID. The correlation between pathologic plasma and tumor human VEGF levels in this animal model supports the potential utility of assessing pathologic plasma human VEGF levels to serve as a mechanism-specific, pharmacodynamic marker of Compound activity in the clinic.

The data shows that Compound #10 dose-dependently reduces intratumoral and pathologically produced plasma human VEGF concentrations in vivo.

9.1.2.3 Compound 1205 Reduces Tumor and Pathologically Produced Plasma Human VEGF Concentrations

This example demonstrates that Compound 1205 reduces intratumoral and pathologically produced plasma human VEGF concentrations in vivo.

Experimental Design. HT1080 cells (5×10⁶ cells/mice) were implanted subcutaneously into male athymic nude mice. Treatment with vehicle alone or Compound 1205 was initiated when the median tumor volume was approximately 311±88 mm³. Table 7 and Table 9 (study design #21 and #23) provide the study design for assessing tumor and plasma pathologic VEGF concentrations—each group in each study included eight (8) mice. When the tumors in vehicle-treated mice had reached the target size of ˜1200 mm³ for study #21 and ˜1500 mm³ for study #23, all mice in the study were sacrificed, and excised tumors were homogenized in buffer containing protease inhibitors. Both intra-tumor and pathologic plasma human VEGF levels were measured using an ELISA that recognizes human VEGF₁₂₁ and VEGF₁₆₅. Intra-tumor VEGF levels were normalized to the total tumor protein concentration and pathologic plasma VEGF levels were expressed in pg/mL. Because smaller tumors produce less VEGF per mg of tumor protein, intra-tumor VEGF levels were normalized to tumor size. Table 9 provides the study design for assessing tumor and pathologic plasma VEGF.

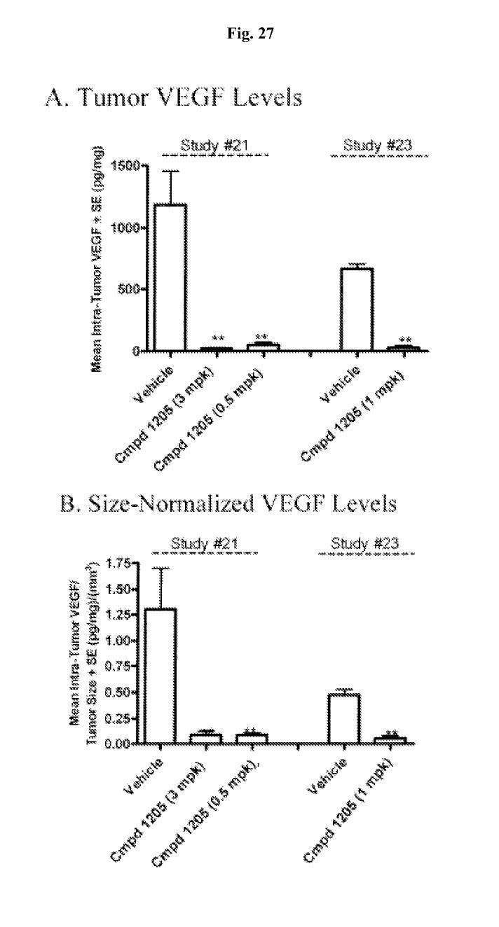

Results. Treatment with Compound 1205 at 0.5 or 3 mg/kg for 14-days significantly reduced the levels of pathologic human VEGF measured in excised tumors (FIG. 27) and in plasma (FIG. 1) compared to levels measured in tumors and plasma from mice treated with vehicle. At the dose of 0.5 or 3 mg/kg QD, Compound 1205 inhibits both tumor and pathologic plasma human VEGF levels by more than 95%. Even with the reduction in tumor size in the treated groups, the volume normalized intra-tumor human VEGF levels were significantly reduced (FIG. 1, Table 7).

TABLE 7
Inhibition of Intra-Tumor and Pathologic Plasma Human VEGF by
Compound 1205

| | Study #21 | | | Study #23 | |
|---|---|---|---|---|---|
| | | Compound | | | Compound |
| | Vehicle | 1205 | | Vehicle | 1205 |
| 1) Dose (mg/kg) | 0 | 0.5 | 3 | 0 | 1 |
| 2) Regimen | QD | QD | QD | QD | QD |
| 3) Test-Compound | 14 | 14 | 14 | 14 | 14 |
| duration (days) | | | | | |
| 4) Mean difference | NA | 95%** | 98%** | NA | 95** |
| in human tumor | | | | | |
| VEGF (%) at Day | | | | | |
| 14 (Compound | | | | | |
| 1205) or Day 18 | | | | | |
| (Compound #10) | | | | | |
| 5) Mean difference | NA | 97%** | 99% | NA | 100%** |
| in human plasma | | | | | |
| VEGF (%) on Day | | | | | |
| 14 (Compound | | | | | |
| 1205) or on Day 18 | | | | | |
| (Compound #10) | | | | | |

**p < 0.05 (ANOVA vs. vehicle).

9.2 Inhibition of Pathological Angiogenesis and Tumor Growth

9.2.1 Compound #10 Inhibits Tumor Angiogenesis

This example demonstrates that Compound #10 reduces the total volume and diameter of tumor vessels.

Experimental Design. HT1080 cells (5×10⁶ cells/mouse) were implanted subcutaneously in male athymic nude mice. At a mean tumor size of 285±45 mm³, mice were divided into 2 groups and treatment was administered as shown in Table 8.

At the end of treatment, the mice were sacrificed. Excised tumors were assayed by ELISA for VEGF content as described in Section 9.1.1.1, and were sectioned and immunostained with an anti murine CD31 antibody that is specific for endothelial cells.

TABLE 8
Study Design for Assessment of Intratumoral Microvessel Density
in Nude Mice Bearing HT1080 Xenografts.

| | Number | | | | | | Dose |
|---|---|---|---|---|---|---|---|
| | of | | | | | Dose | Concen- |
| Test | Animals | | Dose | Administration^{a} | | Volume | tration |
| Compound | M | F | (mg/kg) | Route | Schedule | (mL/kg) | (mg/mL) |
| Vehicle^{b} | 10 | 0 | 0 | Oral | BID | 8 | 0 |
| Racemic | 10 | 0 | 5^{c} | Oral | BID | 8 | 0.625 |
| mixture^{c} | | | | | | | |

^{a}Treatments were administered by oral gavage BID on Monday through Friday and QD on Saturday and Sunday Treatments were administered by oral gavage BID on Monday through Friday and QD on Saturday and Sunday for a total of 10 days. All morning doses were given before 0830 hours. Evening doses were administered after 1630 hours (i.e., ≧8 hours after the morning dose).
^{b}Vehicle was 5% DMSO and 95% PEG 300.
^{c}Racemic material was used for this study at a dose of 10 mg/kg (1.25 mg/mL), resulting in a dose of the active Compound #10 enantiomer of 5 mg/kg (0.625 mg/mL).
Abbreviations:
BID = 2 times per day;
DMSO = dimethyl sulfoxide;
PEG 300 = polyethylene glycol (molecular weight 300);
QD = 1 time per day

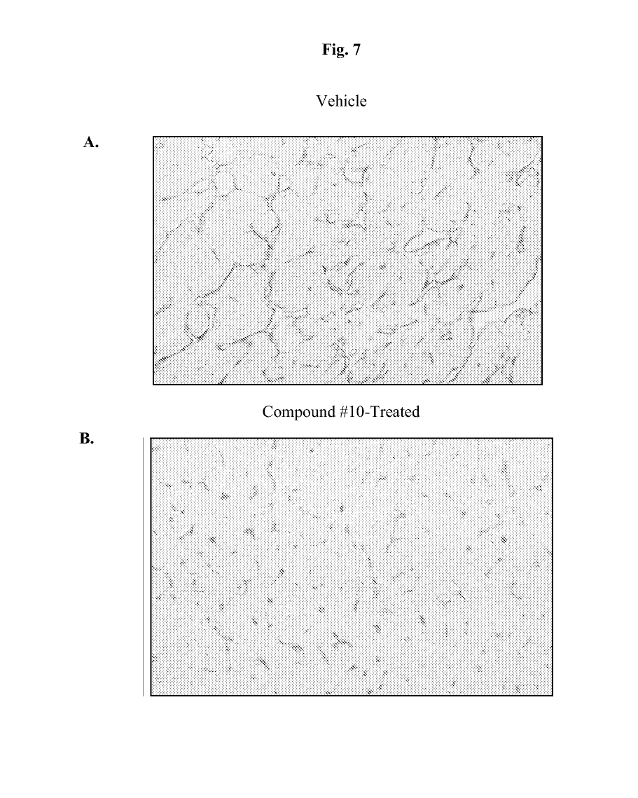

Results. Treatment with Compound #10 resulted in a mean 95% inhibition of tumor VEGF concentration. As shown in FIG. 7, this activity resulted in a profound effect on the architecture of the vasculature. Although the vessel count was unchanged, the total volume of tumor vessels and the diameters of vessels were visibly reduced. These findings are consistent with results from reports describing the effects of antiangiogenic therapies on larger tumors that have an existing vasculature. See Yuan et al., Proc. Natl. Acad. Sci. USA. 1996; 93(25):14765-70.

9.2.2 Compound #10 Inhibits Tumor Growth In Vivo

This example demonstrates that Compound #10 inhibits tumor growth in nude mice bearing HT1080 xenografts.

Experimental Design. The experimental design was reported in Section 9.1.2.2.

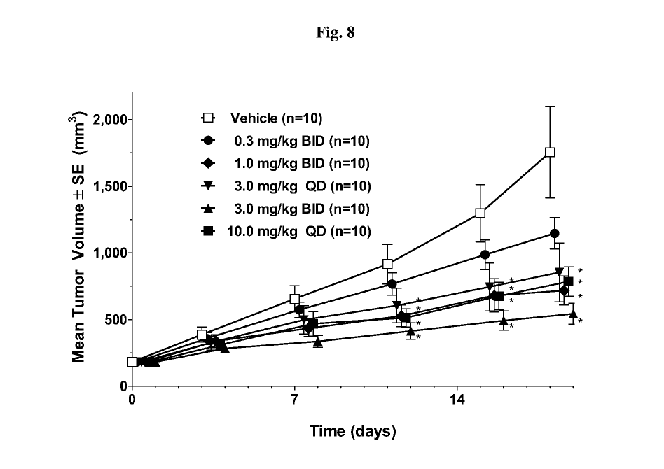

Results. The dose response effect of Compound #10 that correlated with decreases in tumor and pathologic human VEGF concentrations (see FIG. 5 and FIG. 6; Section 9.1.2.2) was also observed when assessing tumor size by treatment group over time. As depicted in FIG. 8, maximum antitumor activity was again observed at Compound #10 dose levels of ≧1 mg/kg BID. The dose of 1 mg/kg BID was associated with mean trough plasma concentrations of 0.13 μg/mL (0.28 μM) at 16 hours after the first day of dosing (n=3), and with steady state mean trough plasma concentrations of 0.82 μg/mL (1.76 μM) at 16 hours after the last dose on Day 18 (n=4). These data provide an indication of trough plasma concentrations that could be targeted when assessing the pharmacokinetics (PK) of a Compound in humans. In observing the animals, there was no overt toxicity associated with Compound #10 treatment. This data shows that Compound #10 inhibits tumor growth in nude mice bearing HT1080 xenografts.

9.2.3 Compound 1205 Inhibits Tumor Growth In Vivo

This example demonstrates that Compound 1205 inhibits tumor growth in nude mice bearing HT1080 xenografts.

Experimental Design. HT1080 cells (5×10⁶ cells/mouse) were implanted subcutaneously in male athymic nude mice. When tumors had become established (i.e., the mean tumor size had reached 311±88 mm³), mice were divided into 5 groups and treatment was administered as shown in Table 9 and 10. Compound 1330 is a relatively inactive (R,S) diastereomer of Compound 1205, which has (S,S) configuration. For comparison, Compound #10 was included in this study.

TABLE 9
Study Design for HT1080 Xenograft Studies Assessing In Vivo
Efficacy of Compound 1205 and Compound #10.

| | # of | | | Dose | Dose | | |
|---|---|---|---|---|---|---|---|
| | Animals | Dose | | Volume | Conc. | | |
| Test Compound | Male | (mg/kg) | Regimen | (mL/kg) | (mg/mL) | Study # | Study Termination |
| Vehicle† | 8 | 0 | QD | 8 | 0 | 21 | All mice were taken off study |
| Compound 1205 | 8 | 0.5 | QD | 8 | 0.0625 | 21 | when tumors in vehicle |
| Compound 1205 | 8 | 3 | QD | 8 | 0.375 | 21 | reached 1200 mm³ |
| Vehicle† | 8 | 0 | QD | 8 | 0 | 22 | (A) Vehicle-treated mice |
| Compound 1205 | 8 | 0.5 | QD | 8 | 0.0625 | 22 | were taken off study when |
| Compound 1205 | 8 | 3 | QD | 8 | 0.375 | 22 | the average tumor size of the |
| | | | | | | | group wais 1500 mm³. (B) |
| | | | | | | | Each treated mouse was taken |
| | | | | | | | off study when its tumor was |
| | | | | | | | 1500 mm³ |
| Vehicle† | 8 | 0 | QD | 8 | 0 | 23 | All mice were taken off study |
| Compound 1205 | 8 | 1 | QD | 8 | 0.125 | 23 | when tumors in vehicle |
| | | | | | | | reached 1500 mm³ |
| Vehicle† | 8 | 0 | QD | 8 | 0 | 24a | A) Vehicle- and Compound |
| Compound 1205 | 8 | 10 | QD | 8 | 1.25 | 24a | 1330-treated mice were taken |
| Compound | 8 | 10 | QD | 8 | 1.25 | 24a | off study when the average |
| 1330Φ | | | | | | | tumor size of the group wais |
| | | | | | | | 1500 mm³. (B) Each treated |
| | | | | | | | mouse was taken off study |
| | | | | | | | when its tumor was 1500 mm³ |
| Vehicle† | 8 | 0 | QD | 8 | 0 | 24b | (A) Vehicle-treated mice |
| Compound 1205 | 8 | 0.3 | QD | 8 | 0.0375 | 24b | were taken off study when |
| | | | | | | | the average tumor size of the |
| | | | | | | | group wais 1500 mm³. (B) |
| | | | | | | | Each treated mouse was taken |
| | | | | | | | off study when its tumor was |
| | | | | | | | 1500 mm³ |

†Vehicle was 0.5% HPMC/1% Tween-80
‡Vehicle was L21 (35% Labrasol, 35% Labrafac, and 30% Solutol).
ΦInactive (R, S) diastereomer of Compound 1205
Abbreviations:
BID = twice per day,
QD = once per day

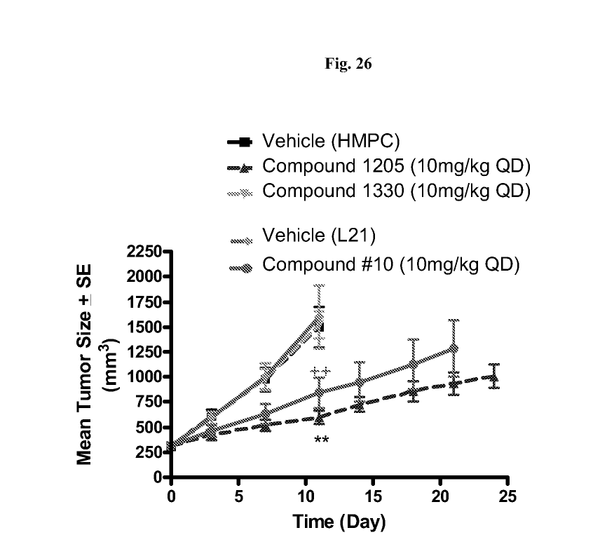

Results. The results of the studies described in Table 10 and are shown in FIG. 26 for study #24a. The data indicate that Compound 1205 (S,S diastereoisomer) inhibits tumor growth in an animal model with a pre-established human tumor. As shown in FIG. 26, treatment with Compound 1205 (S,S), but not with the (R,S) diastereomer Compound 1330, significantly delayed growth of HT1080 tumor cells in vivo. The growth of the tumors in mice treated with Compound 1330 overlapped with the growth of tumors in mice treated with 0.5% HPMC vehicle. This suggests that the relatively inactive (R, S) diastereomer (Compound 1330) does not appreciably isomerize to active Compound 1205 in vivo. Compound 1205 is active at doses as low as 0.3 mg/kg.

TABLE 10
Effect of Compound 1205 and Compound #10 on Growth of
HT1080 Tumor Cells In Vivo.

| | | | | | | | | Compound |
|---|---|---|---|---|---|---|---|---|
| | Compound 1205 | | | | | | | #10 |
| Study #^{A} | 24b | 22 | 21 | 23 | 22 | 21 | 24a | 24a |

Study Information

| | | | | | | | | |
|---|---|---|---|---|---|---|---|---|
| Dose (mg/kg) | 0.3 | 0.5 | 0.5 | 1 | 3 | 3 | 10 | 10 |
| Regimen | QD | QD | QD | QD | QD | QD | QD | QD |
| Dose (mg/kg/week) | 2.1 | 3.5 | 3.5 | 7 | 21 | 21 | 70 | 70 |
| Study design | Xeno | Xeno | PD | PD | Xeno | PD | Xeno | Xeno |
| Number of days that test compound was | 16^{C} | 28^{C} | 14 | 14 | 32^{C} | 14 | 30^{C} | 27^{C} |
| administered | | | | | | | | |
| Initial mean tumor size (mm³) | 204 | 170 | 167 | 157 | 170 | 167 | 311 | 311 |
| Day that vehicle-treated mice were | 15 | 11 | 14 | 14 | 11 | 14 | 11 | 11 |
| taken off study | | | | | | | | |
| Mean tumor size in vehicle-treated mice | 1790 | 1390 | 1210 | 1500 | 1390 | 1210 | 1500 | 1500 |
| when taken off study | | | | | | | | |
| Final mean terminal tumor size in treatment | 1540 | 1750 | 580 | 710 | 1840 | 379 | 1400 | 1460 |
| group (mm³) | | | | | | | | |

Results

| | | | | | | | | |
|---|---|---|---|---|---|---|---|---|
| Mean difference in tumor growth | 28% | 62%** | 61%** | 59%** | 75%** | 80%** | 76%** | 59%** |
| rate at the Day that the vehicle- | | | | | | | | |
| treated tumors taken off study (%)^{B} | | | | | | | | |
| Difference vs. vehicle in median | 0.7 | 11 | NA | NA | 14** | NA | 14** | 8** |
| number of days to reach 1000 mm³ | | | | | | | | |
| (Days) | | | | | | | | |

^{A}See Table 9Table 9, for additional study information.
^{B}% Difference in the rat of growth in compound-treated vs. vehicle-treated
**P < 0.05 (ANOVA vs. vehicle)
^{C}Average time on study.
NA not applicable.
The time to progression could not be calculated for PD (pharmacodynamic) studies.
Xeno Xenograft

9.2.4 Time-Course Effects of Compound #10 on Tumor Size and Pathologically Produced Plasma Human VEGF Concentrations

This example demonstrates that Compound #10 has a rapid onset for reducing xenograft tumor size and pathologically produced plasma human VEGF concentration.

Experimental Design. HT1080 cells (5×10⁶ cells/mouse) were implanted subcutaneously in male athymic nude mice. When tumors had become established (i.e., the mean tumor size had reached 585±150 mm³), mice were divided into 4 treatment groups, as shown in Table 11.

TABLE 11
Study Design for Time Course Assessment in Nude Mice Bearing
HT1080 Xenografts

| | Number | | | | | | |
|---|---|---|---|---|---|---|---|
| | of | | | | | | |
| | Animals | | | | | | |
| | Per | | | | | | Dose |
| | Time | | | Administration^{a} | | Dose | Concen- |
| Test | Point^{a} | | Dose | | Sched- | Volume | tration |
| Compound | M | F | (mg/kg) | Route | ule | (mL/kg) | (mg/mL) |
| Vehicle^{b} | 5 | 0 | 0 | Oral | QD | 4 | 0 |
| Compound | 5 | 0 | 10 | Oral | QD | 4 | 2.50 |
| #10 | | | | | | | |
| Doxorubicin | 5 | 0 | 6 | IP | Single | 8 | 0.75 |
| | | | | | bolus | | |
| Bevacizumab | 5 | 0 | 5 | IP | Single | 8 | 0.625 |
| | | | | | bolus | | |

^{a}Treatments were initiated on Day 0 with 20 mice per group. On each day, 5 mice were sacrificed per group for analysis. Mice were treated with Compound #10 daily. Mice were treated with doxorubicin or bevacizumab on Day 0 only.
^{b}Vehicle was L21 (35% Labrasol, 35% Labrafac, and 30% Solutol).
Abbreviations:
IP = intraperitoneal;
QD = 1 time per day

Tumor size was measured by calipers immediately pre-treatment and at the time of sacrifice on Day 1, 2, or 3 (5 mice per group per day). At sacrifice, the plasma was collected for assay of pathologic human VEGF concentration using an ELISA that recognizes human VEGF₁₂₁ and VEGF₁₆₅ (as described in Section 9.1.1.1).

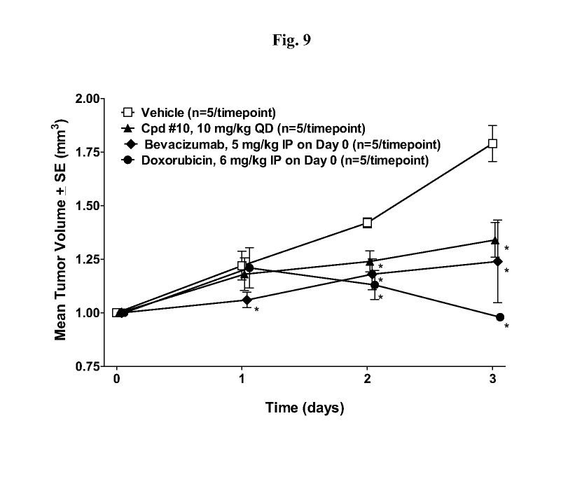

Results: FIG. 9 shows the relative change in tumor size with time. In this short term study, the untreated tumors grew rapidly. Tumors from the vehicle treated mice had grown by 22% on Day 1, 42% on Day 2, and 79% on Day 3 (p<0.05 for each day, paired Student's t-test versus Day 0). All 3 treatments significantly reduced the rate of tumor growth by more than 50% over this 3 day period.

FIG. 6 displays an evaluation of pathologic plasma human VEGF concentrations. In Panel A, absolute values are expressed. In Panel B, values are expressed as a ratio relative to tumor volume because larger tumors tend to produce more VEGF. As shown in Panel A, pathologic plasma human VEGF concentrations from vehicle treated mice rose from Day 0 to Day 3. As indicated in Panel B, increases in pathologic plasma human VEGF in control mice were seen even when adjusting for the increase in tumor size that occurred over this time period. By contrast, pathologic plasma human VEGF levels from mice treated with Compound #10, doxorubicin, or bevacizumab were numerically lower than in control animals by Day 1. Pathologic plasma human VEGF concentrations continued to decline under the influence of Compound #10, consistent with an effect indicating the inhibition of VEGF production, while absolute and relative values in other treatment groups began to increase on Days 2 and 3. Thus, by Day 3 of treatment, Compound #10 was demonstrated to be as active as bevacizumab and more effective than doxorubicin in reducing tumor derived plasma VEGF levels. In addition, these data suggest that Compound #10 regulates tumor VEGF independent of tumor size.

9.2.5 Compound #10 Shows Antitumor Activity in Several Human Tumor Xenograft Models

This example demonstrates that Compound #10 shows antitumor activity in several clinically relevant human tumor xenograft models.

Investigators at the National Cancer Institute (NCI) have shown that compounds that inhibit tumor growth in multiple nonclinical models are more likely to have clinical efficacy. See Johnson et al., Br. J. Cancer 2001, 84(10):1424 31. In each of these studies, human tumor cells were implanted and treatment was initiated some days later, only after tumors had developed a vasculature (i.e., when tumors were >100 mm³). This method of waiting to begin treatment until after tumors are established is considered a more stringent and clinically relevant assessment of efficacy compared to beginning treatment immediately after tumor implantation. See Teicher, ed. Totowa, Tumor models in cancer research. Humana Press, 2002: 593-616.

9.2.5.1 Compound #10 Shows Inhibition of Tumor Growth in an T47D Estrogen-Sensitive Breast Cancer Xenograft Model

This example demonstrates that Compound #10 shows antitumor activity in an T47D estrogen-sensitive breast cancer xenograft model.

Experimental Design. Estrogen pellets (0.72 mg/pellet) were implanted 30 days prior to cell implantation and again 60 days later. T47D estrogen-sensitive breast cancer cells (5×10⁶ cells/mouse mixed 1:1 with MATRIGEL™) were implanted subcutaneously in female athymic nude mice. After 31 days, when the tumors had become established (i.e., the mean tumor size had reached 180±33 mm³), mice were divided into 3 treatment groups, and treatment was administered as shown in. Tamoxifen was included as a positive control.

TABLE 12
Study Design for Assessment of Tumor Growth Inhibition in
Nude Mice Bearing Estrogen Sensitive T47D Xenografts.

| | | | | | | | Dose |
|---|---|---|---|---|---|---|---|
| Test | Number of | | | | | Dose | Concen- |
| Com- | Animals | | Dose | Administration^{a} | | Volume | tration |
| pound | M | F | (mg/kg) | Route | Schedule | (mL/kg) | (mg/mL) |
| Vehicle^{b} | 0 | 10 | 0 | Oral | QD | 4 | 0 |
| Com- | 0 | 10 | 10 | Oral | QD | 4 | 2.5 |
| pound #10 | | | | | | | |
| Tamoxifen | 0 | 10 | 10 | Oral | QD | 4 | 2.5 |

^{a}Treatments were administered by oral gavage QD.
^{b}Vehicle was L21 (35% Labrasol, 35% Labrafac, and 30% Solutol).
Abbreviations: QD = 1 time per day

Tumor size was measured by calipers at periodic intervals. After 74 days of treatment, the mice were sacrificed. The tumors were not analyzed for intratumoral VEGF levels because of their small size at sacrifice.

Results. Results by treatment regimen are shown in Table 13. In this breast cancer xenograft model, Compound #10 resulted in a transient reduction and persistent delay in tumor growth relative to controls. Compound #10 appeared as active as tamoxifen in suppressing growth of this estrogen-sensitive cell line. In observing the animals, there was no evidence of toxicity associated with Compound #10 treatment.

TABLE 13
Efficacy Information for Assessment of Tumor Growth Inhibition
in Nude Mice Bearing Estrogen Sensitive T47D Xenografts.

| | Number of | | | | Dose per | Mean % Inhibition of | Mean % Inhibition of |
|---|---|---|---|---|---|---|---|
| Test | Animals | | Dose | | Week | Intratumoral VEGF vs | Tumor Size vs |
| Compound | M | F | (mg/kg) | Schedule | (mg/kg) | Vehicle at Sacrifice | Vehicle at Day 74^{a} |
| Vehicle^{b} | 0 | 10 | 0 | QD | 0 | ND | NA |
| Compound #10 | 0 | 10 | 10 | QD | 70 | ND | 40 |
| Tamoxifen | 0 | 10 | 10 | QD | 70 | ND | 50 |

^{a}Day 74 was the day on which mice were sacrificed.
^{b}Vehicle was L21 (35% Labrasol, 35% Labrafac, and 30% Solutol).
Abbreviations: NA = Not applicable; ND = not determined; QD = 1 time per day; VEGF = vascular endothelial growth factor

9.2.5.2 Compound #10 Shows Inhibition of Tumor Growth in an MDA-MB 468 Estrogen Insensitive Breast Cancer Xenograft Model

This example demonstrates that Compound #10 shows antitumor activity in an MDA-MB-468 estrogen-insensitive breast cancer xenograft model.

MDA-MB-468 estrogen-insensitive breast cancer cells (5×10⁶ cells/mouse mixed 1:1 with MATRIGEL™) were implanted subcutaneously in female athymic nude mice. After 6 days, tumors had become established (i.e., the mean tumor size had reached 185±26 mm³), mice were divided into 2 treatment groups, and treatment was administered as shown in Table 14.

TABLE 14
Study Design for Assessment of Tumor Growth Inhibition in Nude
Mice Bearing Estrogen-Insensitive MDA-MB-468 Xenografts.

| | | | | | | | Dose |
|---|---|---|---|---|---|---|---|
| Test | Number of | | | | | Dose | Concen- |
| Com- | Animals | | Dose | Administration^{a} | | Volume | tration |
| pound | M | F | (mg/kg) | Route | Schedule | (mL/kg) | (mg/mL) |
| Vehicle^{b} | 0 | 10 | 0 | Oral | QD | 4 | 0 |
| Com- | 0 | 10 | 10 | Oral | QD | 4 | 2.5 |
| pound #10 | | | | | | | |

^{a}Treatments were administered QD continuously by oral gavage for at least 30 days.
^{b}Vehicle was L21 (35% Labrasol, 35% Labrafac, and 30% Solutol).
Abbreviations: QD = 1 time per day

Tumor size was measured by calipers at periodic intervals. When the individual tumor size in a mouse exceeded 1500 mm³, that mouse was sacrificed and both tumor and plasma were assayed for pathologic VEGF concentration as described in Section 9.1.1.1.

Results. Results by treatment regimen are shown in Table 15. Compound #10 at 10 mg/kg significantly reduced intratumoral and plasma pathologic VEGF concentrations on the day on which the animals were sacrificed (range, Day 33 to 53) relative to controls (range, Day 9 to 15). In addition, Compound #10 reduced tumor size and prolonged the time to tumor progression (i.e., the time to reach ≧1000 mm³). In observing the animals, there was no evidence of toxicity associated with Compound #10 treatment.

TABLE 15
Efficacy Information for Assessment of Tumor Growth Inhibition in Nude
Mice Bearing Estrogen Insensitive MDA-MB-468 Breast Cancer Xenografts.

| | Number of | | Dose | Dose per | Mean % Inhibition of | Mean % Inhibition of | Mean % Inhibition of | Median Time to |
|---|---|---|---|---|---|---|---|---|
| Test | Animals | | (mg/kg)/ | Week | Intratumoral VEGF vs | Plasma pathologic VEGF | Tumor Size vs | Tumor Size ≧1000 |
| Compound | M | F | Schedule^{a} | (mg/kg) | Vehicle at Sacrifice | vs Vehicle at Sacrifice | Vehicle at Day 12^{b} | mm³ (days) |
| Vehicle^{c} | 0 | 10 | 0/QD | 0 | — | — | — | 12 |
| Compound | 0 | 10 | 10/QD | 70 | 61* | 75* | 65* | 25 |
| #10 | | | | | | | | |

*p < 0.05 (Student's t test relative to vehicle)
^{a}Treatments were administered QD continuously by oral gavage for at least 30 days.
^{b}Vehicle treated animal tumors reached ≧1500 mm³ between Day 9 and 15 and all vehicle treated animals were sacrificed by Day 15.
^{c}Vehicle was L21 (35% Labrasol, 35% Labrafac, and 30% Solutol).
Abbreviations: M = Male; F = Female; QD = 1 time per day; VEGF = vascular endothelial growth factor; M = Male; F = Female

9.2.5.3 Compound #10 Shows Reduction in Tumor Perfusion as Assessed by Dynamic Contrast-Enhanced Magnetic Resonance Imaging

This example shows that Compound #10 reduces tumor perfusion as assessed by dynamic contrast-enhanced magnetic resonance imaging.

Experimental Design. Dynamic contrast-enhanced magnetic resonance imaging can be used preclinically and clinically to evaluate the anatomy of soft tissues, including the identification and accurate measurement of tumor volumes. In addition, evaluation of the intratumoral pharmacokinetics of contrast agents containing gadolinium can be used to measure vascular permeability characteristics. Coupling gadopentetate dimeglumine gadolinium to a small molecule like bovine serum albumin can reveal information about the necrotic (non-perfused) and non-necrotic (perfused) tumor volumes, and the percentage of vascular blood volume relative to the perfused tumor volume (known as the fractional blood volume [fBV]). Use of a macromolecular tracer, gadopentetate dimeglumine, can reveal information regarding the volume transfer coefficient (K^{trans}), a variable that represents a combination of vascular permeability, vascular surface area, and blood flow.

MDA MB 468 breast cancer cells (5×10⁶ cells/mouse mixed 1:1 with MATRIGEL™) were implanted subcutaneously in female athymic nude mice. After 13 days, when the tumors had become established (i.e., the mean tumor size reached ˜400 mm³), mice were divided into 2 treatment groups, and treatment was administered as shown in Table 16.

TABLE 16
Study Design for Assessment of Tumor Perfusion
in Nude Mice Bearing MDA MB 468 Xenografts

| | | | | | | | Dose |
|---|---|---|---|---|---|---|---|
| Test | Number of | | | | | Dose | Concen- |
| Com- | Animals | | Dose | Administration^{a} | | Volume | tration |
| pound | M | F | (mg/kg) | Route | Schedule | (mL/kg) | (mg/mL) |
| Vehicle^{a} | 0 | 8 | 0 | Oral | QD | 4 | 0 |
| Com- | 0 | 8 | 10 | Oral | QD | 4 | 2.0 |
| pound #10 | | | | | | | |

^{a}Vehicle was L21 (35% Labrasol, 35% Labrafac, and 30% Solutol). Abbreviations: QD = 1 time per day

Before each DCE-MRI scan, mice were injected intravenously with gadolinium-containing contrast dyes (bovine serum albumin-gadopentetate dimeglumine conjugate at ˜0.03 mmol/kg followed by gadopentetate dimeglumine at ˜0.2 mmol/kg). Baseline DCE-MRI measurements were taken on Day −1, test Compounds were administered on Day 0 through Day 5, and additional DCE-MRI measurements were taken on Days 1, 3, and 5. Image analyses were conducted with customized software. Total tumor volumes were measured by semi-automatically segmenting a region of interest around an anatomical image of the tumor. Tumor volumes of necrotic and non-necrotic tissues were measured by applying the same semi-automated segmentation process to a contrast dyed image. fBV and K^{trans} were computed using a standard Kety PK model.

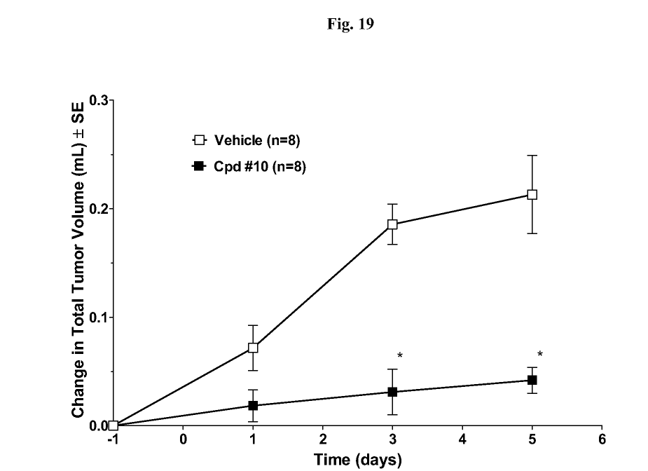

Results. As shown in FIG. 19, vehicle-treated animals had an increase in mean tumor volume from Day −1 to Day 5. By contrast, Compound #10 treated animals had little mean change. Differences in total tumor volumes in vehicle treated versus treated mice were apparent by Day 1 and were statistically significant by Day 3, confirming that Compound #10 begins to impede tumor growth rapidly after treatment initiation.

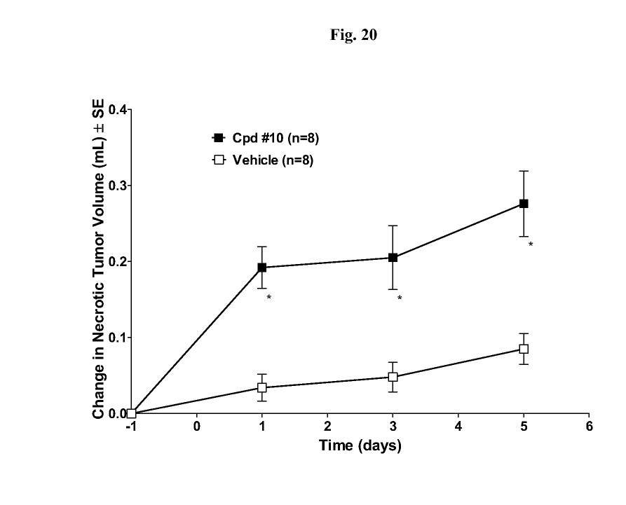

As shown in FIG. 20, vehicle-treated animals had a small mean change in necrotic (non perfused) tumor volume from Day −1 to Day 5. Consistent with an antivascular effect, Compound #10 rapidly increased the mean necrotic tumor volume, resulting in differences in necrotic tumor volumes between vehicle treated and treated groups that were statistically significant by Day 1.

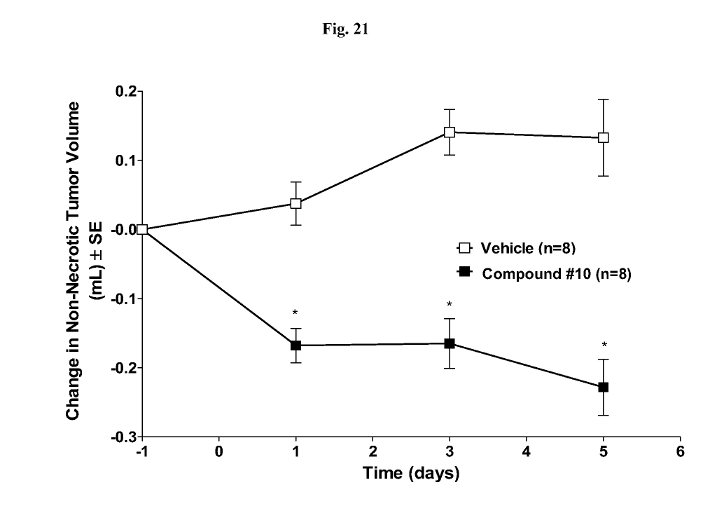

Conversely, as shown in FIG. 21, most of the mean tumor volume increase depicted in FIG. 19 in vehicle-treated animals was due to growth of non-necrotic tumor tissue. By contrast, mean non-necrotic tumor volume in Compound #10-treated animals decreased from Day −1 to Day 5. Differences in non necrotic tumor volumes between vehicle-treated and treated groups were statistically significant by Day 1.

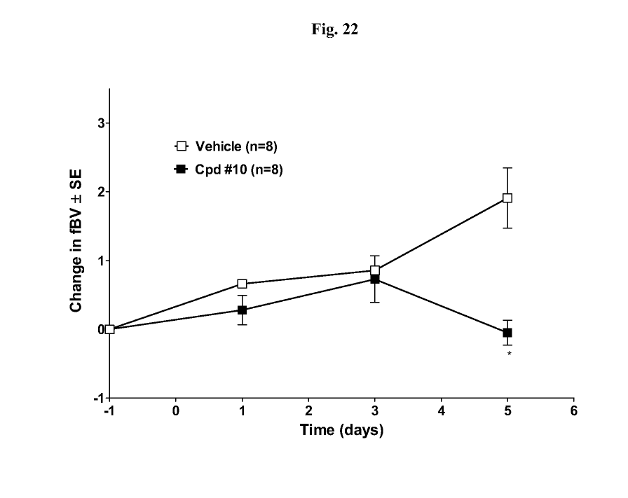

Tissue regions identified as necrotic have no measurable vascular permeability, limiting analysis of fBV to non-necrotic tumor regions (primarily in the tumor rim). As shown in FIG. 22, mean tumor fBV in vehicle-treated animals increased steadily from Day 1 to Day 5. Initially, mean tumor fBV also increased in Compound #10 treated mice but then declined after Day 3, resulting in a statistically significant difference relative to the vehicle-treated values on Day 5. These data indicate that Compound #10 inhibits tumor angiogenesis, increases tumor necrosis, decreases viable tumor, and decreases tumor microvessel density.

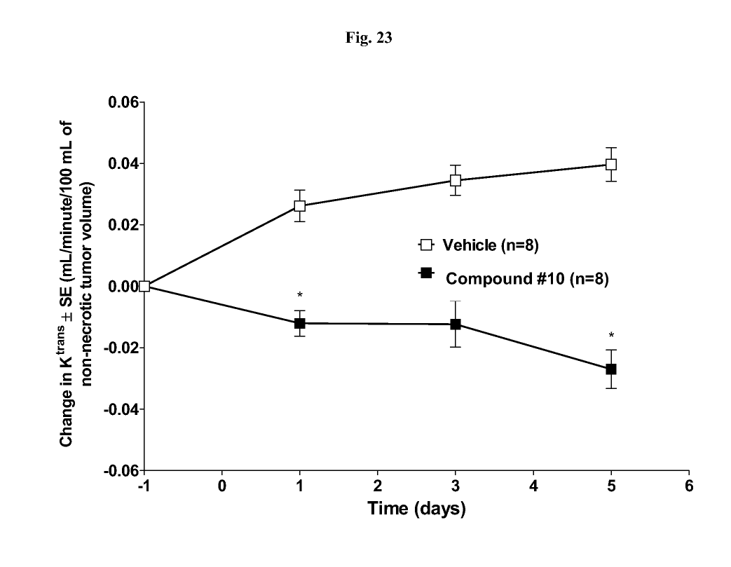

As for fBV, analysis of K^{trans} was necessarily confined to non-necrotic tissue. As shown in FIG. 23, mean K^{trans} increased in vehicle treated mice between Day −1 and Day 5, while the mean K^{trans} decreased in Compound #10 treated mice over this same period. The relative changes in K^{trans} in vehicle-treated compared to treated animals were statistically significant by Day 1. The data are consistent with Compound #10 inhibition of vascular permeability in the non-necrotic tumor rim.

9.2.5.4 Compound #10 Shows Inhibition of Tumor Growth in an SY5Y Neuroblastoma Xenograft Model

This example demonstrates that Compound #10 shows antitumor activity in an SY5Y neuroblastoma xenograft model.

Experimental Design. SY5Y cells are derived from a human neuroblastoma, a childhood tumor arising in neural crest cells. SY5Y cells (1×10⁷ cells/mouse) were implanted subcutaneously in male athymic nude mice. After 7-days, tumors had become established (i.e., the mean tumor size had reached 387±10 mm³), mice were divided into 2 groups, and treatment was administered as shown in Table 17.

TABLE 17
Study Design for Assessment of Tumor Growth Inhibition
in Nude Mice Bearing SY5Y Xenografts

| | | | | | | | Dose |
|---|---|---|---|---|---|---|---|
| Test | Number of | | | | | Dose | Concen- |
| Com- | Animals | | Dose | Administration^{a} | | Volume | tration |
| pound | M | F | (mg/kg) | Route | Schedule | (mL/kg) | (mg/mL) |
| Vehicle^{b} | 6 | 0 | 0 | Oral | QD | 4 | 0 |
| Com- | 6 | 0 | 10 | Oral | QD | 4 | 2.5 |
| pound #10 | | | | | | | |

^{a}Treatments were administered by oral gavage 5 days per week (Monday through Friday) for up to 50 days.
^{b}Vehicle was L22 (35% Labrafil, 35% Labrafac, and 30% Solutol). Abbreviation: QD = 1 time per day

Tumor size was measured by calipers at periodic intervals. When the average tumor size in a group exceeded 2000 mm³, the mice in the group were sacrificed and excised tumors were assayed for intratumoral VEGF concentration as described in Section 9.1.1.1. Animals in which tumors did not reach 2000 mm³ were sacrificed at Day 50.

Results. Results by treatment regimen are shown in Table 18. Compound #10 treatment was associated with a significant reduction in mean intratumoral VEGF concentration and essentially eliminated any increase in mean tumor size through 15-days of dosing, substantially prolonging the mean time until tumor progression (tumor size ≧1000 mm³). In contrast, tumors in many control animals exceeded 2000 mm³ by Day 17 and these animals had to be sacrificed. In view of the dramatic effect of Compound #10 treatment, Compound #10 treatment was stopped on Day 15 to determine whether these effects might be sustained after treatment withdrawal. Tumors from mice treated with Compound #10 continued to be smaller than tumors from vehicle treated mice, even after 28-days without treatment (data not shown). At Day 43, treatment with vehicle or Compound #10 was reinitiated for a further 6 days. There were not enough vehicle mice remaining in the study to assess if Compound #10 would be more effective than vehicle in terms of tumor growth inhibition after treatment reinitiation. However, as summarized in Table 18, even after the cessation of treatment for 28-days and then continued Compound #10 treatment for 6 days, intratumoral levels of VEGF were almost completely suppressed in the treated tumors. In observing the animals, there was no evidence of toxicity associated with Compound #10 treatment.

TABLE 18
Efficacy Information for Assessment of Tumor Growth Inhibition in Nude Mice Bearing SY5Y Xenografts.

| | Number of | | | | Dose per | Mean % Inhibition of | Mean % Inhibition of | Median Time to |
|---|---|---|---|---|---|---|---|---|
| Test | Animals | | Dose | | Week | Intratumoral VEGF vs | Tumor Size vs | Tumor Size ≧1000 |
| Compound | M | F | (mg/kg) | Schedule^{a} | (mg/kg) | Vehicle at Sacrifice | Vehicle at Day 17^{b} | mm³ (days) |
| Vehicle^{c} | 6 | 0 | 0 | QD | 0 | 0 | 0 | 12 |
| Compound | 6 | 0 | 50 | QD | 250 | 96* | 73* | 35 |
| #10 | | | | | | | | |

*p < 0.05 (Student's t-test relative to vehicle)
^{a}Treatments were administered by oral gavage 5 days per week (Monday through Friday) for up to 50 days.
^{b}Day 17 was day on which vehicle treated animal tumors had reached ≧2000 mm³ and the mice were sacrificed.
^{c}Vehicle was L22 (35% Labrafil, 35% Labrafac, and 30% Solutol).
Abbreviations: QD = 1 time per day; VEGF = vascular endothelial growth factor; M = Male; F = Female

9.2.5.5 Compound #10 Shows Inhibition of Tumor Growth in an LNCaP Prostate Cancer Xenograft Model

This example demonstrates that Compound #10 shows antitumor activity in an LNCaP prostate cancer xenograft model.

Experimental Design. The LNCaP cell line is derived from a lymph node metastasis. LNCaP cells (1×10⁶ cells/mouse mixed 1:1 with MATRIGEL™) were implanted subcutaneously in male athymic nude mice. After 43 days, tumors had become established (i.e., the mean tumor size had reached 260±35 mm³), mice were divided into 2 treatment groups, and treatment was administered as shown in Table 19.

TABLE 19
Study Design for Assessment of Tumor Growth Inhibition in
Nude Mice Bearing Androgen-Sensitive LNCaP Xenografts.

| | | | | | | | Dose |
|---|---|---|---|---|---|---|---|
| Test | Number of | | | | | Dose | Concen- |
| Com- | Animals | | Dose | Administration^{a} | | Volume | tration |
| pound | M | F | (mg/kg) | Route | Schedule | (mL/kg) | (mg/mL) |
| Vehicle^{b} | 10 | 0 | 0 | Oral | M-W-F | 4 | 0 |
| Com- | 10 | 0 | 10 | Oral | M-W-F | 4 | 2.5 |
| pound #10 | | | | | | | |

^{a}Treatments were administered M-W-F by oral gavage for at least 35 days.
^{b}Vehicle was L21 (35% Labrasol, 35% Labrafac, and 30% Solutol).
Abbreviations: M-W-F = Monday-Wednesday-Friday

Tumor size was measured by calipers at periodic intervals during the study. When the mean tumor size in a mouse exceeded 1500 mm³, mice in that group were sacrificed and both tumor and plasma were assayed for pathologic VEGF concentration as described in Section 9.1.1.1.

Results. Results by treatment regimen are shown in Table 20. Relative to controls, Compound #10 at 10 mg/kg M-W-F reduced intratumoral VEGF concentrations adjusted for tumor size on the day on which the animals were sacrificed. In addition, Compound #10 prolonged the time to tumor progression (i.e., the time to reach ≧1000 mm³). In observing the animals, there was no evidence of toxicity associated with Compound #10 treatment.

TABLE 20
Efficacy Information for Assessment of Tumor Growth Inhibition in Nude
Mice Bearing Androgen-Insensitive LNCaP Prostate Cancer Xenografts.

| | Number of | | | | Dose per | Mean % Inhibition of | Mean % Inhibition of | Median Time to |
|---|---|---|---|---|---|---|---|---|
| Test | Animals | | Dose | | Week | Intratumoral VEGF vs | Tumor Size vs | Tumor Size ≧1000 |
| Compound | M | F | (mg/kg) | Schedule^{a} | (mg/kg) | Vehicle at Sacrifice | Vehicle at Day 35^{b} | mm³ (days) |
| Vehicle^{c} | 10 | 0 | 0 | M-W-F | 0 | — | — | 27 |
| Compound | 10 | 0 | 10 | M-W-F | 30 | 51^{d} | 36 | 38 |
| #10 | | | | | | | | |

^{a}Treatments were administered M-W-F by oral gavage for at least 35 days.
^{b}Vehicle treated animal tumors reached ≧1500 mm3 by ~Day 30 and all vehicle-treated animals were sacrificed by Day 35.
^{c}Vehicle was L21 (35% Labrasol, 35% Labrafac, and 30% Solutol).
^{d}Adjusted for tumor size
Abbreviations: M-W-F = Monday-Wednesday-Friday; VEGF = vascular endothelial growth factor

9.2.5.6 Compound #10 Shows Inhibition of Tumor Growth in Orthotopic SY5Y Neuroblastoma and SKNEP Ewing Sarcoma Tumor Models

This example demonstrates that Compound #10 shows antitumor activity in orthotopic SY5Y neuroblastoma and SKNEP Ewing sarcoma tumor models.

Experimental Design. In orthotopic tumor models, human tumor cells are implanted into the mouse in an organ that corresponds to the location from which the tumors arise. Such models may provide a better predictor of clinical efficacy than injection of tumors into the flanks of nude mice. See Hoffman, Invest. New Drugs 1999, 17(4):343-59. SY5Y neuroblastoma or SKNEP Ewing sarcoma tumor cells (1×10⁶ cells/mouse) were implanted into the kidney capsule of female athymic nude mice according to published methods. See Huang et al., Proc. Natl. Acad. Sci. USA 2003, 100(13):7785-90. One week after implantation of each type of tumor, mice were divided into 2 groups and were administered a test Compound as shown in Table 21.

TABLE 21
Study Design for Assessment of Tumor Growth Inhibition in
Nude Mice Bearing SKNEP or SY5Y Orthotopic Xenografts.

| | | Number of | | | | | Dose | Dose |
|---|---|---|---|---|---|---|---|---|
| Tumor | Test | Animals | | Dose | Administration^{a} | | Volume | Concentration |
| Type | Compound | M | F | (mg/kg) | Route | Schedule | (mL/kg) | (mg/mL) |
| SY5Y | Vehicle^{b} | 0 | 15 | 0 | Oral | QD | 4 | 0 |
| | Compound | 0 | 15 | 30 | Oral | QD | 4 | 7.5 |
| | #10 | | | | | | | |
| SKNEP | Vehicle^{b} | 0 | 15 | 0 | Oral | QD | 4 | 0 |
| | Compound | 0 | 15 | 30 | Oral | QD | 4 | 7.5 |
| | #10 | | | | | | | |

^{a}Treatments were administered by oral gavage 5 days per week (Monday through Friday) for up to 5 weeks.
^{b}Vehicle was L3 (70% Labrasol, 18.3% Labrafac, and 11.7% Labrafil).
Abbreviation: QD = 1 time per day

After 5 weeks of treatment, the mice were sacrificed, and the weights of the tumors were assessed.

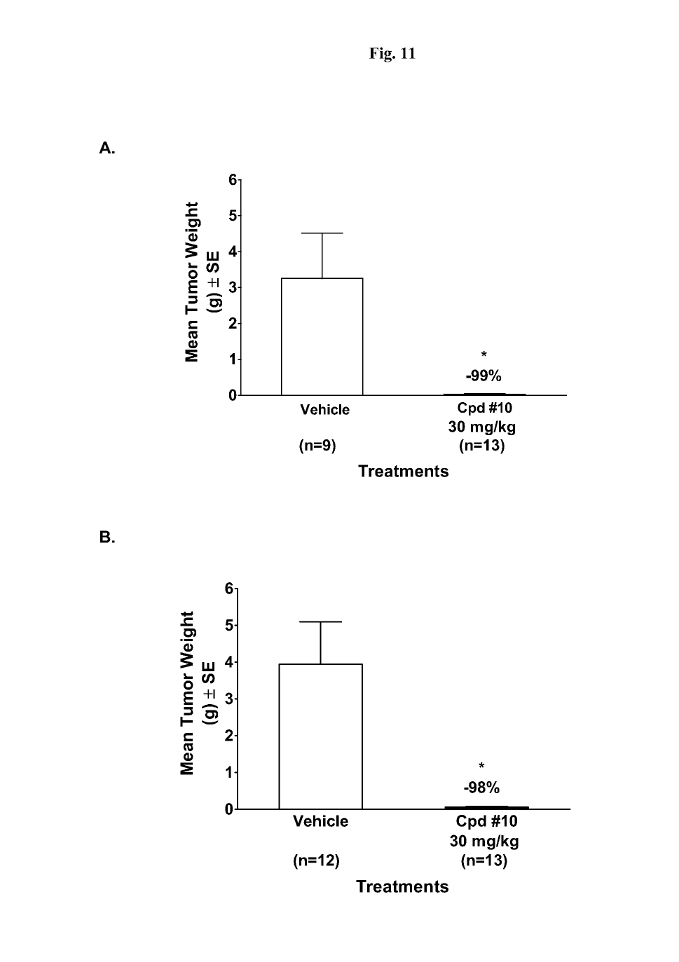

Results. As shown in FIG. 11, tumors from vehicle treated mice weighed about 3 to 4 grams at 5 weeks. By contrast, treatment with Compound #10, when evaluated at the same time point, completely prevented growth of both the SKNEP and the SY5Y tumors. In observing the animals, there was no evidence of toxicity associated with Compound #10 treatment.

9.2.6 Compound #10 Penetrates Disease Relevant Tissues

This example demonstrates that Compound #10 penetrates disease relevant tissues.

Experimental Design. The distribution of ¹⁴C-Compound #10 were evaluated following a single oral gavage administration of 50 mg/kg (˜10 μCi/animal) of ¹⁴C-labeled Compound #10 to rats in a GLP study. For the quantitative whole-body autoradiography (QWBA) analysis, 1 animal/sex/timepoint was sacrificed at 6, 12, 24, 48, and 72 hours postdose as shown in Table 22.

TABLE 22
Study Design for ¹⁴C-Compound #10 Single Dose Tissue Distribution Assessment in Rats

| Number of | | Compound | Dose | Dose | Number of | Dosing | Timepoints |
|---|---|---|---|---|---|---|---|
| Animals | | #10 Dose^{a} | Volume | Concentration | Animals per | Day | Relative to |
| M | F | (mg/kg) | (mL/kg) | (mg/mL) | Timepoint | Sampled | Dose (hours) |
| 5 | 5 | 50 | 1.25 | 40 | 1^{b} | Day 1 | 6, 12, 24, 48, 72 |

^{a14}C-Compound #10 was administered as a single-dose by oral gavage in L23 vehicle (35% Gelucire, 35% Labrafac, and 30% Solutol).
^{b}For 1 animal per sex at each timepoint, a blood sample was collected at the time of sacrifice for assessments of concentrations ¹⁴C-Compound #10 in blood, plasma, and tissues, and for calculation of tissue:plasma concentration ratios at the specified times postdose.
Abbreviations: F = female; M = male

For the QWBA, the carcasses were prepared by immediately freezing them, embedding them in chilled carboxymethylcellulose, and freezing them into blocks. Appropriate cryomicrotome sections of the blocks at 40 μm thickness were collected on adhesive tape. Mounted sections were tightly wrapped and exposed on phosphorimaging screens along with plastic embedded autoradiographic standards. Exposed screens were scanned and the autoradiographic standard image data were sampled to create a calibrated standard curve. Specified tissues, organs, and fluids were analyzed. Tissue concentrations were interpolated from each standard curve as nanocuries per gram and then converted to μg equivalents/gram on the basis of the Compound #10 specific activity.

Results. All animals appeared healthy and exhibited no overt signs of toxicity throughout the study. In this study, absorbed radioactivity was rapidly distributed into the whole body with the Tₘₐₓ in blood and plasma occurring at 4 hours postdose in both sexes. Excluding the gastrointestinal tract, Cₘₐₓ values in most tissues occurred at 6 to 12 hours postdose, with the highest values occurring in lipomatous tissues such as adrenal gland, brown fat, and liver. By 72 hours postdose, discernable residual radioactivity remained concentrated in fatty tissues in both sexes.

As shown in Table 23, the tissue:plasma concentration ratios were greater than 1 in most tissues. At 72 hours postdose, the highest tissue:plasma concentration ratios were in fat with values ranging from 37.1 to 63.9 in both sexes. All other tissues had ratios less than 10 with the exception of female bone marrow, Harderian gland, ovary, and skin, which had values of 18.8, 12.0, 28.1, and 11.4, respectively. There were no remarkable gender related differences in absorption, distribution, and elimination of radioactivity.

TABLE 23
Tissue:Plasma Concentration Ratios Determined by Whole-Body Autoradiography at Specified
Times after a Single Oral Administration of ¹⁴C-Compound #10 to Rats (50 mg/kg)

| | 6 Hours | | 12 Hours | | 24 Hours | | 48 Hours | | 72 Hours | |
|---|---|---|---|---|---|---|---|---|---|---|
| Tissue | M | F | M | F | M | F | M | F | M | F |
| Adrenal gland | 18.5 | 16.2 | 10.8 | 16.7 | 8.96 | 8.93 | 5.89 | 6.59 | 6.02 | 7.16 |
| Blood | 0.569 | 0.577 | 0.601 | 1.00 | NA | 0.613 | NA | NA | NA | 1.80 |
| Bone | NA | 0.362 | NA | 0.497 | NA | NA | NA | NA | NA | NA |
| Bone marrow | 2.71 | 4.85 | 4.01 | 13.0 | 3.48 | 4.63 | 2.91 | 7.05 | NA | 18.8 |
| Cecum | 4.18 | 7.44 | 4.80 | 5.70 | 2.56 | 2.10 | 2.39 | 3.49 | NA | 3.66 |
| Cecum contents | 98.7 | 40.5 | 21.9 | 40.3 | 4.91 | 7.20 | 4.98 | 2.74 | 5.01 | 3.04 |
| Cerebellum | 1.55 | 1.23 | 1.85 | 2.85 | 1.74 | 1.59 | 1.21 | 1.17 | NA | 2.04 |
| Cerebrum | 1.52 | 1.22 | 1.75 | 2.79 | 1.89 | 1.57 | 1.35 | 1.68 | NA | 1.56 |
| Diaphragm | 5.48 | 4.35 | 4.98 | 6.58 | 2.89 | 3.06 | 2.04 | 3.09 | 1.75 | 3.50 |
| Epididymis | 0.862 | NA | 1.22 | NA | 2.13 | NA | 3.09 | NA | 3.09 | NA |
| Esophageal | NA | 0.231 | NA | NA | NA | NA | NA | NA | NA | 2.21 |
| contents | | | | | | | | | | |
| Esophagus | 1.83 | 1.25 | 1.89 | 3.64 | 1.53 | 1.59 | NA | 2.76 | NA | 1.93 |
| Exorbital | 3.46 | 3.45 | 5.56 | 8.15 | 4.72 | 3.85 | 3.44 | 3.90 | 3.91 | 3.51 |
| lacrimal gland | | | | | | | | | | |
| Eye | 0.279 | 0.275 | 0.291 | 0.606 | NA | NA | 0.847 | NA | NA | 1.72 |
| Fat (abdominal) | 13.3 | 4.05 | 20.7 | 9.61 | 27.8 | 38.2 | 47.7 | 58.4 | 62.1 | 60.8 |
| Fat (brown) | 15.5 | 14.2 | 25.4 | 46.1 | 34.4 | 34.0 | 37.0 | 58.4 | 37.1 | 63.9 |
| Fat | 4.66 | 5.11 | 15.4 | 12.9 | 22.9 | 31.7 | 35.6 | 50.0 | 52 2 | 56.6 |
| (subcutaneous) | | | | | | | | | | |
| Gastric mucosa | 5.47 | 5.92 | 6.58 | 6.82 | 3.35 | 3.66 | 2.86 | 4.18 | 2.97 | 4.50 |
| Harderian gland | 3.06 | 2.53 | 5.02 | 7.61 | 8.92 | 7.80 | 10.5 | 14.7 | 9.54 | 12.0 |
| Intra-orbital | 3.12 | 3.33 | 5.47 | 6.21 | 4.46 | 4.11 | 3.67 | 6.13 | NA | 8.76 |
| lacrimal gland | | | | | | | | | | |
| Kidney | 5.98 | 4.50 | 4.44 | 5.82 | 3.20 | 2.72 | 2.36 | 3.23 | 2.04 | 4.09 |
| Large intestinal | 26.2 | 138 | 61.7 | 256 | 21.9 | 20.8 | 12.1 | 5.44 | 5.80 | 7.51 |
| contents | | | | | | | | | | |
| Large intestine | 2.65 | 2.43 | 3.06 | 5.94 | 1.81 | 2.10 | 1.58 | 1.69 | NA | 3.02 |
| Liver | 7.77 | 8.49 | 5.65 | 8.82 | 4.83 | 4.79 | 4.23 | 6.01 | 4.52 | 5.74 |
| Lung | 2.52 | 2.00 | 1.80 | 2.69 | 1.54 | 1.43 | 1.38 | 1.64 | NA | 2.46 |
| Medulla | 1.60 | 1.42 | 1.98 | 3.82 | 1.83 | 1.69 | 1.20 | 2.01 | NA | 1.88 |
| Muscle | 2.65 | 2.11 | 2.81 | 3.55 | 1.70 | 1.82 | 1.47 | 1.73 | NA | 2.54 |
| Myocardium | 5.31 | 5.89 | 3.90 | 7.03 | 2.82 | 2.88 | 2.43 | 3.95 | 1.97 | 4.15 |
| Nasal turbinates | 1.19 | 1.14 | 1.40 | 2.12 | 1.55 | 1.25 | 1.52 | 2.06 | NA | 2.58 |
| Olfactory lobe | 1.42 | 1.38 | 1.35 | 2.45 | 1.23 | 1.13 | 0.967 | NA | NA | 3.33 |
| Ovary | NA | 7.48 | NA | 17.6 | NA | 12.1 | NA | 11.3 | NA | 28.1 |
| Pancreas | 6.95 | 6.25 | 6.28 | 9.58 | 4.54 | 4.79 | 3.25 | 5.08 | 3.21 | 4.96 |
| Pituitary gland | 4.06 | 4.27 | 3.22 | 5.48 | 2.72 | 2.33 | 0.890 | 3.68 | NA | 1.58 |
| Preputial gland | 4.15 | 3.45 | 6.94 | 12.3 | 11.3 | 7.93 | 20.2 | NA | NA | NA |
| Prostate | 2.62 | NA | 2.61 | NA | 2.35 | NA | 1.09 | NA | 1.78 | NA |
| Renal cortex | 6.83 | 5.65 | 4.53 | 6.48 | 3.27 | 2.96 | 2.64 | 3.49 | 2.44 | 4.40 |
| Renal medulla | 5.35 | 3.70 | 4.21 | 5.06 | 3.04 | 2.53 | 1.75 | 2.84 | 1.68 | 3.60 |
| Salivary gland | 5.69 | 4.75 | 4.80 | 7.18 | 3.38 | 3.53 | 2.45 | 3.57 | 1.90 | 3.74 |
| Seminal vesicle | 0.780 | NA | 0.646 | NA | 0.691 | NA | NA | NA | NA | NA |
| Skin | 1.66 | 1.46 | 3.33 | 5.21 | 3.98 | 4.19 | 4.49 | 5.73 | 8.06 | 11.4 |
| Small intestinal | 7.35 | 7.81 | 15.2 | 15.1 | 1.67 | 3.35 | 3.68 | 2.80 | 1.69 | 3.34 |
| contents | | | | | | | | | | |
| Small intestine | 8.46 | 5.01 | 3.02 | 5.09 | 2.93 | 2.45 | 1.21 | 2.62 | 1.80 | 3.36 |
| Spinal cord | 1.14 | 0.898 | 1.24 | 1.92 | 1.75 | 1.60 | 1.43 | 1.60 | 1.84 | 2.75 |
| Spleen | 2.73 | 2.84 | 2.37 | 3.91 | 1.80 | 1.89 | 1.50 | 1.88 | NA | 2.84 |
| Stomach | 4.34 | 3.62 | 3.72 | 5.12 | 2.86 | 1.76 | 1.72 | 2.93 | 2.44 | 4.19 |
| Stomach | 6.51 | 3.36 | 1.10 | 1.01 | NA | NA | NA | NA | NA | NA |
| contents | | | | | | | | | | |
| Testis | 0.642 | NA | 1.17 | NA | 1.88 | NA | 2.13 | NA | 1.90 | NA |
| Thymus | 2.11 | 1.98 | 2.50 | 3.94 | 1.98 | 1.84 | 1.58 | 1.65 | NA | 3.34 |
| Thyroid | 3.18 | 3.77 | 2.57 | 3.61 | 2.76 | 1.38 | 1.14 | 1.87 | NA | 3.05 |
| Urinary bladder | 1.63 | 1.45 | 0.786 | 1.89 | 1.56 | 1.02 | 1.23 | 1.38 | NA | 1.92 |
| Urine | 0.239 | 1.66 | 0.299 | 0.761 | NA | NA | NA | NA | NA | NA |
| Uterus | NA | 1.86 | NA | 4.97 | NA | 3.51 | NA | 3.51 | NA | 7.66 |

Abbreviations: F = female; M = male; NA = not applicable

This example demonstrates that Compound #10 penetrates disease relevant tissues.

9.3 Cell Cycle Delay

9.3.1 Cell Based Assays

9.3.1.1 Compound #10 and Compound 1205 Provoke a Late G₁/Early S-Phase Cell Cycle Delay

This example demonstrates that a Compound induces a cell cycle delay at the G₁/S-phase border.

Experimental Design. During in vitro evaluations of Compound #10 and Compound 1205 effects on VEGF expression, an examination of the effect on tumor cell cycling was performed. HT1080 cells were incubated under normoxic conditions (21% oxygen) for 18 hours with vehicle (0.5% DMSO) alone, or with a range of concentrations of Compound #10 from 0.3 nM to 100 nM, or 10 nM of Compound 1205. Compounds shown in Table 24 were incubated under normoxic conditions for 18 hours with vehicle or Compound #10 at a single dose of 100 nM. After treatment, cells were trypsinized, and stained with propidium iodide (PI) dye to measure DNA content of individual cells by flow cytometry. Output comprised histograms showing relative DNA content in 10,000 cells.

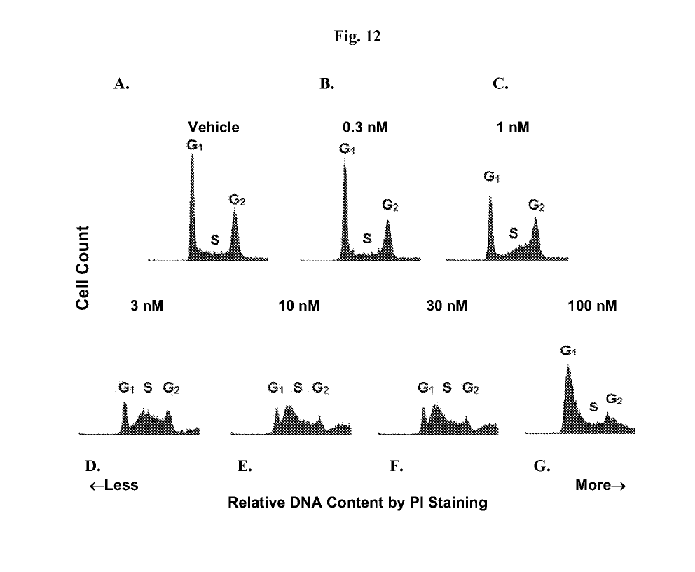

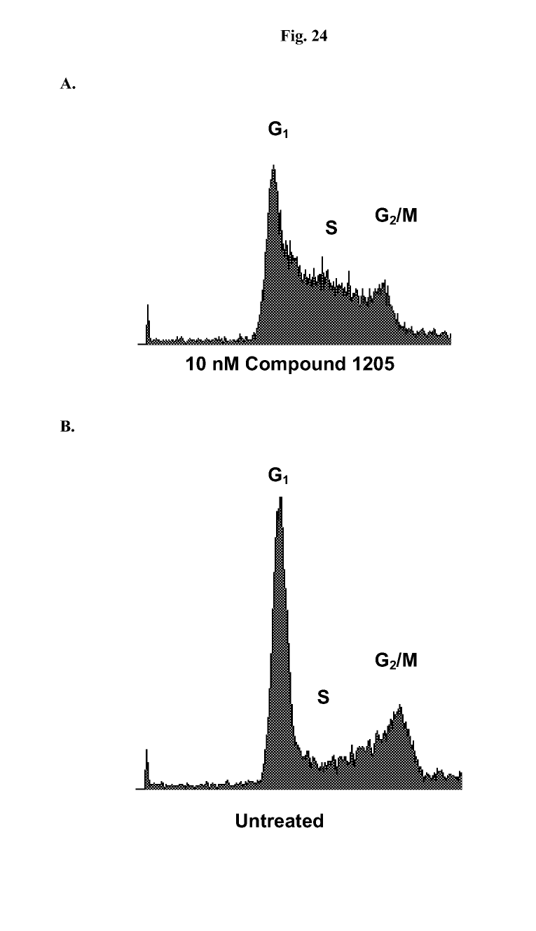

Results. As shown in FIG. 12 and FIG. 24, Compound #10 and Compound 1205 induced a redistribution of the cycling characteristics of the cell population. An apparent dose response was observed for Compound #10. Starting at a concentration of 1 nM for Compound #10, an accumulation of cells in S phase can be observed. With higher concentrations of Compound #10, there is a progressive shift, such that a substantial proportion of the cells show a cell cycle delay at the G₁/S phase border. Concentrations of Compound #10 achieving these effects are consistent with those demonstrating inhibition of VEGF production (FIG. 1).

For additional Compounds shown in Table 24, the test results are expressed as the percentage of cells in the S-phase compared to a DMSO control (17.3% cells in S-Phase). While compounds which cause greater than 20% of the cells to accumulate in S-phase at 100 nM are considered active, a larger percentage of cells may be accumulated in S-phase at lower doses depending on the Compound, as shown in FIG. 12 for example.

TABLE 24

| | % |
|---|---|
| | Cells |
| | In S- |
| Compound | Phase |
| DMSO (Control) | 17.3 |
| 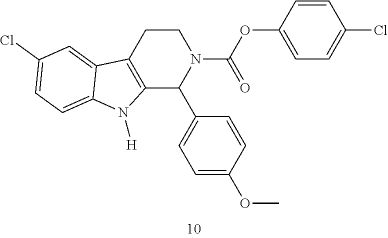 | 15.3 |
| 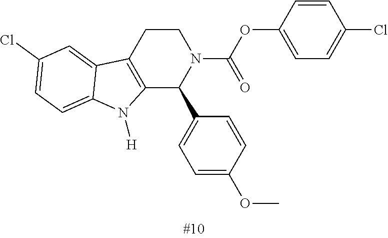 | 26.1 |
| 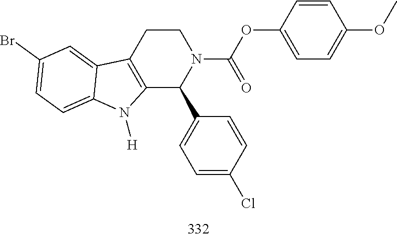 | 26.4 |
| 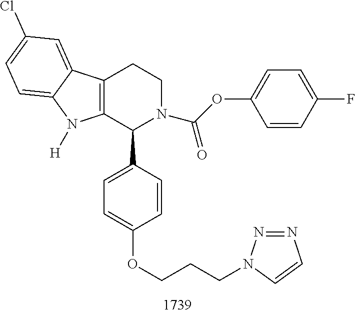 | 25.7 |
| 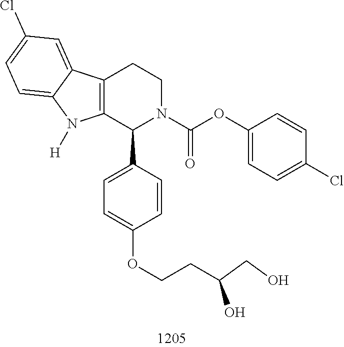 | 20.0 |
| 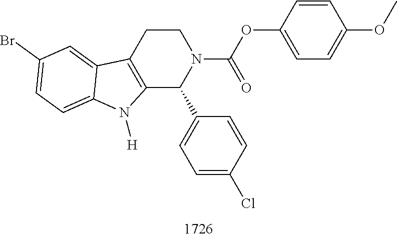 | 16.5 |
| 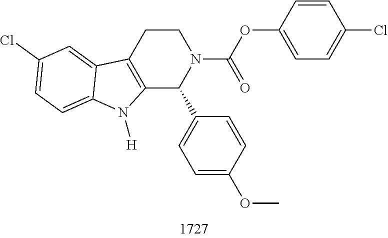 | 16.8 |
| 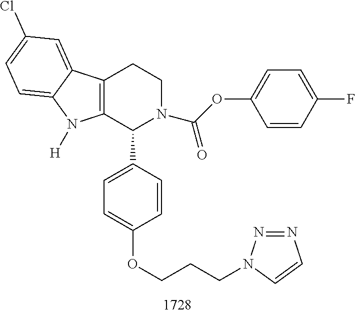 | 16.4 |
| 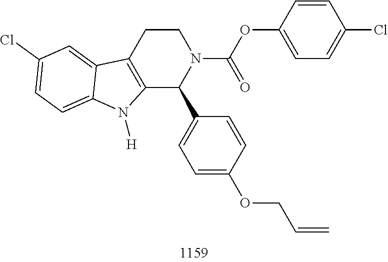 | 17.2 |
| 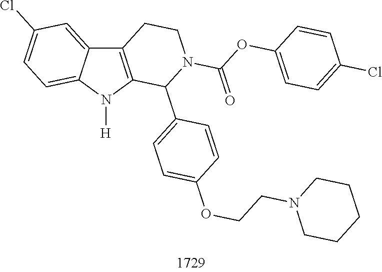 | 16.8 |
| 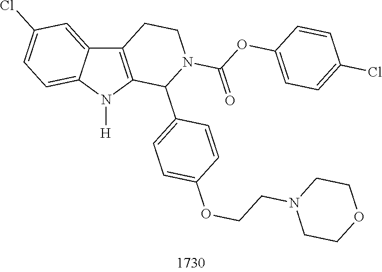 | 16.4 |
| 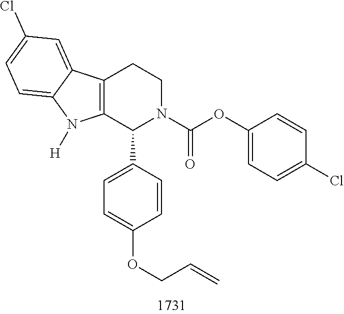 | 17.9 |
| 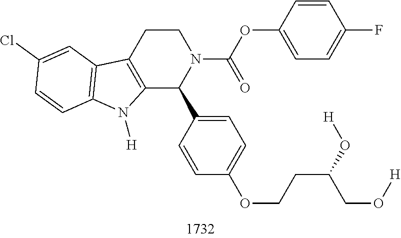 | 20.6 |
| 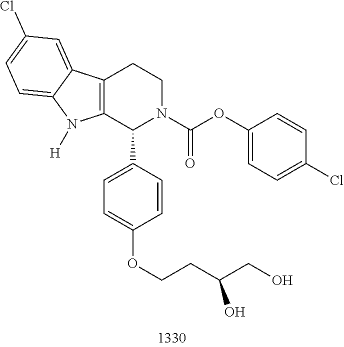 | 17 |

9.3.1.2 The Effect of Compound #10 on the Cell Cycle is Reversible

This example demonstrates that the effect of Compound #10 on cell cycle delay is reversible.

Experimental Design. HT1080 cells were incubated under normoxic conditions (21% oxygen) for 14 hours with Compound #10 (100 nM) or with vehicle (0.5% DMSO) alone. Compound #10 was then washed out of the cultures and cells were harvested and analyzed by PI staining and flow cytometry (as described in Section 9.3.1.1) at 0, 2, 5, 8, and 26 hours after discontinuation of treatment.

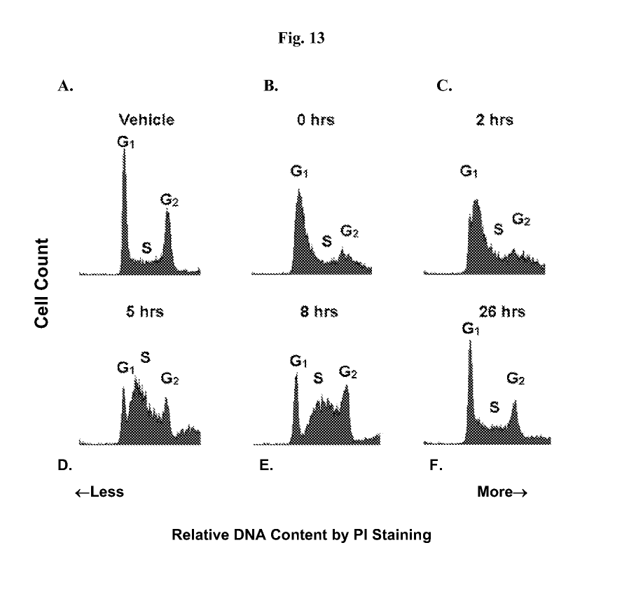

Results. As shown in FIG. 13, treatment with Compound #10 caused the expected increase in the proportion of cells in late G₁/S phase of the cell cycle (Time 0). At 2 hours after Compound #10 removal, a shift was beginning to occur; however, a large percentage of the cells remained delayed in G₁/S. By 5 to 8 hours, cells were clearly redistributing. By 26 hours after Compound #10 washout, the cells had resumed normal cycling.

9.3.1.3 Compound #10 Cell Cycle Delay is Coincident with the Inhibition of VEGF Production

This example demonstrates that Compound #10 cell cycle delay is coincident with the inhibition of VEGF production.

Experimental Design. Several VEGF secreting cell lines were assayed for cell cycle effects. Actively proliferating cells were incubated for 18 hours under normoxic conditions (21% oxygen) with vehicle (0.5% DMSO) alone or with Compound #10 at concentrations of 10 nM or 100 nM. At the completion of treatment, cells were harvested and cellular DNA content was analyzed via PI staining and flow cytometry (as described in Section 9.3.1.1).

Results. In the same cell lines, treatment was undertaken for 48 hours with a range of concentrations of Compound #10 from 0.1 nM to 30 μM or with vehicle (0.5% DMSO) alone. The conditioned media were collected and assayed by ELISA for soluble VEGF₁₂₁ and VEGF₁₆₅ isoforms (as described in Section 9.1.1.1); results were calculated as percentage inhibition relative to vehicle treated controls. EC₅₀ values were calculated from the concentration response curves.

As shown in Table 25, Compound #10 cell cycle delay was coincident with the inhibition of VEGF production in all of the tested tumor types.

TABLE 25
Correlation of VEGF Inhibition and Cell Cycle Delay in Human
Tumor Cell Lines

| | | | Cell Cycle Delay at |
|---|---|---|---|
| | | VEGF Inhibition | VEGF Inhibition |
| Tumor Type | Cell Line | EC₅₀ (nM) | EC₅₀ |
| Cervical | HeLa | 2 | Yes |
| Fibrosarcoma | HT1080 | 10 | Yes |
| Colorectal | HCT116 | 10 | Yes |
| Renal cell | HEK293 | 10 | Yes |
| Lung | NCI H460 | 10 | Yes |
| Glioblastoma | U-87MG | >30,000 | No |
| Pancreas | ASPC-1 | >30,000 | No |
| | PL-45 | >30,000 | No |
| | HPAF-2 | >30,000 | No |
| | PC-3 | >30,000 | No |

Abbreviations:
EC₅₀ = effective concentration achieving 50% of peak activity;
VEGF = vascular endothelial growth factor

9.3.1.4 The Kinetics of S-Phase Transit Employing BrdU Incorporation into DNA

This example demonstrates the rate and number of cells transiting the S-phase of the cell cycle.

Experimental Design. HT 1080 cells are exposed to BrdU (bromodeoxyuridine, a synthetic nucleoside that is an analogue of thymidine and is incorporated into DNA during the S phase of cell division) (FITC BrdU Flow Kit, BD Pharmingen catalog #552598). Cells are grown and treated as described in Section 9.3.1.3 above with the exception that one hour prior to harvesting by trypsinization, BrdU (final concentration 1 μM) is added to each culture for 1 hour. Cells actively replicating DNA during this brief time incorporate the BrdU into the DNA, which can then be quantitated. BrdU content is quantitated with using the FITC BrdU Flow Kit as instructed by the manufacturer. The process includes fixation (paraformaldehyde) and DNA staining with 7-AAD (7-amino-actinomycin D) followed by incubation with a fluoro-tagged anti-BrdU antibody that specifically recognizes BrdU incorporated into DNA. Dual channel FACS analysis permits assessment of both the DNA content of individual cells and the rate of transit across the S-phase, which is assessed based upon BrdU incorporation over the one hour treatment period.

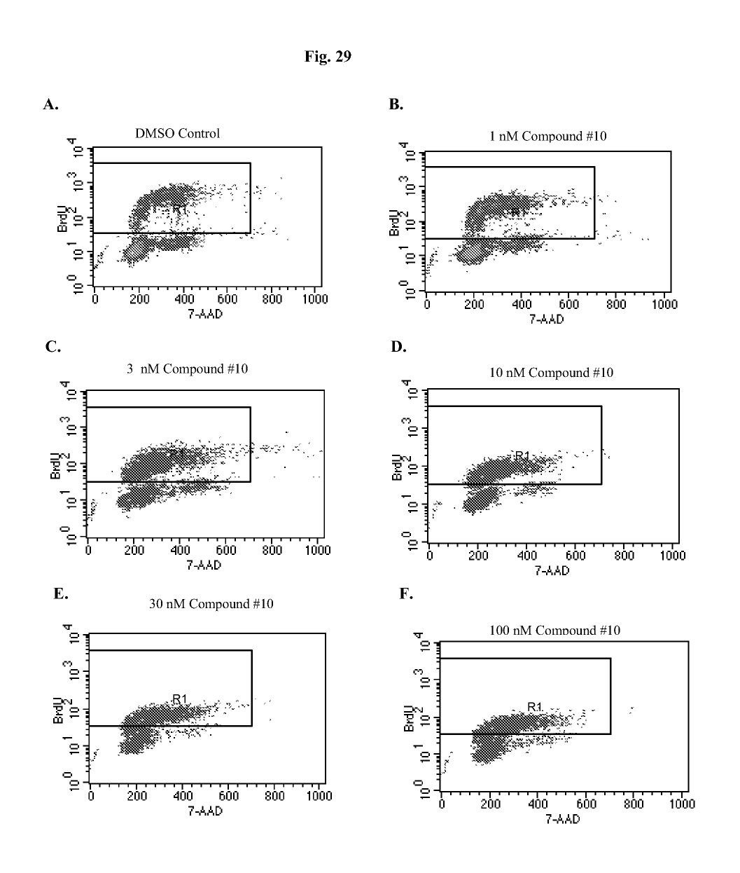

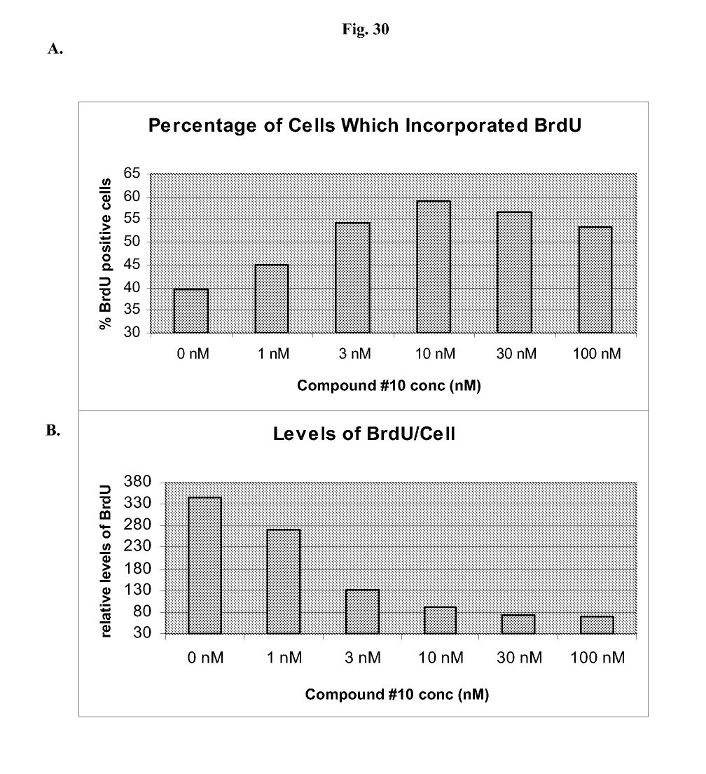

Results: FIG. 29 indicates that an 18-hour treatment with increasing doses of Compound #10 causes a net increase in the percentage of cells residing in S-phase; however, individual cells incorporated less BrdU during the one-hour treatment period compared to DMSO control cells. The percentage of cells incorporating BrdU and the relative level of BrdU at each Compound #10 concentration is shown in FIG. 30. These results suggest that Compound #10 slows the transit of cells through the S-phase of the cell cycle.

9.3.1.5 The Effect of Compound #10 on the 3-Dimensional Growth of HT 1080 cells

This example demonstrates the effect of a Compound provided herein on the 3-dimensional growth of HT1080 cells.

Experimental Design. HT1080 cells grown as a monolayer were trypsinized and seeded onto a 0.75% agar noble base to prevent the cells from attaching to the bottom of the tissue culture plate and to allow/promote the cells to self-adhere and grow as 3-dimensional spheroids. After 4 days the spheroids were established and the liquid growth medium was replaced with medium containing either 0.5% DMSO vehicle, or 10 nM or 50 nM of Compound #10 with 0.5% DMSO vehicle. The cells were incubated for 22 and 45 hours at 37° C., in the presence of a 10% CO₂ atmosphere. Spheroids were visually checked daily for morphological changes and a medium was replenished two times per week. At 22 and 45 hours after exposure to Compound #10, BrdU was added to a subset of the wells designated for FACS analysis and then returned to the incubator for 3 hours to permit cells synthesizing DNA (i.e. cells in S-phase) to incorporate the BrdU into the nascent strands of DNA. These pulse labeled spheroids were then harvested, washed and trypsinized (triple action solution, Gibco), pelleted and prepared for FACS analysis with a FITC BrdU Flow Kit, (BD Pharmingen). Cells were fixed and permeabilized with paraformadehyde and DNA stained with 7-AAD followed by incubation with an antibody which specifically recognizes BrDV incorporated into DNA. As described in Section 9.3.1.4. Cells were analyzed and sorted by 7-AAD signal (DNA content) to determine cell cycle phase, and BrdU content (percent actively synthesizing DNA).

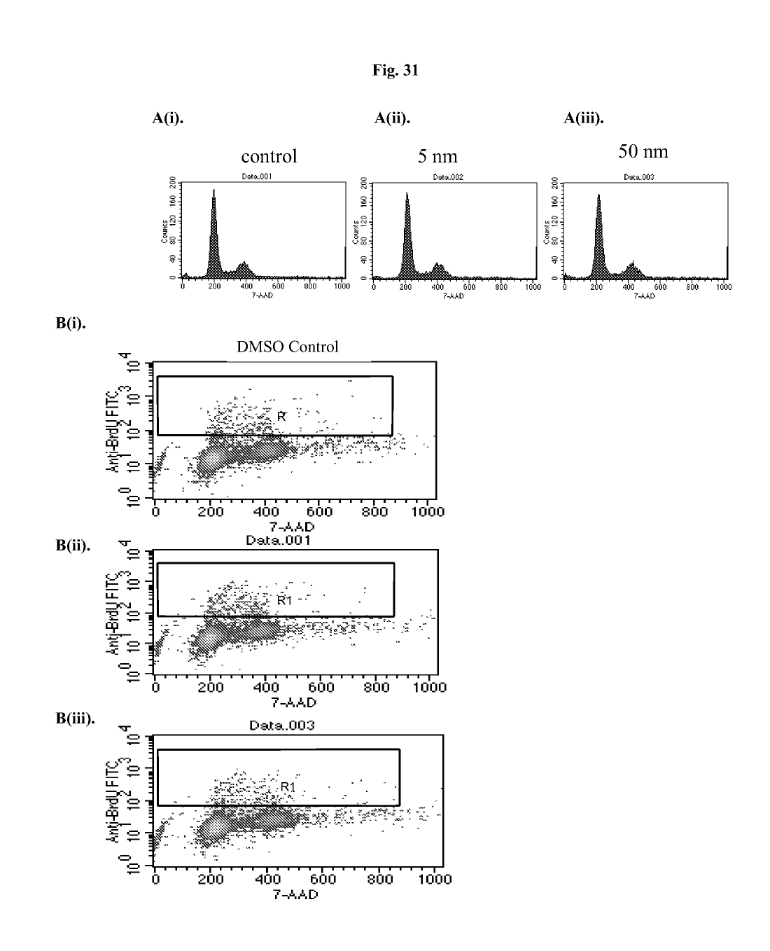

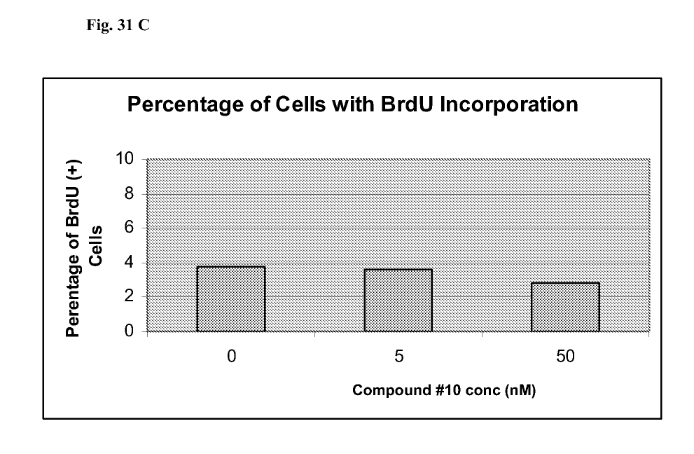

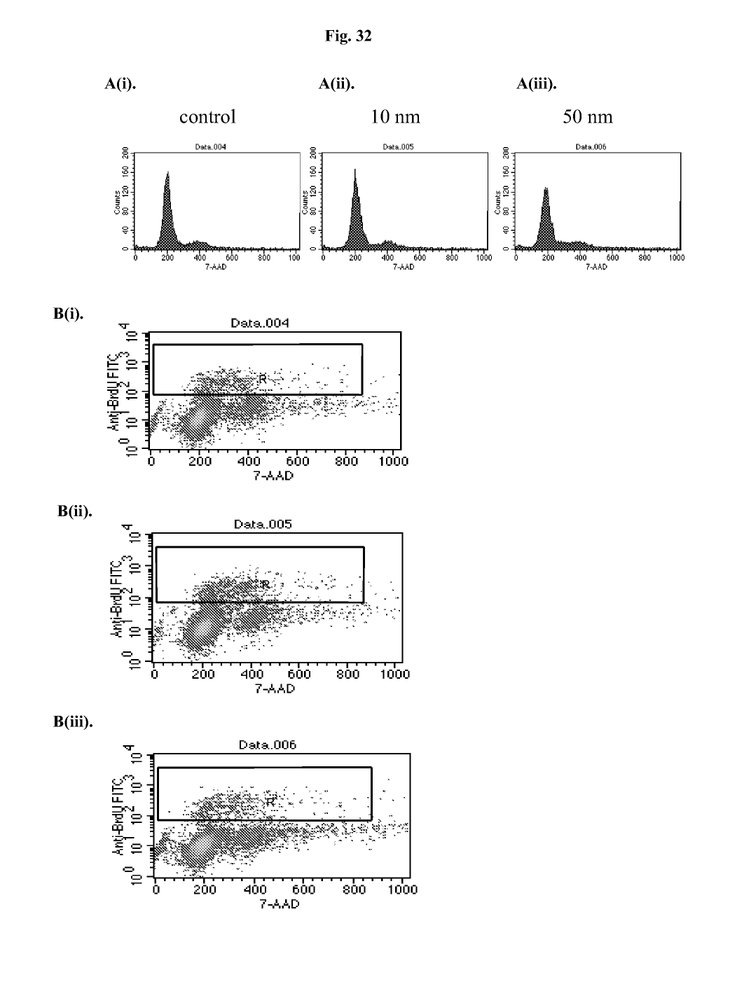

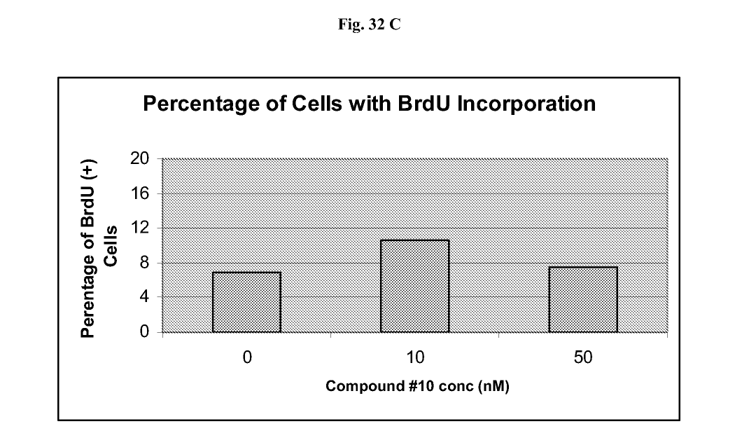

Results. HT1080 spheroids prepared as above were treated with a Compound provided herein for 24 (FIG. 31) or 48 hours (FIG. 32). FIG. 31 and FIG. 32 show: (A) a histogram of DNA content demonstrating that the cell cycle distribution is not affected by exposure to the Compound provided herein; (B) BrdU quantification indicating the fraction of cells actively synthesizing DNA; and (C) a graphical representation of the percentage of cells that incorporated BrdU (i.e., the cells in S-phase), indicating that the percentage is not significantly altered by compound #10 treatment.

Spheroids, prepared as above, were treated with either vehicle alone (0.5% DMSO v/v final) added to the media or a Compounds provided herein (10 nM or 50 nM final concentration) in media to which vehicle has been added. The cells were photographed on day 5 of treatment to assess any gross morphological differences caused by exposure to Compound #10. Spheroids from all treatment groups looked indistinguishable from one another (data not shown). In addition, spheroids maintained in the presence of Compound #10 provided herein for three weeks also display no obvious morphological changes (data not shown).

9.3.1.6 Effect of Compound #10 on HT1080 Cell Viability and Mobility

This example demonstrates that Compound #10 inhibits or reduces the ability of cells to migrate out of spheroids of HT1080 cells.

Experimental Design. To assess the viability and motility of HT 1080 cells exposed to Compound #10, spheroids of HT1080 cells were prepared as in Section 9.3.1.5. The cells were cultured in media with vehicle only (0.5% DMSO) or in the presence of 50 nM Compound #10 present in media with vehicle added. After three weeks of treatment, treated spheroids were re-plated into wells without an agar base, thus allowing cells to migrate out onto the coated surface and grow as a two-dimensional (2-D) monolayer in the presence or absence of Compound #10 at 50 nM. Pictures were then taken 48 hours to assess the migration and proliferation of the cells across the well's surface.

Results. Cells from vehicle treated spheroids plated out in the absence of Compound #10 migrate to cover the entire surface of the tissue culture plate within the 48 hours. Spheroids grown for 3 weeks in the presence of Compound #10 and re-plated in the absence of the compound also migrate out of the spheroid to cover the surface of the tissue culture plate within 48 hours. This indicates that a three-week exposure to Compound #10 does not reduce either the proliferative or the migratory capacity of HT1080 cells.

Cells from control spheroids grown in the absence of Compound #10 and subsequently re-plated in the presence of 50 nM of Compound #10 are blocked in their ability to migrate out of the spheroid, and do not cover the surface of the tissue culture plate. Similarly, cells grown as spheroids in tissue culture media containing 50 nM of Compound #10 herein and re-plated in the presence of Compound #10 migrate much less than other groups. The data suggests that, even after three weeks of growth in three dimensions (3-D), the cell cycle delay and migratory inhibition of Compound #10 herein are still intact once the cells move into 2-D culture. The data further suggests that Compound #10 can act to inhibit the metastasis of cells from tumors.

9.3.1.7 Effect of Compound #10 on Anchorage-Independent Colony Formation in HT1080 Cells

This example demonstrates that Compound #10 may reduce formation of colonies from HT1080 cells treated with Compound #10.

Experimental Design. HT1080 cells growing in monolayer were trypsinized, counted and suspended in a 0.35% agar noble/1× complete DMEM solution at 37° C. at a concentration of 2,500 cells/mL. One ml of this solution was layered over a semisolid base consisting of 0.5 mL of 0.75% agar noble/1× complete DMEM in a six well tissue culture plate. The top layer was permitted to solidify at room temperature, whereupon 1.5 mL of liquid medium (complete DMEM) containing 0.5% DMSO and 0, 5, 20 or 100 nM of Compound #10 was added to achieve a final concentration of 0, 2.5, or 50 nM of Compound #10. Tissue culture plates were then returned to the incubator and colonies were allowed to form. The top medium layer was replaced periodically (every 3-4 days) with complete DMEM containing either 0.5% DMSO or Compound #10 (0, 2.5, 10 or 50 nm) and 0.5% DMSO. On day 18 the vehicle-treated wells had colonies of sufficient size to count (>50 cells/colony). At this time, for increased visualization, 1.5 mL of a 2× working volume of (3-(4,5-Dimethylthiazol-2-yl)-2,5-diphenyltetrazolium bromide, a tetrazole) (MTT, Invitrogen, Cat #C35007) was added and the plates were returned to the incubator for 2 hours until colonies were stained by conversion of the MTT to purple formazan crystals. Colonies were then visually counted under a dissecting microscope.

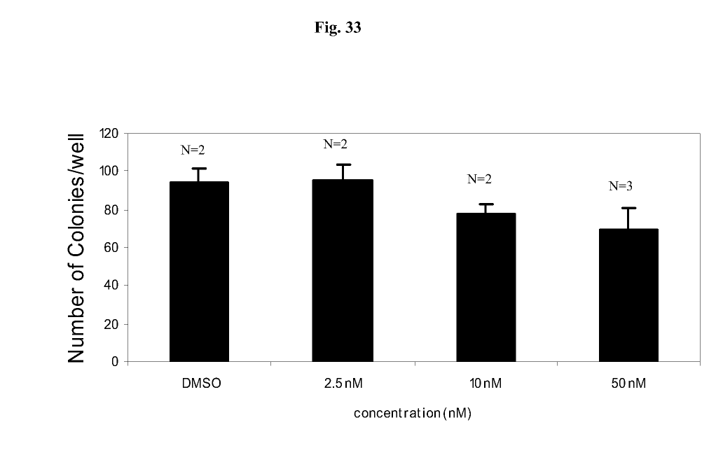

Results: FIG. 33 is a graphical representation of the average for each treatment group, which consists of two or three wells per group. There was a modest trend toward a reduced number of colonies formed from cells treated with 10 and 50 nM of Compound #10, but the results do not reach statistical significance (P=0.29 and 0.07, respectively).

9.3.2 Animal Model Systems

9.3.2.1 Compound #10 Induces S-phase Cell Delay in Dividing Tumor Cells In Vivo.

This example demonstrates that Compound #10 induces a S-phase cell delay in dividing tumor cells in vivo.

Experimental Design. HT1080 cells (5×10⁶ cells/mouse) were implanted subcutaneously in male athymic nude mice. When tumors had become established (i.e., the mean tumor size had reached 585±150 mm³), mice were divided into 4 treatment groups, as shown in Table 26. Positive and negative controls for effects on tumor cell cycling included doxorubicin and bevacizumab, respectively.

After 1, 2, or 3 days of treatment with Compound #10, mice were injected with BrdU, a synthetic nucleoside that is an analogue of thymidine and is incorporated into DNA during the S phase of cell division. The mice were sacrificed 3 hours later, and the tumors collected. A single cell suspension was prepared from the tumor cells. The cells were permeabilized and an antibody to BrdU was used to stain cells that had entered S phase during the labeling period. The proportion of cells actively synthesizing DNA was determined by cell sorting.

TABLE 26
Study Design for Cell Cycle Effect Assessment
in Nude Mice Bearing HT1080 Xenografts

| | Number of Animals | | | | | Dose | Dose |
|---|---|---|---|---|---|---|---|
| Test | Per Time Point^{a} | | Dose | Administration^{a} | | Volume | Concentration |
| Compound | M | F | (mg/kg) | Route | Schedule | (mL/kg) | (mg/mL) |
| Vehicle^{b} | 5 | 0 | 0 | Oral | QD | 4 | 0 |
| Compound #10 | 5 | 0 | 10 | Oral | QD | 4 | 2.50 |
| Doxorubicin | 5 | 0 | 6 | IP | Single | 8 | 0.75 |
| | | | | | bolus | | |
| Bevacizumab | 5 | 0 | 5 | IP | Single | 8 | 0.625 |
| | | | | | bolus | | |

^{a}Treatments were initiated on Day 0 with 20 mice per group. On each day, 5 mice were sacrificed per group for analysis. Mice were treated with Compound #10 daily. Mice were treated with doxorubicin or bevacizumab on Day 0 only.
^{b}Vehicle was L21 (35% Labrasol, 35% Labrafac, and 30% Solutol).
Abbreviations: IP = intraperitoneal; QD = 1 time per day

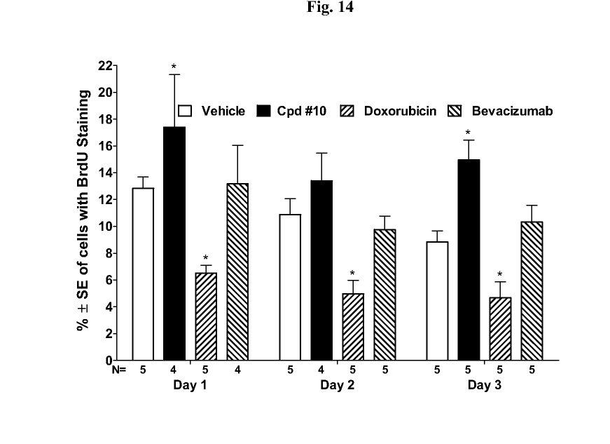

As shown in FIG. 14, approximately 7 to 12% of the tumor cells from vehicle-treated mice were in S phase as indicated by the amount of BrdU incorporation. As the size of the tumors from vehicle treated mice increased with each succeeding treatment day, the percentage of cells showing BrdU incorporation decreased. On each treatment day, tumor cells from mice treated with Compound #10 demonstrated increased BrdU staining, consistent with a higher fraction of cells delayed in S phase. By contrast, treatment with doxorubicin decreased the percentage of tumor cells staining with BrdU, consistent with the arrest in the G1 phase of the cell cycle that is expected with this type of DNA-damaging agent. As also expected, bevacizumab had no effect on the proportion of cells in S phase.

When taken together with reductions in tumor derived plasma VEGF in these same animals (Section 9.2.3), these results are consistent with the previous in vitro results for Compound #10, suggesting that Compound #10 selectively induces a S phase cell delay in rapidly dividing tumor cells.

### 10. EXAMPLE

### Clinical and Pre-Clinical Studies Compound #10

10.1 Pre-clinical Studies

In vitro and in vivo safety pharmacology studies with Compound #10 demonstrate a favorable safety profile. Based on the safety pharmacology studies and results of electrocardiograms (ECGs) and blood pressures collected during 7- and 28-day toxicity studies in dogs, Compound #10 is unlikely to cause serious adverse effects on the central nervous, cardiovascular, and respiratory systems.

A functional observation battery in Sprague Dawley rats dosed daily for 7-days by oral gavage at dose levels of 40, 120, and 400 mg/kg revealed no adverse behavioral or neurological effects at any dose level.

Compound #10 was considered negative for meaningful inhibition of human-ether-a-go-go-related gene (hERG) current in a higher throughput hERG assay. In a cardiovascular safety pharmacology study in awake telemeterized male beagle dogs, single oral doses of 30, 60, and 120 mg/kg of Compound #10 induced no meaningful changes in cardiovascular or electrocardiographic (including QT interval) parameters. In addition, ECG analysis and blood pressure assessments were performed as part of 2 GLP toxicity and toxicokinetic studies of Compound #10 in beagle dogs, one with 7-days of dosing and one with 28-days of dosing followed by a 15-day recovery period. In these studies, oral dosing with Compound #10 at dose levels through 120 mg/kg/day for 7-days and through 60 mg/kg/day for 28-days did not have any toxicological effects on ECG or blood pressure results in dogs. At the end of dosing in the 28-day toxicity study in dogs, males in the 60 mg/kg/day group had a slightly higher (7%) mean uncorrected QT value which also was statistically significant in comparison to controls. However, QTc (QT interval corrected for heart rate) values in males in the 60 mg/kg/day group were comparable to controls.

In a respiratory safety pharmacology study in awake telemeterized male beagle dogs, single oral doses of 30, 60, and 120 mg/kg of Compound #10 induced no dose dependent or biologically significant changes in respiratory rate, core body temperature, arterial blood gases, arterial pH, or arterial bicarbonate.

10.1.1 Pharmacokinetics and Compound Metabolism in Animals

The absorption of Compound #10 was evaluated in nude mice, C57BL/6 mice, Sprague Dawley rats, and beagle dogs dosed by the oral route. The pharmacokinetic evaluations in mice were adjuncts to the primary pharmacodynamic xenograft studies. The evaluations in rats included toxicokinetic assessments in single-dose, 7-day, and 28-day toxicology studies as well as a mass-balance study after a single oral dose of ¹⁴C-Compound #10. The evaluations in dogs included toxicokinetic assessments in 7-day and 28-day toxicology studies. In the studies performed, rodents were dosed once daily with Compound #10 formulated in vehicle and administered via oral gavage. Dogs were dosed BID at ˜12-hour intervals between doses with Compound #10 formulated in vehicle and loaded into gelatin capsules that were administered orally.

The results of the PK studies demonstrate that Compound #10 is orally bioavailable in mice, rats, and dogs. Compound #10 pharmacokinetic parameters have been evaluated in mice at the 1-mg/kg dose level that, when given BID, was associated with maximal antitumor activity in the HT1080 human tumor xenograft model. At Day 1, Compound #10 plasma trough concentration of ˜0.10 to 0.15 μg/mL at 24 hours was established as the minimal mean target plasma concentration to be achieved in pharmacokinetic studies.

In all mice, rats, and dogs, the relationship between Compound #10 dose and plasma exposure describes a “bell-shaped curve,” i.e., plasma exposures initially rise with dose but then decrease despite further increases in dose. These bell-shaped dose-exposure relationships are consistent with absorption saturation and/or possible precipitation of the Compound within the gastrointestinal tract at the highest dose levels. The dose exposure curves were used in the dose selection for the rat and dog toxicology studies and in the interpretation of the No-Observed-Adverse-Effect Levels (NOAELs) from these studies. In both rat and dog toxicology species, Cₘₐₓ and AUC values at the NOAELs exceed those expected in subjects to be enrolled to the proposed Phase 1b clinical study in patients with advanced breast cancer.

In vitro plasma protein binding for ¹⁴C-radiolabeled Compound #10 was determined from plasma samples obtained from mice, rats, dogs, monkeys, and humans. ¹⁴C-radiolabeled Compound #10 was highly bound to proteins in the plasma in vitro, with an overall mean of ≧99.5% for all species. Protein binding was independent of concentration over the range of 0.05 to 50 μg/mL of ¹⁴C-radiolabeled Compound #10. Given the similarities in protein binding across species, these data suggest that cross-species exposure comparisons do not need to be adjusted to take protein binding into account.

When evaluated in human hepatic microsomes or in assays using human recombinant cytochrome P450 (CYP) isoenzymes, Compound #10 inhibits the activity of the CYP2D6 isoenzyme. No meaningful inhibition of CYP3A4, CYP1A2, CYP2C9, or CYPC19 was observed. These data suggest the possibility that Compound #10 may slow or alter the clearance of drugs that are primarily metabolized by CYP2D6. It is possible that in certain clinical trial subjects, such agents may need to be adjusted for dosing or replaced by alternative agents that are not metabolized by CYP2D6, particularly when such agents may have a low therapeutic index.

10.1.2 Toxicology

A comprehensive toxicology program has been completed for Compound #10, consisting of a single-dose oral study in rats, 7-day oral studies in rats and dogs, and 28-day oral studies in rats and dogs each with a 2-week recovery period. A battery of genotoxicity studies was also performed. For the toxicology studies conducted in vivo, the study design consisted of a vehicle control group and 3 dose levels of Compound #10. The L23 vehicle was used. In rats, the vehicle or Compound #10 formulated in vehicle was administered by oral gavage. In dogs, the vehicle alone or Compound #10 formulated in vehicle was loaded into gelatin capsules for oral administration of 2 equal doses ˜12 hours apart (BID). All studies in the toxicology program were conducted according to GLP regulations.

In rats given single oral gavage doses of Compound #10 at doses of 100, 200, or 400 mg/kg, no notable clinical or clinical pathological toxicities were observed at any dose level. Because maximal exposure occurred at 100 mg/kg, this dose is considered the NOAEL for 1 day of dosing.

In the subsequent 7-day study, rats administered oral gavage Compound #10 doses of 40, 120, and 400 mg/kg/day. Maximal exposures occurred at a dose of 120 mg/kg/day. At this dose, notable changes included increases in mean prothrombin time (PT) and mean activated partial thromboplastin time (aPTT) in males but not in females. Elevations of about ˜2.5-fold to about 3-fold in mean cholesterol levels and about 1.3-fold in mean glucose levels were also noted in males and females receiving Compound #10. Based on the collective toxicity and toxicokinetic findings, the NOAEL for 7-days of Compound #10 administration for male rats is 40 mg/kg/day and for female rats is 120 mg/kg/day.

In the 28-day study (with a 14-day recovery period), rats received oral gavage Compound #10 doses of 12, 40, and 120 mg/kg/day. Exposures were maximal at 120 mg/kg/day. Consistent with the 7-day study, the 28-day study showed reversible increases in mean PT and aPTT at Compound #10 doses of 40 and 120 mg/kg/day in males but not in females. Other chemistry changes included about 2- to about 3-fold elevations in mean cholesterol levels in all Compound #10 dose groups, and minimally increased glucose and alkaline phosphatase values in females and minimally increased chloride and minimally decreased potassium values in males dosed with Compound #10 at 40 and 120 mg/kg/day. Increased adrenal weights were observed at all dose levels; these changes correlated with adrenal cortical hypertrophy that was observed in males and females. The findings indicate an NOAEL for 28-days of Compound #10 administration in rats of 12 mg/kg/day.

In dogs given Compound #10 at doses of 10, 30, or 60 mg/kg/dose BID (20, 60, and 120 mg/kg/day) orally in L23 gelatin capsules for 7 consecutive days, exposures were maximal at 30 mg/kg/dose BID. Animals receiving Compound #10 had an increased incidence and frequency of soft stools in both males and females but no other notable treatment-related effects. Considering exposure values, the NOAEL for 7-days is considered to be 30 mg/kg/dose BID (60 mg/kg/day).

In the 28-day study (with a 15-day recovery period), dogs were administered Compound #10 doses of 5, 15, and 30 mg/kg/dose BID (10, 30, or 60 mg/kg/day) in gelatin capsules. Maximal exposures occurred at 30 mg/kg/dose BID (60 mg/kg/day). Compound #10 was clinically well tolerated in male and female dogs at the low- and mid-dose levels but at the high dose, adverse clinical findings, and decreased food consumption resulting in decreased body weights were observed. The target organ of toxicity was the small intestine. Microscopic findings of erosion, necrosis and/or ulceration of the mucosa, submucosal inflammation, epithelial hyperplasia of the mucosa of the crypts, and/or congestion of the Peyer's patches in the small intestine were seen in several dogs at the high dose. The findings in the small intestine did not reverse at the end of the 15-day recovery period. Based on the findings, the NOAEL for 28-days of Compound #10 administration in dogs is considered to be 15 mg/kg/dose BID (30 mg/kg/day).

Genotoxicity was assessed in a battery of in vitro and in vivo studies that included a bacterial reverse mutation study, a chromosome aberration study in Chinese hamster ovary (CHO) cells, and a micronucleus study in rats by the oral route. The in vitro studies were performed in the presence and absence of an exogenous metabolic activation system. There was no evidence of genotoxic effects with Compound #10 in these studies.

10.2 Clinical Studies:

Compound #10 has been evaluated in a Phase 1, escalating multiple-dose, safety, tolerability and pharmacokinetic (PK) study in healthy adult volunteers.

The study was performed under the oversight of the French health authorities. The study was not performed under an IND. The primary objective of the study was to determine a dose range and regimen for Compound #10 that safely achieves and maintains pharmacologically active target plasma concentrations (as determined from xenograft studies) and would be appropriate for use in subsequent Phase 1 or Phase 2 studies in patients with cancer. The secondary objective was to evaluate the safety profile of multiple doses of Compound #10 administered 2 times per day (BID) (Stage 1) or 3 times per day (TID) (Stage 2) in oral capsules, to characterize the multiple dose PK profile of Compound #10, and to assess the effect of Compound #10 on plasma and serum physiological VEGF concentrations.

Methods. The trial was a Phase 1, randomized, escalating multiple dose, single center study conducted in 2 stages. Stage 1 comprised a double blind, placebo controlled dose escalation with Compound #10 given BID. Stage 2 comprised a double blind, placebo controlled escalation of Compound #10 given TID. The number of subjects planned and enrolled for stage 1: 24 subjects as 3 cohorts of 8 subjects, with each cohort comprising 4 males (3 Compound #10, 1 placebo) and 4 females (3 Compound #10, 1 placebo). The number of subjects planned and enrolled for stage 2: 1 cohort of 8 subjects comprising 4 males (3 Compound #10, 1 placebo) and 4 females (3 Compound #10, 1 placebo).

Diagnosis and Main Criteria for Inclusion: Subjects were required to be healthy males or females, 18 to 65 years old, weighing 41 to 90 kg. Female subjects were required to be surgically sterile or post menopausal (as documented by an absence of menses for ≧1 year before screening).

Test and Reference Products: In Stage 1, Compound #10 was provided in gelatin capsules for oral administration. Capsules contained 2 mg or 20 mg of active substance. Cohorts of subjects assigned to active treatment received progressively higher Compound #10 doses of 0.3, 0.6, and 1.2 mg/kg BID (0.6, 1.2, and 2.4 mg/kg/day).

In Stage 2, Compound #10 was provided in gelatin capsules for oral administration. Capsules contained 20 mg or 25 mg of active substance. The cohort of subjects assigned to active treatment received a Compound #10 dose of 1.6 mg/kg TID (4.8 mg/kg/day).

Placebo gelatin capsules for oral administration were used as the reference product in both Stage 1 and Stage 2 of the study.

Duration of Treatment: Stage 1: Compound #10 or placebo was administered orally BID for 7 days (Day 1 through Day 7). Stage 2: Compound #10 or placebo was administered orally TID for 7 days (Day 1 through Day 7).

Criteria for Evaluation: Maximum tolerated dose; Safety as characterized by type, frequency, severity, timing, and relationship to study treatment of any adverse events, laboratory abnormalities, or electrocardiogram (ECG) abnormalities; PK profile of Compound #10 as described by plasma concentration time curves and by derived PK parameters; Plasma and serum VEGF concentrations.

Statistical Methods: The results were summarized by study stage, treatment, and dose.

Pharmacokinetics: Compound #10 concentrations and PK parameters were presented descriptively. Noncompartmental methods were used to compute Tₘₐₓ, Cₘₐₓ, and AUC. Dose proportionality and sex effect were evaluated using ANOVA on log transformed PK parameters using dose, sex, and dose by sex as fixed factors.

Plasma VEGF Concentrations: Plasma and serum VEGF concentrations and concentration changes from baseline were presented descriptively.

Results. As planned, 32 subjects were included in the study. In Stage 1, 8 subjects were enrolled to each of the 3 dose groups (3 males and 3 females receiving Compound #10 and 1 male and 1 female receiving placebo) resulting in enrollment of 24 subjects (12 males and 12 females). In Stage 2, 8 subjects (3 males and 3 females receiving Compound #10 and 1 male and 1 female receiving placebo) completed their participation in the study. No subject discontinued prematurely and all subjects completed the study. Subject characteristics for Stage 1 and Stage 2 are described in Table 27 below. Demographic characteristics in Stage 1 were generally similar between the Compound #10 and placebo groups. Characteristics in Stage 2 were generally similar to those in Stage 1.

TABLE 27
Subject Characteristics: Stage 1 and Stage 2 of Multiple-dose Study

| | Stage 1 | | Stage 2 | |
|---|---|---|---|---|
| | Compound | | Compound | |
| | #10 | Placebo | #10 | Placebo |
| Characteristic | N = 18 | N = 6 | N = 6 | N = 2 |
| Gender, n | | | | |
| Male:Female | 9:9 | 3:3 | 3:3 | 1:1 |
| Median age, years | | | | |
| [range] | | | | |
| Males | 34 [25-62] | 32 [21-38] | 38 [33-46] | 31 [NA] |
| Females | 57 [44-64] | 56 [53-62] | 56 [54-65] | 58 [NA] |
| Mean body weight, | | | | |
| kg [range] | | | | |
| Males | 73 [67-90] | 88 [80-90] | 66 [52-70] | 78 [NA] |
| Females | 62 [46-72] | 55 [52-77] | 66 [51-67] | 70 [NA] |
| Race, n (%) | | | | |
| Caucasian | 14 (78) | 3 (50) | 5 (83) | 2 (100) |
| African/West Indian | 2 (11) | 2 (33) | — | — |
| Other | 2 (11) | 1 (17) | 1 (17) | — |

Abbreviations:
BID = 2 times per day,
TID = 3 times per day

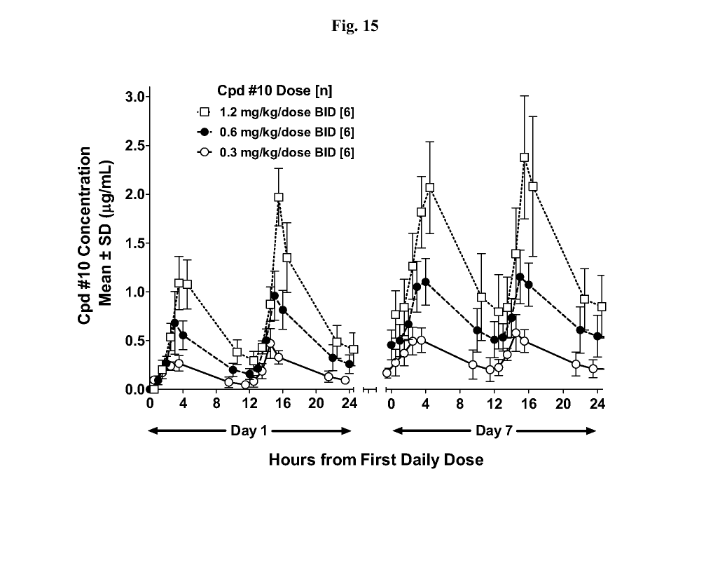

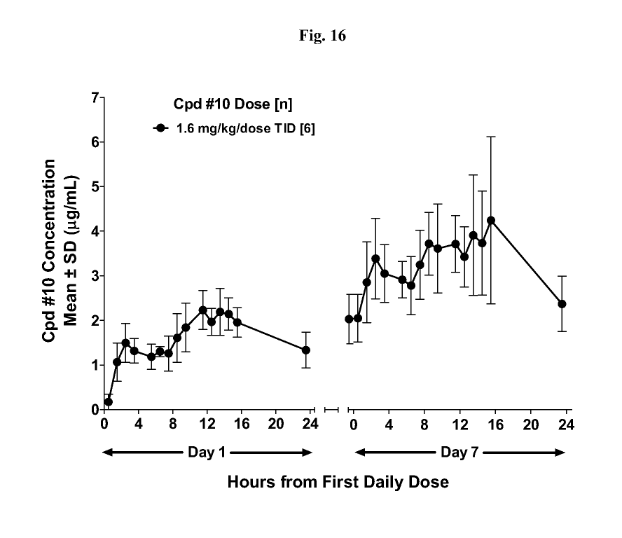

Pharmacokinetics: Mean plasma concentration time profiles for Compound #10 are shown in FIG. 15 for Stage 1 and FIG. 16 for Stage 2. Compound #10 appeared in plasma after a ˜30 minute lag time. On Day 1, mean maximum concentration (Cₘₐₓ) values after the second dose were almost double those of the first dose, while by Day 7, the mean Cmax values of the first and second daily doses appeared similar; this pattern suggests accumulation of Compound #10 concentrations over time rather than diurnal variation in exposures. At all dose levels, the target trough plasma concentration of ˜0.1 to 0.15 μg/mL established as maximally active in the HT1080 animal tumor model was achieved.

PK parameters for Compound #10 in plasma are shown in Table 28 below. The mean Tₘₐₓ was in the range of ˜3 hours. During Stage 1 and Stage 2, increases in mean values for Cₘₐₓ and area under the concentration time curve over 24 hours (AUC₀₋₂₄) were generally dose proportional. When comparing Day 1 to Day 7, there was an increase in the mean Cₘₐₓ and AUC₀₋₂₄ over time at all dose levels, indicating accumulation (˜2-fold) when Compound #10 was dosed continuously. A 2-compartment model could be readily fit to all of the individual subject data throughout the 7 day course of treatment.

TABLE 28
Mean (SD) Compound #10 Pharmacokinetic Parameters:
Stage 1 and Stage 2 Multiple dose Study

| | | | | | | | Stage 2 | |
|---|---|---|---|---|---|---|---|---|
| | Stage 1 | | | | | | Compound #10 | |
| | Compound #10 Dose mg/kg BID | | | | | | Dose mg/kg TID | |
| | 0.3 N = 6 | | 0.6 N = 6 | | 1.2 N = 6 | | 1.6 N = 6 | |
| Parameter, units | Day 1 | Day 7 | Day 1 | Day 7 | Day 1 | Day 7 | Day 1 | Day 7 |
| Tₘₐₓ (after PM dose), | 3.16 | 3.33 | 3.17 | 3.33 | 3.00 | 3.33 | 2.50 | 2.33 |
| hours | (0.41) | (0.52) | (0.41) | (0.52) | (0.00) | (0.52) | (1.05) | (1.37) |
| Cₘₐₓ (after PM dose), | 0.48 | 0.59 | 0.97 | 1.16 | 1.97 | 2.47 | 2.36 | 4.65 |
| μg/mL | (0.15) | (0.18) | (0.24) | (0.27) | (0.29) | (0.57) | (0.46) | (1.86) |
| C_{24 h}, μg/mL | 0.094 | 0.21 | 0.26 | 0.54 | 0.41 | 0.85 | 1.33 | 2.37 |
| | (0.036) | (0.09) | (0.095) | (0.21) | (0.17) | (0.32) | (0.40) | (0.62) |
| AUC₀₋₂₄, μg · hr/mL | 4.31 | 8.44 | 10.1 | 18.6 | 18.0 | 32.9 | 37.2 | 78.6 |
| | (1.20) | (2.84) | (2.60) | (4.85) | (3.97) | (9.43) | (5.90) | (19.4) |
| Dose-normalized Cₘₐₓ, | 0.79 | 0.99 | 0.81 | 0.97 | 0.82 | 1.03 | 0.51 | 0.98 |
| μg/mL/mg/kg | (0.24) | (0.29) | (0.20) | (0.22) | (0.12) | (0.24) | (0.10) | (0.38) |
| Dose-normalized | 7.2 | 14.1 | 8.4 | 15.5 | 7.5 | 13.7 | 7.7 | 16.4 |
| AUC₀₋₂₄, | (2.0) | (4.7) | (2.2) | (4.1) | (1.6) | (3.9) | (1.2) | (4.0) |
| μg · hr/mL/mg/kg | | | | | | | | |

Values represent male and female subjects combined.
Abbreviations: AUC = area under the concentration-time curve, C₂₄ = concentration at 24 hours after first daily dose, Cₘₐₓ = maximum concentration, Tₘₐₓ = time of maximum concentration; BID = 2 times per day, TID = 3 times per day

Gender related differences were analyzed by ANOVA. In this study, no significant differences in Cₘₐₓ or AUC₀₋₂₄ values were observed between males and females.

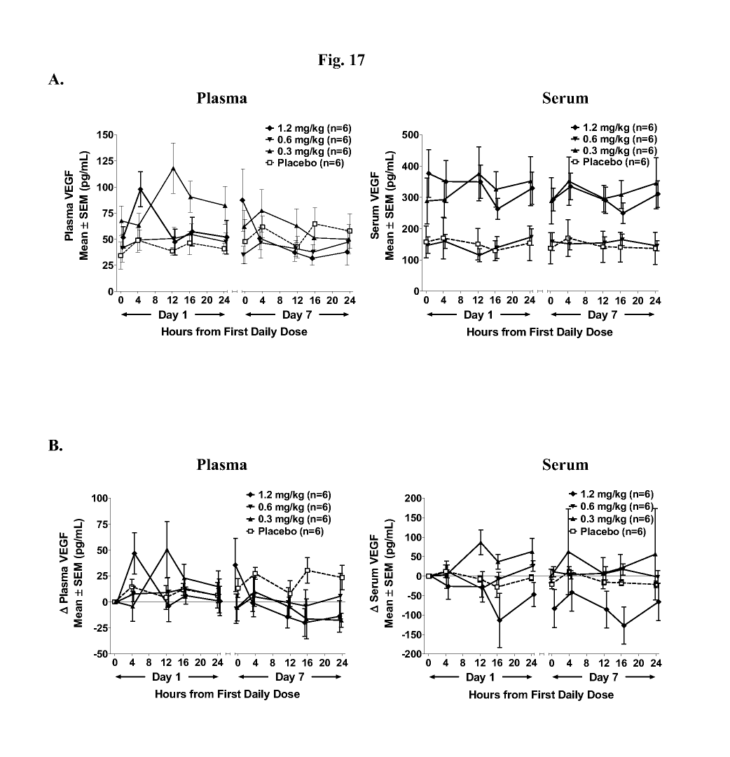

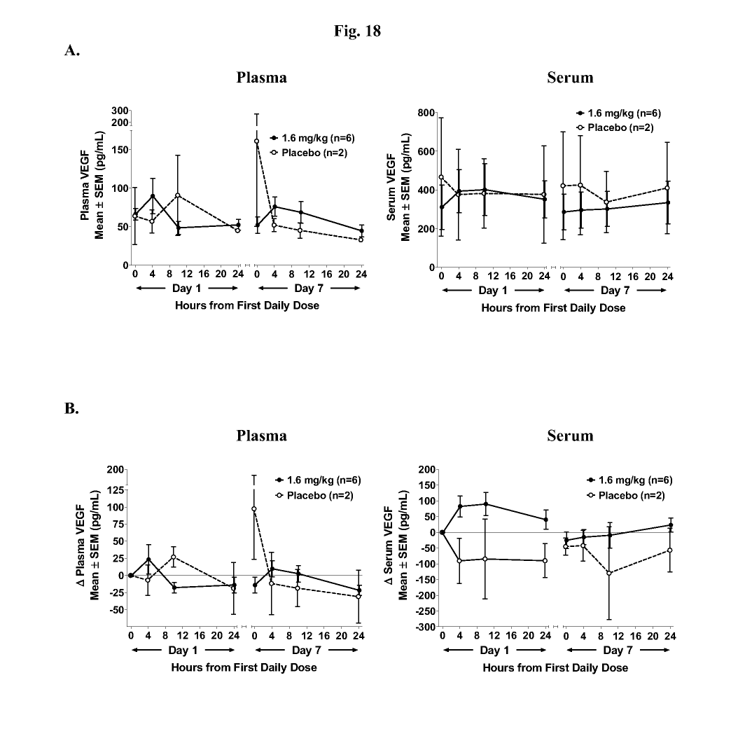

Circulating VEGF Concentrations: Plasma and serum VEGF A concentrations were assayed in all subjects. Mean absolute values and changes from baseline in plasma and serum VEGF A concentrations are plotted in FIG. 17A and FIG. 17B Error! Reference source not found. for Stage 1 and in FIG. 18A and FIG. 18B Error! Reference source not found. for Stage 2. When considering both stages of the study, no clear dose dependent effects of Compound #10 on physiological concentrations of circulating VEGF A were noted.

Results: In this Phase 1 dose study of Compound #10 in healthy volunteer males and females, administration of Compound #10 for 7 consecutive days at doses of 0.3, 0.6, and 1.2 mg/kg BID (0.6, 1.2, and 2.4 mg/kg/day) and at 1.6 mg/kg TID (4.8 mg/kg/day) was well tolerated. Treatment emergent adverse events and laboratory abnormalities were generally Grade 1. The incidence or severity of these findings was not clearly greater in the Compound #10 group than in the placebo group and no dose dependency was apparent. Frequent ECG evaluations revealed no concerning rhythm, waveform, or interval changes. In particular, no meaningful QTc prolongation was observed. No serious adverse events or premature discontinuations due to adverse events occurred. Interventions for adverse events were minimal. None of the safety findings were deemed clinically significant by the investigator. No MTD was established and no dose limiting toxicities were observed through the highest dose level tested (1.6 mg/kg TID).

PK data indicated that Compound #10 is orally bioavailable. The mean Tₘₐₓ was in the range of ˜3 hours. Increases in Cₘₐₓ and AUC were generally proportional with dose. There was ˜2 fold accumulation when Compound #10 was dosed continuously. In this study, no significant differences in Cₘₐₓ or AUC₀₋₂₄ values were observed between males and females. Target trough plasma concentrations of ≧100 to 150 ng/mL derived from preclinical human tumor xenograft models were achieved and maintained at all dose levels in the current study.

No significant alterations in plasma or serum physiological VEGF-A concentrations were observed at any of the Compound #10 doses tested in this multiple dose study. The finding that Compound #10 did not affect physiological plasma or serum VEGF levels in healthy volunteers appears consistent with in vitro results suggesting that Compound #10 does not perturb physiological VEGF production, but acts selectively to inhibit pathological VEGF production (induced by hypoxia or tumor transformation). Lack of changes in circulating VEGF concentrations may correlate with the lack of Compound #10 toxicities (e.g., hypertension, bleeding, proteinuria) in this trial. Such toxicities have been classically associated with currently used drugs that inhibit VEGF signaling at endothelial cells.

Collectively, the safety and PK findings of this study in healthy volunteers indicate that the dosing regimens tested in this study can readily attain target trough plasma concentrations known to be active in nonclinical models of human disease and that oral BID administration of Compound #10 may offer safety and ease of use advantages over existing clinical methods of inhibiting VEGF signaling.

### 11. EXAMPLE

### Protocol for Treating Patients

Subjects with NF2 may receive continuous daily treatment with a Compound administered at 100 mg per dose, 2 times a day (BID) for up to 1 year, or longer as appropriate, or until tumor progression. In a specific embodiment, the Compound is Compound #10 or Compound #1205. Tumor shrinkage or an improvement in hearing are indicators of efficacy.

Clinical Objectives: Efficacy of a Compound for treating NF2 may be assessed by determining the effects of the Compound on tumor volume and/or word recognition in patients with NF2. The efficacy of a Compound for treating NF2 may also be assessed by: (i) determining the effect on pure tone thresholds and BAERs and OAEs in patients with NF2; (ii) determining whether there is an alteration in the perception of tinnitus; (iii) evaluating effects on tumor blood flow, or peritumoral inflammation or edema; (iv) determining effects on concentrations of angiogenic factors or cytokines; (v) describing the Compound's safety profile, (vi) evaluating compliance with treatment with the Compound; and (vii) determining the Compound's plasma exposure over time.

Clinical Endpoints: A primary clinical endpoint for efficacy of a Compound for treating NF2 includes response rate as demonstrated by reduction in tumor volume (e.g., defined as a ≧20% decrease in tumor volume as documented by MRI) and/or an improvement in word recognition, e.g., defined as an increase in percent word recognition that meets predefined 95% critical difference criteria (see, e.g., Thornton et al., J. Speech Hear. Res., September 1978, 21(3): 507-18). Preferably, a response (radiographic or hearing) should persist for ≧8 weeks to be scored.

Other clinical endpoints for the efficacy of a Compound for treating NF2 may include:
1. changes in hearing function as measured by pure-tone average, latency of wave V on BAERs, and OAEs;
2. changes in tinnitus loudness and psychological distress associated with tinnitus as assessed by the Klockhoff-Lindblom tinnitus severity scale (see, e.g., Klockhoff et al., Acta Otolaryngol., April 1967, 63(4): 347-65) and the Tinnitus Reaction Questionnaire (TRQ) (Wilson et al., J. Speech Hear. Res. February 1991, 34(1):197-201);
3. change in tumor perfusion as assessed by changes in DCE-MRI volume transfer coefficient (Kₜᵣₐₙₛ), area under the tumor uptake curve over the first 90 seconds post injection, normalized by the area under the plasma uptake curve over the same period (AUCBN₉₀) in a target tumor lesion;
4. anti-angiogenic or anti-inflammatory activity as documented by changes in the blood concentrations of VEGF, VEGF-C, VEGF-D, P1GF, VEGFR-1, VEGFR-2, IL-6, and IL-8;
5. overall safety profile of a Compound characterized in terms of the type, frequency, severity, timing, and relationship to the therapy of any adverse events or abnormalities of physical findings, laboratory tests, or ECGs; treatment discontinuations due to adverse events; or serious adverse events;
6. trough and peak (4-hour samples) of a Compound's plasma concentrations as assessed by a validated bioanalytical method; and
7. peritumoral inflammation or edema which may be assessed by CT scan, MRI scan, or PET scan.

Evaluation of Clinical Endpoints:

Antitumor activity: Previously used radiographic response and progression criteria (see, e.g., Widemann et al., J. Clin. Oncol., January 2006, 24(3):507-16; and di Tomaso, “Preliminary success with anti-angiogenic therapy of NF2-related tumors” (Meeting abstract) 2008 NF Conference, Children's Tumor Foundation, Bonita Springs, Fla., Jun. 6-10, 2008) can be used to evaluate the ability of a Compound to specifically induce tumor shrinkage. Three-dimensional volumetric measurement performed by MRI has been shown to be sensitive and consistent in assessing tumor size (see, e.g., Harris et al., Neurosurgery, June 2008, 62(6): 1314-9), and thus may be employed to assess tumor shrinkage induced by treatment with a Compound.

Hearing function: Assessments of word recognition and pure tone thresholds may be direct measures of patient functioning that define symptomatic consequences of the presence of VS (see, e.g., Halpin et al., Otol Neurotol., January 2006, 27(1):110-6). Standard clinical criteria for definition of hearing response (H-R) based on a 50-word hearing test (see, e.g., Thornton et al., J. Speech Hear. Res., September 1978, 21(3): 507-18) can be employed for word recognition tests. Measurement of BAERs and OAEs can provide ancillary information regarding nerve transmission of auditory signals and cochlear activity that can supplement functional assessments (see, e.g., Lalwani et al., Am. J. Otol., 1998, 19(3):352-357; and Telischi et al., Laryngoscope., 1995, 105(7): 675-82).

Tinnitus: Improvements in tinnitus may constitute a direct benefit to patients with symptomatic VS (see, e.g., Baguley et al., J. Laryngol. Otol., April 1992, 106(4): 329-31). Two components of the impact of tinnitus relate to its loudness and the extent to which the symptom is annoying. The hearing loss and vertigo that are characteristic in NF2 are known to accentuate the degree of annoyance associated with tinnitus (see, e.g., Hiller et al., Arch. Otolaryngol. Head Neck Surg. December 2006, 132(12):1323-30; and Hiller et al., Audiol Neurootol., 2007, 12(6):391-400). Standardized scales for the evaluation of tinnitus loudness has been used in clinical trials for many years (see, e.g., Klockhoff et al., Acta Otolaryngol. April 1967, 63(4): 347-65; and McCombe et al., Clin Otolaryngol Allied Sci., October 2001, 26(5): 388-93), and have been shown to be responsive to surgical or medical intervention (see, e.g., Baguley et al., J. Laryngol. Otol., April 1992, 106(4): 329-31; Klockhoff et al., Acta Otolaryngol. April 1967, 63(4): 347-65; and Sparano et al., Int. Tinnitus J., 2004, 10(1):73-7). The development of the Tinnitus reaction Questionnaire (“TRQ”) has simplified the assessment of psychological distress associated with tinnitus. A standard TRQ (Table 29) offering a quick and valid assessment of subjective tinnitus distress (see, e.g., Wilson et al., J Speech Hear Res., February 1991, 34(1):197-201) may be administered to evaluate the potential impact on the quality of life of the subjects with treatment with a Compound.

TABLE 29
Tinnitus questionnaire
Tinnitus Reaction Questionnaire (TRQ)^{a}

| Number | Item | Scores^{b} | | | | |
|---|---|---|---|---|---|---|
| 1 | My tinnitus has made me unhappy. | □ 0 | □ 1 | □ 2 | □ 3 | □ 4 |
| 2 | My tinnitus has made me feel tense. | □ 0 | □ 1 | □ 2 | □ 3 | □ 4 |
| 3 | My tinnitus has made me feel irritable. | □ 0 | □ 1 | □ 2 | □ 3 | □ 4 |
| 4 | My tinnitus has made me feel angry. | □ 0 | □ 1 | □ 2 | □ 3 | □ 4 |
| 5 | My tinnitus has led me to cry. | □ 0 | □ 1 | □ 2 | □ 3 | □ 4 |
| 6 | My tinnitus has led me to avoid quiet situations. | □ 0 | □ 1 | □ 2 | □ 3 | □ 4 |
| 7 | My tinnitus has made me feel less interested in going out. | □ 0 | □ 1 | □ 2 | □ 3 | □ 4 |
| 8 | My tinnitus has made me feel depressed. | □ 0 | □ 1 | □ 2 | □ 3 | □ 4 |
| 9 | My tinnitus has made me feel annoyed. | □ 0 | □ 1 | □ 2 | □ 3 | □ 4 |
| 10 | My tinnitus has made me feel confused. | □ 0 | □ 1 | □ 2 | □ 3 | □ 4 |
| 11 | My tinnitus “driven me crazy”. | □ 0 | □ 1 | □ 2 | □ 3 | □ 4 |
| 12 | My tinnitus interfered with my enjoyment of life. | □ 0 | □ 1 | □ 2 | □ 3 | □ 4 |
| 13 | My tinnitus made it hard for me to concentrate. | □ 0 | □ 1 | □ 2 | □ 3 | □ 4 |
| 14 | My tinnitus has made it hard for me to relax. | □ 0 | □ 1 | □ 2 | □ 3 | □ 4 |
| 15 | My tinnitus has made feel distressed. | □ 0 | □ 1 | □ 2 | □ 3 | □ 4 |
| 16 | My tinnitus has me feel helpless. | □ 0 | □ 1 | □ 2 | □ 3 | □ 4 |
| 17 | My tinnitus has made me feel frustrated with things. | □ 0 | □ 1 | □ 2 | □ 3 | □ 4 |
| 18 | My tinnitus has interfered with my ability to work. | □ 0 | □ 1 | □ 2 | □ 3 | □ 4 |
| 19 | My tinnitus has led me to despair. | □ 0 | □ 1 | □ 2 | □ 3 | □ 4 |
| 20 | My tinnitus has led me to avoid noisy situations. | □ 0 | □ 1 | □ 2 | □ 3 | □ 4 |
| 21 | My tinnitus has led me to avoid social situations. | □ 0 | □ 1 | □ 2 | □ 3 | □ 4 |
| 22 | My tinnitus has made me feel hopeless about the future. | □ 0 | □ 1 | □ 2 | □ 3 | □ 4 |
| 23 | My tinnitus has interfered with my sleep. | □ 0 | □ 1 | □ 2 | □ 3 | □ 4 |
| 24 | My tinnitus led me to think about suicide. | □ 0 | □ 1 | □ 2 | □ 3 | □ 4 |
| 25 | My tinnitus has made me feel panicky. | □ 0 | □ 1 | □ 2 | □ 3 | □ 4 |
| 26 | My tinnitus has made me feel tormented. | □ 0 | □ 1 | □ 2 | □ 3 | □ 4 |

^{a}From Wilson et al., J. Speech Hear. Res. February 1991, 34(1): 197-201
^{b}0 = not at all, 1 = a little of the time, 2 = some of the time, 3 = a good deal of the time, and 4 = almost all of the time.

Tumor perfusion using DCE-MRI: Assessing tumor blood flow offers an additional parameter of a Compound's action that can confirm the downstream consequences of decreasing tumor VEGF. Measurement of blood flow in target lesions provides direct evidence of a Compound's action on tumors that can be correlated with plasma VEGF changes. Assessment of tumor perfusion using DCE-MRI may be used to evaluate the efficacy of a Compound using standard protocols (see., e.g., Morgan et al., J. Clin. Oncol., Nov. 1, 2003, 21(21):3955-64; Leach et al., Br. J. Cancer, May 9, 2005, 92(9):1599-610; Liu et al., J. Clin. Oncol., August 2005, 23(24): 5464-73; and Thomas et al., J. Clin. Oncol., Jun. 20, 2005, 23(18):4162-71).

Anti-angiogenic activity: Assessing VEGF concentrations provides a relevant and convenient mechanism-specific marker of Compound activity. Appropriate methods for the measurement of VEGF concentrations have been determined (see, e.g., Jelkmann et al., Clin. Chem., April 2001, 47(4):617-23.), and such methods may be used to evaluate the effects of a Compound. For example, clinically validated ELISA kits (e.g., from R&D Systems, Minneapolis, Minn.) may be used to measure concentrations of VEGF, VEGF-C, P1GF, VEGFR, and inflammatory mediators such as IL-6 and IL-8. CT scan and MRI scan may also be used to assess peritumoral inflammation or edema.

Safety: Adverse medical events that may be encountered in patients receiving the Compound may be monitored. For consistency of interpretation, adverse events may be coded using the standard Medical Dictionary for Regulatory Activities (MedDRA), and the severity of these events may be graded using the well-defined Common Terminology Criteria for Adverse Events (CTCAE). Standard definitions for seriousness may be applied.

Subject Selection: The following eligibility criteria may be used to select subjects for whom treatment with a Compound is considered appropriate.

Subjects should meet the following conditions to be eligible for the treatment protocol:
1. Diagnosis of NF2 by National Institutes of Health (NIH) criteria (see NIH. Neurofibromatosis. Conference statement. National Institutes of Health Consensus Development Conference. Arch Neurol. 1988 May 45(5): 575-8) with evidence of either:
a. Bilateral VS, or
b. First-degree family relative with NF2 and either unilateral VS or any 2 of: meningioma, schwannoma, glioma, neurofibroma, and juvenile posterior subcapsular lens opacity;
2. Evidence of disease progression defined by any of the following features:
a. Progressive VS growth (≧20% increase in either volume [if volumetric measurement performed] or ≧2 mm increase in greatest linear dimension) based on serial MRI studies in subjects who are at elevated risk for surgical complications (e.g., deafness, lower cranial nerve injury, or facial weakness) or who refuse surgery; and
b. Progressive hearing loss related to VS (i.e., not due to surgery or radiation) with a word recognition score of <85% in at least 1 affected ear;
3. In the judgment of the physician, use of a Compound offers acceptable benefit:risk when considering current NF2 disease status, medical condition, and the potential benefits of and risks of surgery or irradiation;
4. Discontinuation of all therapies (except corticosteroids) for the treatment of NF2≧4 weeks before initiation of treatment protocol;
5. All acute toxic effects (excluding alopecia or neurotoxicity) of any prior therapy resolved to Common Terminology Criteria for Adverse Events (“CTCAE”) Version 3.0 Grade≦1 before initiation of treatment with a Compound; and
6. Adequate functional status (Karnofsky Performance Score≧60).

Compound Administration: A Compound may be orally administered each day on a BID schedule at approximately the same times each day. Ideally doses should be taken at ˜12-hour intervals (e.g., at ˜7:00 AM and at ˜7:00 PM). If convenient for the subject, a Compound may be taken during or within ˜30 minutes after a meal; however, administration with food is not required. Subjects may continue receiving repeated 4-week cycles of a Compound indefinitely or until termination. Compound administration may be terminated because of, e.g., tumor progression or other progression of NF2, or a dose-limiting toxicity.

The dosage administered to a subject may be reduced to 80 mg/dose BID, 60 mg/dose BID, or 40 mg/dose if a dose-limiting toxicity (DLT) occurs. The dosage may be successively reduced if a DLT occurs. In other words, if a DLT occurs at 100 mg/dose BID, then the dosage may first be reduced to 80 mg/dose BID, and if a DLT occurs again then the dosage may be reduced to 60 mg/dose BID. A DLT may be defined as the occurrence of any of the following:

1. Grade≧2: a Compound-related vomiting despite maximal oral antiemetic therapy, or a requirement for intravenous antiemetics to control a Compound-related nausea and vomiting.

2. Grade≧2: proteinuria.

3. Other Grade≧3: a Compound-related toxicity.

Schedule of Events and Procedures

β-Human Chorionic Gonadotropin. Women of childbearing potential may have serum beta human chorionic gonadotropin (β-HCG) testing prior to initial administration of a Compound.

Vital Signs. Vital signs (pulse and blood pressure) may be monitored prior to initial administration of a Compound, and at other times as clinically indicated.

Height, Body Weight, and Performance Status. Height (in cm) can be measured once prior to initial administration of a Compound. Body weight and Karnofsky performance status may also be assessed.

Hematology Laboratory Assessment. Hematology laboratory assessments may include, but are not limited to, white blood cell count with differential, hemoglobin, hematocrit, other red cell parameters, and platelet count. These parameters may be monitored prior to initial administration of a Compound, and during the treatment protocol as necessary.

Biochemistry Laboratory Assessment. Biochemistry laboratory assessments may include sodium, potassium, chloride, bicarbonate, blood urea nitrogen, creatinine, calcium, phosphorus, uric acid, glucose, total protein, albumin, globulin, albumin:globulin ratio, bilirubin (direct and indirect), aspartate aminotransferase, alanine aminotransferase, gamma glutamyl transferase, alkaline phosphatase, lactate dehydrogenase, total cholesterol, triglycerides, low-density lipoprotein, and high-density lipoprotein. These parameters can be monitored prior to, and/or at various times during, the treatment protocol. To the extent possible, all samples for biochemistry parameter analysis should be taken after an overnight fast.

Coagulation Laboratory Assessment. Coagulation laboratory assessments may include prothrombin time (“PT”) and activated partial thromboplastin time (“aPTT”). These parameters can be monitored prior to, and/or at various times during, the treatment protocol.

Urinalysis. Urinalyses may include dipstick analysis for pH, specific gravity, glucose, ketones, blood, protein, urobilinogen, and bilirubin. These parameters can be monitored prior to initiation of the treatment protocol and/or at various times during the treatment protocol.

12-Lead Electrocardiogram. A 12-lead ECG can be obtained prior to initiation of the treatment protocol and/or at various times during the treatment protocol.

Blood for a Compound's Plasma Concentrations. Blood samples for a Compound's plasma concentration assessments can be collected immediately pre-dose and at ˜4 hours after administration of the AM dose at various time points during the treatment protocol. If a heparinized venous catheter is placed for sample collection in order to avoid repeated needle sticks, at least 2 mL of blood may be removed and discarded prior to each sample collection in order to avoid heparin contamination of the sample. All attempts should be made to collect the blood samples at, or within ±5 minutes of, the scheduled time. The timing of the blood draw can be in relation to the Compound's dosing time and not the time of the preceding meal.

Each sample may comprise 3 mL of venous blood drawn into a VACUTAINER® tube with K₂-ethylenediaminetetraacetic acid (EDTA) as the anticoagulant. Immediately after collection, the tube may be gently inverted 8 to 10 times to mix the anticoagulant with the blood sample. The tube may be stored upright on ice until centrifugation; centrifugation and sample processing may be performed within 1 hour of sample collection. The plasma fraction may be separated by placing the collection tube into a refrigerated centrifuge (4 to 8° C.) in a horizontal rotor (with a swing-out head) for a minimum of 15 minutes at 1500 to 1800 relative centrifugal force (RCF). The plasma fraction can be withdrawn by pipette and divided into 2 polypropylene freezing tubes (with each tube receiving approximately equal aliquots). After processing, samples should preferably be placed into a freezer at approximately −70° C.

Analyses of a Compound's plasma concentrations can be performed using a validated LC-MS/MS method. Plasma samples collected for analysis can be preserved for future metabolite analysis, as appropriate.

Blood for VEGF, VEGFR, and Cytokines. Two blood samples (1 for plasma and 1 for serum) may be obtained for assessment of VEGF, VEGFR, and cytokine levels.

Each sample for plasma collection may comprise 3 mL of venous blood drawn into a VACUTAINER® tube with K₂EDTA as the anticoagulant. Immediately after collection, the tube may be gently inverted 8 to 10 times to mix the anticoagulant with the blood sample. The tube may be stored upright at room temperature until centrifugation; centrifugation and sample processing may be performed within 30 minutes of sample collection. The plasma fraction may be separated by placing the collection tube into a room-temperature (18 to 25° C.) horizontal rotor (with a swing-out head) for 15 minutes at 1000 to 2500 RCF. Immediately following the completion of centrifugation, the plasma fraction may be withdrawn by pipette and divided into 2 polypropylene freezing tubes (with each tube receiving approximately equal aliquots).

Each sample for serum collection may comprise 4 mL of venous blood drawn into a VACUTAINER® SST™ Tube. After collection, the tube may be stored upright at room temperature for 30 minutes to allow the sample to clot prior to centrifugation. The serum fraction may be separated by placing the collection tube into a room-temperature (18 to 25° C.), horizontal rotor (with a swing-out head) for 15 minutes at 1000 to 2500 RCF. Immediately following the completion of centrifugation, the serum fraction may be withdrawn by pipette and divided into 2 polypropylene freezing tubes (with each tube receiving approximately equal aliquots).

After processing, samples may be placed into a freezer at approximately −70° C. An ELISA-based multiplex system may be used to measure plasma VEGF and cytokine levels.

Tinnitus Assessments. The loudness/severity of the tinnitus may be rated according to the criteria modified from Klockhoff-Lindblom scale (Klockhoff et al., Acta Otolaryngol. April 1967, 63(4): 347-651967). Patients may be asked to score their tinnitus in the past week consistent with the scale shown in Table 30 below:

TABLE 30
Grading of Tinnitus Severity

| Score | Description |
|---|---|
| 0 | Tinnitus is not perceived at all. |
| 1 | Tinnitus is perceived slightly^{a} and periodically. |
| 2 | Tinnitus is perceived slightly^{a} and continuously; |
| | or moderately^{b} and periodically. |
| 3 | Tinnitus is perceived slightly^{a} to moderately^{b} |
| | and continuously. |
| 4 | Tinnitus is perceived moderately^{b}, or slightly^{a} to |
| | severly^{c}, and continuously. |
| 5 | Tinnitus is perceived from moderately^{b} to |
| | severely^{c} and continuously. |
| 6 | Tinnitus is perceived severely^{c} and continuously. |

^{a}Only in quiet environment,
^{b}In ordinarily noisy environment but divertible, i.e., not observed when attention focused on work, etc,
^{c}Constantly noticed in all ordinary acoustic environments, and even when concentrating on work, etc.

Immediately thereafter, the TRQ (Table 29) (see, e.g., Wilson et al., J. Speech Hear. Res. February 1991, 34(1):197-201) may be administered to assess psychological distress associated with tinnitus in the past week.

Hearing Tests. Subjects may undergo hearing tests at screening, and at various times during the treatment protocol. Hearing assessment may be determined in standard clinical fashion and may include:

Determination of word recognition scores using a standardized list of test words, i.e., the 50-item Central Institute for the Deaf [CID] list W-22, recorded) (Thornton et al., J. Speech Hear Res., September 1978, 21(3): 507-18);

Pure-tone averages calculated as the average of the pure-tone thresholds at 500, 1000, 2000, and 3000 Hz;

Brainstem auditory evoked response (BAERs) reported as the latency of wave V, if present; and

Otoacoustic emission (OAE) levels at frequencies above 1 KHz, if present.

These parameters may be collected for target and contralateral VS (if present).

Definition of Hearing Response for Target VS and Contralateral VS:

Hearing response (H-R) may be defined as an increase in the word recognition score above the 95% critical difference threshold (Table 31), taking as a reference the baseline word discrimination score (Thornton et al., J. Speech Hear Res., September 1978, 21(3): 507-18).

Hearing progressive disease (H-PD) may be defined as a decrease in the word recognition score below the 95% critical difference threshold (Table 31), taking as a reference the baseline word discrimination score.

Hearing stable disease (H-SD) may defined as word recognition score within the 95% critical difference threshold (Table 31), taking as a reference the baseline word discrimination score (Thornton et al., J. Speech Hear Res., September 1978, 21(3): 507-18).

TABLE 31
Clinical Criteria for Definition of Hearing Response Based On a
50-Word Hearing Test^{a}

| Baseline Word | 95% Critical | Hearing | Progressive |
|---|---|---|---|
| Recognition Score | Difference^{b} | Response^{c} | Disease^{d} |
| (%) | (%) | (%) | (%) |
| 0 | 0-4 | ≧6 | n/a |
| 2 | 0-10 | ≧12 | n/a |
| 4 | 0-14 | ≧16 | n/a |
| 6 | 2-18 | ≧20 | 0 |
| 8 | 2-22 | ≧24 | 0 |
| 10 | 2-24 | ≧26 | 0 |
| 12 | 4-26 | ≧28 | ≦2 |
| 14 | 4-30 | ≧32 | ≦2 |
| 16 | 6-32 | ≧34 | ≦4 |
| 18 | 6-34 | ≧36 | ≦4 |
| 20 | 8-36 | ≧38 | ≦6 |
| 22 | 8-40 | ≧42 | ≦6 |
| 24 | 10-42 | ≧44 | ≦8 |
| 26 | 12-44 | ≧46 | ≦10 |
| 28 | 14-46 | ≧48 | ≦12 |
| 30 | 14-48 | ≧50 | ≦12 |
| 32 | 16-50 | ≧52 | ≦14 |
| 34 | 18-52 | ≧54 | ≦16 |
| 36 | 20-54 | ≧56 | ≦18 |
| 38 | 22-56 | ≧58 | ≦20 |
| 40 | 22-58 | ≧60 | ≦20 |
| 42 | 24-60 | ≧62 | ≦22 |
| 44 | 26-62 | ≧64 | ≦24 |
| 46 | 28-64 | ≧66 | ≦26 |
| 48 | 30-66 | ≧68 | ≦28 |
| 50 | 32-68 | ≧70 | ≦30 |
| 52 | 34-70 | ≧72 | ≦32 |
| 54 | 36-72 | ≧74 | ≦34 |
| 56 | 38-74 | ≧76 | ≦36 |
| 58 | 40-76 | ≧78 | ≦38 |
| 60 | 42-78 | ≧80 | ≦40 |
| 62 | 44-78 | ≧80 | ≦42 |
| 64 | 46-80 | ≧82 | ≦44 |
| 66 | 48-82 | ≧84 | ≦46 |
| 68 | 50-84 | ≧86 | ≦48 |
| 70 | 52-86 | ≧88 | ≦50 |
| 72 | 54-86 | ≧88 | ≦52 |
| 74 | 56-88 | ≧90 | ≦54 |
| 76 | 58-90 | ≧92 | ≦56 |
| 78 | 60-92 | ≧94 | ≦58 |
| 80 | 64-92 | ≧94 | ≦62 |
| 82 | 66-94 | ≧96 | ≦64 |
| 84 | 68-94 | ≧96 | ≦66 |
| 86 | 70-96 | ≧98 | ≦68 |
| 88 | 74-96 | ≧98 | ≦72 |
| 90 | 76-98 | 100 | ≦74 |
| 92 | 78-98 | 100 | ≦76 |
| 94 | 82-98 | 100 | ≦80 |
| 96 | 86-100 | n/a | ≦84 |
| 98 | 90-100 | n/a | ≦88 |
| 100 | 96-100 | n/a | ≦94 |

^{a}Adapted from Thornton and Raffin, 1978
^{b}Upper and lower limits for the 95% critical differences for percentage scores. Changes in word recognition within the 95% critical difference are not statistically different from the baseline score.
^{c}Improved hearing
^{d}Decreased hearing

Confirmation of Hearing Response: To be assigned a status of H-R, changes in word discrimination may be confirmed by a repeat hearing assessment performed ≧8 weeks after the criteria for response are first met. In the case of H-SD, follow-up measurements may meet the H-SD criteria at least once after administration of a Compound at a minimum interval of 8 weeks.

Tumor Perfusion with DCE-MRI. Subjects may undergo DCE-MRI for the target lesion of interest during the screening period and between 1 to 8 hours after the AM dose at various times throughout the treatment protocol.

Timing of Tumor Volume Assessment: Subjects may undergo tumor volume measurement at screening, and at various times throughout the treatment protocol.

Method of Assessment: The determination of antitumor efficacy may be based on objective tumor assessments made according to volumetric measurement (see Widemann et al., J Clin Oncol. 2006 Jan. 20; 24(3):507-16; di Tomaso et al., Preliminary success with anti-angiogenic therapy of NF2-related tumors. (Meeting abstract). 2008 NF Conference, Children's Tumor Foundation, Bonita Springs, Fla., Jun. 6-10, 2008; Harris et al., Neurosurgery. 2008 June 62(6): 1314-9) and treatment decisions by the physician can be based on these assessments.

The same method of assessment and the same technique may be used to characterize each identified and reported lesion at baseline and during follow-up. Imaging-based evaluation is preferred to evaluation by clinical examination when both methods have been used to assess the antitumor effect of treatment.

The MRI examinations may be performed using a standard head coil with 1.5-T scanners. The standard imaging acquisition protocol include conventional pre- and post-gadolinium contrast, spin-echo, T1-weighted, 3-mm thin (contiguous, no gap), axial and coronal series covering the internal auditory canal. For VS quantitative assessment, the MRI scans may be transmitted to a Vitrea2 workstation (Vital Images, Minnetonka, Minn.) for image processing and volume calculation.

Measurability of Tumor Lesions: At baseline, tumor lesions may be categorized by the physician as measurable or non-measurable as described below.
a. Measurable: Lesions that can be accurately measured volumetrically.
b. Non-Measurable: Previously irradiated lesions, and lesions that cannot be measured volumetrically due to the presence of artifacts from cochlear or auditory brainstem implants or due to ill-defined tumor margins resulting from the juxtaposition of tumors abutting each other.

Recording Tumor Measurements: In patients with bilateral VS, the target lesion corresponds to the VS associated with the fastest decline in word recognition score (unless loss of auditory function resulted from surgery). In the rare circumstance where both lesions are equivalent, the physician can choose a target lesion. In patients with unilateral VS (e.g., when a contralateral tumor has been resected previously), the target lesion corresponds to the remaining VS. The baseline volume for the target lesion may be recorded and used as reference to characterize the degree of tumor response during treatment. If possible, serial assessments of the target lesion prior to Compound treatment may be accessed to establish a pre-treatment growth rate. For volumetric analyses, the size may be recorded in centimeter cubed (cm³) to the nearest tenth (0.1 cm³). For unidimensional analyses, the size may be recorded in centimeter (cm) to the nearest tenth (0.1 cm). The sequence parameters used to measure the target VS on these scans (e.g., post-gadolinium, contrast, spin-echo, T1-weighted, 3-mm, thin [continuous, no gap] axial series through the internal auditory canal) may be recorded. Non-target lesions may include: (1) contralateral VS (if present), (2) total volume of meningioma above the foramen magnum, and (3) total volume of non-VS above the foramen magnum. Non-target lesions may also be recorded at baseline and may be assessed volumetrically; however, measurements are not required and these lesions may be followed as “present” or “absent.”

Definitions of Tumor Response

Target Lesions:
Radiographic response (R-R) may be defined as a ≧20% decrease in the volume of the target lesion, taking as a reference the baseline volume.
Radiographic progressive disease (R-PD) may be defined as a ≧20% increase in the volume of the target lesion, taking as a reference the baseline volume.
Radiographic stable disease (R-SD) may be defined as neither sufficient shrinkage to qualify for RR nor sufficient increase to qualify for progressive disease (PD), taking as a reference the baseline volume. As a subset of the SD response category, a minor response (MR) may be assigned to target lesions that decrease between −5% and −20% in volume, taking as a reference the baseline volume.

Non-Target Lesions:
R-R may be defined as a ≧20% decrease in the volume of the non-target lesions, taking as a reference the baseline volume.
R-PD may be defined as a ≧20% increase in the volume of the non-target lesions, taking as a reference the baseline volume. Schwannomas and meningiomas may be classified separately under non-target lesions.
R-SD may be defined as neither sufficient shrinkage to qualify for R-R nor sufficient increase to qualify for R-PD, taking as a reference the baseline volume. As a subset of the R-SD response category, an R-MR can be assigned to target lesions that decrease between −5% and −20% in volume, taking as a reference the baseline volume.

Confirmation of Tumor Response: To be assigned a status of R-R, changes in tumor measurements in subjects with responding tumors are preferably confirmed by repeat studies performed ≧8 weeks after the criteria for response are first met. In the case of R-SD, follow-up measurements preferably meet the R-SD criteria at least once after administration of a Compound at a minimum interval of 8 weeks.

Determination of Overall Response: When both target and non-target lesions are present, individual assessments may be recorded separately. The overall assessment of response may involve all parameters as depicted in Table 32.

TABLE 32
Radiographic Response Criteria

| Target | Non-target | Overall |
|---|---|---|
| Lesion^{a} | Lesions^{b} | Response |
| R-R | R-R | R-R |
| R-R | R-SD | R-R |
| R-SD | R-R | R-R |
| R-SD | R-SD | R-SD |
| R-MR | R-SD | R-MR |
| R-SD | R-MR | R-MR |
| R-PD | Any response | R-PD |
| Any response | PD | PD |

^{a}Measurable lesions only
^{b}May include measurable lesions not followed as target lesions or non-measurable lesions
Abbreviations:
R-MR = radiographic minor response,
R-PD = radiographic progressive disease,
RR = radiographic response,
R-SD = radiographic stable disease

The best overall response is the best response recorded from the start of the treatment until disease progression/recurrence (taking as a reference the baseline measurements). The subject's best response assignment depends on the achievement of both measurement and confirmation criteria. Subjects may be defined as not evaluable if a post enrollment oncologic assessment is not performed. These subjects may be counted as failures in the analysis of tumor response data. Subjects with a global deterioration of health status requiring discontinuation of treatment without objective evidence of disease progression at that time may be reported as having “symptomatic deterioration”. Every effort should be made to document the objective progression even after discontinuation of treatment.

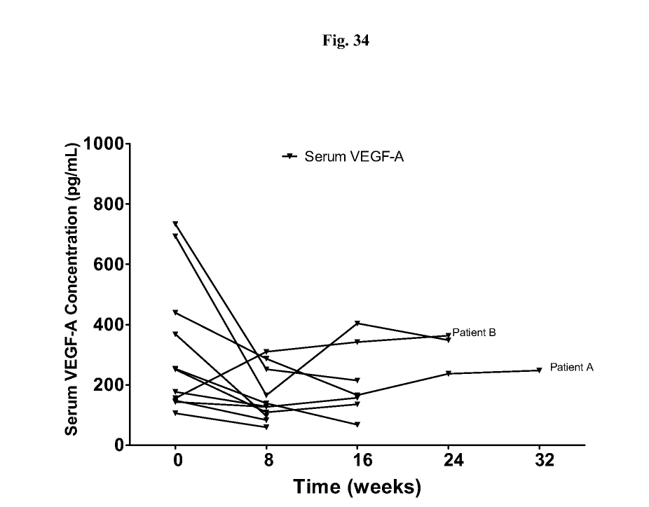

FIG. 34 shows that Compound #10 administration reduced serum VEGF-A levels in patients.

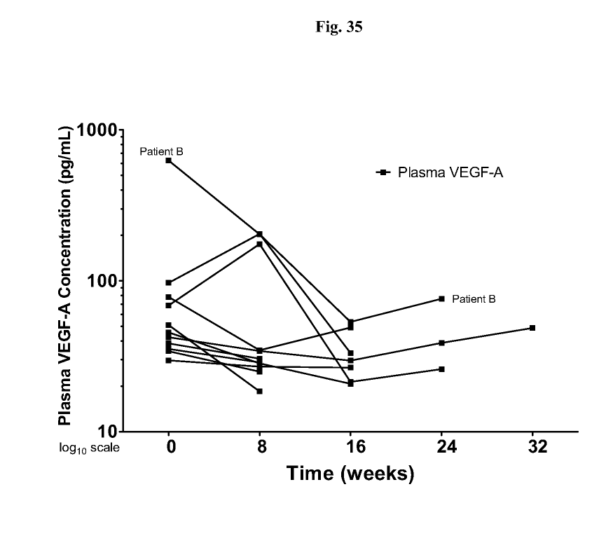

FIG. 35 shows that Compound #10 administration reduced plasma VEGF-A levels in patients.

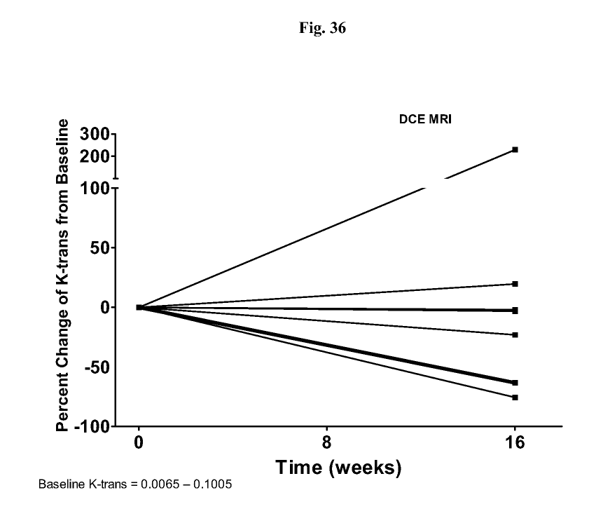

FIG. 36 shows the effect of Compound #10 administration reduced tumor perfusion in patients.

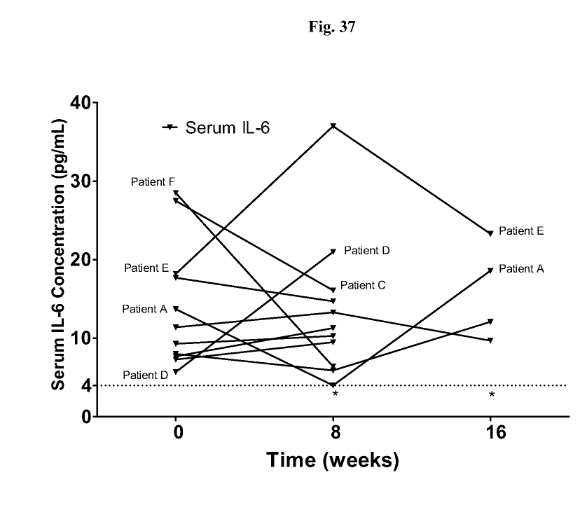

FIG. 37 shows that #10 administration reduced serum IL-6 levels in patients.

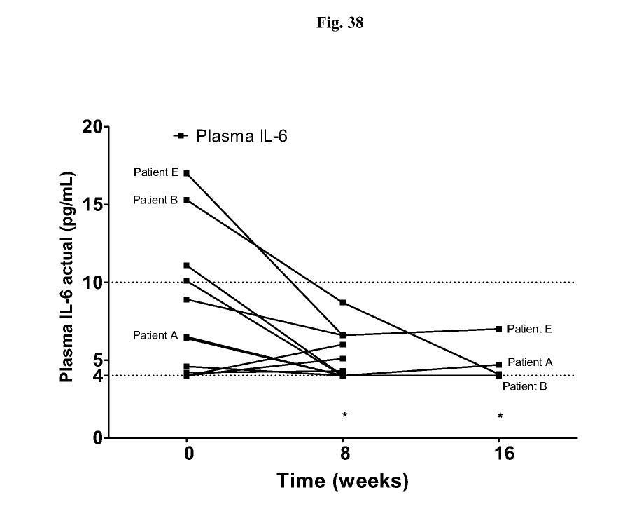

FIG. 38 shows that Compound #10 administration reduced plasma IL-6 levels in patients.

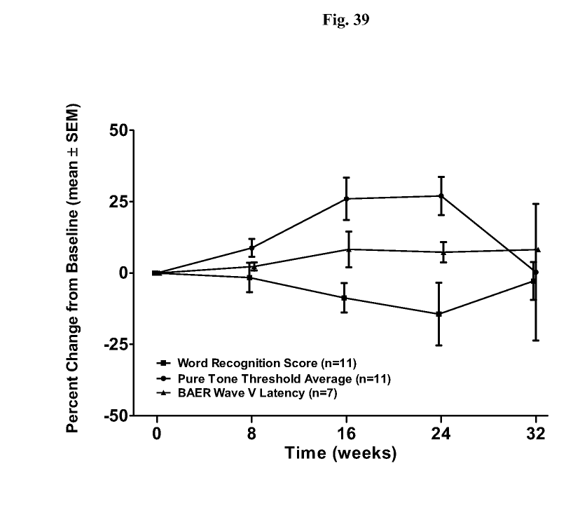

FIG. 39 shows that Compound #10 administration stabilized hearing function in patients while maintaining average word recognition score and average pure tone threshold.

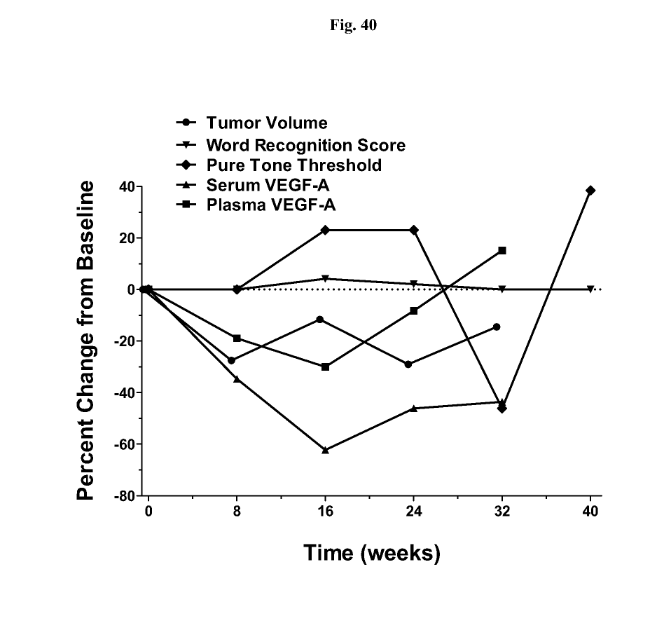

FIG. 40 shows combined data for one patient showing the effect of Compound #10 administration in reducing and stabilizing tumor volume and maintaining average word recognition score and average pure tone threshold.

### 12. EXAMPLE

### Treatment in Disease Model

Confirmation that NF1 tumor angiogenesis can be inhibited by a Compound may be determined using a sciatic nerve xenograft model. In a specific embodiment, the Compound is Compound #10 or Compound #1205.

The sciatic nerve xenograft model, which shows robust growth of a human NF1 malignant peripheral nerve sheath tumor (“MPNST”) cell line within the nerve compartment, resulting in visibly-enlarged nerves within 8 weeks (FIG. 3, from Perrin et al., Laboratory Investigation 87:1092-1102 (2007)), may be employed. This provides a model similar to a rapidly-growing plexiform neurofibroma, with histology better resembling an MPNST. This xenograft has never shown signs of metastasis. This cell line, sNF96.2, secretes VEGF into tissue culture media. The xenograft is vascular, expressing von Willebrand factor (FIG. 4; from Perrin et al., Laboratory Investigation 87:1092-1102 (2007)) and Flk-1 (a VEGF receptor), compared to normal nerve, which has very little vascularity (Perrin et al., Laboratory Investigation 87:1092-1102 (2007)). Thus, this is an excellent model for testing a Compound because it has all the properties needed to evaluate efficacy within a reasonably short time, and for testing its efficacy in an intraneural tumor (which neurofibromas and schwannomas are).

Each of five scid (immunocompromised) female mice (8-12 weeks old) have sNF96.2 cells xenografted into one sciatic nerve. After two weeks, the mice are treated with a Compound via gavage, once daily at a dose of 10 mg/kg (0.2 mg in 0.25 mL of L21 vehicle) for the remainder of the experiment. Five mice also are given unilateral xenografts but do not receive a Compound, as a control. 12 weeks after xenografting, the mice are humanely euthanized and their sciatic nerves dissected out. Equal lengths of nerve are weighed, measured, and photographed for all nerves (control xenografts, normal nerves of treated mice, xenografts of treated mice). In addition, the nerves are fixed and longitudinally embedded in paraffin, then cut into 7-micron sections. Immunohistochemical staining is done for Ki67 (proliferation index) and von Willebrand factor (to analyze vascularity, number of blood vessels per microscopic field), to compare the treated with untreated xenografts (2-tailed t test for comparing immunostain outcomes). Detection of a clear reduction in xenograft size in the treated mice, compared to untreated, and of a decreased proliferation index and decreased vascularity is an indication of anti-tumor efficacy of a Compound.

The surgery is intricate; 1-2 mice are operated on per day, xenografted with the same cell line at the same passage. Thus, this effort may be carried out slightly staggered, with follow-up daily treatment and harvest also staggered following the 3-month timeframe. The xenografts are allowed to go 3 months to have as robust a readout as possible. Previous studies indicates that the mice do not have any overt illness or neurological deficit due to the xenograft growth up through that time period (i.e., 3 months). Post-operative deaths with this procedure are rare, but it is possible. Batch immunostaining may be performed. Other assays that can be carried out include, but are not limited to, an ELISA to measure levels of VEGF in pieces of xenograft controlled for size, measurement of serum levels of a Compound and test for its presence in the nerve and brain.

Additional NF1 or NF2 cell lines may be tested in similar xenograft mouse models with appropriate adaptations made by those skilled in the art. Schwannomatosis cell lines may be tested in similar xenograft mouse models with appropriate adaptations made by those skilled in the art.

Neurofibroma Schwann cells immortalized into cell line cultures and then implanted into mice may provide longer-term xenograft models to confirm if more slowly-growing NF1 tumors respond to a Compound. Alternatively, genetically engineered mice that form neurofibromas (e.g. the Dhh-Cre Nf1 knockout mice) can be treated with a Compound to assess the efficacy of long-term Compound therapy in prevention of tumor formation in the mouse model. Efficacy of a Compound for treating NF can be assessed/confirmed in some of the same xenografts (e.g., mice implanted with sNF96.2 cells), but using scid mice having an NF1 heterozygous background. Efficacy of a Compound for treating NF can also be assessed using a transgenic animal expressing human VEGF.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references cited herein are incorporated herein by reference in their entirety and for all purposes to the same extent as if each individual publication or patent or patent application was specifically and individually indicated to be incorporated by reference in its entirety for all purposes.

What is claimed is:

1. A method for treating neurofibromatosis (NF), comprising administering to a human in need thereof an effective amount of a compound having Formula (I): 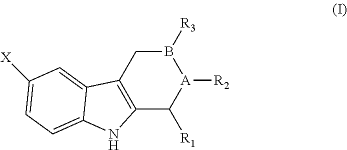
or a pharmaceutically acceptable salt, racemate or stereoisomer thereof, wherein,
X is hydrogen; C₁ to C₆ alkyl optionally substituted with one or more halogen substituents; hydroxyl; halogen; or C₁ to C₅ alkoxy optionally substituted with aryl;
A is CH or N;
B is CH or N, with the proviso that at least one of A or B is N, and that when A is N, B is CH;
R₁ is hydroxyl; C₁ to C₈ alkyl optionally substituted with alkylthio, 5 to 10 membered heteroaryl, or aryl optionally substituted with one or more independently selected Rₒ substituents; C₂ to C₈ alkyenyl; C₂ to C₈ alkynyl; 3 to 12 membered heterocycle optionally substituted with one or more substituents independently selected from halogen, oxo, amino, alkylamino, acetamino, thio, or alkylthio; 5 to 12 membered heteroaryl optionally substituted with one or more substituents independently selected from halogen, oxo, amino, alkylamino, acetamino, thio, or alkylthio; or aryl, optionally substituted with one or more independently selected Rₒ substituents;
Rₒ is a halogen; cyano; nitro; sulfonyl optionally substituted with C₁ to C₆ alkyl or 3 to 10 membered heterocycle; amino optionally substituted with C₁ to C₆ alkyl, —C(O)—R_{b}, —C(O)O—R_{b}, sulfonyl, alkylsulfonyl, 3 to 10 membered heterocycle optionally substituted with —C(O)O—Rₙ; —C(O)—NH—R_{b}; 5 to 6 membered heterocycle; 5 to 6 membered heteroaryl; C₁ to C₆ alkyl optionally substituted with one or more substituents independently selected from hydroxyl, halogen, amino, or 3 to 12 membered heterocycle wherein amino and 3 to 12 membered heterocycle are optionally substituted with one or more C₁ to C₄ alkyl substituents optionally substituted with one or more substituents independently selected from C₁ to C₄ alkoxy, amino, alkylamino, or 5 to 10 membered heterocycle; —C(O)—Rₙ; or —ORₐ;
Rₐ is hydrogen; C₂ to C₈ alkylene; —C(O)—Rₙ; —C(O)O—R_{b}; —C(O)—NH—R_{b}; C₃-C₁₄cycloalkyl; aryl; heteroaryl; heterocyclyl; C₁ to C₈ alkyl optionally substituted with one or more substituents independently selected from hydroxyl, halogen, C₁ to C₄ alkoxy, amino, alkylamino, acetamide, —C(O)—R_{b}, —C(O)O—R_{b}, aryl, 3 to 12 membered heterocycle, or 5 to 12 membered heteroaryl, further wherein the alkylamino is optionally substituted with hydroxyl, C₁ to C₄ alkoxy, or 5 to 12 membered heteroaryl optionally substituted with C₁ to C₄ alkyl, further wherein the acetamide is optionally substituted with C₁ to C₄ alkoxy, sulfonyl, or alkylsulfonyl, further wherein the 3 to 12 membered heterocycle is optionally substituted with C₁ to C₄ alkyl optionally substituted with hydroxyl, —C(O)—Rₙ, —C(O)O—Rₙ, or oxo, further wherein the amino is optionally substituted with C₁ to C₄ alkoxycarbonyl, imidazole, isothiazole, pyrazole, pyridine, pyrazine, pyrimidine, pyrrole, thiazole or sulfonyl substituted with C₁ to C₆ alkyl, wherein pyridine and thiazole are each optionally substituted with C₁ to C₄ alkyl;
R_{b} is hydroxyl; amino; alkylamino optionally substituted with hydroxyl, amino, alkylamino, C₁ to C₄ alkoxy, 3 to 12 membered heterocycle optionally substituted with one or more independently selected C₁ to C₆ alkyl, oxo, —C(O)O—Rₙ, or 5 to 12 membered heteroaryl optionally substituted with C₁ to C₄ alkyl; C₁ to C₄ alkoxy; C₂ to C₈ alkenyl; C₂ to C₈ alkynyl; aryl, wherein the aryl is optionally substituted with one or more substituents independently selected from halogen or C₁ to C₄ alkoxy; 5 to 12 membered heteroaryl; 3 to 12 membered heterocycle optionally substituted with one or more substituents independently selected from acetamide, —C(O)O—Rₙ, 5 to 6 membered heterocycle, or C₁ to C₆ alkyl optionally substituted with hydroxyl, C₁ to C₄ alkoxy, amino, or alkylamino; or C₁ to C₈ alkyl optionally substituted with one or more substituents independently selected from C₁ to C₄ alkoxy, aryl, amino, or 3 to 12 membered heterocycle, wherein the amino and 3 to 12 membered heterocycle are optionally substituted with one or more substituents independently selected from C₁ to C₆ alkyl, oxo, or —C(O)O—Rₙ;
R₂ is hydrogen; hydroxyl; 5 to 10 membered heteroaryl; C₁ to C₈ alkyl optionally substituted with hydroxyl, C₁ to C₄ alkoxy, 3 to 10 membered heterocycle, 5 to 10 membered heteroaryl, or aryl; —C(O)—R_{c}; —C(O)O—R_{d}; —C(O)—N(R_{d}R_{d}); —C(S)—N(R_{d}R_{d}); —C(S)—O—Rₑ; —S(O₂)—Rₑ; —C(NRₑ)—S—Rₑ; or —C(S)—S—R_{f};
R_{c} is hydrogen; amino optionally substituted with one or more substituents independently selected from C₁ to C₆ alkyl or aryl; aryl optionally substituted with one or more substituents independently selected from halogen, haloalkyl, hydroxyl, C₁ to C₄ alkoxy, or C₁ to C₆ alkyl; —C(O)—Rₙ; 5 to 6 membered heterocycle optionally substituted with —C(O)—Rₙ; 5 to 6 membered heteroaryl; thiazoleamino; C₁ to C₈ alkyl optionally substituted with one or more substituents independently selected from halogen, C₁ to C₄ alkoxy, phenyloxy, aryl, —C(O)—Rₙ, —O—C(O)—Rₙ, hydroxyl, or amino optionally substituted with —C(O)O—Rₙ;
R_{d} is independently hydrogen; C₂ to C₈ alkenyl; C₂ to C₈ alkynyl; aryl optionally substituted with one or more substituents independently selected from halogen, nitro, C₁ to C₆ alkyl, —C(O)O—Rₑ, or —ORₑ; or C₁ to C₈ alkyl optionally substituted with one or more substituents independently selected from halogen, C₁ to C₄ alkyl, C₁ to C₄ alkoxy, phenyloxy, aryl, 5 to 6 membered heteroaryl, —C(O)—Rₙ, —C(O)O—Rₙ, or hydroxyl, wherein the aryl is optionally substituted with one or more substituents independently selected from halogen or haloalkyl;
Rₑ is hydrogen; C₁ to C₆ alkyl optionally substituted with one or more substituents independently selected from halogen or alkoxy; or aryl optionally substituted with one or more substituents independently selected from halogen or alkoxy;
R_{f} is C₁ to C₆ alkyl optionally substituted with one or more substituents independently selected from halogen, hydroxyl, C₁ to C₄ alkoxy, cyano, aryl, or —C(O)—Rₙ, wherein the alkoxy is optionally substituted with one or more C₁ to C₄ alkoxy substituents and the aryl is optionally substituted with one or more substituents independently selected from halogen, hydroxyl, C₁ to C₄ alkoxy, cyano, or C₁ to C₆ alkyl;
Rₙ is hydroxyl, C₁ to C₄ alkoxy, amino, or C₁ to C₆ alkyl;
R₃ is hydrogen or —C(O)—R_{g}; and
R_{g} is hydroxyl; amino optionally substituted with cycloalkyl or 5 to 10 membered heteroaryl; or 5 to 10 membered heterocycle, wherein the 5 to 10 membered heterocycle is optionally substituted with —C(O)—Rₙ.

2. The method of claim 1, wherein the NF is NF Type 1.

3. The method of claim 1, wherein the NF is NF Type 2.

4. The method of claim 3, wherein bilateral vestibular schwannomas or unilateral vestibular schwannomas are present in the human in optional conjunction with the presence of one or more NF2-associated tumors selected from a meningioma, schwannoma, ependymoma, glioma or neurofibroma.

5. The method of claim 1, wherein the NF is Schwannomatosis.

6. The method of claim 1, wherein the effective amount is in a range of from about 0.001 mg per kg per day to about 1500 mg per kg per day.

7. The method of claim 1, wherein the compound is administered during or within about 30 minutes after a meal.

8. The method of claim 1, wherein the effective amount of the compound is administered two times per day at a time interval of from about 12 hours to about 18 hours between doses.

9. The method of claim 8, wherein the effective amount of the compound is administered two times per day at a time interval of about 12 hours between doses.

10. The method of claim 1, wherein the effective amount of the compound is administered three times per day at a time interval of from about 8 hours to about 12 hours between doses.

11. The method of claim 10, wherein the effective amount of the compound is administered three times per day at a time interval of about 8 hours between doses.

12. The method of claim 1, wherein the compound has the Formula (II): 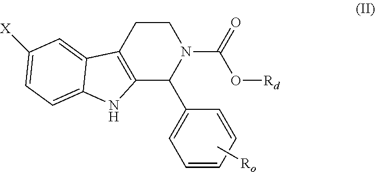
or a pharmaceutically acceptable salt, racemate or stereoisomer thereof, wherein,
X is hydrogen; C₁ to C₆ alkyl optionally substituted with one or more halogen substituents; hydroxyl; halogen; or C₁ to C₅ alkoxy optionally substituted with phenyl;
Rₒ is halogen; cyano; nitro; sulfonyl substituted with C₁ to C₆ alkyl or morpholinyl; amino optionally substituted with C₁ to C₆ alkyl, C(O)R_{b}, —C(O)O—R_{b}, alkylsulfonyl, morpholinyl or tetrahydropyranyl; C₁ to C₆ alkyl optionally substituted with one or more substituents independently selected from hydroxyl, halogen or amino; C(O)—Rₙ; or —ORₐ;
Rₐ is hydrogen; C₂ to C₈ alkenyl; —C(O)—Rₙ; —C(O)O—R_{b}; —C(O)—NH—R_{b}; C₁ to C₈ alkyl optionally substituted with one or more substituents independently selected from hydroxyl, halogen, C₁ to C₄ alkoxy, C₁ to C₄ alkoxy C₁ to C₄ alkoxy, amino, alkylamino, dialkylamino, acetamide, —C(O)—R_{b}, —C(O)O—R_{b}, aryl, morpholinyl, thiomorpholinyl, pyrrolidinyl, piperidinyl, piperazinyl, 1,3-dioxolan-2-one, oxiranyl, tetrahydrofuranyl, tetrahydropyranyl, 1,2,3-triazole, 1,2,4-triazole, furan, imidazole, isoxazole, isothiazole, oxazole, pyrazole, thiazole, thiophene or tetrazole;
wherein amino is optionally substituted with C₁ to C₄ alkoxycarbonyl, imidazole, isothiazole, pyrazole, pyridine, pyrazine, pyrimidine, pyrrole, thiazole or sulfonyl substituted with C₁ to C₆ alkyl, wherein pyridine and thiazole are each optionally substituted with C₁ to C₄ alkyl;
wherein alkylamino and dialkylamino are each optionally substituted on alkyl with hydroxyl, C₁ to C₄ alkoxy, imidazole, pyrazole, pyrrole or tetrazole; and,
wherein morpholinyl, thiomorpholinyl, pyrrolidinyl, piperidinyl, piperazinyl and oxiranyl are each optionally substituted with —C(O)—Rₙ, —C(O)O—Rₙ or C₁ to C₄ alkyl, wherein C₁ to C₄ alkyl is optionally substituted with hydroxyl;
R_{b} is hydroxyl; amino; alkylamino, optionally substituted on alkyl with hydroxyl, amino, alkylamino or C₁ to C₄ alkoxy; C₁ to C₄ alkoxy; C₂ to C₈ alkenyl; C₂ to C₈ alkynyl; aryl optionally substituted with one or more substituents independently selected from halogen and C₁ to C₄ alkoxy; furan; or C₁ to C₈ alkyl optionally substituted with one or more substituents independently selected from C₁ to C₄ alkoxy, aryl, amino, morpholinyl, piperidinyl or piperazinyl;
R_{d} is aryl optionally substituted with one or more substituents independently selected from halogen, nitro, C₁ to C₆ alkyl, —C(O)O—Rₑ, and —ORₑ;
Rₑ is hydrogen; C₁ to C₆ alkyl optionally substituted with one or more substituents independently selected from halogen and alkoxy; or phenyl, wherein phenyl is optionally substituted with one or more substituents independently selected from halogen and alkoxy; and
Rₙ is hydroxyl, C₁ to C₄ alkoxy, amino or C₁ to C₆ alkyl.

13. The method of claim 1, wherein the compound has the Formula (II): 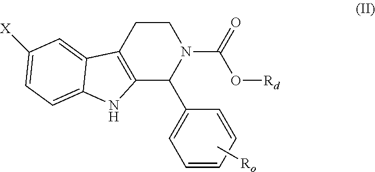
or a pharmaceutically acceptable salt, racemate or stereoisomer thereof, wherein,
X is halogen;
Rₒ is halogen, substituted or unsubstituted C₁ to C₈ alkyl or ORₐ;
Rₐ is H, C₁ to C₈ alkyl optionally substituted with one or more substituents independently selected from hydroxyl and halogen; and
R_{d} is phenyl optionally substituted with one or more alkoxy or halogen substituents.

14. The method of claim 1, wherein the compound has the Formula (II): 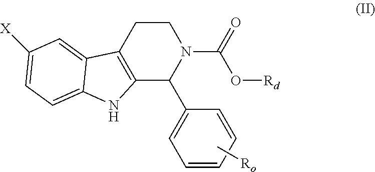
or a pharmaceutically acceptable salt, racemate or stereoisomer thereof, wherein,
X is halogen;
Rₒ is halogen, substituted or unsubstituted C₁ to C₈ alkyl or ORₐ;
Rₐ is H, or C₁ to C₈ alkyl optionally substituted with one or more substituents independently selected from hydroxyl and halogen; and
R_{d} is phenyl optionally substituted with one or more halogen substituents.

15. The method of claim 1, wherein the compound has the Formula (III): 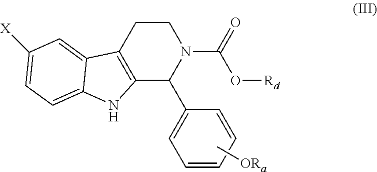
or a pharmaceutically acceptable salt, racemate or stereoisomer thereof, wherein,
X is halogen;
Rₐ is H, C₁ to C₈ alkyl optionally substituted with one or more substituents independently selected from hydroxyl and halogen; and
R_{d} is phenyl substituted with one or more halogen substituents.

16. The method of claim 1, wherein the compound has the Formula (IV): 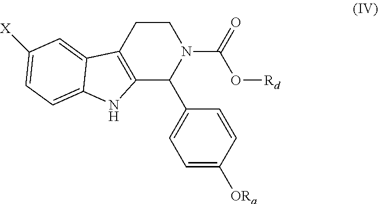
or a pharmaceutically acceptable salt, racemate or stereoisomer thereof, wherein,
X is halogen;
Rₐ is H, C₁ to C₈ alkyl optionally substituted with one or more substituents independently selected from hydroxyl and halogen; and
R_{d} is phenyl substituted with one or more halogen substituents.

17. The method of claim 1, wherein the compound has the Formula (IV): 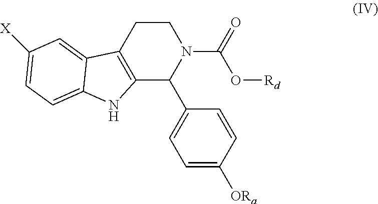
or a pharmaceutically acceptable salt, racemate or stereoisomer thereof, wherein,
X is halogen;
Rₐ is H, C₁ to C₈ alkyl optionally substituted with one or more substituents independently selected from hydroxyl and halogen; and
R_{d} is phenyl substituted on a para position with a halogen substituent.
